SEQUENCE LISTING (467 sequences)

SEQ ID NO: 1 - protein, 1080 aa, unidentified
MSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPNGSMPIVTKFETDDLISDNQRKAMISNFTKNAAAKAAKKPIPYLDRLKEHMVSLCDEYNVYAWAAAITNSNADVTARNTRNLTFIGEQNSRRKELSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHADEIAAVNIAMYEVCCQTGAFGKKQKKSDELPG

SEQ ID NO: 2 - protein, 1061 aa, unidentified
MSSDVVRPYNTKLLPDNRKYNMFLQTFKRLNLISSNHFDLLVCLYAAITNKKAEEYKSEKEDHVTADSLCAINWFRPMSKRYIKYATTTFKMLELFKEYSGHEPDTYSKNYLMSNIVSDRFVWVDCRKFAKDFANQMELSFHEFTTLSETLLANSILVLNESTKANWAWGAVSALYGGGDKEDSTLKSKILLAFVDALNNPELKTRREILNHVCESLKYQSYQDMYVDFRSVVDDKGNKKSPNGSMPIVTKFESDDLIGDNQRKTMISSFTKNAAAKASKKPIPYLDILKDHMISLCEEYNVYAWAAAITNSNADVTARNTRNLTFIGEQNTRRKELSVLQTSTNEKAKDILNKINDNLIPEVRYTPAPKHLGRDLANLFEMFKEKDINQIGNEEEKQNVINDCIEQYVDDCRSLNRNPVAALLKHISGYYEDFSAKNFLDGAKLNVLTEVVNRQKAHPTICSEKAYTWISKIDKNRRQANSSLVGWVVPPEEVHKEKIAGQQSMMWVTLTLLDDGKWVKHHIPFADSRYYSEVYAYNPNLPYLEGGIPRQSKFGNKPTTNLTAESQALLANSKHKKANKTFLRAKENITHNVRVSPNTSLCIRPLKDSAGNQMFDNIGNMLFGMQINHRITVGKPNYKIEVGDRFLGFDQNQSENHTYAVLQRVSESSHGTHHFNGWDVKVIEKGKVTSDVVVRDEVYDQLSYEGVPYDSPKFTEWREKRRKFVLENMSIQIEEGKTFLTEFDKLNKDSLYRWNMNYMKLLRKAIRAGGKEFAKITKAEIFELGVMRFGPMNLGSLSQVSLKMIAAFKGVIQSYFSVSGCIDDASKKAHDSMLFAFLCSADEKRTNKREEKTNRAASFILQKAYSHGCKMIVCEDDLPIADGKVGKAQNADRMDWCARSLAKKVNDGCVAMSICYRAIPAYMSSHQDPFTHMQDKKTSVLRPRFMEVGKDSIRDHHVAGLRRMLNSKGNTGTSVYYREAALRFCEALGVLPELVKNKKTHASELGKHMGSAMLMPWRGGRIYVASKKLTSDAKSIKYCGEDMWQYHADEIAAINIAMYEV

SEQ ID NO: 3 - protein, 1075 aa, unidentified
MGISISRPYGTKLRPDARKKEMLDKFFTTLAKGQRVFADLGLCIYGSLTLEMVKRLEPESDSELVCAIGWFRLVDKVTWSENEIKQENLVRQYETYSGKEASEVIKTYLSSPSSDKYVWIDCRQKFLRFQRDLGTRNLSEDFECMLFEQYLRLTKGELDGHTAMSNMFGTKTKEDRATKLRYAARMKEWLEANEEITWEQYHQALQDKLDANTLEEAVDNYKGKAGGSNPFFSYTLLNRGQIDKKTHEQQLKKFNKVLKTKSKNLNFPNKEKLKQYLETAIGIPVDAQVYGQMFNNGVSEVQPKTTRNMSFSMEKLELLNELKSLNKTDGFERANEVLNGFFDSELHTTEDKFNITSRYLGGDRNNRLPKLYELWKKEGVDREEGIQQFSQAIQDKMGQIPVKNVLRYIWEFRETVSAEDFEAAAKANQLEEKITRTKAHPVVISNRYWTFGSSALVGNIMPADKMHKDQYAGQSFKMWLEAELHYDGKKVKHHLPFYNARFFEEVYCYHPSVAEVTPFKTKQFGYAIGKDIPADVSVVLKDNPYKKATKRFLRAISNPVANTVDVNKPTVCSFMIKRENDEYKLVINRKIGVDRPKRIKVGRKVMGYDRNQTASDTYWIGELVPHGTTGAYRIGEWSVQYIKSGPVLSSTQGVNDSTTDQLIYNGMPSSSERFKAWKKSRMSFIRKLIRQLNAEGLESKGQDYVPENPSSFDVRGETLYVFNSNYMKALVSKHRKAKKPVEGILEEIEALTSKAKDSCSLMRLSSLSDAAMQGIASLKSLINSYFNKNGCKTIEDKEKFNPDLYVKLVEVEQKRTNKRKEKVGRIAGSLEQLALLNGVDVVIGEADLGEVKKGKSKKQNSRNMDWCAKQVAERLEYKLTFHCIGYFGVNPMYTSHQDPFEHRRVADHLVMRARFEEVNVSNVSEWHMRNFSNYLRADSGTGLYYKQATLDFLKHYDLEEHADDLEKQNIKFYDFRKILEDKQLTSVIVPKRGGRIYMATNPVTSDSTPVTYAGKTYNRCNADEVAAANIAISVLAPHSKKEEKEDKIPIISKKPKSKNTPKARKNLKTSQLPQK

SEQ ID NO: 4 - protein, 1003 aa, unidentified
MASKHVVRPFNGKVTATGKRLAYLEETFHYLEKAAGGVSTLFAALGSYLDATTISNLINKNQDLAVVIFRYHVVPKGEAHTLPVGTDMVSRFVADYGMEPNEFQRAYLDSPIDQEKYCWQDNRDVGCWLGEQLGVSEADMRAIAVTFYNNQMLYDCVKGTGSGNAVSLLFGSGKKSDYSMKGVIAGKAASVLAKYRPATYQDARKMILEANGFTSVKDLVTSYGITGRSSALQIFMEGIESGPISSKTLDARIKKFTEDSERNGRKNLVPHAGAIRNWLIEQAGSSVENYQMAWCEVYGNVSADWNAKVESNFNFVAEKVKALTELSNIQKSTPDLGKALKLFEEYLTTCQDEFAIAPYHFSVMEEVRMEMATGREFNDAYDDALNSLDMESKQPIQPLCKFLIERGGSISFDTFKSAAKYLKTQSKIAGRYPHPFVKGNQGFTFGSKNIWAAINDPMMEYADGRIAGGSAMMWVTATLLDGKKWVRHHIPFANTRYFEEVYASKKGLPVLPCARDGKHSFKLGNNLSVERVEKVKEGGRTKATKAQERILSNLTHNVQFDSSTTFIIRRQEESFVICVNHRHPAPLMKKEMEVGDKIIGIDQNVTAPTTYAIVERVASGGIERNGKQYKVTAMGAISSVQKTRGGEVDVLSYMGVELSDSKNGFQSLWNKCLDFVTKHGTENDVKYYNNTAVWANKLYVWHKMYFRLLKQLMRRAKDLKPFRDHLQHLLFHPNLSPLQRHSLSLTSLEATKIVRNCIHSYFSLLGLKTLDERKAADINLLEVLEKLYAGLVERRKERTKLTAGLLVRLCNEHGISFAAIEGDLPVVGEGKSKAANNTQQDWTARELEKRLSEMAEVVGIKVIAVLPHYTSHQDPFVYSKNTKKMRCRWNWRTTKTFTDRDALSIRRILSKPETGTNLYYQKGLKAFAEKHGLDLAEMKKRKDAQWYLERIQDKNFLVPMNGGRVYLSSVKLAGKETIDMGGEILYLNDADQVAALNVLLVKI

SEQ ID NO: 5 - protein, 1018 aa, unidentified
MAKKEHIIRPFKGTLPLRGDRLRYLQDTMKYMKKVEDTITELCAAVIAYAKPTIIQQILGEEIETTSTFCSFRLVGIHENFTMPLTTNMIKHFQKTFNINPSEKQAIYLSSGFDSDKYRWQDTSEVSRNFANKCRLTNQEFQEFAEQALLNMCFIGCSGSPGATNAVSQIFGTGEKSDYQRKSQIAKIAADTLENHKPSTYESARLMVLNTLGHKTIEDCVNDYGAIGAKSAFRLFMESKEIGPITSEQLTTKIKKFREDHKKNSIKKQLPHVEKVRNALLSQFKEQYLPSAWAEAWCNIMGEFNSKLSNNNNFIDQKTKMVNDCDNIKKSNPQLDKAVNMLDEWKYKNWDDNSAIHPYHIGDLKKLMAIFNINNEGTFDERFSASWEQFSTSLEYGEKPPVRDLLAHIIKNMNDLTYTDVINAAKFLKLQDNIRNKYPHPFVMPNKGCTFGKDNLWGEINDPTAKIKSTEEVAGQRPMMWLTAKLLDNGKWVEHHIPFASSRYFAEVYYTNPALPTLPIARDGKHSYKLTKTIDANTAKTLVNNPRDKAAKLIARTKANTTHNVKWIKPTYRIQKENNQFVITINHRHPCITPPKEIILGDRILSFDQNETAPTAFSILEKTTKGTEFCGHHIKVLKTGMLEAKIKTSKKSIDAFTYMGPMEDDHASGFPTLLNICEKFISENGDEKDKSFSSRKLPFKRSLYFFHGSHFDLLKKMIRKAKNDPKKLKLVRIHINEILFNSNLSPIKLHSLSIHSMENTKKVIAAISCYMNVHEWKTIDEQKNADITLYNAKEKLYNNLVNRRKERVKVTAGMLIRLARENNCRFMVGEAELPTQQQGKSKKNNNSKQDWCARDIAQRCEDMCEVVGIKWNGVTPHNTSHQNPFIYKNTSGQQMRCRYSLVKKSEMTDKMAEKIRNILHAEPVGTTAYYREGILEFAKHHGLDLGMMKKRRDAKYYDNLPDEFLLPTRGGRIYLSENQLGGNETIVINGKKYFVNQADQVAAVNIGLLYLLPKKNQS

SEQ ID NO: 6 - protein, 1047 aa, unidentified
MSEKKFHIRPYRCSISPNARKADMLKATISYLDSLTSVFRSGFTALLAGIDPSTVSRLAPSGAVGSPDLWSAVNWFRIVPLAEAGDARVGQASLKNLFRGYAGHEPDEEASIYMESRVDDKRHAWVDCRAMFRAMALECGLEEAQLASDVFALASREVIVFKDGEINGWGIASLLFGEGEKADSQKKVALLRSVRLALEGDYATYEELSGLMLAKTGASSGSDLLDEYKRSEKGGSSGGRHPFFDEVFRRGGRVKQEERERLLKSCDTAIQKQGQALPLSHVASWRQWFLRRVTLLRNRRQESFAVCITNALMDLQPKNLRNVHYVTNPKSEKDKGVLELRVDVKNNEGPDVAGAQAVFDAYMARLAPDLRFSVMPRHLGSLKDLYALWAKLGRDEAIEEYLEGYEGPFSKRPIAGILQIIHAHRGKVGHDSLLRAARLNRAMDRLERKRAHACAAGNKGYVYGKSSMVGRINPQSLEVGGRKSGRSPMMWVTLDLVDGDRFAQHHLPFQSARFFSEVYCHGDGLPATRVPGMVRNRRNGLAIGNGLGEGGLSALRAGSDRRKRANKRTLRALENITHNVEIDPSTSFTLREDGIIISHRIEKIEPKLVAFGDRALGFDLNQTGAHTFAVLQKVDSGGLDVGHSRVSIVLTGTVRSICKGNQASGGRDYDLLSYDGPERDDGAFTAWRSDRQAFLMSAIRELPTPAEGEKDYKADLLSQMASLDHYRRLYAYNRKCLGIYIGALRRATRRQAVAAFKDEILSIANHRCGPLMRGSLSVNGMESLANLKGLATAYLSKFKDSKSEDLLSKDEEMADLYRACARRMTGKRKERYRRAASEIVRLANEHGCLFVFGEKELPTTSKGNKSKQNQRNTDWSARAIVKAVKEACEGCGLGFKPVWKEYSSLTDPFERDGDGRPALRCRFAKVAAPDSELPPRLTKAVGSYVKNALKADKAEKKQTCYQRGAIEFCSRHGIDVRKATDKAIRKAVRGSSDLLVPFDGGRTFLLSTRLSPESRKVEWAGRTLYEFPSDMVAAINIACRGLEPRKA

SEQ ID NO: 7 - protein, 1029 aa, unidentified
MDEQAVVSSGSDKTLKIVRPYRAKVTATGIRLEGIKNTLNYLKRTEICLSRLNAACGAFLTPAIVEQICKDDPALVCAIARFQLVPVGSEATLSDSGLMRHFKAALGELTPLQEAYLNSSYNDELYAWQDTLVLARQIIAETGLTEDQFRAFAHACFKNGNIIGCAGGPGASNAISGIFGEGIKSDYSLRSEMTAAVAKVFEEKRPITYEEARALALEATGHASVQSFVEAFGKQGRKGTLILFMEDTKTGAFPSNEFDYKLKKLKEDAERVGRKGIIPHRDVIASYLRNQTGADIEYNSKAWCESYCCAVSEYNSKMSNNVRFATEKSLDLTKLDETIRETPKISEAMLVFENYMARIDADLRFIVSKHHLGNLAKFRQTMMHVSASEFEEAFKAMWADYLAGLEYGEKPAICELVRYVLTHGNDLPVEAFYAACKFLSLDDKIKNRYPHPFVPGNKGYTFGAKNLWAEINDPFKPIRQGNPEVAGQRPMMWATADLLDNNKWVLHHIPFASSRYFEEVYYTDPSLPTAQKARDGKHGYRLGKVLDEAARERLKANNRQRKAAKAIERIKANCEHNVAWDPTTTFMLQLDSEGNVKMTINHRHIAYRAPKEIGVGDRVIGIDQNETAPTTYAILERTENPRDLEYNGKYYRVVKMGSVTSPNVSKYRTVDALTYDGVSLSDDASGAVNFVVLCREFFAAHGDDEGRKYLERTLGWSSSLYSFHGNYFKCLTQMMRRSARSGGDLTVYRAHLQQILFQHNLSPLRMHSLSLRSMESTMKVISCMKSYMSLCGWKTDADRIANDRSLFEAARKLYTSLVNRRTERVRVTAGILMRLCLEHNVRFIHMEDELPVAETGKSKKSNGAKMHWCARELAVRLSQMAEVTSVKFTGVSPHYTSHQDPFVHSKTSKVMRARWSWRNRADFTDKDAERIRTILGGDDAGTKAYYRSALAEFASRYGLDMEQMRKRRDAQWYQERLPETFIIPQRGGRVYLSSHDLGSGQKVDGIYGGRAFVNHADEVAALNVALVRL

SEQ ID NO: 8 - protein, 1029 aa, unidentified
MKTETLIRPYPGKLNLQPRRAQFLEDSIQYHQKMTEFFYQFLQAVGGATTHQNISDFIDNKATDEHQATLLFQVVSKDSTTPECPAEELLARFAQYTGKQPNEAVTHYLTSRINTDKYRWQDNRLLAQNIASQLNISETQFQEIAHAILSNNLYIGQTASNAAANFISQVTGTGQKAPKAARLDVLFQTNQALAKTQPTTFGQLQQIIVQACGESTTDAVLAKFGNKGAATSLQLALKTDPNTTLDQKKYEALQKKFAEDETKYRNKVDIPHKTQLRNLILNTSNQFCNWHTKPAIEAFKCAIADIQSKVSNNLRIMQEKAKLYEAFRNVDPQVQIAVQALENHMNTLEEPYAPYAHSFGSVKDFYEDLNNGSNLDEAIQTIVHDSDNFNRKPDPNWLRIIAPLHSSHSASQIMEAVKYLSSKQDYELRKPFPFVATNLPATYGKFNIPGTLNPPTDSLHGRLNGSHSNMWLTALLLDGRDWKNHHLCFASSRYFEEVYFTNPSLPTTDKVRSPKCGFTLKSVLDSEAKDRIRNAPKSRTKAVKAIERIKANSTHNVAWNPETSFQMQKRNDEFYITINHRIEMEKIPGQKKTDDGFTIHPKGLFAILKEGDRILSQDLNQTAATHCAVYEVAKPDQNTFNHHGIHLKLIATEELKMPLKTKKSTIPDALSYQGIHAHDRENGLQQLKDACGAFISPRLDPKQKATWDNSVSKKENLYPFITAYMKLLKKVMKAGRQELKLFRTHLDHILFKHNLSPLKLHGVSMIGLESSRATKSVINSFFNLQNAKTEQQQIALDRPLFEAGKTLINNQTRRRQERVRLETSLTMRLAHKYNAKAIIIEGELPHSSTGTSQYQNNVRLDWSAKKSAKLKTESANCAGIAICQIDPCHTSHQNPFRHTPTNPDLRPRFAQVKKGKMFQYQLNGLQRLLNPRSKSSTAIYYRQAVQSFCAHHNLTERDITSAKFPSDLEKKIKDDTYLIPQRGGRIYISSFPVTSCARPCTSNHYFGGGQFECNADAVAAVNIMLKVHP

SEQ ID NO: 9 - protein, 1011 aa, unidentified
MPIRGYKCTVVPNVRKKKLLEKTYSYLQEGSDVFFDLFLSLYGGIAPKMIPQDLGINEQVICAANWFKIVEKTKDCIADDALLNQFAQYYGEKPNEKVVQFLTASYNKDKYVWVDCRQKFYTLQKDLGVQNLENDLECLIREDLLPVGSDKEVNGWHSISKLFGCGEKEDRTIKAKILNGLWERIEKEDILTEEDARNELLHSAGVLTPKEFRKVYKGAAGGRDCYHTLLVDGRNFTFNLKTLIKQTKDKLKEKSVDVEIPNKEALRLYLEKRIGRSFEQKPWSEMYKTALSAVMPKNTLNYCFAIDRHAQYTKIQTLKQPYDSAITALNGFFESECFTGSDVFVISPSHLGKTLKKLYNYKDVESGISEIVEDEDNSLRSGVNVNLLRYIFTLKDMFSAEDFIKAAEYNVVFERYNRQKVHPTVKGNQSFTFGNSALSGKVIPPSKCLSNLPGQMWLAINLLDQGEWKEHHIPFHSARFYEEIYATSDNQNNPVDLRTKRFGCSLNKTFSAADIEKVKESAKKKHGKAAKRILRAKNTNTAVNWVDCGFMLEKTEVNFKITVNYKLPDQKLGKFEPIVGTKILAYDQNQTAPDAYAILEICDDSEAFDYKGYKIKCLSTGDLASKSLTKQTEVDQLAYKGVDKTSNFYKKWKQQRRLFVKSLNIPDALKSFENINKEYLYGFNNSYLKLLKQILRGKFGPILVDIRPELIEMCQGIGSIMRLSSLNHDSLDAIQSLKSLLHSYFDLKVKEEIKTEELREKADKEVFKLLQQVIQKQKNKRKEKVNRTVDAILTLAADEQVQVIVGEGDLCVSTKGTKKRQNNRTIDWCARAVVEKLEKACKLHGLHFKEIPPHYTSHQDCFEHNKDIENPKEVMKCRFNSSENVAPWMIKKFANYLKCETKYYVQGMQDFLEHYGLVEYKDHIKKGKISIGDFQKLIKLALEKVGEKEIVFPCKGGRIYLSTYCLTNESKPIVFNGRRCYVNNADHVAAINVGICLLNFNARAKVAEKTP

SEQ ID NO: 10 - protein, 1093 aa, unidentified
MAKKDFIARPYNSFLLPNDRKLAYLEETWTAYKSIKTVLHRFLIAAYGAIPFQTFAKTIENTQEDELQLAYAVRMFRLVPKDFSKNENNIPPDMLISKLASYTNINQSPTNVLSYVNSNYDPEKYKWIDSRNEAISLSKEIGIKLDELADYATTMLWEDWLPLNKDTVNGWGTTSGLFGAGKKEDRTQKVQMLNALLLGLKNNPPKDYKQYSTILLKAFDAKSWEEAVKIYKGECSGRTSSYLTEKHGDISPETLEKLIQSIQRDIADKQHPINLPKREEIKAYLEKQSGTPYNLNLWSQALHNAMSSIKKTDTRNFNSTLEKYEKEIQLKECLQDGDDVELLGNKFFSSPYHKTNDVFVICSEHIGTNRKYNVVEQMYQLASEHADFETVFTLLKDEYEEKGIKTPIKNILEYIWNNKNVPVGTWGRIAKYNQLKDRLAGIKANPTVECNRGMTFGNSAMVGEVMRSNRISTSTKNKGQILAQMHNDRPVGSNNMIWLEMTLLNNGKWQKHHIPTHNNKFFEEVHAFNPELKQSVNVRNRMYRSQNYSQLPTSLTDGLQGNPKAKIFKRQYRALNNMTANVIDPKLSFIVNKKDGRFEISIIHNVEVIRARRDVLVGDYLVGMDQNQTASNTYAVMQVVQPNTPDSHEFRNQWVKFIESGKIESSTLNSRGEYIDQLSHDGVDLQEIKDSEWIPAAEKFLNKLGAINKDGTPISISNTSKRAYTFNSIYFKILLNYLRANDVDLNLVREEILRIANGRFSPMRLGSLSWTTLKMLGNFRNLIHSYFDHCGFKEMPERESKDKTMYDLLMHTITKLTNKRAERTSRIAGSLMNVAHKYKIGTSVVHVVVEGSLSKTDKSSSKGNNRNTTDWCSRAVVKKLEDMCVFYGFNLKAVSAHYTSHQDPLVHRADYDDPKLALRCRYSSYSRADFEKWGEKSFAAVIRWATDKKSNTCYKVGAVEFFKNYKIPEDKITKKLTIKEFLEIMCAESHYPNEYDDILIPRRGGRIYLTTKKLLSDSTHQRESVHSHTAVVKMNGKEYYSSDADEVAAINICLHDWVVPLNWTNHCLPAGWCSDHLKECVQCHTPDPVRISM

SEQ ID NO: 11 - RNA, 36 nt, unidentified
  source 1..36: mol_type = other RNA
ctagcaatgactcagaaatgtgtccccagttgacac

SEQ ID NO: 12 - RNA, 36 nt, unidentified
  source 1..36: mol_type = other RNA
atcgcaacatcttagaaatccgtccttagttgacgg

SEQ ID NO: 13 - RNA, 36 nt, unidentified
  source 1..36: mol_type = other RNA
tctcaacgatagtcagacatgtgtccccagtgacac

SEQ ID NO: 14 - RNA, 36 nt, unidentified
  source 1..36: mol_type = other RNA
ctcaaagtgtcaaaagaatgtccctgctaatgggac

SEQ ID NO: 15 - RNA, 37 nt, unidentified
  source 1..37: mol_type = other RNA
tcccaaagtggcaaaagaatctccctgttaatgggag

SEQ ID NO: 16 - RNA, 36 nt, unidentified
  source 1..36: mol_type = other RNA
gtctaactgccatagaatcgtgcctgcaattggcac

SEQ ID NO: 17 - RNA, 35 nt, unidentified
  source 1..35: mol_type = other RNA
tcggggcaccaaaataatctccttggtaatgggag

SEQ ID NO: 18 - RNA, 36 nt, unidentified
  source 1..36: mol_type = other RNA
ccacaacaaccaaaagaatgtccctgaaagtgggac

SEQ ID NO: 19 - RNA, 36 nt, unidentified
  source 1..36: mol_type = other RNA
gtaacagtggctaagtaatgtgtcttccaatgacac

SEQ ID NO: 20 - RNA, 24 nt, unidentified
  source 1..24: mol_type = other RNA
gagagaatgtgtgcaaagtcacac

SEQ ID NO: 21 - DNA, 3243 bp, unidentified
  source 1..3243: mol_type = other DNA
atgtctagtgatgtcgttcgtccatataacaccaaactgcttccagataatcgcaaacacaatatgtttttgcaaactttcaagcgacttaattctatttctcttaatcattttgatctcttaatttgtctttatgctgccattaccaacaagaaggcagaagaatataagtctgaaaaagaagctcatgtaaccgctgatagcctttgtgctatcaattggttccgtcctatgtccaagcgttacagcaaatacgcaactacaactttcaatatgcttgaattgttcaaagaatactctgggcatgaaccagatgcttattccaagaattatcttatgtccaatattgactcagacaggtttgtctgggttgattgccgtaaatttgccaaagattttgcgtatcaaatggaacttggtttccatgaatttacagtcttggcagaaaccttgttggcaaatagtattcttgtactcaacgaatcaactaaggcaaattgggcatggggcaccgtttctgcactttacggtggaggcgataaggaagattctacgctgaagtcgaaaatccttttggcttttgttgatgcactcaataaccacgaacttaaaactaagcgtgaaattctcaatcaagtttgtgaatcactaaaatatcaatcataccaagacatgtatgttgatttccgttctgttgttgacgaaaatggaaacaagaagtctcccaatggctcaatgccaatcgtcaccaagtttgaaacagatgatttgatttctgataatcaacgcaaagcaatgatttctaatttcacaaagaatgctgctgctaaagcggctaaaaaacctattccctacctagacagactcaaggaacatatggtttccttgtgcgatgaatataatgtttatgcttgggcagcagctatcactaactctaatgccgatgtaacagctaggaatactcgcaatttaacattcatcggggaacaaaattctcgaaggaaagaactatcggttttacaaactacaacaaacgaaaaagcaaaagatatcttgaataagattaatgacaatcttattcaagaagtaaggtatacccctgcccccaagcacttggggcgtgatcttgccaatctttttgatactctgaaagaaaaagatatcaataatattgaaaacgaagaagagaagcagaatgtaattaatgattgcattgagcaatatgttgatgattgccgttcactgaaccgcaatcccattgctgctttgctcaagcacattagccgatactatgaagatttttcagccaagaatttcttggatggtgccaagttgaatgtcttgactgaagttgtaaatcgtcaaaaggcacatccaactatttggtctgaaaaggcttatacttggatttccaagtttgacaagaataggcgacaagcaaactcttctttggttggatgggttgttccaccagaagaagtccataaagagaagattgctggtcaacaaagcatgatgtgggtcactttgactctgcttgatgatggcaagtgggtaaagcaccatattcctttttcagattccagatattattctgaagtctatgcctacaatccaaatttgccatatcttgatggtggtattccacgccagtcaaagtttggcaataaaccaaccactaatctgactgctgaaagtcaagcgttacttgcaaacagcaagtataaaaaggcaaataagtcatttctccgtgccaaggaaaatgctactcacaatgtccgtgttagtccaaacacttccttgtgcattcgtttgctcaaggatagtgctggtaatcaaatgtttgataagattggcaatgttctgtttggaatgcagatcaaccataaaatcaccgttggcaagcccaactacaagatcgaagttggtgataggttccttggtttcgaccagaaccaaagtgaaaaccacacttatgctgtcttgcaacgagtctctgaaagctctcatgacactcatcattttaatggatgggatgtcaaggttcttgaaaagggcaaagtaacaagtgatgtcatcgttagagatgaggtctatgaccaacttagctatgagggcgttccttatgattcttcaaagtttgcagaatggagagacaagaggagaaggtttgttttggaaaacttgtctatccagttggaagaaggcaaaacattcttgactgaattcgacaaattaaataaagattctctttatcgttggaatatgaattatctgaaactgctcaggaaagctattcgtgccggtggcaaggaatttgccaagattgctaagactgagatttttgaattggcagttgaaaggtttggaccaatcaaccttggtagtttgtcacaaattagcttgaagatgattgcatctttcaagggagtggttcagtcttacttttctgtatctggttgtgttgatgacgcatccaagaaggcacatgattccatgctcttcactttcatgtgtgcagcagaagaaaaaaggacaaacaaaagagaagaaaagactaatcgtgcagcatcttttatcttgcagaaagcatatttgcatggctgcaagatgattgtttgcgaagacgatcttcctgttgctgatggaaaaacaggcaaggcacaaaatgcggatcgtatggactggtgtgcccgtgctttggcaaagaaagtcaacgatggttgtgtggcaatgtctatctgctatcgtgccattccagcttatatgtctagccaccaagatccatttgttcacatgcaagacaaaaagacttctgttttgcgtccaaggttcatggaagttaacaaggatagcatcagggattatcatgttgctggtttgcggagaatgctgaacagcaagagtgatgcaggcacttccgtttactatcgtcaggcagctttgcatttctgcgaagcgttgggcgtgtctccagaattagtcaagaacaaaaagactcatgctgccgaattaggaaagcatatgggttctgccatgttgatgccttggcggggtggcagggtttatattgccagcaagaagttgacttcggatgctaaaagtgtaaaatactgtggagaagatatgtggcagtatcatgctgatgagattgctgctgtcaatatcgcaatgtatgaagtttgctgccagacaggtgcgtttggcaagaagcaaaagaagagtgatgaactaccgggataa

SEQ ID NO: 22 - DNA, 3244 bp, unidentified
  source 1..3244: mol_type = other DNA
catgtctagtgatgttgttcgtccatataacactaagctgcttcctgataatcgcaaatacaatatgtttttgcaaactttcaaaagactcaatttgatttcatcaaatcattttgatctcttggtttgtctttatgctgctatcaccaacaaaaaagctgaagaatataagtcagaaaaagaagatcatgtaaccgctgatagcctttgcgccatcaattggttccgtcctatgtccaagcgttatatcaaatacgcaaccactacttttaagatgcttgaattgtttaaggagtactctggtcatgaaccagatacttattccaagaattatctcatgtccaatatcgtctcagataggtttgtttgggttgattgccgcaaatttgccaaagattttgccaatcaaatggaacttagtttccacgaatttaccactttgtcagagactttgttggcaaatagtatccttgtactcaatgagtcaaccaaggcaaattgggcatggggtgctgtttcagcactttatggtggaggcgacaaagaagattctacgctgaagtccaaaatccttttggcttttgttgatgctctcaataatcctgaacttaaaactaggcgggaaattctcaatcatgtttgtgaatcactaaaatatcaatcataccaagatatgtatgttgattttcgatctgtcgttgatgataagggaaacaagaagtctcccaatggctcaatgccaatcgtcactaagtttgaatcagatgatttgattggtgacaatcaacgcaaaactatgatttctagtttcacaaaaaacgccgctgccaaagcgtctaagaagcccattccatatctagacattctaaaagaccacatgatttccttgtgcgaggaatacaatgtctatgcttgggcagcagctattaccaattccaatgctgatgtaactgctagaaacactcgcaatctgacattcatcggggaacaaaatacccgaaggaaagaactatcggttttacaaacttctacaaacgaaaaagcaaaagatatcttaaataagattaacgacaatcttattccagaagtaaggtacacccctgctcccaagcacttggggcgtgatcttgccaatctttttgaaatgttcaaagaaaaagatataaatcagattggaaatgaagaagaaaagcaaaatgtgatcaatgattgcattgagcaatatgtcgatgattgccgttcattgaaccgcaatcctgttgcagctttgctcaagcatattagcggatattatgaagatttttcagccaagaatttcttggatggtgccaagttgaatgtcttgacggaagttgtcaatcgtcaaaaggcacatccaactatttgttctgaaaaggcttatacttggatttccaagattgacaagaataggcgacaagcaaactcttctttggttggatgggttgttccaccggaggaagtccataaggaaaaaattgccggtcaacaaagcatgatgtgggtcactttgactttgcttgatgacggcaagtgggtaaagcatcatattccttttgcagactcaagatattattctgaagtctatgcctataatccaaatttgccatatcttgaaggtggtattccacgacaatcaaagtttggcaataaaccaacaactaatttgaccgctgaaagccaagcattacttgccaacagtaagcacaagaaagccaacaagacatttctccgtgccaaggagaatatcactcacaatgttcgtgttagtccaaatacttcattgtgcattcgtcccctcaaggatagtgctggtaatcaaatgtttgacaacattggtaatatgttgtttggaatgcagatcaatcacagaattactgtcggcaagccaaactacaagatcgaagttggtgatcggttccttggttttgaccagaaccaaagcgaaaaccacacctatgcagttcttcaacgagtatccgaaagctctcatggcactcatcatttcaatggttgggatgtcaaagtgattgagaagggcaaggtgacaagtgatgtcgtcgtcagagatgaagtctatgatcaattaagctacgagggtgtcccttacgattctccaaagtttacagaatggagagagaagaggcgaaagtttgtcttggaaaatatgtcaatccagattgaagaaggcaaaacattcttgactgaatttgacaagttaaacaaagactctttgtatcgttggaacatgaattacatgaaattgcttaggaaggcaattcgtgctggtggcaaggaatttgccaagattacaaaggctgagatttttgaactaggagttatgagatttggaccaatgaacttgggcagcttgtcgcaagtcagcttgaagatgattgctgcttttaagggagttattcagtcttacttttccgtatctggttgcattgatgacgcatccaagaaagctcatgattcgatgttattcgctttcttgtgttcagcagatgagaaaaggacaaacaagagggaagaaaagacaaatcgtgcagcatctttcatattgcagaaagcatactcgcatggttgcaagatgattgtttgcgaggatgatcttcccattgccgatggcaaggtgggcaaggcacaaaatgcggatcgcatggactggtgcgcccgttcattggcaaagaaagtcaacgatggttgtgtggctatgtccatatgttatcgtgccattccagcatatatgtcaagccatcaagatccatttactcatatgcaagataaaaagacttctgttttgcgtccaaggttcatggaagtcggcaaggatagcattagggatcatcatgttgctggtctgcggagaatgctgaacagtaaaggtaatactggcacttctgtttactatcgtgaggcagctttgcgtttctgcgaagcgttgggtgtgcttcccgaattagtcaagaacaaaaagactcatgcttcggaattaggaaagcatatgggttctgccatgttgatgccttggcggggtggcaggatctatgtcgccagcaagaaattgacttcggatgccaagagtataaaatattgtggagaagatatgtggcaatatcatgctgatgagattgctgctatcaatatcgcaatgtatgaggtctgctgtcagacaggtgcttttggcaaaaaacaaaagaagagtgatgaactaccgggataa

SEQ ID NO: 23 - DNA, 3225 bp, unidentified
  source 1..3225: mol_type = other DNA
atgggtattagcatttcacgtccgtacggtacaaagttgcgtcctgatgctcgtaagaaggaaatgttggataagtttttcaccacgctagcaaaaggtcagcgtgtttttgcggatctgggactgtgcatttacggcagccttactttagaaatggtaaagcggcttgagccagaatccgattctgaacttgtctgtgcaattggttggtttcgtcttgtagataaggtaacttggtctgagaatgaaattaaacaagagaacctggttagacaatatgagacgtattcaggaaaagaagcgtctgaggttatcaagacttacctaagctctccaagttcagacaagtatgtgtggatagactgccgacaaaagtttcttaggtttcaaagggatctgggaacacgtaatctgtctgaagactttgagtgcatgctttttgaacagtacctcagactcacaaagggagagcttgatgggcataccgctatgtccaacatgtttggaacaaaaacaaaagaagatcgcgccacaaaactgagatatgccgcaaggatgaaagaatggctcgaggctaacgaagaaattacttgggaacaatatcaccaagcgttgcaagataaattagacgccaatactttagaggaggctgttgataattacaaaggcaaagcgggaggctctaatccattttttagttacacgcttttaaacagaggtcagattgataaaaaaactcacgagcagcaattaaagaaattcaacaaagttctaaaaaccaaatccaaaaatttaaattttccaaacaaagagaagttaaaacaatatttagaaacagcaattggtattcctgttgatgctcaggtctacggtcagatgtttaataacggcgtttctgaagttcaaccaaagacaacgcgcaacatgtctttttctatggagaagcttgagcttttaaacgagttgaaaagtctcaacaagactgacggttttgaacgcgctaatgaagtcttgaatggtttctttgattctgaacttcacactactgaagacaagttcaacatcacttccaggtatttgggtggagacagaaacaatcggctaccaaagctgtacgagctttggaaaaaggaaggagtagatcgtgaggaaggtatccagcaattcagccaagcaatccaagataagatgggtcagatacctgttaagaatgtccttaggtatatttgggaatttcgtgagactgtttctgccgaagactttgaagcggcagcgaaagcgaatcagttggaagaaaaaatcacgcgtaccaaagcgcaccccgttgttatatctaacaggtattggacatttggctcttcggctcttgttggtaatatcatgccagcagacaagatgcacaaagaccagtacgcaggtcaaagtttcaagatgtggcttgaagccgaactgcactacgacggtaagaaagtcaaacatcacttgccgttctacaacgccaggttctttgaagaggtctactgctatcacccgagcgtagctgaagttacaccattcaaaaccaagcagtttggttatgcaattggaaaagatattccagctgacgtttcggttgtactgaaagacaatccttataaaaaggcaaccaagcgcttccttcgggctatcagcaatccagtcgccaacacagtggatgtaaacaagcctacagtttgctcattcatgattaaacgagaaaatgacgaatacaaactagtcattaatcgaaagatcggtgttgatcgcccaaagcgtattaaagtaggtaggaaggtcatgggctatgaccgtaaccaaactgcttctgatacttactggattggagagcttgttccacatggaacaaccggagcgtaccgtattggagaatggagcgtccagtatatcaagagcggtcccgtgttgtcttctacgcaaggcgtaaatgacagtactacggatcaacttatatacaacggaatgccgagctccagcgaacgttttaaagcttggaagaaatctaggatgtctttcattcgtaagttgatacgccaactgaacgccgaaggcttggaaagtaaaggacaggactatgttcctgaaaatccaagtagctttgatgttaggggcgaaacactttacgtattcaacagcaactatatgaaagctttggtgtctaagcatcgaaaagccaagaaacctgttgaaggtattcttgaagaaatagaagccttgacaagcaaagctaaagattcttgttcgttgatgcgtttgagttctttgtctgatgcggctatgcaaggtattgcttcgttgaagagtttgatcaactcatacttcaacaagaatggttgcaaaacaattgaagacaaagaaaagtttaacccagatctgtatgtgaaacttgttgaagttgagcaaaagagaactaacaagagaaaagaaaaagttggtcgaatcgccggttctcttgaacagttagctttgcttaacggtgttgacgttgttatcggtgaagctgatcttggcgaagtcaagaaaggcaaatccaaaaaacaaaatagtcgaaacatggactggtgtgccaagcaagtcgctgagcggcttgagtacaagctgaccttccattgtattggttattttggtgtcaacccgatgtatacgtctcatcaagatccatttgaacatcgtcgcgttgctgaccacctagtaatgcgtgcgaggtttgaagaagtgaatgtaagtaatgtttcggaatggcacatgcgaaacttctcaaactatctgcgtgcggactcaggtactggtttgtattacaaacaagctaccttggatttcctcaagcattatgatttggaagagcacgccgatgatttggaaaagcagaatatcaaattctatgacttcaggaaaattcttgaagacaaacaattgacttctgttattgttccaaaacgtggcggtcgcatttacatggcgactaacccggtaacttccgatagtacgcctgtcacttatgccggtaaaacttacaaccggtgtaatgctgacgaagtggctgcggctaacatcgctatcagcgtcttagctcctcactctaagaaagaagaaaaggaagataagatcccgattatttctaagaagcctaagtctaagaatactcccaaggcccggaagaatttaaagacttctcaacttcctcagaaa

SEQ ID NO: 24 - DNA, 3012 bp, unidentified
  source 1..3012: mol_type = other DNA
atggctagcaaacatgtagtgcgtccctttaatggcaaagtaacagctactggcaagcgtttggcatacttggaagaaacttttcattatttggaaaaagctgctggtggtgttagtactttgtttgctgcccttggttcttatcttgatgcaaccacaataagcaatttaattaataaaaatcaagatttagccgttgtaatatttcgttatcatgtggttcccaaaggtgaggctcatactttacctgtaggtacagacatggttagtcgttttgttgccgactatggtatggagccgaatgagtttcagagagcttatttggacagtccgattgaccaagaaaagtattgttggcaggataatagggatgttggttgttggttgggtgagcaattgggtgttagcgaagcggacatgcgggcaatagcagtaactttttataacaatcagatgctttatgattgtgtaaaaggtactgggagtggtaatgctgtgagtcttttgtttggcagtggtaaaaagtctgattacagtatgaagggcgttatagcaggtaaggctgcttcagtactggcaaaatatcgcccagctacctatcaagatgcccgaaagatgattttggaagctaatggtttcacctcagtaaaagatttggttacttcttatggaataactggaaggtctagtgctttgcagatatttatggaagggattgaaagtggtcctattagcagcaagacattagatgctcgtattaagaagttcacagaggattcggagcgcaatggcaggaagaatctagtccctcatgctggggctatacgaaattggctgattgagcaagctggtagtagtgtagaaaactatcagatggcatggtgcgaggtttacggtaatgtgtctgccgactggaatgccaaagtagaaagtaatttcaatttcgtagcggagaaagtaaaggcattaacagaattatccaacattcagaaatcgactcctgatttgggtaaggctttgaaattatttgaagaatatttgactacttgtcaggatgaatttgctattgcgccttatcattttagcgtcatggaagaggtgcgaatggaaatggcaacaggcagggaattcaatgatgcttatgatgacgccctaaatagcttggacatggagtctaagcagcccattcagcctttgtgtaagtttttgattgagcgtggaggtagtatcagttttgatactttcaagagtgcagccaagtatttgaaaacacagagcaagattgctggtcgatatccacatccatttgtaaaaggtaatcagggatttacttttggttccaaaaacatttgggcagccatcaacgatcctatgatggagtatgcagatggtcgtattgctggtggttctgcaatgatgtgggtgacggctacattgttggatgggaaaaagtgggttcgccatcatatcccatttgccaatactcgatactttgaggaggtttatgctagcaagaaagggttgcctgtattgccttgtgctagagatggcaaacactcatttaaattgggcaataatttgagtgtagagagagttgaaaaggtcaaagaaggcggtagaactaaagcaaccaaggcacaagagcgtattttaagcaacttgactcacaatgtgcagtttgacagttcgacaacttttattattcgtcgtcaggaagaaagttttgtaatttgcgtgaatcatcgacatccagctccgctcatgaagaaggagatggaagttggcgacaaaatcattggtatcgaccagaatgtgacggcacccacaacctatgccatagttgagcgtgtggcttctggcggcattgagcgtaacggcaagcagtacaaagtgacggcgatgggagccatttccagcgttcagaagaccagaggcggtgaggtggatgttttgagttatatgggggttgaactttctgacagcaaaaatggatttcaaagcttgtggaataaatgtttggactttgttaccaaacatggcactgaaaatgatgttaaatattataacaacactgctgtctgggccaacaagctgtatgtgtggcacaagatgtatttccggcttttgaagcagttgatgcgtcgggcaaaggacttgaaacctttcagggaccatttacagcatctattattccatcctaatcttagtcccttgcaacgccatagcttgtccttaacaagtctggaagcaactaagatagtgcggaattgcattcattcgtatttcagtctattggggttgaagaccttggatgaacgcaaagccgctgacatcaatttattggaagttttggaaaagctgtatgctggtttggttgagaggcgaaaagaaagaaccaaactaaccgctgggctattggttcgcttatgtaatgagcatgggatttcttttgcagctattgagggtgatttgccggtcgttggagagggcaaatctaaagctgccaacaatacacaacaggattggacagccagagagttagagaagcgattatctgagatggcggaggtggttggcatcaaggtaatagctgttttgccccactataccagtcatcaggacccatttgtttatagtaaaaataccaagaaaatgagatgtcgttggaactggaggaccaccaagaccttcactgatcgtgatgctttgagtatacgcaggatattaagcaagcctgagacgggtacaaatttgtattatcagaagggcttgaaagcatttgctgaaaagcatggtctggatttggcagagatgaagaagcgcaaggatgctcaatggtatcttgagcgcattcaagacaagaattttttggtgccaatgaatggtggtagagtttatttgagttctgtcaaattagccgggaaagaaacaattgacatgggtggcgaaattttatatcttaacgatgccgatcaagtcgcagcgttgaatgttttgttagtgaagatttga

SEQ ID NO: 25 - DNA, 3058 bp, unidentified
  source 1..3058: mol_type = other DNA
atggctaagaaagaacatattataagaccattcaaaggaacactaccacttcgtggtgatagactaaggtatcttcaagataccatgaaatatatgaaaaaggttgaagatactatcacagaactctgcgccgctgttatcgcctatgccaaacccaccatcattcaacaaatacttggcgaagaaattgaaaccaccagcacattttgtagcttccgcttagtaggcattcatgaaaactttaccatgccactaaccacaaatatgataaaacacttccagaaaacctttaacataaacccatcagaaaaacaagcaatctatctctccagtggattcgattcagataaatatcgctggcaagatacttccgaagtatccagaaacttcgccaacaaatgccgacttactaatcaagaattccaagaatttgccgaacaagcactactcaatatgtgcttcataggttgctctggtagccccggtgcaactaatgccgtctcacaaatctttggcacaggcgaaaaaagcgattaccaacgcaaaagccaaatcgctaaaattgctgctgataccctcgaaaaccacaaacctagcacctatgagtctgctagattaatggttcttaatacacttggacacaaaacaatagaagattgtgtcaatgactatggcgcaataggagccaaatccgccttccgactattcatggaatcaaaagaaataggaccaattacatctgaacaactcacaaccaaaattaagaagttcagagaagatcataaaaagaactccatcaagaaacaacttccacatgtagaaaaagttcgtaacgctttgctatcacaattcaaagaacaatacctgccctcagcatgggcagaagcatggtgcaatatcatgggcgaatttaactccaaattatcaaataataataacttcatcgaccaaaaaacaaaaatggtcaatgactgcgataatattaaaaaatctaatccacaactagacaaagctgttaatatgctcgatgaatggaaatataaaaactgggatgataattctgctatacacccatatcatattggcgatcttaaaaaactcatggcaatattcaatatcaataacgaaggaaccttcgacgaaagattttcagctagctgggaacaattctccacatcactagaatacggggagaaaccacccgttcgtgatctactagcccatatcatcaaaaatatgaatgacctcacctacacagacgtaatcaacgccgcaaaatttctcaaacttcaagataatataagaaataaatacccacaccctttcgttatgccaaataaaggatgtacctttggtaaagataacctttggggcgaaattaatgaccccacagccaaaatcaaatcaacagaagaagttgctggacaaagacctatgatgtggctgacagccaaacttctcgataatggaaaatgggtagaacaccacatccctttcgcctccagtagatactttgccgaagtttattataccaatccagcactccccactctaccaatagctagagatggaaaacattcatacaaattaacaaaaactatagatgccaatactgcaaaaactctagtaaataatcctagagataaagcagctaaactaatcgcacgaactaaagccaatactacacacaatgtaaaatggattaaacctacatacagaatccaaaaagaaaataaccaattcgttattactatcaatcatcgacacccatgcataacaccaccaaaggaaatcatactcggagatcgtatcctatccttcgaccaaaacgaaacagcccccacagcattctccattctcgaaaaaacaaccaaaggtacagaattctgtggccaccacattaaagtgctaaagactggtatgctagaagctaaaattaaaaccagtaagaaatcaatagatgcattcacatacatgggaccaatggaagatgatcatgcgtctggcttcccaacactactcaacatatgtgaaaaattcatatcagagaatggagatgaaaaagacaaaagtttctcttctcgtaaattgccctttaaaaggtctttgtacttctttcatggctcacacttcgatttactaaagaaaatgatcagaaaggccaaaaatgaccccaagaaattgaagttagtaagaattcatatcaatgaaattctattcaattccaatttgtcaccaataaaactacacagtctgtctattcacagcatggaaaataccaaaaaagttatagctgctattagctgctatatgaatgttcatgaatggaaaactatcgatgaacaaaagaatgctgatataacattgtataatgctaaagaaaaactatacaacaaccttgttaaccgccgtaaagaaagagtaaaagtaactgcaggtatgttgattcgattagctagagaaaacaattgcagattcatggtcggggaagcagaattacccacccaacaacaaggcaaatcaaaaaagaacaataactccaaacaggattggtgcgccagagatatagcacaacgatgtgaagatatgtgcgaagtcgtaggtataaaatggaatggcgttactccgcataataccagccatcaaaacccattcatctataaaaatactagtggacaacaaatgcgatgccgttatagtctcgtaaagaagtcagaaatgacagacaagatggcagaaaaaattagaaatattttacacgctgaacctgtaggcactacagcatactaccgtgaaggcattttggaattcgccaaacatcatggattagatctgggaatgatgaaaaaacgaagagatgctaagtattatgataatcttccagatgagtttctgcttcctactagaggtggtagaatctatctgtccgaaaatcaactaggcggaaacgaaaccattgttattaatgggaaaaaatattttgtcaatcaggcagatcaagtcgctgccgtaaatattggcctgctttatcttctgccgaagaaaaaccagagttaag

SEQ ID NO: 26 - DNA, 3144 bp, unidentified
  source 1..3144: mol_type = other DNA
atgtccgagaagaagttccacatcaggccctaccgctgctcgataagcccgaacgcccgcaaggccgatatgctcaaggcgacgatctcctaccttgactccctgacctccgtgttcaggtcgggattcaccgcactacttgcgggcatagacccgtcgacggtgagccgcctggcgccttcgggggccgtcggcagcccggacctgtggagcgccgtcaactggttccgcatcgtgccgctcgcagaggccggcgacgcccgagtcggccaggcatcgctcaagaacctcttccgtggctacgcaggccacgagcccgacgaagaggcgtcgatctatatggagtcgagagtggacgataagaggcacgcgtgggtggactgccgtgccatgttcagggcgatggcgctcgagtgcgggctggaggaggcccagctcgcctccgacgtgttcgccctcgcctcaagggaggtcatagtcttcaaggacggcgagatcaacggctggggcatagcctccctgctgttcggcgagggcgagaaggccgactcgcaaaagaaggtcgccctgctccgctccgtgaggctggcccttgagggggactacgcgacctacgaggaactctccgggctcatgctggccaagaccggagcctccagcggctccgacctccttgacgagtacaagaggagcgagaagggcggcagcagcggcggcaggcaccccttcttcgacgaggtcttccggaggggcggcagggtcaagcaggaggagcgcgagaggctgctgaagagctgcgacacagcgatccagaagcaggggcaggcgctgccgctgtcgcacgtcgcatcttggaggcaatggttcctgcgcagggtcacgctgctgcgcaaccgcaggcaagagtcgttcgcagtctgcatcaccaacgccctcatggacctacagcccaagaacctacgcaacgtccactacgtgacgaaccccaagagcgagaaggacaagggcgtgctcgagctgcgcgtcgacgtcaagaacaacgaggggccggacgtggcgggcgcgcaggcggtcttcgacgcctacatggcgaggctggcacccgacctgcgcttctccgtgatgccacggcacctcggctccctcaaggacctctacgccctttgggccaagctcgggcgggacgaggccatcgaggagtacctcgagggctacgagggaccattcagcaagaggcccatcgcaggcattctacaaatcatccacgcacaccgtggcaaggtgggctacgatagcctgttgcgtgcggcgaggctcaacagggcgatggacaggctggagaggaagagggcccacgcctgcgcagccggcaacaagggttacgtctacggcaagagctcgatggtcggccgcatcaacccgcagagcctcgaggtcggcggccgcaagtcgggccgaagcccgatgatgtgggtgaccctcgacctggtggacggcgacaggttcgcgcagcaccaccttcccttccagagcgcccgcttcttctccgaggtctactgccacggcgacgggctcccggccacccgtgtccccggcatggtcaggaaccgtcgcaacgggctggcgatagggaacgggctcggggagggtggactctcagcgctgcgcgcaggcagcgacaggaggaagagggccaacaagaggacgctgcgcgccctcgagaacatcacgcacaacgtggagatcgaccccagcacctccttcacgctgcgggaggacgggataatcatttcgcacaggatcgagaagattgagccgaagcttgtcgccttcggggacagggcgctcggcttcgacctcaaccagacaggggctcatacgtttgcggtgctccagaaggtggactcgggcggcctagacgtcggccactctcgcgtgtcgatcgtgctcaccggcactgttcgcagcatctgcaagggcaaccaggcgagcggcggacgggactacgacctgctttcctacgacggccccgagcgcgacgacggggcgttcacggcatggaggtcggacaggcaggccttcctgatgtctgccatacgggagctgcccacgcccgccgagggggaaaaggactacaaggcagacctcctctcccagatggcgagccttgaccactacaggcgactgtacgcgtacaacaggaagtgcctcggcatctacatcggggccttgagacgcgcgaccaggaggcaggccgtggccgcattcaaggacgagatactctcgatcgcgaatcaccgctgcgggcctctcatgcgtgggagcctttcggtgaacggcatggagtccctcgcgaacctcaagggcctagccacggcatacctgagcaagttcaaggacagcaagtccgaggacctgctgtcgaaggacgaggagatggccgacctgtacagggcttgcgcgcgcagaatgactggcaagcgcaaggagaggtacaggagggcggctagcgagatcgtccggctggccaacgagcacggctgcctgttcgtcttcggcgagaaagagctgcccaccaccagcaagggcaacaagagcaagcagaaccagaggaacaccgactggtcggcccgtgccatagtgaaggcggtcaaggaggcctgcgagggctgcggtctcggcttcaagcccgtgtggaaggagtactcgagcctcacggacccgttcgagagggacggggacggaaggcctgccctccgctgccggttcgccaaggtggccgcacccgactccgaactcccgcctcgcctgacgaaggccgtcggctcctatgtgaagaacgccctcaaggccgacaaggcggagaagaagcagacctgctaccagcgtggcgccatcgagttctgctcaaggcacggcatcgacgtccggaaggcgaccgacaaggccattcgcaaggcagtccgtggctcctccgacctgcttgtgccgttcgacggggggaggaccttcctgctctcgacgaggctgtccccggagtcgcgaaaggtggagtgggccgggcgcaccctgtacgagttccccagcgacatggtcgccgcaatcaacatcgcctgcaggggcctagagccacgcaaggcctag

SEQ ID NO: 27 - DNA, 3090 bp, unidentified
  source 1..3090: mol_type = other DNA
atggacgagcaagctgttgtttcctctggttccgacaagaccctcaagatcgtacgcccttacagggcaaaagtaaccgctactggaattcgccttgagggaattaaaaataccctgaattacctgaagcgtacagaaatttgtctgtcacgcctgaatgcagcttgtggagcttttctcactcctgccatcgtggagcagatctgtaaggacgatcctgccctagtttgtgccattgctcgctttcaattggttccggttggtagtgaagccactttgtccgacagtgggctaatgcgtcattttaaggctgctctcggtgaattgaccccgctacaagaagcctacctgaatagcagctataacgacgaattgtacgcatggcaggatactcttgtcttagcgcgacagattattgctgaaaccggattgactgaagatcaattccgcgcctttgctcatgcctgtttcaagaacggcaatattatcgggtgcgctggtggtcccggtgccagcaacgccatctctggcatttttggcgagggaattaaatccgattattcactccgaagtgaaatgaccgctgccgttgcaaaggtgtttgaagagaaacgtcctatcacttacgaagaagctcgggctctcgctctggaagcaactggacacgccagcgttcagtctttcgtggaagcatttggtaaacaggggcgtaaaggcactctgattcttttcatggaagataccaagacaggcgcattcccaagcaatgaattcgattacaagctcaagaaactgaaggaggatgcagagcgtgtcgggcgtaagggtatcatcccgcaccgcgatgtgattgcttcttatctccgcaatcagactggtgctgatattgaatacaactccaaggcatggtgcgagtcctactgttgtgccgtgagcgaatacaactcaaagatgagcaacaatgttcgatttgccacggaaaaaagtcttgatttgaccaagcttgatgaaacgatcagggaaacgcccaagatcagtgaagccatgcttgtttttgaaaactacatggcgcgaattgatgccgatctccggttcattgtgagcaagcatcatctcggcaatctcgccaaattccgtcagaccatgatgcatgtctctgcatcagaatttgaagaggcttttaaggcgatgtgggctgattacttggctggtctggaatacggtgaaaaacccgcgatctgtgaactggtgcggtatgtcctgacccatggcaacgatttgcctgtcgaagcgttttacgctgcgtgcaagttccttagcttggatgacaagatcaagaatcgttaccctcacccatttgttccgggtaacaaaggctacacctttggcgcgaaaaacttgtgggcagaaatcaatgatcccttcaagcccatccgtcaaggcaacccagaggttgctggtcaacgccccatgatgtgggctaccgccgaccttctggacaacaacaaatgggtcttgcatcacatcccctttgcctccagcaggtatttcgaggaagtgtactacaccgatccctcgcttcctacggctcaaaaggcgcgagacggcaagcatggctatcggttgggcaaagtgctggatgaggctgctcgggagcgtttaaaagcaaataatcgccagcgcaaggcagctaaagccatcgagcggatcaaagccaactgtgagcacaatgtggcttgggatccgaccaccaccttcatgcttcagttggattctgagggtaatgtgaaaatgacgatcaatcatcgtcacattgcctatcgcgcacccaaggaaattggtgttggggacagggtgattggcatcgaccaaaacgagactgctcctacaacctacgccattcttgagcgcacggaaaatcctcgcgatcttgaatacaacggcaagtattaccgtgtagtcaagatgggtagtgtgacttcaccgaatgtcagcaagtatcgcacggtggacgctttgacttacgatggcgtgtccttgtcggatgatgcttctggtgctgtgaactttgtggtattgtgtcgcgagttttttgcagcacatggcgacgatgagggtcgcaagtaccttgagaggactttggggtggagttcaagcctgtattccttccatggaaactatttcaagtgccttacgcagatgatgcgtcgatccgctcgttctggtggtgatttgacggtctatcgcgcccatttgcagcagatcctgttccaacacaatctgtcgcccttgaggatgcacagcttgtctttaaggagcatggaatcgacgatgaaggtcatcagttgcatgaagagctacatgtctctttgtggctggaagaccgacgcggatcggattgccaatgataggtcgctgtttgaggctgctcgtaagctttacaccagtttggtaaatcgtcggacggagcgggttcgtgtgactgctggcattctgatgcgtctgtgcttggagcacaacgttaggtttattcacatggaggatgaacttcctgtggctgaaacgggcaaaagcaagaaaagcaatggcgcgaagatgcattggtgtgcccgggagcttgccgttcgtttgtcccagatggcagaggtgacgagcgtcaagttcacaggtgtgtcaccgcattacactagccatcaagacccatttgtgcattccaagactagtaaggtaatgcgtgcccgttggagttggcggaatcgtgccgatttcacggacaaggatgcggagcgtattcggacgattctgggtggtgatgacgcagggacgaaggcttattatcgctcggcgttggctgaatttgcctcgcgctatggtctggacatggagcagatgcggaagaggcgcgatgctcagtggtatcaagagagactgccagaaacctttattattcctcagcggggtggtagagtgtacttgtcttctcacgatctgggatcaggtcaaaaagttgacgggatttatggtggtcgtgctttcgtgaatcacgctgacgaggttgctgcgctgaatgtggcgttggtcaggctgtga

SEQ ID NO: 28 - DNA, 3090 bp, unidentified
  source 1..3090: mol_type = other DNA
atgaagactgaaactcttatccgtccctaccccggcaaactcaacctccaaccccgtcgagcacaattcctcgaagactccattcaatatcaccagaaaatgacggaatttttctaccaattcctccaagcagtcggcggtgccaccacgcaccaaaacatcagcgatttcatcgacaataaagccaccgatgaacaccaagccactctcctcttccaagtagtctccaaagacagcacaacaccagaatgccccgcagaagaactcctagcccgatttgcccaatacaccggcaaacaacccaatgaggctgtcacccactacctgaccagcagaatcaatacagataaataccgctggcaggacaatcgactcctcgcccaaaacatcgcttcacaactgaacatctccgaaactcaattccaagagatcgctcacgcaatcctgtccaacaacctatacatcggtcaaactgcatccaacgcagcagccaacttcatcagccaagtcacaggcacaggccagaaagcccccaaggcagcacggctcgatgtcctgttccagaccaaccaagccctcgccaaaacacaacccacaaccttcggccaactccaacagatcatcgtacaagcctgcggtgaatccaccaccgatgcagtcctcgccaaattcggcaacaaaggcgctgcaaccagccttcaactggcccttaaaaccgaccccaacacaacgctggatcagaagaagtacgaagccctgcaaaagaaatttgcagaggacgaaaccaaatatcgcaacaaggtcgatatcccccacaagacccaactgcgcaacctcatcctcaacacctcaaaccaattctgcaactggcacaccaagccagccatcgaagcctttaagtgcgccatcgctgacatccagtccaaagtcagcaacaacctccgcatcatgcaggaaaaggccaaactctacgaggcattcagaaatgtcgatccacaagtccagatcgccgtccaagctcttgaaaaccacatgaacacacttgaggaaccctacgcaccctacgcccactcgttcggcagcgtcaaagacttctacgaagacctcaacaacggctccaacttagatgaggccattcaaaccatcgtccacgattccgacaacttcaacaggaagccagaccccaactggctccgcatcatcgcacctctccactcatcccattccgcaagccaaatcatggaggcagtaaaatacctgtccagcaaacaggattacgaactccgtaaacccttcccattcgtcgccactaacctgccagcaacctacgggaaatttaacattcccggcaccctcaacccacccaccgacagccttcacggcagactgaacggtagccactccaatatgtggctcacagccctgctcctcgacggcagggattggaaaaaccaccacctttgcttcgcctcaagccgctacttcgaggaggtctacttcacaaaccccagcctgcccactacagacaaagtccgtagccccaaatgcggcttcacactcaagagcgtgctcgactccgaagccaaagacaggattcgcaacgctcccaaatcccgcaccaaggccgtgaaagccatcgaacgcatcaaggccaactccacccacaatgtggcgtggaaccccgaaacctctttccagatgcagaaaagaaacgatgagttctacatcaccatcaaccaccgcatcgaaatggaaaaaatccccggtcagaaaaagaccgatgacggtttcacaatccaccccaaaggtctcttcgccatcctcaaggaaggcgacagaatcctgtcacaagacctcaaccagaccgcagccacacattgcgccgtctatgaagtcgccaaacccgaccagaacaccttcaaccaccacggcattcacctcaagctgattgccacagaagaactcaaaatgcccctcaagaccaaaaagtccacaatcccagatgccctctcctaccaaggcatccacgcccacgaccgtgaaaacggcttacaacaactcaaagatgcctgcggagctttcatcagccccagactcgatcccaaacaaaaggctacttgggacaactccgtctccaagaaggagaatctctatccattcatcaccgcctacatgaaactcctcaagaaggtcatgaaggcaggtcgtcaagaactgaaacttttcaggacacaccttgaccacatcctctttaaacacaacctcagccccctcaagctgcacggtgtgtccatgatcggtctggaatcatccagagcaaccaaatccgtcatcaacagcttcttcaaccttcagaacgccaagacggaacagcagcagatcgccctcgaccgacccctgtttgaggccggtaaaaccctcatcaacaaccaaacccgccgacgacaggaaagggtcaggttagaaaccagtctcaccatgagactggcacacaaatacaacgccaaggcaatcatcatcgagggtgaactgccacactccagcaccggaacctcgcagtaccagaacaatgtccgtctggactggtctgccaagaaatccgcaaagctgaaaaccgaatcagccaactgtgcaggcattgccatatgccagatcgatccgtgccacacaagccaccaaaatcccttccggcacactccaactaacccagacctcagaccacgatttgcgcaagtcaaaaagggcaaaatgttccagtatcaactcaatggactacagaggctgctcaaccccagaagcaaatcctcaactgccatctactacaggcaggcagtccaaagtttctgcgcccaccacaacctgacggagagggacatcacctctgccaaattccccagcgatctggagaaaaaaatcaaggatgacacctatctgattccccagagaggtggtagaatatacatcagcagcttccccgtcactagctgcgcccgtccctgcaccagcaaccattatttcgggggtggacaattcgagtgcaatgctgacgctgtcgcagccgtcaacatcatgctgaaggttcacccgtaa

SEQ ID NO: 29 - DNA, 3036 bp, unidentified
  source 1..3036: mol_type = other DNA
atgcccattcgcggatataaatgcactgttgtcccaaacgtacgcaaaaagaaactcttggaaaaaacctatagctacttacaagagggttctgatgtattttttgatcttttcttgagtctgtatggtgggatcgccccaaaaatgattccacaagacctggggatcaatgaacaagtaatttgtgctgccaattggttcaaaattgttgaaaaaacgaaagattgcatcgctgatgatgcgttgttgaatcaatttgctcaatattatggggaaaaacccaatgaaaaggttgttcaatttttgacggcatcttacaataaagacaaatatgtttgggtcgattgtcgtcaaaaattttacactctgcaaaaggatttgggagtccaaaacctagaaaacgacctggagtgtttgattcgagaagatttgttgcccgtaggaagcgacaaagaagttaatggatggcactcgatatcaaaattgtttggttgtggagaaaaagaagacagaacaattaaggctaaaattctgaatggcctatgggaaagaattgagaaagaagatattctaacagaagaagacgcaagaaatgaactattgcactctgctggggtgttgactccaaaagaatttagaaaagtatataaaggggctgctggtgggcgtgattgttatcacacgttgctggtagatgggagaaacttcacttttaaccttaaaacactcattaagcagaccaaggataaattaaaagaaaagtctgttgatgttgaaatccccaataaagaagcattgcgtctatatctcgaaaaacgaattggacggtctttcgagcaaaagccatggagcgaaatgtataaaacggccctctcagccgttatgccaaaaaatacgctaaattattgtttcgccattgataggcacgcccaatatacaaaaattcaaacactaaagcagccatatgattcggcaattactgccctaaatgggttttttgagtctgaatgctttacaggctcagatgtttttgttatttctccctcccatttggggaaaactcttaaaaaactttataattacaaagatgttgaatctggcattagcgaaattgttgaagatgaagacaatagtttgcgatctggggtaaatgtaaatttacttagatatatttttactcttaaagatatgttttctgctgaggatttcatcaaagcggcagaatataatgttgtatttgaacgctacaacaggcaaaaagtccaccctacagtaaaagggaatcaatcgttcactttcggcaattccgcattgagcggtaaagttattcctccatcaaaatgcttgtccaatttgcctggacaaatgtggctggccattaatctacttgaccagggcgaatggaaagaacatcacattccttttcacagtgcaagattctatgaagaaatctatgcaacaagtgacaatcaaaataatcccgtagatttgcgaactaaacgttttggctgctctcttaacaagactttttctgctgctgacatcgaaaaggtgaaagaaagtgccaagaaaaaacatggcaaagcagctaaacgtattttgagagccaaaaacaccaatacagccgtaaattgggttgattgcggttttatgttggaaaaaacagaggttaactttaaaattactgttaactacaaacttccagaccaaaagttgggaaaatttgaaccaattgttgggacgaagattttggcttatgaccaaaatcaaaccgctcctgatgcttatgcgattcttgaaatttgcgatgatagcgaagcttttgattacaagggatataaaatcaaatgtttgtctactggtgatttggcttcaaagtcattgaccaaacaaacagaagttgatcagctagcttataagggtgtggacaaaactagcaatttttacaaaaagtggaaacagcaacgaaggctttttgtcaaaagtcttaacattccagatgccctaaagagttttgaaaacatcaataaagaatatctttatgggttcaacaattcgtatctgaagttgcttaaacaaattttacggggcaaatttggaccaattcttgttgatattcgaccagaacttattgaaatgtgtcagggaattggctctatcatgcgattgtctagtctaaaccatgatagtttggacgcaattcaatctctcaaatccttgcttcactcctattttgatctcaaagtaaaggaagaaatcaaaacagaagaattgagagaaaaagcagataaagaggtttttaagttgcttcaacaagtgattcaaaaacaaaagaataaacgcaaagaaaaagttaatagaactgttgatgccattttgactttggcggctgatgagcaagtacaagtcattgtaggagagggagatctttgtgtttccaccaaaggaacaaaaaagagacaaaacaacagaaccattgattggtgtgccagagcagttgtggaaaaactagaaaaagcatgcaaactacatgggttgcattttaaggaaattccaccacattacacttcacatcaagattgttttgaacacaacaaggatattgaaaatccaaaagaagtcatgaagtgtcgtttcaatagcagcgaaaatgtagctccttggatgatcaagaaattcgcaaattatcttaaatgcgaaacaaaatattatgttcaaggaatgcaagattttctagagcattatggtctagtagaatacaaagatcacatcaaaaagggaaaaatctcaattggggattttcaaaaacttatcaaacttgctcttgagaaagttggagaaaaagagattgtttttccatgtaaaggtggtagaatctatttgtcaacctattgcttaacaaatgagtctaaacccattgttttcaatggcagaagatgctatgttaataatgcagaccatgttgctgcgattaatgttggcatttgtcttttgaattttaatgcgagagccaaggtggcggaaaaaaccccttga

SEQ ID NO: 30 - DNA, 3282 bp, unidentified
  source 1..3282: mol_type = other DNA
atggctaagaaggattttatcgctcgtccctacaattcattcctgctccccaacgacagaaagcttgcttatctggaagaaacttggactgcctacaagtcaatcaaaacagtactgcaccgtttcctcatcgcagcatacggcgctattcccttccagacctttgcaaaaaccatcgaaaacacacaagaagacgaattgcaattggcatatgccgttagaatgttcagactagttccaaaagacttctccaagaatgaaaacaacataccccccgatatgctcattagcaagcttgctagctatacaaatataaatcaatcaccaaccaatgtcttgagctatgtaaacagcaactacgatccagaaaagtataagtggatcgactcacgcaacgaagccatctcattgtccaaagaaatcggcatcaaactcgatgagttggcagactacgctaccaccatgctttgggaggactggcttccacttaacaaagacacagtcaacggttggggcaccactagcggcctattcggcgcaggaaaaaaagaggatcgtacccaaaaggtacaaatgctcaacgcattgcttttggggcttaaaaacaaccctcccaaggactacaaacagtattcgaccatccttctcaaggcatttgatgccaaatcatgggaagaggctgttaaaatttataaaggcgaatgctcaggtagaaccagtagctacctgacagaaaagcatggagacatttccccagaaactttggaaaaactaattcaaagtattcagagagatattgctgacaaacaacaccccatcaatctacctaaaagagaagaaattaaggcatacttggaaaagcagagtggtactccatacaatctcaatctctggtcacaagccctacacaacgctatgtcttctatcaagaagacagatactcgcaatttcaatagcacactagaaaaatatgaaaaagaaattcaactcaaggagtgcttgcaagatggtgatgatgtagaattacttggcaacaaattcttttcatctccatatcataagaccaacgatgtctttgtcatttgctctgagcatatcggcaccaatcgcaaatacaatgtcgttgagcagatgtaccaactcgctagcgaacatgccgattttgaaacagtgttcactctcctcaaagatgaatacgaagaaaaaggtatcaaaaccccaatcaaaaacattcttgaatacatttggaacaacaagaatgtgcctgtaggcacttggggtagaattgccaaatacaatcagctgaaagatagattggctggaatcaaagccaatcctaccgttgaatgcaaccgtggcatgacatttggcaattctgcgatggttggcgaagttatgcgatccaatcgcatttcgaccagcacgaagaataaaggccagattttggcccaaatgcacaacgataggcccgttgggtcaaacaacatgatctggctggaaatgacgcttttaaacaacgggaaatggcaaaaacaccacatcccgacccacaataataagttctttgaagaagtccatgctttcaatccagaactgaagcaatccgtgaatgtgcgaaatagaatgtatcgttctcaaaactattcgcaacttccaacatctctgaccgatgggctgcaaggcaacccaaaagccaagattttcaagcgtcaatatcgtgcgctcaataacatgacggcaaacgtgattgatccaaagttgagttttattgttaacaaaaaggatggcagattcgaaattagcatcattcacaatgttgaagtgatcagggccagacgagatgttctggtcggggattacttggtcggcatggatcaaaaccagactgccagcaacacttacgctgtcatgcaggtggttcagccaaacactcctgactcccatgaatttcgcaaccaatgggtgaagtttattgagagtggcaagattgaatcttctactctcaattctagaggcgaatacattgaccagttgagtcatgatggcgtggatttgcaagaaatcaaggattctgaatggattccagctgctgagaaattcttaaacaagttgggagcaatcaacaaggacggcactccaatcagcatctctaatacttcaaagagggcttacaccttcaactccatatatttcaaaatcttattgaattatcttcgtgctaatgatgttgatctgaatttggtgagagaggagattctgcgtattgccaacggcaggttttcgcccatgcgtctgggtagtctgtcgtggactactcttaagatgttgggcaactttagaaatttgattcatagttatttcgatcactgtggtttcaaggaaatgcctgaaagggaatctaaagacaaaaccatgtacgatctgttgatgcataccatcacaaagctgacaaacaagcgtgccgaaagaacgagtaggattgctggttctttgatgaatgtagcccataagtataaaattggcacaagcgttgtgcatgttgtcgttgaaggcagtctaagcaagaccgacaaatccagcagcaagggtaataaccgaaataccactgattggtgctcaagggctgtagtcaaaaagctggaagacatgtgcgtcttttatgggttcaatttgaaagcagtttcggcgcattacactagtcaccaagacccattggttcatcgggctgattatgatgatcccaagcttgctttgcggtgtcgatattcgtcgtatagtcgggctgattttgaaaagtggggtgagaagtcgtttgctgctgtgattcgttgggctaccgacaaaaagagcaatacttgttacaaggttggggctgtggagttctttaaaaattataaaatcccagaggacaagatcaccaagaagctgaccataaaggaattccttgagataatgtgtgcagagtcacactatccgaatgagtatgacgatattttgattcctcgccgtggaggcaggatttatctgacaacgaagaagttgctaagtgattcgacccaccaaagagaaagtgtgcatagtcacacggctgttgtcaaaatgaacgggaaagagtattattcctcagatgcagatgaggtggctgcgatcaacatctgcctacatgactgggttgtcccactgaattggaccaatcactgcctacctgctggctggtgctctgaccacctgaaagaatgtgtgcaatgtcacactccagacccagtacgaatatccatgtaa

SEQ ID NO: 31 - DNA, 3240 bp, synthetic construct
  source 1..3240: mol_type = other DNA
atgagttctgatgtggtgcggccttataacacaaagctgctcccagataacagaaagcacaatatgttcctgcagaccttcaagcggctgaacagcatctctctgaaccacttcgacctgctgatctgcctgtacgctgcaatcaccaacaagaaggccgaggaatacaagtctgaaaaggaagcccacgtgaccgccgatagcctgtgtgccatcaattggttcagacccatgagcaagagatacagcaaatacgccaccaccaccttcaacatgttagaactgtttaaggagtacagcggccacgagcctgatgcctattccaagaactacctgatgagcaatatcgacagcgacagattcgtgtgggtggattgtaggaagttcgctaaggactttgcctatcagatggaactgggtttccacgagttcaccgtgttggccgaaaccctgctggctaattctatcctggtgctgaacgagagcaccaaggccaattgggcttggggaaccgtgtctgccctgtacggcggcggagataaggaggacagcacactgaagagcaagattctgctggccttcgtggacgccctgaacaaccacgagctgaaaacaaagagagaaatcttgaatcaagtgtgtgaatctctgaaataccagagctaccaggacatgtacgtggattttagaagcgtggttgacgaaaacggcaacaagaagtctcctaacggctctatgcctatcgtgaccaagttcgagacagacgacctgatcagcgacaaccaaagaaaggccatgatcagcaacttcactaagaacgccgctgccaaggcagctaagaaacctatcccttacttggaccgcctgaaggagcacatggtgtccctgtgcgacgagtacaatgtgtatgcctgggccgcggccatcacaaacagcaacgccgacgtgaccgcccggaataccagaaacctgacattcatcggcgaacagaacagcagacgaaaggaactgagcgtgctgcagacaacaaccaacgagaaggctaaggacatcctgaacaagatcaacgacaacctgattcaggaggtgcggtacacccctgcccctaagcacctgggcagagatctggccaacctgtttgatacactgaaggaaaaggacatcaacaacatcgagaacgaagaagagaaacagaacgtgatcaatgactgtatcgagcagtacgtggacgattgcagaagcctcaaccggaaccccatcgcagccctcctgaagcacatctctaggtactacgaggatttcagcgccaagaatttcctggacggcgccaagctgaacgtgctgactgaggtggtgaaccggcagaaggcccaccccaccatctggagcgagaaggcttacacctggatcagcaagttcgacaagaaccggagacaggccaacagcagcctggtcggatgggttgtgccccccgaggaggtgcacaaggagaaaatcgccggacagcagagcatgatgtgggtgaccctcaccctgctggacgacggcaagtgggtcaaacatcacatccccttcagcgacagcagatactacagcgaagtgtacgcctacaaccctaatctgccttatctggacggaggcatcccaagacagagcaagttcggcaacaaaccaacaaccaacctgacagccgagtcccaggccctcctggctaattctaagtacaagaaagccaacaagagcttcctgcgggctaaagagaatgccacacacaacgtgcgggtgtcccctaacacctctctgtgcattagactgctgaaggacagcgccggaaaccagatgttcgacaaaatcggcaacgtgctcttcggcatgcagatcaaccacaagatcaccgtgggaaaacctaactacaagatcgaggtgggcgacagattcctgggcttcgatcagaaccagagcgagaaccacacctacgccgtgctgcagagagtgtccgagagcagtcacgacacccaccactttaacggctgggacgtgaaggtgctggaaaagggcaaagtgaccagcgatgtgatcgtgcgggacgaggtctacgaccaactgtcttacgagggcgtcccctacgatagcagcaagttcgccgagtggcgggacaagcgcagaagatttgtgcttgagaacctgagcatccagctggaagagggcaagaccttcctgacagagttcgacaagctgaataaggacagcctgtaccgctggaacatgaactacctgaaactgctgagaaaggccatccgggccggaggcaaagagttcgccaagatcgctaagacagagatcttcgagctggcggtggaaagattcggccctattaacctgggcagcctgtcccagatcagccttaagatgattgcctcctttaagggcgtggtccagtcctacttctccgtgagcggctgcgtggatgatgcctccaaaaaggcccatgattctatgctgttcacatttatgtgcgccgccgaagaaaagcggaccaacaagagagaagaaaagaccaacagagccgccagctttatcctgcaaaaagcctacctgcatggctgcaagatgatcgtgtgcgaggacgaccttcctgtggccgacggcaagacaggcaaagcccagaatgccgaccggatggactggtgcgccagagccctggccaagaaggtgaacgacggctgtgttgccatgagcatctgctacagagctatccctgcctacatgagcagccaccaggacccctttgtgcacatgcaggataagaaaaccagcgtgctgcggcctagattcatggaagttaataaggatagcatcagagactaccacgtggcgggcctgagaagaatgctgaacagcaagagtgacgctggcaccagtgtttattaccggcaagctgccctgcatttctgcgaagccctgggcgtgagccctgaactggtgaaaaacaagaaaacccacgccgccgaactgggcaagcacatgggcagcgctatgctgatgccctggagaggcggtagagtgtacatcgccagcaaaaagctgacctccgatgccaaatcagtgaagtactgcggcgaggatatgtggcagtaccacgccgatgagatcgccgctgttaacatcgccatgtatgaggtgtgctgccagaccggcgctttcggaaagaaacagaaaaaatcggacgagctgcctgga

SEQ ID NO: 32 - DNA, 3183 bp, synthetic construct
  source 1..3183: mol_type = other DNA
atgagctctgacgtggtgcggccttacaataccaagctgctgccagacaaccggaagtacaacatgtttctgcagaccttcaagagactgaacctgatctccagcaaccacttcgacctgctggtgtgcctgtacgccgctatcaccaacaagaaagctgaggaatacaagagcgaaaaagaggatcacgttacagccgacagcctgtgtgccatcaactggttccggcctatgtctaagcggtacatcaagtacgctacaaccacctttaagatgctggaactgttcaaggagtacagcggccacgagcctgacacctacagcaagaactacctgatgtctaatatcgtgagcgataggttcgtgtgggtggactgccggaaattcgctaaggacttcgccaatcaaatggaactgtccttccacgagttcaccaccctgagtgaaaccctgctggctaacagcatcctggtgctaaatgagtctacaaaggccaactgggcctggggcgccgtgagtgctctgtacggcggcggcgacaaagaggactctacactgaaaagcaagatccttctggcctttgtggacgccctgaacaaccctgaactgaaaacacgtagagaaattctgaaccacgtgtgcgaatctctgaagtatcagagctaccaggacatgtacgtcgatttcagaagcgtggtcgatgataagggcaacaagaagagcccaaacggcagcatgcctatcgtgaccaagttcgagagcgatgatctgatcggcgataaccagagaaagacaatgatctctagctttacgaagaacgccgccgccaaggccagcaagaagcccatcccatacctggacatcctcaaggaccacatgatcagcctgtgtgaagagtacaacgtgtatgcctgggccgctgccatcaccaacagcaacgccgacgtgacagcccgcaacaccagaaacctgacattcatcggagaacagaacacccggaggaaggaactgagcgtgctgcagacaagcaccaacgagaaggctaaagacatcctgaacaaaatcaacgacaacctgatccctgaggtgcggtacacacctgcccctaagcacctgggtcgggacctggccaatctgttcgagatgttcaaggaaaaggacatcaaccagatcggcaacgaggaggagaagcagaacgtgatcaacgactgcatcgaacagtacgtggacgactgtagaagcctgaacagaaacccagtggccgccctgctaaagcacatcagcggatactacgaggatttcagcgccaaaaatttcctggacggcgccaagctgaatgtgctgaccgaagtggtcaacagacagaaggctcatcctacaatctgcagcgaaaaggcctacacctggattagcaagatcgataagaaccggcggcaggccaattcctccctggtcggatgggtggtgccccccgaggaagtgcacaaggaaaagattgccggccagcagagcatgatgtgggtgacactgacactgctggacgacggcaagtgggttaagcaccacatccccttcgccgattctagatactacagcgaggtgtatgcctataatcctaacctgccttatctcgagggcggcatccccagacagtctaagtttggcaacaaacctaccaccaacctgaccgccgaatctcaggccctgttggccaactccaagcacaaaaaagccaacaagaccttcctgagggccaaagagaacatcacccacaacgtgagagtgtctcctaataccagcctgtgcatcagaccactgaaggactctgctggcaatcaaatgttcgacaacatcggcaacatgctgttcggtatgcagatcaaccatagaatcaccgtaggaaaacccaactacaagatagaggtgggcgatagatttctcggattcgaccagaatcagagcgagaaccacacctacgcagtgctgcaaagagtatctgagagcagccacggcacacaccactttaacggctgggacgtgaaagtgatcgagaagggcaaggtgaccagcgacgtggtggtgcgggacgaggtgtacgatcagctgtcctacgaaggcgttccttacgactcccctaagtttaccgaatggcgggaaaaacggagaaagttcgtgctggaaaacatgagcatccagatcgaggagggcaagacttttctgaccgagttcgataagctgaataaagacagcctgtatagatggaacatgaactacatgaaactgctgaggaaggccatcagagccggcggaaaagagttcgccaagatcaccaaggccgagatcttcgaactgggcgtgatgagattcgggcctatgaacctgggcagcctgagccaagtgagtctcaagatgatcgccgccttcaagggagtgatccagagctacttctctgtgtctggctgcatcgatgatgcttccaagaaggcccacgacagcatgctgttcgccttcctgtgtagcgccgatgaaaagcggaccaacaagcgggaagaaaagaccaatcgggccgccagcttcatccttcaaaaggcctactcccacggctgtaaaatgattgtgtgcgaggacgaccttcctatcgccgatggcaaagtgggaaaggcccagaacgccgacagaatggactggtgcgcccggagcctggctaagaaagtgaacgatggctgcgtggccatgtccatctgctacagagccatccccgcctacatgagctcccaccaggaccccttcacccatatgcaggataagaaaaccagcgtgctgcggcctagatttatggaagttggcaaggacagcatccgggaccaccacgtggctggcctgagacggatgctgaatagcaagggcaacacaggcaccagcgtgtactacagagaggccgcactgcgcttctgcgaggccctgggcgtgctgcctgagctggtgaagaataagaaaacacacgccagcgagctgggaaagcatatgggcagcgcaatgctgatgccttggagaggcggcagaatctacgtggccagcaagaaactgacaagcgacgccaaatctatcaagtactgcggcgaggatatgtggcagtaccacgccgacgagatcgctgctatcaacatcgccatgtacgaggtc

SEQ ID NO: 33 - DNA, 3225 bp, synthetic construct
  source 1..3225: mol_type = other DNA
atgggcatctctatcagcagaccttacggcaccaaactgcggcctgatgccagaaagaaagaaatgctggataaattcttcaccaccctggccaaaggccagagagtgttcgccgacctgggcctgtgcatctacggcagcctgacactggagatggtgaaaagactggagcctgagagcgacagcgagctggtgtgcgccatcggctggttccggctggtggataaagtgacctggagcgaaaacgagatcaagcaggaaaacctggtgcggcagtacgaaacctactctggcaaggaagccagcgaggtgatcaagacctatctgagcagtccctcttctgataagtacgtgtggatagattgcagacagaagtttctgcggttccagcgggacctgggcacaagaaacctgtccgaggatttcgagtgcatgctgttcgagcagtatctgagactgactaagggcgagctggatggacacaccgccatgagcaatatgttcggcaccaagacaaaggaggatagagccaccaagctgcgatacgccgccagaatgaaggagtggctggaagctaatgaggagatcacctgggaacagtaccaccaggccctgcaggataagctcgacgcgaacactctggaggaagccgtggataactacaagggcaaggctggcggaagcaaccctttctttagctacaccctgctgaaccgaggacagatcgacaagaaaacccacgagcagcagctgaagaagttcaacaaggtgctgaaaaccaagtctaagaacctgaacttccctaacaaagagaagctaaagcagtacctcgagacagcgatcggaatccccgtggacgctcaggtgtacggccagatgtttaacaacggcgtgtctgaggttcaacctaagacaaccagaaacatgtcctttagcatggaaaagctggagctcctgaacgaactgaagagcctgaacaagaccgacggattcgagagagccaacgaggtgctcaatggcttcttcgacagcgaactgcacacaacagaggacaaattcaatatcacaagcagatacctgggcggcgacagaaacaaccggctccctaagctgtatgagttgtggaagaaggagggcgtggacagagaggagggcatccagcaattttcccaagccatccaggacaagatgggccaaatccctgttaagaacgtgctccgctacatctgggagttccgggaaaccgtgagcgcagaagatttcgaggctgctgccaaggccaaccagctggaggaaaagatcacccggaccaaagcccaccccgtcgtgatcagcaacagatactggaccttcgggtccagcgccctggtgggcaacatcatgcctgccgacaagatgcacaaggaccagtacgccggccagagctttaagatgtggctggaagctgagctgcactacgacggcaagaaggtgaagcaccacctgcccttctacaatgccagattcttcgaggaggtgtactgctaccacccatcagtggccgaagtgaccccttttaagaccaagcagttcggatatgccatcggcaaggacatcccagctgacgtgtctgtggtgctgaaagataacccctacaagaaggccaccaagagatttctgagggccatcagcaatccagtcgccaacactgtggacgtgaacaagcctacagtgtgtagcttcatgatcaagcgggaaaacgacgagtacaagctggtgatcaacagaaagatcggagtggacagacccaagagaatcaaggtgggcagaaaagtgatgggctacgacagaaaccagaccgccagcgacacatattggatcggcgagctggttcctcatgggaccacaggcgcctacagaatcggagaatggagcgtgcaatacattaaaagcggccctgtgctttcttctacacagggcgtgaacgattctaccaccgatcagctgatctacaacggaatgcccagcagcagcgagcggttcaaggcctggaagaagtccagaatgagcttcatccggaagctgatcagacagctgaatgccgaaggcctggaaagcaaaggacaggactacgtgcccgagaaccctagcagcttcgacgtcagaggagaaacactgtacgtgtttaacagcaactacatgaaagccctggtgtccaagcacaggaaggccaagaagcccgtggaaggcatcctggaagaaatcgaggctctgacctccaaagccaaggacagctgcagcctgatgcgcctgagctctctgagcgacgccgccatgcagggcatcgccagcctgaagtccctgatcaactcttatttcaacaagaatggctgtaaaaccatcgaggacaaggaaaagttcaaccccgacctgtacgtgaagctggtcgaggtcgaacagaaaagaaccaacaagcggaaggagaaggtgggccggatcgccggcagcctggaacagctcgccctgctgaatggtgttgacgtggtgatcggcgaggccgatctgggggaagtcaagaaaggcaagtctaagaagcagaatagcagaaacatggactggtgcgccaagcaggtcgctgagcgcctggaatacaaactgaccttccactgtatcggctacttcggcgtgaaccctatgtacacaagccaccaagatccttttgaacaccggagagtggccgaccacctggtgatgagagctaggttcgaagaggtgaacgttagcaacgtaagcgaatggcacatgagaaacttcagcaattacctgcgggccgacagcggcacaggtctgtactacaagcaagccaccctggactttctgaaacattacgacctggaggagcacgccgacgacctggagaaacagaatatcaagttctacgatttcagaaagatcctggaggacaagcagctgacatctgttatagtgcctaagcggggcggcagaatctacatggccacaaaccccgtgacatcagacagcacccctgtgacctacgccggcaagacctacaatagatgcaacgccgatgaggtggctgccgctaatatcgctatttctgtgctggcccctcacagcaagaaggaagagaaagaggataagatccctatcatcagcaagaagcctaagtccaagaacaccccaaaggctagaaagaacctgaaaacaagccagctgcctcagaag

SEQ ID NO: 34 - DNA, 3009 bp, synthetic construct
  source 1..3009: mol_type = other DNA
atggccagcaaacacgtggtgcggccttttaacggcaaagtgaccgctaccggcaagcggctggcctacctggaggaaacctttcattacctggagaaggccgccggcggcgtgtctaccctgttcgccgctctgggcagctacctcgacgccacaaccatcagcaacctgatcaacaagaaccaggacttggctgtcgtgatcttccggtaccacgtggtgcctaagggcgaagcccacacactgcccgtgggcaccgacatggtgtcaaggttcgtggccgactacggcatggagcctaatgagttccaaagagcctacctggatagccccatcgatcaggagaagtactgctggcaggacaatcgggacgtgggatgttggctgggcgaacagctgggtgtttctgaggccgacatgcgggctatcgccgtgactttttacaacaaccagatgctgtacgactgtgtgaagggaactggcagcggcaatgccgtctctctgctgtttggcagcggcaagaagtccgactacagcatgaagggagtcattgccggcaaggctgcctcagtgctggctaagtatagacctgccacctaccaggatgccagaaagatgatcctggaagctaatggcttcaccagcgtgaaagatctggtcacatcttacggcatcaccggcagaagcagcgccctgcaaatcttcatggaaggcattgaaagcggacctatctcctccaaaacattggacgccagaatcaagaagttcacggaagatagtgagcggaacggccgcaagaacctggtcccccacgccggcgccattagaaattggctgatcgagcaggccggttcttctgtggaaaactaccaaatggcctggtgcgaggtttacggcaacgtgagcgctgactggaacgccaaggtggaaagcaacttcaacttcgtggccgagaaggtgaaagccctgaccgagctgagcaatatccagaagagcacccctgatctgggcaaggctctgaaactgtttgaggagtacctgaccacatgccaggacgagttcgccatcgccccataccacttcagcgtgatggaagaggtgcggatggaaatggccacaggcagagagtttaacgatgcatacgacgacgctctgaacagcctggacatggaaagcaagcagcctatccagcctctgtgtaaattcctgatcgagcggggcggaagcatcagcttcgacaccttcaagagcgccgccaaatacctgaaaacccagagcaagattgccggcagataccctcatccattcgtgaagggaaaccagggcttcacattcggctccaagaacatctgggccgccataaacgaccccatgatggagtacgccgacggccggatcgccggcggctctgccatgatgtgggtcaccgctaccctgctggacggcaagaagtgggtgagacaccacatccccttcgccaacacaagatacttcgaggaggtttacgccagcaagaagggcctgcctgtcctgccgtgcgccagagatggcaagcacagctttaagctgggtaacaacctgagcgtggagagagtggaaaaggtgaaggaaggcggcagaacaaaggccacaaaggctcaggagagaatcctgagcaacctgacacacaacgtgcagttcgacagcagcaccaccttcatcatccggagacaggaggaatcctttgtgatctgcgtgaaccacagacaccccgcccctctgatgaagaaggagatggaagtgggcgacaagatcatcggcatcgaccagaacgtgaccgcccctaccacctacgccatcgtggagagggtggccagcggaggcatcgagcggaacggcaaacagtacaaggtgacagccatgggcgccatctcctctgtgcagaaaaccagaggcggagaggtggacgtgctgagctacatgggtgtggagctgtccgactcgaagaacggattccagagcctgtggaacaagtgtctggacttcgtgaccaagcacggcacagagaacgacgtgaagtactacaacaacacagccgtgtgggccaacaagctttacgtgtggcacaagatgtacttcagactgctcaagcaactgatgagaagagccaaggacctgaagcctttcagagatcacctgcaacacctgctgttccaccctaacctgtctcctctgcagcggcatagcctgtctcttacaagcctggaggctaccaagatcgtgcgcaattgcatccacagctatttcagccttctcgggctgaaaaccctggatgagagaaaggcagccgacatcaacctgctcgaggtgctggaaaagctgtatgccggccttgtggaaagaaggaaggagagaaccaagctgacagccggcctgctggtcagactgtgcaacgagcacggaattagctttgccgccatcgaaggcgacctgcctgtggtgggcgaaggcaagagcaaggccgctaacaacacccagcaggactggaccgcccgggaactggagaagagactgagcgaaatggctgaggtggtgggcatcaaggtgatcgctgttctaccacactacaccagccaccaggaccctttcgtttactccaagaataccaagaaaatgcggtgcagatggaattggcggaccaccaagaccttcaccgatagagatgccctgagcatccggagaatcctgagcaagcccgaaaccggaaccaacctgtattaccagaagggactgaaggccttcgccgagaagcacggcctggatctggccgaaatgaagaagcggaaggacgcccagtggtacctggaaagaatccaggataagaacttcctggtgcccatgaacggcggaagagtgtacctgagcagcgtgaagctggccggcaaagagacaatcgacatgggcggcgagattctgtacctgaacgacgccgatcaggtggccgccctcaacgtgctgctggtgaagatc

SEQ ID NO: 35 - DNA, 3054 bp, synthetic construct
  source 1..3054: mol_type = other DNA
atggccaaaaaggaacacattatcagacctttcaagggcaccctgccactgcggggggacagactgagatacctgcaggacaccatgaagtacatgaagaaggttgaggacaccatcaccgagctgtgcgccgccgtgatcgcctacgccaagcctacaatcatccagcagattctgggagaagaaatcgagactacctccaccttctgcagcttcagactggttgggattcatgagaacttcactatgcccctgacaaccaatatgatcaagcacttccagaaaaccttcaacatcaatccttctgagaagcaggccatctatctgagcagcggatttgatagcgacaaatacagatggcaggatacaagcgaggtgtctagaaatttcgctaataagtgccgcctgaccaaccaggagttccaggagttcgccgagcaagctctgttaaacatgtgctttatcggctgtagcggatctcctggcgccacaaacgccgtgtcccagatcttcggcaccggcgaaaagtctgattaccagcggaagtctcagatcgccaagatcgccgctgataccctcgagaaccacaaacctagcacatacgagtctgctaggctgatggtgctgaacacactgggacacaagacgatcgaagattgcgtgaacgactacggcgctattggagccaagtccgccttccggctgtttatggaaagtaaagaaatcggcccaatcaccagcgaacaactgaccacaaaaatcaagaaattcagagaggaccacaagaagaacagcatcaagaagcagctgcctcatgtggaaaaggtgcggaacgcactactgagccagttcaaggagcagtacctgccaagcgcctgggccgaggcctggtgtaacatcatgggagagttcaatagcaagctgtccaacaacaacaatttcatcgaccaaaaaaccaagatggtcaacgactgcgacaacatcaaaaaatctaacccccagctggataaggccgtgaatatgctggacgaatggaagtacaagaattgggacgacaattctgccatccacccctaccacatcggcgatctgaaaaagctgatggccatcttcaacatcaacaatgagggcaccttcgacgagagattcagcgccagctgggagcagttttctaccagcctggagtacggcgagaagccccccgtgcgggacctgctggcccacatcatcaagaacatgaacgacctgacttacaccgacgtgatcaatgccgctaagttcctgaagctgcaagataatatcagaaacaagtatcctcacccttttgtgatgcctaacaagggatgtaccttcggcaaggataacctgtggggcgagatcaatgatcctacagctaagatcaagtccacagaggaagtggccggccagcggcctatgatgtggctgaccgccaagctcctggacaacggcaaatgggtcgagcaccatatccccttcgcctctagcagatacttcgccgaagtgtactacaccaaccccgccctgcctaccttacccatcgcccgcgacggcaagcacagctacaagctgaccaagaccatcgacgccaacaccgccaaaaccctggtgaacaaccctagagacaaggccgccaagctcattgccagaacaaaggcgaacaccacccacaacgtgaagtggatcaaacctacatacagaatccagaaagagaacaaccagttcgtgatcaccatcaatcacagacacccatgtatcacccctcctaaggaaatcatcttgggcgatagaatcctgtcattcgaccaaaacgagacagcccctaccgcctttagcatcctggaaaagaccaccaagggcacagagttctgcggccaccacatcaaagtgctgaaaaccggcatgctggaagccaagatcaagacatcgaagaaatccatcgacgccttcacctacatgggccctatggaggacgaccacgccagcggtttccccaccctgctgaacatctgtgaaaagttcatcagcgagaacggcgacgagaaggacaagagcttcagcagcagaaagctgccttttaagagaagcctgtattttttccacggcagccacttcgacctgctgaagaagatgatccggaaggctaaaaatgaccctaagaaactgaagctggtgagaatccacatcaacgagatcctattcaacagcaacctgtcccctatcaagctgcacagcctgagcatccactctatggagaacacaaaaaaggtgatcgctgccatctcttgctacatgaacgtacacgagtggaaaaccatcgatgagcaaaaaaacgccgacatcacactgtacaacgccaaggaaaagctgtacaacaacctggttaatagaagaaaggaaagagtgaaggtgaccgctggcatgctgatccggctggcccgggaaaacaactgcagattcatggtgggcgaagccgaactgccaacacagcagcagggcaagagcaagaagaacaacaacagcaagcaggactggtgcgccagagacatcgcacagagatgcgaggatatgtgcgaggtggtgggcatcaaatggaacggcgtgacacctcacaacaccagccaccagaatccattcatctacaagaacacctccggccagcagatgcggtgcagatacagcctggtcaaaaagtctgagatgaccgataagatggctgagaagatccggaacattctgcacgccgagcctgtgggcacaaccgcttattacagagagggcatcctggagtttgccaagcaccacggactggacctgggcatgatgaagaaaagaagagatgccaagtattacgacaacctgcccgacgaatttctgctgccgacaagaggcggaagaatatacctgtcggaaaaccagctgggcggcaacgagacaatcgtgatcaacggcaagaaatacttcgtgaatcaggccgaccaggtggccgccgtgaacatagggctgctgtacctgctgcctaagaagaaccagagc

SEQ ID NO: 36 - DNA, 3141 bp, synthetic construct
  source 1..3141: mol_type = other DNA
atgagcgagaagaaattccacatcagaccctacagatgcagcatctcccctaacgcccggaaggccgacatgctgaaggctaccatctcctacctggacagcctgacctctgtgttcagaagcgggtttaccgccctgctggctggaatcgatcctagcaccgtgtccaggctggctcctagcggcgccgtgggcagccccgacctgtggagcgccgtgaactggttcagaatcgtgcccctggccgaagccggcgatgccagagtcggccaggcaagcctgaaaaacctgtttagaggctacgccgggcacgaacctgacgaggaagccagcatctacatggaaagcagagtggacgacaaacggcacgcctgggtcgactgcagggccatgttcagagctatggccctcgagtgcggcctggaggaagcccagctggcttccgacgtgttcgccctggccagcagagaggtgatcgtgttcaaggacggcgaaatcaacggctggggcatcgccagtctgctgttcggcgaaggagagaaggctgattctcagaaaaaggtggccctgctgagaagcgtgagactggccctcgagggcgattacgctacctacgaggagctgtctggcctgatgctggccaagaccggcgccagctctggctccgatctgctggacgagtacaaacggtccgaaaaaggtggctcttctggaggcagacatcctttctttgacgaggtgtttcggagaggcggcagagttaaacaggaggaaagagagagactcctgaaaagctgcgacaccgcaatccagaagcagggacaggccctgcctctgtctcacgtggccagctggcggcagtggttcctgagaagagtgaccctgctgaggaatagacggcaggagagcttcgctgtgtgcatcacaaacgccctgatggacctgcaacccaagaacctgagaaatgtgcactacgtgaccaaccccaagagcgagaaggataagggggttctggaactgcgggtggacgtcaaaaacaacgagggccctgatgtggctggcgcccaagccgtgtttgacgcctacatggccagacttgccccagatctgagattcagcgtgatgcctagacatctgggctcactgaaggacctgtacgccttgtgggccaagctgggaagagatgaggcgatcgaggagtacctggaaggctatgagggccctttcagcaaaagaccaatcgccggcatcctgcagatcatccacgcccatcggggcaaggtggggcacgacagcctgttgagagccgccagacttaacagagctatggatagactggagagaaaaagagcccacgcctgtgccgccggcaacaagggatatgtgtacggcaagagcagcatggtgggccggatcaaccctcagagccttgaagtgggcggacggaagtctggccggagccccatgatgtgggtgacactggacctggtcgacggcgacagattcgcccagcaccacctgccctttcaatctgcccggttcttcagcgaagtgtactgccacggagacggcctgcccgccaccagagtgccaggcatggtcagaaaccggagaaatggcctggccatcggaaatggcctgggcgagggaggactgagtgctctgagagccggaagcgaccggagaaagcgggctaacaagagaacactgagagccctggagaatatcacccacaacgtggaaatcgatcctagcacatccttcacactgagagaggacggcatcatcatcagccacagaatcgagaagatcgagcctaagctggtggcttttggagacagagctctgggcttcgacctgaaccagaccggcgcccacacctttgccgtgctgcagaaggtggacagcggcgggctggatgtgggtcacagccgggtcagcattgtgctgaccggcaccgtgcggagcatctgcaagggcaatcaggccagcgggggccgggactacgacctgctgtcttacgacggccccgagagagatgatggcgcttttaccgcctggaggtctgacagacaggcctttctgatgagcgccattcgggaactgcctacccctgccgagggcgagaaagattacaaggccgacctgctgtcccagatggccagcctggaccactaccggaggctgtacgcctacaacagaaagtgcctgggcatctacatcggtgccctgcggcgcgccacaagacggcaggccgttgccgccttcaaggacgagattctgtccatcgccaaccacagatgcggccccctgatgagaggctccctgagcgtcaacggcatggaaagcctggccaacctgaagggcctggcaaccgcttatctgtctaagttcaaggacagcaagtccgaggacctgctgagtaaggacgaagaaatggccgacctgtacagagcttgcgccagacgcatgaccggaaaaagaaaggaacggtaccggcgtgctgccagcgaaatcgtgagactggctaacgagcacggctgtctgttcgtgttcggcgagaaggaactgcctacaaccagcaagggcaacaagtctaaacagaaccagcggaacaccgactggtcggcccgggccatcgtgaaggccgtgaaggaggcctgcgagggatgtggcctgggcttcaagccggtgtggaaggaatactctagcttgaccgaccccttcgagagggacggcgatggccggcctgctctgagatgtagattcgccaaggtggctgctcccgacagcgagctcccacctagactgacaaaggccgtgggaagctatgtgaagaacgccctaaaggccgataaggccgagaagaaacaaacatgttaccagagaggagccatcgagttctgcagcaggcacggcatcgacgtccggaaagctacagataaggccattcggaaagctgtgcggggtagcagtgacctattagtgcctttcgatggaggcagaaccttcctgctatcaacaagactgagccctgagagcagaaaggtggaatgggccggaagaacactgtacgagttcccttctgatatggtggccgccatcaacatcgcctgccggggcctggaacctagaaaggca

SEQ ID NO: 37 - DNA, 3087 bp, synthetic construct
  source 1..3087: mol_type = other DNA
atggacgagcaggccgtggtgagcagcggctctgataagaccctgaagatcgtgaggccctacagagctaaggtgaccgctactggaatcagattggaagggatcaaaaacaccctgaattacctgaagagaacagagatttgtctgtccagactgaacgccgcttgcggcgcctttctgacccctgccatcgtggagcagatctgtaaagacgatcccgccctggtgtgcgccatagctagattccagctggtgcctgtgggcagcgaagctaccctgagcgatagcggactgatgcggcacttcaaggcggcgctgggcgaactgacccctctgcaggaagcctacctgaacagcagttataacgatgagctgtacgcctggcaggataccctggtgctggccagacagatcatcgcggaaaccggcctgaccgaggaccagttccgggcatttgcccacgcctgcttcaagaacggtaatatcatcggttgtgccggaggccctggcgcaagcaatgccattagcggcatcttcggcgagggaatcaagagcgactacagcctccgcagcgagatgacagccgctgtggctaaggtgttcgaggaaaagcggcccatcacatacgaggaagccagagccctggccctcgaagccaccggccacgcctctgtgcagagctttgtcgaggcctttggcaaacagggcagaaagggcaccctgatcctgttcatggaggacaccaaaacaggcgccttcccctccaacgagttcgactataagctgaagaagctgaaggaggacgcagagcgggtgggcagaaagggcatcatcccacatcgggacgtgatcgcctcttacctccggaaccagaccggagccgacatcgagtacaacagcaaggcctggtgcgaaagctactgctgcgccgtttctgaatacaacagcaagatgagcaacaacgtgcggttcgctacagagaagagcctggacctgactaagctggacgagacaatcagggaaaccccaaagatcagcgaggccatgctggtgttcgagaactacatggccagaatcgatgccgacctgaggttcatcgtgtcgaagcaccacctgggaaacctggccaagttccggcaaacaatgatgcacgtgtccgccagcgagttcgaggaagccttcaaggccatgtgggccgattacctggctggcttggagtatggcgagaaacctgctatctgcgagctggttagatacgtgctgacccacggcaatgacctgcctgtggaagccttttacgccgcctgcaagtttctgtccctggacgacaagatcaagaacagataccctcatcctttcgtgcccggcaacaagggctatacattcggcgcaaagaacctctgggccgagatcaacgaccctttcaagcctatcagacagggcaatcctgaggtagccggccaaagacccatgatgtgggccacagctgatctgctggacaacaacaagtgggtgctgcaccatattccttttgcctcgagcagatactttgaggaagtgtactacacagacccatctctcccaaccgcccagaaggccagagacggcaagcacggctacagactgggaaaggtgctggatgaggccgccagagaaagactgaaggccaacaacagacaaagaaaggccgccaaggccatcgagcggatcaaggccaattgcgagcacaatgtggcctgggaccctaccaccaccttcatgctgcaactggacagcgagggcaacgtgaagatgaccatcaaccacagacacatcgcctaccgggctcctaaggaaatcggcgtgggcgaccgggttatcggcatcgaccagaacgaaaccgcccctacaacatacgccatcttggaaagaacggaaaacccccgggacctggaatataacggcaagtactacagagtggtgaagatgggcagcgtgacctctcctaacgtgtccaaatacagaaccgtggacgccctgacttacgacggcgtgtctctgagcgacgacgccagcggagccgtgaacttcgtcgtgctgtgcagagagttcttcgccgctcatggcgacgacgagggccggaaatacctggagagaaccctgggctggagctccagcctgtatagcttccacggcaactacttcaagtgcctgacccagatgatgcggagaagcgcccgctctggcggcgatctgaccgtgtaccgcgctcacctgcagcagatcctgtttcagcacaacctgtcccctctgagaatgcacagcctgagcctgcggagcatggaatctaccatgaaggtgatcagctgcatgaagtcttacatgagcctgtgcggctggaaaaccgatgctgacagaatcgccaacgaccggagcctgttcgaagccgccagaaagctgtacacatctctggtcaatcggcggaccgaaagagtgcgggtgacagcaggcatccttatgagactgtgtctggagcacaatgtgcggtttatccacatggaggacgagctgcctgtggctgaaaccggcaaaagcaaaaaaagcaacggcgccaagatgcactggtgtgcccgggagctggcagttagactgtctcagatggccgaagtgaccagcgttaagttcaccggagtgagcccccactacactagtcaccaggaccccttcgtgcactctaaaaccagcaaagtgatgcgcgccagatggtcctggcggaaccgggccgacttcacagataaggacgccgagagaatccggactatcctgggcggcgatgacgccgggaccaaagcttactacagaagcgccctggccgagttcgccagcagatacggcctggatatggagcaaatgagaaagagacgggatgcccagtggtaccaggagagactgcctgaaaccttcatcatcccccagagaggcgggagagtgtacctgagctcccacgacctgggcagcggccagaaagtggacggcatctacggcggaagggccttcgtgaatcacgctgatgaggtggccgcccttaacgtggctctggtccgcctc

SEQ ID NO: 38 - DNA, 3087 bp, synthetic construct
  source 1..3087: mol_type = other DNA
atgaaaacagagacactgatccgcccttaccccggcaagctgaacctgcagcctcggcgggcccaattcctggaggattcaatccagtaccaccagaaaatgaccgagttcttctaccagttcctgcaggccgtaggcggcgcgaccacacatcagaacatcagcgatttcattgacaacaaggccactgatgagcaccaggccacccttctcttccaggtcgtgtccaaggacagcaccacccctgagtgccctgccgaggaactgctggccagattcgcccagtacaccggcaaacagcccaacgaggccgtgacccactacctgaccagcagaatcaacaccgacaagtacagatggcaggacaatagactactggcccagaacatcgccagccaacttaacatctccgagacacaattccaggaaatcgcgcacgctatcctcagcaacaacctgtacatcggacagaccgccagcaacgctgccgccaacttcatctctcaggtgaccggcaccggccagaaagccccaaaggctgccagactggacgtgctgttccagacgaaccaagccctggccaaaacccagcctacaacctttggccagctccagcagattatcgtgcaggcttgtggagaaagcaccaccgacgccgtgctggccaagttcggcaacaaaggtgccgccacctcgctgcagctggctctgaaaaccgaccccaacaccaccctggatcagaaaaagtatgaggccctgcaaaagaaattcgccgaggacgaaacaaagtaccggaacaaggttgacattccccacaaaacgcagctgagaaatctgatcctgaacacaagcaatcaattttgcaactggcacacaaagcctgccatcgaggcttttaagtgcgccatcgccgacatccagagcaaggtgtccaacaacctgaggatcatgcaggagaaggccaagctgtacgaggccttcagaaacgtggacccccaggtgcagatcgctgtccaagccctggagaatcacatgaacaccctcgaagaaccctacgccccttacgcccacagcttcggcagcgtgaaggacttctatgaggacctgaacaacggcagcaatctggacgaggcaattcagaccatcgtgcacgattctgataacttcaaccggaagcctgatcctaactggctgagaatcatcgccccactgcactctagccacagcgcctctcagatcatggaagctgtgaaatacctgagcagcaagcaggactacgaactgaggaagcccttcccattcgtggccaccaacctgcctgccacatacggcaagttcaatatccccggcaccctgaaccctcctacagactctctgcacggcagactgaacggctctcacagcaacatgtggctgacagccctgctgctggacggcagagactggaagaaccaccacctgtgcttcgccagcagcagatacttcgaagaagtctacttcaccaaccctagcctgcccaccaccgataaagtgcggtccccaaagtgcggctttaccctgaagagcgtgctggacagcgaggctaaggatagaatccgtaatgcccctaagagcagaaccaaggccgtgaaggccatcgagagaattaaggctaattctacccacaacgtggcctggaaccccgagacaagcttccagatgcagaagagaaacgacgagttctacatcacaatcaaccacaggatcgagatggaaaagatccccggccaaaagaaaacagacgacggcttcaccatccaccccaagggcctgtttgctatcctgaaggaaggagatagaatcctgagccaggatctgaatcagacagccgctacacactgcgccgtgtacgaggtggccaagcctgaccagaacaccttcaaccaccatggcatccacctgaagctgatcgccaccgaagaactgaagatgcctctgaaaaccaagaagtctaccatcccagatgccctgtcataccagggcatccacgcccacgaccgggaaaacggcctgcagcagctgaaggacgcttgcggagccttcatctcacctagactggaccccaagcagaaggccacctgggacaacagcgtgtccaagaaagaaaacctgtaccctttcatcaccgcctacatgaagctgctgaagaaggtgatgaaggcgggccggcaggagctgaagctgtttcggactcatctggatcacatcctgttcaaacacaatctcagccctctgaaactgcacggcgtgagcatgatcggcctggagagcagcagagctacaaaaagcgtgatcaacagcttcttcaacctgcagaacgctaagactgagcagcagcagatcgccttagacagacccctgttcgaggccggcaagacactgatcaataatcagaccagaagaaggcaggaaagagtgcggctggaaacatctctgaccatgagactggcccataagtataacgctaaagccatcatcattgagggagagctgcctcacagctccaccggcacatctcagtaccagaacaacgtgcggctggattggagtgccaagaagagcgccaagctgaaaaccgaaagcgccaactgcgctggaatcgccatctgccagatcgacccttgtcacacctcccaccagaacccttttcggcacacccctacaaaccctgacctgcggccacggttcgcccaggtgaagaaaggcaagatgttccagtaccagcttaatggcctccagcggctgctgaatcctagatcaaagtctagcacagcaatctactaccggcaggccgtgcaaagcttttgtgcccaccacaacctgaccgagagagacatcacctctgccaaatttcccagcgacctggaaaagaagatcaaggacgacacctacctgatccctcagagaggcggccggatctacatcagtagcttccctgttacaagctgcgccagaccttgcacaagcaaccattatttcggcggaggccagttcgagtgtaatgctgatgccgtggccgccgtgaacatcatgctgaaggtccaccct

SEQ ID NO: 39 - DNA, 3033 bp, synthetic construct
  source 1..3033: mol_type = other DNA
atgcctatccggggctataagtgcaccgtggtgcctaatgtgcggaaaaagaaactgctggagaaaacatacagctacctgcaggagggcagcgacgtgtttttcgatctgttcctgtcactgtatggcggcatcgcccctaagatgatccctcaggatctgggcatcaacgagcaagtgatctgtgccgcaaactggttcaagatcgtggaaaagaccaaggactgcatcgccgacgacgccctgctgaaccagtttgcccagtactacggcgagaagcctaacgagaaggttgtgcagtttctgacagcttcttataacaaagataagtacgtgtgggtcgactgccgtcaaaagttctacaccctgcagaaagacctgggagtgcagaacctcgagaacgacctggagtgcctgatccgcgaggacctgctgcctgtgggatctgataaggaagtgaatggatggcacagcatcagcaaactcttcggctgcggcgagaaggaggacagaaccatcaaggccaagattctgaacggcctgtgggagcggatcgagaaggaagatattctgaccgaggaggacgccagaaacgagctgctgcatagcgctggcgtgctgacccctaaggagttcagaaaggtgtacaagggcgccgccggcggacgggactgctaccacaccctgctggttgacggcagaaacttcaccttcaacctgaaaaccctgatcaagcagaccaaggacaagctcaaggaaaagtccgtggatgtggaaatccccaacaaggaggccctgaggctgtacctggaaaagcgaatcggaagatctttcgagcagaagccttggtccgagatgtacaaaaccgccctgagcgctgttatgcccaagaacaccctgaattactgctttgccatcgatagacacgcccagtacacgaagatccagaccctgaagcaaccttacgactctgccatcaccgccctgaacggcttcttcgagagcgaatgcttcaccgggagcgacgtgttcgtgatcagccctagccacctgggaaaaaccctgaagaagctgtacaactacaaggacgttgagagcggaatcagcgagatcgtcgaggacgaggataatagcctgcggagcggcgtgaacgtgaatctgcttcggtacatcttcacactgaaggatatgttcagcgccgaggacttcatcaaggccgccgagtacaacgtagtgtttgagagatacaatagacagaaagtccaccctacagtgaagggcaatcaaagcttcacatttggcaacagcgctctgtctggcaaggtgatccctccatctaagtgtctgagcaacctgcctggacagatgtggctggccatcaatctgctggaccagggcgagtggaaggagcaccacattcccttccacagcgccagattctacgaggaaatctacgctacatctgataaccagaacaaccccgtggacctgcggaccaagagattcggctgttctctgaacaagaccttcagcgccgctgacatcgagaaggtgaaggagtctgccaagaaaaagcacggaaaggccgctaagagaatcctgcgtgccaagaacacaaacaccgccgtgaactgggtggattgcggcttcatgctggaaaagaccgaagtgaacttcaaaatcaccgtcaattacaaactgcccgatcagaagctgggcaagttcgagcctatcgtgggcacaaaaatcctggcttatgaccagaatcagaccgccccagatgcctacgccatcctggaaatttgcgacgattctgaagccttcgactacaagggctacaaaatcaaatgtctgagcaccggggacctggccagcaagtccctgacaaagcagacagaagtggaccagctggcatataagggcgtagacaaaaccagcaacttctacaagaagtggaagcagcagcggagactttttgtgaagagcctgaatatcccagacgccctgaaatcttttgaaaacatcaacaaggagtacctgtacggctttaacaatagttacctgaagctactgaagcaaattctgagaggcaaattcggacctatcctggtggacatcagacctgagctgatcgagatgtgccagggcatcggcagcatcatgcggctgtccagcttgaaccacgacagcctggacgccattcagtccctgaagagcctgctgcactcttacttcgacctgaaggtgaaggaagaaatcaagaccgaagagctgagagagaaggccgataaggaagtgtttaagctgctgcaacaggtgatccagaagcagaagaataagagaaaggaaaaggtgaacagaacagtggatgctatcctgacactggccgccgacgagcaagtgcaggtgatcgtgggcgaaggcgacctgtgcgtgtccaccaagggcaccaaaaagagacagaacaaccggacaatcgactggtgcgcgagagccgtggtcgagaaactggaaaaagcctgcaagctgcacggcctgcacttcaaggaaatccccccccactacaccagccaccaggactgtttcgagcacaacaaggacatcgagaatcctaaggaagtgatgaagtgtagattcaacagcagcgagaacgtggccccttggatgattaagaagttcgccaactaccttaaatgcgagacaaaatactacgtgcagggcatgcaggacttcctggaacattacggcctggtggaatacaaggaccatatcaagaagggaaagatcagtatcggcgattttcagaaactgatcaagctggccctggaaaaagtaggcgagaaggaaatcgtgtttccttgcaaaggcggcagaatctacctgagcacctactgtctgaccaacgagtccaaacccatcgtgttcaacggcagacggtgctatgtgaacaacgccgaccacgtggccgctatcaacgtgggcatctgcctgttgaatttcaacgccagagctaaggtggctgaaaagacacca

SEQ ID NO: 40 - DNA, 3279 bp, synthetic construct
  source 1..3279: mol_type = other DNA
atggccaagaaggacttcatcgccagaccttacaacagctttctgctgcctaacgacagaaagctggcttacctggaagaaacatggaccgcctacaagagcatcaagaccgtgctgcacagatttctgatcgcggcctatggcgccatccccttccagacattcgccaaaaccattgaaaacacccaagaggacgagctgcaactggcctatgccgtgcggatgttcagactggtgcccaaggacttcagcaagaacgagaacaacattccacctgacatgctgatcagcaagctggccagctacaccaatatcaaccagtccccaacaaacgttctcagctacgtgaatagcaactacgacccagagaaatacaagtggatcgattctagaaacgaggccatcagcctgagcaaggagatcggcatcaagctggacgagctcgctgattacgccaccaccatgctgtgggaggattggctgcccctgaacaaggacacagtgaacggctggggaaccacctctggcctgttcggcgccggcaaaaaagaggataggacccaaaaggtgcagatgctgaacgccctgctgctgggcctgaaaaacaacccccccaaggattacaagcagtacagcaccatcctactgaaggcatttgatgccaagagctgggaagaggccgtgaagatttacaaaggcgagtgttctggccgaacaagtagttacctgactgagaagcacggtgacatcagccctgagacactggaaaagctgatccagagcatccagcgggacatcgccgacaaacagcacccaatcaacctgccaaagagagaagaaatcaaagcctacctggagaaacagtctggcaccccatacaacctgaacctgtggagccaggccctgcacaacgccatgagctctatcaagaaaaccgacaccagaaatttcaactctaccctggagaagtacgagaaggaaatccagctgaaggagtgccttcaagatggcgacgatgtggagctgctggggaacaagtttttctcttctccttaccacaagacaaatgatgtgttcgtgatctgctctgaacacatcggaacaaatagaaagtacaacgtggtcgagcagatgtatcagctggccagcgagcacgccgacttcgagacagttttcaccctgctgaaggacgagtatgaggaaaagggcatcaagacacccatcaaaaacatcctggagtacatctggaacaacaagaacgtccctgtgggcacatggggccggatcgctaaatacaaccagctgaaggacagattagcagggatcaaggccaatcccacagtggaatgcaacagaggcatgacatttggcaacagcgccatggtgggcgaagtgatgcgctccaaccggatcagcaccagcaccaagaacaagggccagatcttggcccagatgcacaacgaccggcctgtgggcagcaacaacatgatttggctggaaatgaccctcctgaacaacggcaagtggcagaagcaccacatccccacacacaacaacaaatttttcgaggaagtgcacgccttcaaccctgaactgaagcagagcgtgaacgtgagaaacagaatgtacagaagccagaactactcacagctgcctaccagcctgaccgacggcctgcagggaaatcctaaggccaagatcttcaagagacagtacagagccctgaacaacatgaccgctaatgtgatcgaccctaagctgtccttcatcgtgaacaagaaagatggaagattcgagatcagcatcatccacaacgtggaagtgatccgagccagacgggacgtgctggtcggcgactacctggtgggcatggaccaaaaccagacggcttctaatacctacgccgtcatgcaggtggtgcagcctaacacccccgacagccatgagttcagaaaccagtgggtcaagttcatcgagagcggcaagatcgagagctcaacactgaactcccggggtgagtacatcgaccagctgagccacgatggcgtcgacctgcaggagattaaggattctgagtggattcctgccgccgaaaaattcctgaacaagctaggagctatcaacaaagacggcacccccatcagcatctccaacaccagcaaacgggcctacacattcaatagcatctatttcaaaatcctgctgaattatctgagagccaacgacgtggacctgaatctggtgcgggaagagatcctgcggatcgccaacggcagattcagccctatgcggctgggatctctgtcctggaccacactaaaaatgctgggcaatttccggaacctaattcacagctacttcgaccactgtggctttaaggaaatgcctgagagagaaagcaaggacaagaccatgtacgatctgctgatgcacaccatcaccaagctgaccaacaagcgggccgagcgcaccagcagaatcgctggaagcctgatgaacgtggctcacaagtacaagatcggcacaagcgtggtccacgtggtggtggaaggctctctgagcaaaaccgacaagagcagctccaagggcaacaatcggaataccacagactggtgcagccgggccgtggtgaagaagcttgaagatatgtgcgtgttctacggcttcaacctgaaagccgtgagcgcccactacaccagccaccaggaccctctggttcatagagccgattacgatgatcctaagttggccctgagatgcagatactcttcttacagcagagctgattttgagaagtggggcgaaaaatctttcgccgccgtgatcagatgggccacagacaagaagagcaacacctgctacaaggtgggagccgtagagttcttcaagaactacaaaatccctgaggacaagatcaccaaaaagctgaccatcaaagagttcctggaaattatgtgcgctgagagccactaccctaatgagtacgacgacattctgatccctagaaggggcggcagaatctacctcacaactaagaagctgctgtccgatagcacccaccagagagagtctgtgcatagccataccgccgtggtgaagatgaacggcaaggaatactatagcagcgacgccgatgaggtggctgctatcaatatctgcctgcacgactgggtggtccccctgaattggacaaatcactgcctgcctgccggatggtgtagcgaccacctgaaggaatgcgtgcaatgtcacacccctgatcctgtgagaatcagcatg

SEQ ID NO: 41 - DNA, 1979 bp, synthetic construct
  source 1..1979: mol_type = other DNA
atgagcgagctgattaaggagaacatgcacatgaagctgtatatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaggacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacactgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacatacgtcgagcagcacgaggtggcagtggccagatactgcgacctccctagcaaactggggcacaagctgaatgaattcgagggcaggggcagcctgctgacctgcggcgacgtggaggagaaccccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacaccggatccgtgtctttcccattacagtaggagcatacgggagacaagctttggccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 42 - DNA, 50 bp, synthetic construct
  source 1..50: mol_type = other DNA
ggatccgtgtctttcccattacagtaggagcatacgggagacaagctttg

SEQ ID NO: 43 - DNA, 20 bp, synthetic construct
  source 1..20: mol_type = other DNA
ccattacagtaggagcatac

SEQ ID NO: 44 - DNA, 20 bp, synthetic construct
  source 1..20: mol_type = other DNA
gtatgctcctactgtaatgg

SEQ ID NO: 45 - RNA, 20 nt, synthetic construct
  source 1..20: mol_type = other RNA
ccattacagtaggagcatac

SEQ ID NO: 46 - RNA, 20 nt, synthetic construct
  source 1..20: mol_type = other RNA
ggtcttcgataagaagacct

SEQ ID NO: 47 - protein, 1368 aa, Streptococcus pyogenes [sequence omitted]

SEQ ID NO: 48 - RNA, 96 nt, synthetic construct
  source 1..96: mol_type = other RNA
ccattacagtaggagcatacgttttagagctagaaatagcaagttaaaataaggctagtccgttatcaacttgaaaaagtggcaccgagtcggtgc

SEQ ID NO: 49 - protein, 1228 aa, unidentified
  source 1..1228: note = Lachnospiraceae bacterium MA2020
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 50 - RNA, 41 nt, synthetic construct
  source 1..41: mol_type = other RNA
taatttctactaagtgtagatccattacagtaggagcatac

SEQ ID NO: 51 - RNA, 56 nt, synthetic construct
  source 1..56: mol_type = other RNA
ctagcaatgactcagaaatgtgtccccagttgacacccattacagtaggagcatac

SEQ ID NO: 52 - RNA, 56 nt, synthetic construct
  source 1..56: mol_type = other RNA
atcgcaacatcttagaaatccgtccttagttgacggccattacagtaggagcatac

SEQ ID NO: 53 - RNA, 56 nt, synthetic construct
  source 1..56: mol_type = other RNA
tctcaacgatagtcagacatgtgtccccagtgacacccattacagtaggagcatac

SEQ ID NO: 54 - RNA, 56 nt, synthetic construct
  source 1..56: mol_type = other RNA
ctcaaagtgtcaaaagaatgtccctgctaatgggacccattacagtaggagcatac

SEQ ID NO: 55 - RNA, 57 nt, synthetic construct
  source 1..57: mol_type = other RNA
tcccaaagtggcaaaagaatctccctgttaatgggagccattacagtaggagcatac

SEQ ID NO: 56 - RNA, 56 nt, synthetic construct
  source 1..56: mol_type = other RNA
gtctaactgccatagaatcgtgcctgcaattggcacccattacagtaggagcatac

SEQ ID NO: 57 - RNA, 55 nt, synthetic construct
  source 1..55: mol_type = other RNA
tcggggcaccaaaataatctccttggtaatgggagccattacagtaggagcatac

SEQ ID NO: 58 - RNA, 56 nt, synthetic construct
  source 1..56: mol_type = other RNA
ccacaacaaccaaaagaatgtccctgaaagtgggacccattacagtaggagcatac

SEQ ID NO: 59 - RNA, 56 nt, synthetic construct
  source 1..56: mol_type = other RNA
gtaacagtggctaagtaatgtgtcttccaatgacacccattacagtaggagcatac

SEQ ID NO: 60 - RNA, 44 nt, synthetic construct
  source 1..44: mol_type = other RNA
gagagaatgtgtgcaaagtcacacccattacagtaggagcatac

SEQ ID NO: 61 - RNA, 10 nt, synthetic construct
  source 1..10: mol_type = other RNA
ccattacagt

SEQ ID NO: 62 - RNA, 12 nt, synthetic construct
  source 1..12: mol_type = other RNA
ccattacagtag

SEQ ID NO: 63 - RNA, 14 nt, synthetic construct
  source 1..14: mol_type = other RNA
ccattacagtagga

SEQ ID NO: 64 - RNA, 15 nt, synthetic construct
  source 1..15: mol_type = other RNA
ccattacagtaggag

SEQ ID NO: 65 - RNA, 16 nt, synthetic construct
  source 1..16: mol_type = other RNA
ccattacagtaggagc

SEQ ID NO: 66 - RNA, 17 nt, synthetic construct
  source 1..17: mol_type = other RNA
ccattacagtaggagca

SEQ ID NO: 67 - RNA, 18 nt, synthetic construct
  source 1..18: mol_type = other RNA
ccattacagtaggagcat

SEQ ID NO: 68 - RNA, 19 nt, synthetic construct
  source 1..19: mol_type = other RNA
ccattacagtaggagcata

SEQ ID NO: 69 - RNA, 21 nt, synthetic construct
  source 1..21: mol_type = other RNA
ccattacagtaggagcatacg

SEQ ID NO: 70 - RNA, 22 nt, synthetic construct
  source 1..22: mol_type = other RNA
ccattacagtaggagcatacgg

SEQ ID NO: 71 - RNA, 23 nt, synthetic construct
  source 1..23: mol_type = other RNA
ccattacagtaggagcatacggg

SEQ ID NO: 72 - RNA, 24 nt, synthetic construct
  source 1..24: mol_type = other RNA
ccattacagtaggagcatacggga

SEQ ID NO: 73 - RNA, 26 nt, synthetic construct
  source 1..26: mol_type = other RNA
ccattacagtaggagcatacgggaga

SEQ ID NO: 74 - RNA, 27 nt, synthetic construct
  source 1..27: mol_type = other RNA
ccattacagtaggagcatacgggagac

SEQ ID NO: 75 - RNA, 28 nt, synthetic construct
  source 1..28: mol_type = other RNA
ccattacagtaggagcatacgggagaca

SEQ ID NO: 76 - RNA, 30 nt, synthetic construct
  source 1..30: mol_type = other RNA
ccattacagtaggagcatacgggagacaag

SEQ ID NO: 77 - RNA, 32 nt, synthetic construct
  source 1..32: mol_type = other RNA
ccattacagtaggagcatacgggagacaagct

SEQ ID NO: 78 - RNA, 35 nt, synthetic construct
  source 1..35: mol_type = other RNA
ccattacagtaggagcatacgggagacaagctttg

SEQ ID NO: 79 - RNA, 40 nt, synthetic construct
  source 1..40: mol_type = other RNA
ccattacagtaggagcatacgggagacaagctttggccac

SEQ ID NO: 80 - RNA, 45 nt, synthetic construct
  source 1..45: mol_type = other RNA
ccattacagtaggagcatacgggagacaagctttggccacctacg

SEQ ID NO: 81 - RNA, 50 nt, synthetic construct
  source 1..50: mol_type = other RNA
ccattacagtaggagcatacgggagacaagctttggccacctacggcaag

SEQ ID NO: 82 - DNA, 20 bp, synthetic construct
  source 1..20: mol_type = other DNA
caaaaacccaaaatatttta

SEQ ID NO: 83 - DNA, 20 bp, synthetic construct
  source 1..20: mol_type = other DNA
gctcctactcagactgttac

SEQ ID NO: 84 - DNA, 19 bp, synthetic construct
  source 1..19: mol_type = other DNA
tgtcaccagagtaacagtc

SEQ ID NO: 85 - DNA, 20 bp, synthetic construct
  source 1..20: mol_type = other DNA
cctcgctggactggtatttg

SEQ ID NO: 86 - DNA, 20 bp, synthetic construct
  source 1..20: mol_type = other DNA
tgtctgaagctggccccgcg

SEQ ID NO: 87 - DNA, 21 bp, synthetic construct
  source 1..21: mol_type = other DNA
ccttcgactcttcctcctttg

SEQ ID NO: 88 - DNA, 21 bp, synthetic construct
  source 1..21: mol_type = other DNA
ctcctttgcctcgctggactg

SEQ ID NO: 89 - DNA, 20 bp, synthetic construct
  source 1..20: mol_type = other DNA
accatcagaggacatttgga

SEQ ID NO: 90 - DNA, 20 bp, synthetic construct
  source 1..20: mol_type = other DNA
gattctccagcaccctgggc

SEQ ID NO: 91 - DNA, 20 bp, synthetic construct
  source 1..20: mol_type = other DNA
cagccacgtctacagcaggg

SEQ ID NO: 92 - DNA, 20 bp, synthetic construct
  source 1..20: mol_type = other DNA
acagccacgtctacagcagg

SEQ ID NO: 93 - DNA, 20 bp, synthetic construct
  source 1..20: mol_type = other DNA
gaacacttttacagccacgt

SEQ ID NO: 94 - DNA, 20 bp, synthetic construct
  source 1..20: mol_type = other DNA
aaaaagacctctgagggatc

SEQ ID NO: 95 - DNA, 20 bp, synthetic construct
  source 1..20: mol_type = other DNA
aacacttttacagccacgtc

SEQ ID NO: 96 - DNA, 20 bp, synthetic construct
  source 1..20: mol_type = other DNA
tagaaggagtgtacagagta

SEQ ID NO: 97 - DNA, 20 bp, synthetic construct
  source 1..20: mol_type = other DNA
gcatttccccgttccatgaa

SEQ ID NO: 98 - DNA, 20 bp, synthetic construct
  source 1..20: mol_type = other DNA
tcatctgtggtgagcccgtg

SEQ ID NO: 99 - DNA, 20 bp, synthetic construct
  source 1..20: mol_type = other DNA
gtgtccagttctactctgta

SEQ ID NO: 100 - DNA, 20 bp, synthetic construct
  source 1..20: mol_type = other DNA
cagtacgatttggtgtccag

SEQ ID NO: 101 - DNA, 20 bp, synthetic construct
  source 1..20: mol_type = other DNA
tacaaacttctcatctgtgg

SEQ ID NO: 102 - DNA, 20 bp, synthetic construct
  source 1..20: mol_type = other DNA
cacagccaacgactctggcc

SEQ ID NO: 103 - DNA, 20 bp, synthetic construct
  source 1..20: mol_type = other DNA
acagccaacgactctggcca

SEQ ID NO: 104 - DNA, 22 bp, synthetic construct
  source 1..22: mol_type = other DNA
ctgacgacagccgtggtgctgt

SEQ ID NO: 105 - DNA, 22 bp, synthetic construct
  source 1..22: mol_type = other DNA
aaaaagacctctgagggatcct

SEQ ID NO: 106 - DNA, 22 bp, synthetic construct
  source 1..22: mol_type = other DNA
agaaaggctgctgatgacacct

SEQ ID NO: 107 - DNA, 22 bp, synthetic construct
  source 1..22: mol_type = other DNA
tagaagggatatacaaagtgga

SEQ ID NO: 108 - DNA, 22 bp, synthetic construct
  source 1..22: mol_type = other DNA
caccacggctgtcgtcaccaat

SEQ ID NO: 109 - DNA, 22 bp, synthetic construct
  source 1..22: mol_type = other DNA
aatccaagtgtcctctgatggt

SEQ ID NO: 110 - DNA, 22 bp, synthetic construct
  source 1..22: mol_type = other DNA
aatgtggccgtgcatgtgttca

SEQ ID NO: 111 - DNA, 22 bp, synthetic construct
  source 1..22: mol_type = other DNA
tagatgctgtccgaggcagtcc

SEQ ID NO: 112 - DNA, 22 bp, synthetic construct
  source 1..22: mol_type = other DNA
gcatgggctcacaactgaggag

SEQ ID NO: 113 - DNA, 22 bp, synthetic construct
  source 1..22: mol_type = other DNA
tatacaaagtggaaatagacac

SEQ ID NO: 114 - DNA, 22 bp, synthetic construct
  source 1..22: mol_type = other DNA
ctggaaggcacttggcatctcc

SEQ ID NO: 115 - DNA, 22 bp, synthetic construct
  source 1..22: mol_type = other DNA
gcatctccccattccatgagca

SEQ ID NO: 116 - DNA, 22 bp, synthetic construct
  source 1..22: mol_type = other DNA
acagccaacgactccggccccc

SEQ ID NO: 117 - DNA, 19 bp, synthetic construct
  source 1..19: mol_type = other DNA
cctggcacctacgtggtgg

SEQ ID NO: 118 - DNA, 18 bp, synthetic construct
  source 1..18: mol_type = other DNA
catggccttcttcctggc

SEQ ID NO: 119 - DNA, 20 bp, synthetic construct
  source 1..20: mol_type = other DNA
ttcctggcttcctggtgaag

SEQ ID NO: 120 - skipped

SEQ ID NO: 121 - DNA, 20 bp, synthetic construct
  source 1..20: mol_type = other DNA
aagttgccccatgtcgacta

SEQ ID NO: 122 - DNA, 20 bp, synthetic construct
  source 1..20: mol_type = other DNA
cccagagcatcccgtggaac

SEQ ID NO: 123 - DNA, 20 bp, synthetic construct
  source 1..20: mol_type = other DNA
cagatacgaacctaaacttt

SEQ ID NO: 124 - DNA, 20 bp, synthetic construct
  source 1..20: mol_type = other DNA
gagtctctcagctggtacac

SEQ ID NO: 125 - DNA, 20 bp, synthetic construct
  source 1..20: mol_type = other DNA
tctgtgatatacacatcaga

SEQ ID NO: 126 - skipped

SEQ ID NO: 127 - DNA, 22 bp, synthetic construct
  source 1..22: mol_type = other DNA
cagcaggcttctacaaagtgga

SEQ ID NO: 128 - DNA, 22 bp, synthetic construct
  source 1..22: mol_type = other DNA
taaaagggatatacaatatgta

SEQ ID NO: 129 - DNA, 22 bp, synthetic construct
  source 1..22: mol_type = other DNA
tagaagggatatagaaagtatc

SEQ ID NO: 130 - DNA, 22 bp, synthetic construct
  source 1..22: mol_type = other DNA
tagaagggatatacaaagtgga

SEQ ID NO: 131 - DNA, 23 bp, synthetic construct
  source 1..23: mol_type = other DNA
tgtctgaggctggccctacggtg

SEQ ID NO: 132 - DNA, 23 bp, synthetic construct
  source 1..23: mol_type = other DNA
accatcagaggacacttggattc

SEQ ID NO: 133 - DNA, 23 bp, synthetic construct
  source 1..23: mol_type = other DNA
tgaacacatgcacggccacattg

SEQ ID NO: 134 - DNA, 23 bp, synthetic construct
  source 1..23: mol_type = other DNA
cctctgggtaagttgccaaagaa

SEQ ID NO: 135 - DNA, 23 bp, synthetic construct
  source 1..23: mol_type = other DNA
gcaacttacccagaggcaaatgg

SEQ ID NO: 136 - DNA, 23 bp, synthetic construct
  source 1..23: mol_type = other DNA
acaccttataggaaaaccagtga

SEQ ID NO: 137 - DNA, 23 bp, synthetic construct
  source 1..23: mol_type = other DNA
ctataaggtgtgaaagtctggat

SEQ ID NO: 138 - DNA, 23 bp, synthetic construct
  source 1..23: mol_type = other DNA
cctataaggtgtgaaagtctgga

SEQ ID NO: 139 - DNA, 23 bp, synthetic construct
  source 1..23: mol_type = other DNA
tagaagggatatacaaagtggaa

SEQ ID NO: 140 - DNA, 23 bp, synthetic construct
  source 1..23: mol_type = other DNA
tatatcccttctacaaattcctc

SEQ ID NO: 141 - DNA, 23 bp, synthetic construct
  source 1..23: mol_type = other DNA
cactttgtatatcccttctacaa

SEQ ID NO: 142 - DNA, 23 bp, synthetic construct
  source 1..23: mol_type = other DNA
gtgtctatttccactttgtatat

SEQ ID NO: 143 - DNA, 23 bp, synthetic construct
  source 1..23: mol_type = other DNA
tgtctgaggctggccctacggtg

SEQ ID NO: 144 - DNA, 23 bp, synthetic construct
  source 1..23: mol_type = other DNA
accatcagaggacacttggattc

SEQ ID NO: 145 - DNA, 23 bp, synthetic construct
  source 1..23: mol_type = other DNA
tgaacacatgcacggccacattg

SEQ ID NO: 146 - DNA, 23 bp, synthetic construct
  source 1..23: mol_type = other DNA
cctctgggtaagttgccaaagaa

SEQ ID NO: 147 - DNA, 23 bp, synthetic construct
  source 1..23: mol_type = other DNA
gcaacttacccagaggcaaatgg

SEQ ID NO: 148 - DNA, 23 bp, synthetic construct
  source 1..23: mol_type = other DNA
acaccttataggaaaaccagtga

SEQ ID NO: 149 - DNA, 23 bp, synthetic construct
  source 1..23: mol_type = other DNA
ctataaggtgtgaaagtctggat

SEQ ID NO: 150 - DNA, 23 bp, synthetic construct
  source 1..23: mol_type = other DNA
cctataaggtgtgaaagtctgga

SEQ ID NO: 151 - DNA, 23 bp, synthetic construct
  source 1..23: mol_type = other DNA
tagaagggatatacaaagtggaa

SEQ ID NO: 152 - DNA, 23 bp, synthetic construct
  source 1..23: mol_type = other DNA
tatatcccttctacaaattcctc

SEQ ID NO: 153 - DNA, 23 bp, synthetic construct
  source 1..23: mol_type = other DNA
cactttgtatatcccttctacaa

SEQ ID NO: 154 - DNA, 23 bp, synthetic construct
  source 1..23: mol_type = other DNA
gtgtctatttccactttgtatat

SEQ ID NO: 155 - DNA, 20 bp, synthetic construct
  source 1..20: mol_type = other DNA
ccacctatgagagaagacag

SEQ ID NO: 156 - DNA, 20 bp, synthetic construct
  source 1..20: mol_type = other DNA
ggctgtcgtcaccaatccca

SEQ ID NO: 157 - DNA, 20 bp, synthetic construct
  source 1..20: mol_type = other DNA
gacgacagccgtggtggaat

SEQ ID NO: 158 - DNA, 20 bp, synthetic construct
  source 1..20: mol_type = other DNA
ctgaacacatgcacggccac

SEQ ID NO: 159 - DNA, 20 bp, synthetic construct
  source 1..20: mol_type = other DNA
cacccagggcaccggtgaat

SEQ ID NO: 160 - DNA, 20 bp, synthetic construct
  source 1..20: mol_type = other DNA
aatggtgtagcggcgggggc

SEQ ID NO: 161 - DNA, 20 bp, synthetic construct
  source 1..20: mol_type = other DNA
ctttggcaacttacccagag

SEQ ID NO: 162 - DNA, 20 bp, synthetic construct
  source 1..20: mol_type = other DNA
tgtctgaggctggccctacg

SEQ ID NO: 163 - DNA, 20 bp, synthetic construct
  source 1..20: mol_type = other DNA
tttgtgtctgaggctggccc

SEQ ID NO: 164 - DNA, 20 bp, synthetic construct
  source 1..20: mol_type = other DNA
attggtgacgacagccgtgg

SEQ ID NO: 165 - DNA, 20 bp, synthetic construct
  source 1..20: mol_type = other DNA
aggagaagtccctcattcct

SEQ ID NO: 166 - DNA, 20 bp, synthetic construct
  source 1..20: mol_type = other DNA
ccaagtgccttccagtaaga

SEQ ID NO: 167 - DNA, 20 bp, synthetic construct
  source 1..20: mol_type = other DNA
acacaaataccagtccagca

SEQ ID NO: 168 - DNA, 20 bp, synthetic construct
  source 1..20: mol_type = other DNA
ccagtccagcaaggcagagg

SEQ ID NO: 169 - DNA, 20 bp, synthetic construct
  source 1..20: mol_type = other DNA
gaagtccactcattcttggc

SEQ ID NO: 170 - DNA, 20 bp, synthetic construct
  source 1..20: mol_type = other DNA
aaagttctagatgctgtccg

SEQ ID NO: 171 - DNA, 20 bp, synthetic construct
  source 1..20: mol_type = other DNA
cccagaggcaaatggctccc

SEQ ID NO: 172 - DNA, 20 bp, synthetic construct
  source 1..20: mol_type = other DNA
ttctttggcaacttacccag

SEQ ID NO: 173 - DNA, 20 bp, synthetic construct
  source 1..20: mol_type = other DNA
actgaggaggaatttgtaga

SEQ ID NO: 174 - DNA, 20 bp, synthetic construct
  source 1..20: mol_type = other DNA
cccattccatgagcatgcag

SEQ ID NO: 175 - DNA, 20 bp, synthetic construct
  source 1..20: mol_type = other DNA
gcatgggctcacaactgagg

SEQ ID NO: 176 - DNA, 20 bp, synthetic construct
  source 1..20: mol_type = other DNA
aataggagtaggggctcagc

SEQ ID NO: 177 - DNA, 20 bp, synthetic construct
  source 1..20: mol_type = other DNA
gacgacagccgtggtggaat

SEQ ID NO: 178 - DNA, 20 bp, synthetic construct
  source 1..20: mol_type = other DNA
ggctgtcgtcaccaatccca

SEQ ID NO: 179 - DNA, 20 bp, synthetic construct
  source 1..20: mol_type = other DNA
gtcaccaatcccaaggaatg

SEQ ID NO: 180 - DNA, 20 bp, synthetic construct
  source 1..20: mol_type = other DNA
tgtgtctgaggctggcccta

SEQ ID NO: 181 - DNA, 20 bp, synthetic construct
  source 1..20: mol_type = other DNA
agcctttctgaacacatgca

SEQ ID NO: 182 - DNA, 20 bp, synthetic construct
  source 1..20: mol_type = other DNA
cagaggacacttggattcac

SEQ ID NO: 183 - DNA, 20 bp, synthetic construct
  source 1..20: mol_type = other DNA
cattgatggcaggactgcct

SEQ ID NO: 184 - DNA, 20 bp, synthetic construct
  source 1..20: mol_type = other DNA
cttctctacacccagggcac

SEQ ID NO: 185 - DNA, 20 bp, synthetic construct
  source 1..20: mol_type = other DNA
aatggtgtagcggcgggggc

SEQ ID NO: 186 - DNA, 20 bp, synthetic construct
  source 1..20: mol_type = other DNA
cccctactcctattccacca

SEQ ID NO: 187 - DNA, 20 bp, synthetic construct
  source 1..20: mol_type = other DNA
gcagggcggcaatggtgtag

SEQ ID NO: 188 - DNA, 20 bp, synthetic construct
  source 1..20: mol_type = other DNA
ggagtaggggctcagcaggg

SEQ ID NO: 189 - DNA, 20 bp, synthetic construct
  source 1..20: mol_type = other DNA
gtattcacagccaacgactc

SEQ ID NO: 190 - DNA, 20 bp, synthetic construct
  source 1..20: mol_type = other DNA
tcacagaaacactcaccgta

SEQ ID NO: 191 - DNA, 20 bp, synthetic construct
  source 1..20: mol_type = other DNA
aaaggctgctgatgacacct

SEQ ID NO: 192 - DNA, 20 bp, synthetic construct
  source 1..20: mol_type = other DNA
cttggattcaccggtgccct

SEQ ID NO: 193 - DNA, 20 bp, synthetic construct
  source 1..20: mol_type = other DNA
gccgtggtggaataggagta

SEQ ID NO: 194 - DNA, 20 bp, synthetic construct
  source 1..20: mol_type = other DNA
gcggcaatggtgtagcggcg

SEQ ID NO: 195 - DNA, 20 bp, synthetic construct
  source 1..20: mol_type = other DNA
ggagaagtccctcattcctt

SEQ ID NO: 196 - DNA, 20 bp, synthetic construct
  source 1..20: mol_type = other DNA
ggcggcaatggtgtagcggc

SEQ ID NO: 197 - DNA, 20 bp, synthetic construct
  source 1..20: mol_type = other DNA
tcaccaatcccaaggaatga

SEQ ID NO: 198 - DNA, 20 bp, synthetic construct
  source 1..20: mol_type = other DNA
gcaacttacccagaggcaaa

SEQ ID NO: 199 - DNA, 20 bp, synthetic construct
  source 1..20: mol_type = other DNA
aagtgccttccagtaagatt

SEQ ID NO: 200 - DNA, 20 bp, synthetic construct
  source 1..20: mol_type = other DNA
acctctgcatgctcatggaa

SEQ ID NO: 201 - DNA, 20 bp, synthetic construct
  source 1..20: mol_type = other DNA
tactcacctctgcatgctca

SEQ ID NO: 202 - DNA, 20 bp, synthetic construct
  source 1..20: mol_type = other DNA
tgtagaagggatatacaaag

SEQ ID NO: 203 - DNA, 20 bp, synthetic construct
  source 1..20: mol_type = other DNA
aggagaagtccctcattcct

SEQ ID NO: 204 - DNA, 20 bp, synthetic construct
  source 1..20: mol_type = other DNA
attggtgacgacagccgtgg

SEQ ID NO: 205 - DNA, 20 bp, synthetic construct
  source 1..20: mol_type = other DNA
gcggcgggggccggagtcgt

SEQ ID NO: 206 - DNA, 20 bp, synthetic construct
  source 1..20: mol_type = other DNA
gggattggtgacgacagccg

SEQ ID NO: 207 - DNA, 20 bp, synthetic construct
  source 1..20: mol_type = other DNA
ggggctcagcagggcggcaa

SEQ ID NO: 208 - DNA, 23 bp, synthetic construct
  source 1..23: mol_type = other DNA
tgtctgaggctggccctacggtg

SEQ ID NO: 209 - DNA, 23 bp, synthetic construct
  source 1..23: mol_type = other DNA
accatcagaggacacttggattc

SEQ ID NO: 210 - DNA, 23 bp, synthetic construct
  source 1..23: mol_type = other DNA
tgaacacatgcacggccacattg

SEQ ID NO: 211 - DNA, 23 bp, synthetic construct
  source 1..23: mol_type = other DNA
cctctgggtaagttgccaaagaa

SEQ ID NO: 212 - DNA, 23 bp, synthetic construct
  source 1..23: mol_type = other DNA
gcaacttacccagaggcaaatgg

SEQ ID NO: 213 - DNA, 23 bp, synthetic construct
  source 1..23: mol_type = other DNA
acaccttataggaaaaccagtga

SEQ ID NO: 214 - DNA, 23 bp, synthetic construct
  source 1..23: mol_type = other DNA
ctataaggtgtgaaagtctggat

SEQ ID NO: 215 - DNA, 23 bp, synthetic construct
  source 1..23: mol_type = other DNA
cctataaggtgtgaaagtctgga

SEQ ID NO: 216 - DNA, 23 bp, synthetic construct
  source 1..23: mol_type = other DNA
tagaagggatatacaaagtggaa

SEQ ID NO: 217 - DNA, 23 bp, synthetic construct
  source 1..23: mol_type = other DNA
tatatcccttctacaaattcctc

SEQ ID NO: 218 - DNA, 23 bp, synthetic construct
  source 1..23: mol_type = other DNA
cactttgtatatcccttctacaa

SEQ ID NO: 219 - DNA, 23 bp, synthetic construct
  source 1..23: mol_type = other DNA
gtgtctatttccactttgtatat

SEQ ID NO: 220 - DNA, 20 bp, synthetic construct
  source 1..20: mol_type = other DNA
ctggaaggcacttggcatct

SEQ ID NO: 221 - DNA, 20 bp, synthetic construct
  source 1..20: mol_type = other DNA
taggaaaaccagtgagtctg

SEQ ID NO: 222 - DNA, 20 bp, synthetic construct
  source 1..20: mol_type = other DNA
tcatcgtctgctcctcctct

SEQ ID NO: 223 - DNA, 20 bp, synthetic construct
  source 1..20: mol_type = other DNA
ttggcaggatggcttctcat

SEQ ID NO: 224 - DNA, 20 bp, synthetic construct
  source 1..20: mol_type = other DNA
accggtgccctgggtgtaga

SEQ ID NO: 225 - DNA, 20 bp, synthetic construct
  source 1..20: mol_type = other DNA
agaaaggctgctgatgacac

SEQ ID NO: 226 - DNA, 20 bp, synthetic construct
  source 1..20: mol_type = other DNA
tagatgctgtccgaggcagt

SEQ ID NO: 227 - DNA, 20 bp, synthetic construct
  source 1..20: mol_type = other DNA
tctacacccagggcaccggt

SEQ ID NO: 228 - DNA, 20 bp, synthetic construct
  source 1..20: mol_type = other DNA
tttggcaacttacccagagg

SEQ ID NO: 229 - DNA, 20 bp, synthetic construct
  source 1..20: mol_type = other DNA
cagtaagatttggtgtctat

SEQ ID NO: 230 - DNA, 20 bp, synthetic construct
  source 1..20: mol_type = other DNA
catgagcatgcagaggtgag

SEQ ID NO: 231 - DNA, 20 bp, synthetic construct
  source 1..20: mol_type = other DNA
ctcctcagttgtgagcccat

SEQ ID NO: 232 - DNA, 20 bp, synthetic construct
  source 1..20: mol_type = other DNA
tacaaattcctcctcagttg

SEQ ID NO: 233 - DNA, 20 bp, synthetic construct
  source 1..20: mol_type = other DNA
acagccaacgactccggccc

SEQ ID NO: 234 - DNA, 20 bp, synthetic construct
  source 1..20: mol_type = other DNA
caccacggctgtcgtcacca

SEQ ID NO: 235 - DNA, 20 bp, synthetic construct
  source 1..20: mol_type = other DNA
cttgggattggtgacgacag

SEQ ID NO: 236 - DNA, 20 bp, synthetic construct
  source 1..20: mol_type = other DNA
tctcataggtggtattcaca

SEQ ID NO: 237 - DNA, 20 bp, synthetic construct
  source 1..20: mol_type = other DNA
ctggactggtatttgtgtct

SEQ ID NO: 238 - DNA, 20 bp, synthetic construct
  source 1..20: mol_type = other DNA
gcaggatggcttctcatcgt

SEQ ID NO: 239 - DNA, 20 bp, synthetic construct
  source 1..20: mol_type = other DNA
atggcaggactgcctcggac

SEQ ID NO: 240 - DNA, 20 bp, synthetic construct
  source 1..20: mol_type = other DNA
gattcaccggtgccctgggt

SEQ ID NO: 241 - DNA, 20 bp, synthetic construct
  source 1..20: mol_type = other DNA
gcatctccccattccatgag

SEQ ID NO: 242 - DNA, 20 bp, synthetic construct
  source 1..20: mol_type = other DNA
tgagcccatgcagctctcca

SEQ ID NO: 243 - DNA, 20 bp, synthetic construct
  source 1..20: mol_type = other DNA
ccgccctgctgagcccctac

SEQ ID NO: 244 - DNA, 20 bp, synthetic construct
  source 1..20: mol_type = other DNA
gctgtgaataccacctatga

SEQ ID NO: 245 - DNA, 20 bp, synthetic construct
  source 1..20: mol_type = other DNA
ggattggtgacgacagccgt

SEQ ID NO: 246 - DNA, 20 bp, synthetic construct
  source 1..20: mol_type = other DNA
gtgacgacagccgtggtgga

SEQ ID NO: 247 - DNA, 20 bp, synthetic construct
  source 1..20: mol_type = other DNA
gtgtctgaggctggccctac

SEQ ID NO: 248 - DNA, 20 bp, synthetic construct
  source 1..20: mol_type = other DNA
gaccatcagaggacacttgg

SEQ ID NO: 249 - DNA, 20 bp, synthetic construct
  source 1..20: mol_type = other DNA
gcctctgggtaagttgccaa

SEQ ID NO: 250 - DNA, 20 bp, synthetic construct
  source 1..20: mol_type = other DNA
ggcaacttacccagaggcaa

SEQ ID NO: 251 - DNA, 20 bp, synthetic construct
  source 1..20: mol_type = other DNA
ggtgtctatttccactttgt

SEQ ID NO: 252 - DNA, 20 bp, synthetic construct
  source 1..20: mol_type = other DNA
gtatatcccttctacaaatt

SEQ ID NO: 253 - DNA, 23 bp, synthetic construct
  source 1..23: mol_type = other DNA
tgtctgaggctggccctacggtg

SEQ ID NO: 254 - DNA, 23 bp, synthetic construct
  source 1..23: mol_type = other DNA
accatcagaggacacttggattc

SEQ ID NO: 255 - DNA, 23 bp, synthetic construct
  source 1..23: mol_type = other DNA
tgaacacatgcacggccacattg

SEQ ID NO: 256 - DNA, 23 bp, synthetic construct
  source 1..23: mol_type = other DNA
cctctgggtaagttgccaaagaa

SEQ ID NO: 257 - DNA, 23 bp, synthetic construct
  source 1..23: mol_type = other DNA
acaccttataggaaaaccagtga

SEQ ID NO: 258 - DNA, 23 bp, synthetic construct
  source 1..23: mol_type = other DNA
ctataaggtgtgaaagtctggat

SEQ ID NO: 259 - DNA, 23 bp, synthetic construct
  source 1..23: mol_type = other DNA
cctataaggtgtgaaagtctgga

SEQ ID NO: 260 - DNA, 23 bp, synthetic construct
  source 1..23: mol_type = other DNA
tagaagggatatacaaagtggaa

SEQ ID NO: 261 - DNA, 23 bp, synthetic construct
  source 1..23: mol_type = other DNA
tatatcccttctacaaattcctc

SEQ ID NO: 262 - DNA, 23 bp, synthetic construct
  source 1..23: mol_type = other DNA
cactttgtatatcccttctacaa

SEQ ID NO: 263 - DNA, 23 bp, synthetic construct
  source 1..23: mol_type = other DNA
gtgtctatttccactttgtatat

SEQ ID NO: 264 - DNA, 20 bp, synthetic construct
  source 1..20: mol_type = other DNA
ctggaaggcacttggcatct

SEQ ID NO: 265 - DNA, 20 bp, synthetic construct
  source 1..20: mol_type = other DNA
taggaaaaccagtgagtctg

SEQ ID NO: 266 - DNA, 20 bp, synthetic construct
  source 1..20: mol_type = other DNA
tcatcgtctgctcctcctct

SEQ ID NO: 267 - DNA, 20 bp, synthetic construct
  source 1..20: mol_type = other DNA
ttggcaggatggcttctcat

SEQ ID NO: 268 - DNA, 20 bp, synthetic construct
  source 1..20: mol_type = other DNA
accggtgccctgggtgtaga

SEQ ID NO: 269 - DNA, 20 bp, synthetic construct
  source 1..20: mol_type = other DNA
agaaaggctgctgatgacac

SEQ ID NO: 270 - DNA, 20 bp, synthetic construct
  source 1..20: mol_type = other DNA
tagatgctgtccgaggcagt

SEQ ID NO: 271 - DNA, 20 bp, synthetic construct
  source 1..20: mol_type = other DNA
tttggcaacttacccagagg

SEQ ID NO: 272 - DNA, 20 bp, synthetic construct
  source 1..20: mol_type = other DNA
cagtaagatttggtgtctat

SEQ ID NO: 273 - DNA, 20 bp, synthetic construct
  source 1..20: mol_type = other DNA
catgagcatgcagaggtgag

SEQ ID NO: 274 - DNA, 20 bp, synthetic construct
  source 1..20: mol_type = other DNA
ctcctcagttgtgagcccat

SEQ ID NO: 275 - DNA, 20 bp, synthetic construct
  source 1..20: mol_type = other DNA
tacaaattcctcctcagttg

SEQ ID NO: 276 - DNA, 20 bp, synthetic construct
  source 1..20: mol_type = other DNA
acagccaacgactccggccc

SEQ ID NO: 277 - DNA, 20 bp, synthetic construct
  source 1..20: mol_type = other DNA
caccacggctgtcgtcacca

SEQ ID NO: 278 - DNA, 20 bp, synthetic construct
  source 1..20: mol_type = other DNA
cttgggattggtgacgacag

SEQ ID NO: 279 - DNA, 20 bp, synthetic construct
  source 1..20: mol_type = other DNA
tctcataggtggtattcaca

SEQ ID NO: 280 - DNA, 20 bp, synthetic construct
  source 1..20: mol_type = other DNA
ctggactggtatttgtgtct

SEQ ID NO: 281 - DNA, 20 bp, synthetic construct
  source 1..20: mol_type = other DNA
gcaggatggcttctcatcgt

SEQ ID NO: 282 - DNA, 20 bp, synthetic construct
  source 1..20: mol_type = other DNA
atggcaggactgcctcggac

SEQ ID NO: 283 - DNA, 20 bp, synthetic construct
  source 1..20: mol_type = other DNA
gattcaccggtgccctgggt

SEQ ID NO: 284 - DNA, 20 bp, synthetic construct
  source 1..20: mol_type = other DNA
gcatctccccattccatgag

SEQ ID NO: 285 - DNA, 20 bp, synthetic construct
  source 1..20: mol_type = other DNA
tgagcccatgcagctctcca

SEQ ID NO: 286 - DNA, 20 bp, synthetic construct
  source 1..20: mol_type = other DNA
ccgccctgctgagcccctac

SEQ ID NO: 287 - DNA, 20 bp, synthetic construct
  source 1..20: mol_type = other DNA
gctgtgaataccacctatga

SEQ ID NO: 288 - DNA, 20 bp, synthetic construct
  source 1..20: mol_type = other DNA
ggattggtgacgacagccgt

SEQ ID NO: 289 - DNA, 20 bp, synthetic construct
  source 1..20: mol_type = other DNA
gtgacgacagccgtggtgga

SEQ ID NO: 290 - DNA, 20 bp, synthetic construct
  source 1..20: mol_type = other DNA
gtgtctgaggctggccctac

SEQ ID NO: 291 - DNA, 20 bp, synthetic construct
  source 1..20: mol_type = other DNA
gaccatcagaggacacttgg

SEQ ID NO: 292 - DNA, 20 bp, synthetic construct
  source 1..20: mol_type = other DNA
gcctctgggtaagttgccaa

SEQ ID NO: 293 - DNA, 20 bp, synthetic construct
  source 1..20: mol_type = other DNA
ggcaacttacccagaggcaa

SEQ ID NO: 294 - DNA, 20 bp, synthetic construct
  source 1..20: mol_type = other DNA
ggtgtctatttccactttgt

SEQ ID NO: 295 - DNA, 20 bp, synthetic construct
  source 1..20: mol_type = other DNA
gtatatcccttctacaaatt

SEQ ID NO: 296 - DNA, 23 bp, synthetic construct
  source 1..23: mol_type = other DNA
tgtctgaggctggccctacggtg

SEQ ID NO: 297 - DNA, 23 bp, synthetic construct
  source 1..23: mol_type = other DNA
accatcagaggacacttggattc

SEQ ID NO: 298 - DNA, 23 bp, synthetic construct
  source 1..23: mol_type = other DNA
tgaacacatgcacggccacattg

SEQ ID NO: 299 - DNA, 23 bp, synthetic construct
  source 1..23: mol_type = other DNA
cctctgggtaagttgccaaagaa

SEQ ID NO: 300 - DNA, 23 bp, synthetic construct
  source 1..23: mol_type = other DNA
acaccttataggaaaaccagtga

SEQ ID NO: 301 - DNA, 23 bp, synthetic construct
  source 1..23: mol_type = other DNA
ctataaggtgtgaaagtctggat

SEQ ID NO: 302 - DNA, 23 bp, synthetic construct
  source 1..23: mol_type = other DNA
cctataaggtgtgaaagtctgga

SEQ ID NO: 303 - DNA, 23 bp, synthetic construct
  source 1..23: mol_type = other DNA
tagaagggatatacaaagtggaa

SEQ ID NO: 304 - DNA, 23 bp, synthetic construct
  source 1..23: mol_type = other DNA
tatatcccttctacaaattcctc

SEQ ID NO: 305 - DNA, 23 bp, synthetic construct
  source 1..23: mol_type = other DNA
cactttgtatatcccttctacaa

SEQ ID NO: 306 - DNA, 23 bp, synthetic construct
  source 1..23: mol_type = other DNA
gtgtctatttccactttgtatat

SEQ ID NO: 307 - DNA, 20 bp, synthetic construct
  source 1..20: mol_type = other DNA
ctggaaggcacttggcatct

SEQ ID NO: 308 - DNA, 20 bp, synthetic construct
  source 1..20: mol_type = other DNA
taggaaaaccagtgagtctg

SEQ ID NO: 309 - DNA, 20 bp, synthetic construct
  source 1..20: mol_type = other DNA
tcatcgtctgctcctcctct

SEQ ID NO: 310 - DNA, 20 bp, synthetic construct
  source 1..20: mol_type = other DNA
ttggcaggatggcttctcat

SEQ ID NO: 311 - DNA, 20 bp, synthetic construct
  source 1..20: mol_type = other DNA
accggtgccctgggtgtaga

SEQ ID NO: 312 - DNA, 20 bp, synthetic construct
  source 1..20: mol_type = other DNA
agaaaggctgctgatgacac

SEQ ID NO: 313 - DNA, 20 bp, synthetic construct
  source 1..20: mol_type = other DNA
tagatgctgtccgaggcagt

SEQ ID NO: 314 - DNA, 20 bp, synthetic construct
  source 1..20: mol_type = other DNA
tttggcaacttacccagagg

SEQ ID NO: 315 - DNA, 20 bp, synthetic construct
  source 1..20: mol_type = other DNA
cagtaagatttggtgtctat

SEQ ID NO: 316 - DNA, 20 bp, synthetic construct
  source 1..20: mol_type = other DNA
catgagcatgcagaggtgag

SEQ ID NO: 317 - DNA, 20 bp, synthetic construct
  source 1..20: mol_type = other DNA
ctcctcagttgtgagcccat

SEQ ID NO: 318 - DNA, 20 bp, synthetic construct
  source 1..20: mol_type = other DNA
tacaaattcctcctcagttg

SEQ ID NO: 319 - DNA, 20 bp, synthetic construct
  source 1..20: mol_type = other DNA
acagccaacgactccggccc

SEQ ID NO: 320 - DNA, 20 bp, synthetic construct
  source 1..20: mol_type = other DNA
caccacggctgtcgtcacca

SEQ ID NO: 321 - DNA, 20 bp, synthetic construct
  source 1..20: mol_type = other DNA
cttgggattggtgacgacag

SEQ ID NO: 322 - DNA, 20 bp, synthetic construct
  source 1..20: mol_type = other DNA
tctcataggtggtattcaca

SEQ ID NO: 323 - DNA, 20 bp, synthetic construct
  source 1..20: mol_type = other DNA
ctggactggtatttgtgtct

SEQ ID NO: 324 - DNA, 20 bp, synthetic construct
  source 1..20: mol_type = other DNA
gcaggatggcttctcatcgt

SEQ ID NO: 325 - DNA, 20 bp, synthetic construct
  source 1..20: mol_type = other DNA
atggcaggactgcctcggac

SEQ ID NO: 326 - DNA, 20 bp, synthetic construct
  source 1..20: mol_type = other DNA
gattcaccggtgccctgggt

SEQ ID NO: 327 - DNA, 20 bp, synthetic construct
  source 1..20: mol_type = other DNA
gcatctccccattccatgag

SEQ ID NO: 328 - DNA, 20 bp, synthetic construct
  source 1..20: mol_type = other DNA
tgagcccatgcagctctcca

SEQ ID NO: 329 - DNA, 20 bp, synthetic construct
  source 1..20: mol_type = other DNA
ccgccctgctgagcccctac

SEQ ID NO: 330 - DNA, 20 bp, synthetic construct
  source 1..20: mol_type = other DNA
gctgtgaataccacctatga

SEQ ID NO: 331 - DNA, 20 bp, synthetic construct
  source 1..20: mol_type = other DNA
ggattggtgacgacagccgt

SEQ ID NO: 332 - DNA, 20 bp, synthetic construct
  source 1..20: mol_type = other DNA
gtgacgacagccgtggtgga

SEQ ID NO: 333 - DNA, 20 bp, synthetic construct
  source 1..20: mol_type = other DNA
gtgtctgaggctggccctac

SEQ ID NO: 334 - DNA, 20 bp, synthetic construct
  source 1..20: mol_type = other DNA
gaccatcagaggacacttgg

SEQ ID NO: 335 - DNA, 20 bp, synthetic construct
  source 1..20: mol_type = other DNA
gcctctgggtaagttgccaa

SEQ ID NO: 336 - DNA, 20 bp, synthetic construct
  source 1..20: mol_type = other DNA
ggcaacttacccagaggcaa

SEQ ID NO: 337 - DNA, 20 bp, synthetic construct
  source 1..20: mol_type = other DNA
ggtgtctatttccactttgt

SEQ ID NO: 338 - DNA, 20 bp, synthetic construct
  source 1..20: mol_type = other DNA
gtatatcccttctacaaatt

SEQ ID NO: 339 - DNA, 23 bp, synthetic construct
  source 1..23: mol_type = other DNA
tgtctgaggctggccctacggtg

SEQ ID NO: 340 - DNA, 23 bp, synthetic construct
  source 1..23: mol_type = other DNA
accatcagaggacacttggattc

SEQ ID NO: 341 - DNA, 23 bp, synthetic construct
  source 1..23: mol_type = other DNA
tgaacacatgcacggccacattg

SEQ ID NO: 342 - DNA, 23 bp, synthetic construct
  source 1..23: mol_type = other DNA
cctctgggtaagttgccaaagaa

SEQ ID NO: 343 - DNA, 23 bp, synthetic construct
  source 1..23: mol_type = other DNA
acaccttataggaaaaccagtga

SEQ ID NO: 344 - DNA, 23 bp, synthetic construct
  source 1..23: mol_type = other DNA
ctataaggtgtgaaagtctggat

SEQ ID NO: 345 - DNA, 23 bp, synthetic construct
  source 1..23: mol_type = other DNA
cctataaggtgtgaaagtctgga

SEQ ID NO: 346 - DNA, 23 bp, synthetic construct
  source 1..23: mol_type = other DNA
tagaagggatatacaaagtggaa

SEQ ID NO: 347 - DNA, 23 bp, synthetic construct
  source 1..23: mol_type = other DNA
tatatcccttctacaaattcctc

SEQ ID NO: 348 - DNA, 23 bp, synthetic construct
  source 1..23: mol_type = other DNA
cactttgtatatcccttctacaa

SEQ ID NO: 349 - DNA, 23 bp, synthetic construct
  source 1..23: mol_type = other DNA
gtgtctatttccactttgtatat

SEQ ID NO: 350 - DNA, 20 bp, synthetic construct
  source 1..20: mol_type = other DNA
ctggaaggcacttggcatct

SEQ ID NO: 351 - DNA, 20 bp, synthetic construct
  source 1..20: mol_type = other DNA
taggaaaaccagtgagtctg

SEQ ID NO: 352 - DNA, 20 bp, synthetic construct
  source 1..20: mol_type = other DNA
tcatcgtctgctcctcctct

SEQ ID NO: 353 - DNA, 20 bp, synthetic construct
  source 1..20: mol_type = other DNA
ttggcaggatggcttctcat

SEQ ID NO: 354 - DNA, 20 bp, synthetic construct
  source 1..20: mol_type = other DNA
accggtgccctgggtgtaga

SEQ ID NO: 355 - DNA, 20 bp, synthetic construct
  source 1..20: mol_type = other DNA
agaaaggctgctgatgacac

SEQ ID NO: 356 - DNA, 20 bp, synthetic construct
  source 1..20: mol_type = other DNA
tagatgctgtccgaggcagt

SEQ ID NO: 357 - DNA, 20 bp, synthetic construct
  source 1..20: mol_type = other DNA
tttggcaacttacccagagg

SEQ ID NO: 358 - DNA, 20 bp, synthetic construct
  source 1..20: mol_type = other DNA
cagtaagatttggtgtctat

SEQ ID NO: 359 - DNA, 20 bp, synthetic construct
  source 1..20: mol_type = other DNA
catgagcatgcagaggtgag

SEQ ID NO: 360 - DNA, 20 bp, synthetic construct
  source 1..20: mol_type = other DNA
ctcctcagttgtgagcccat

SEQ ID NO: 361 - DNA, 20 bp, synthetic construct
  source 1..20: mol_type = other DNA
tacaaattcctcctcagttg

SEQ ID NO: 362 - DNA, 20 bp, synthetic construct
  source 1..20: mol_type = other DNA
acagccaacgactccggccc

SEQ ID NO: 363 - DNA, 20 bp, synthetic construct
  source 1..20: mol_type = other DNA
caccacggctgtcgtcacca

SEQ ID NO: 364 - DNA, 20 bp, synthetic construct
  source 1..20: mol_type = other DNA
cttgggattggtgacgacag

SEQ ID NO: 365 - DNA, 20 bp, synthetic construct
  source 1..20: mol_type = other DNA
tctcataggtggtattcaca

SEQ ID NO: 366 - DNA, 20 bp, synthetic construct
  source 1..20: mol_type = other DNA
ctggactggtatttgtgtct

SEQ ID NO: 367 - DNA, 20 bp, synthetic construct
  source 1..20: mol_type = other DNA
gcaggatggcttctcatcgt

SEQ ID NO: 368 - DNA, 20 bp, synthetic construct
  source 1..20: mol_type = other DNA
atggcaggactgcctcggac

SEQ ID NO: 369 - DNA, 20 bp, synthetic construct
  source 1..20: mol_type = other DNA
gattcaccggtgccctgggt

SEQ ID NO: 370 - DNA, 20 bp, synthetic construct
  source 1..20: mol_type = other DNA
gcatctccccattccatgag

SEQ ID NO: 371 - DNA, 20 bp, synthetic construct
  source 1..20: mol_type = other DNA
tgagcccatgcagctctcca

SEQ ID NO: 372 - DNA, 20 bp, synthetic construct
  source 1..20: mol_type = other DNA
ccgccctgctgagcccctac

SEQ ID NO: 373 - DNA, 20 bp, synthetic construct
  source 1..20: mol_type = other DNA
gctgtgaataccacctatga

SEQ ID NO: 374 - DNA, 20 bp, synthetic construct
  source 1..20: mol_type = other DNA
ggattggtgacgacagccgt

SEQ ID NO: 375 - DNA, 20 bp, synthetic construct
  source 1..20: mol_type = other DNA
gtgacgacagccgtggtgga

SEQ ID NO: 376 - DNA, 20 bp, synthetic construct
  source 1..20: mol_type = other DNA
gtgtctgaggctggccctac

SEQ ID NO: 377 - DNA, 20 bp, synthetic construct
  source 1..20: mol_type = other DNA
gaccatcagaggacacttgg

SEQ ID NO: 378 - DNA, 20 bp, synthetic construct
  source 1..20: mol_type = other DNA
gcctctgggtaagttgccaa

SEQ ID NO: 379 - DNA, 20 bp, synthetic construct
  source 1..20: mol_type = other DNA
ggcaacttacccagaggcaa

SEQ ID NO: 380 - DNA, 20 bp, synthetic construct
  source 1..20: mol_type = other DNA
ggtgtctatttccactttgt

SEQ ID NO: 381 - DNA, 20 bp, synthetic construct
  source 1..20: mol_type = other DNA
gtatatcccttctacaaatt

SEQ ID NO: 382 - DNA, 20 bp, synthetic construct
  source 1..20: mol_type = other DNA
cacataggccattcagaaac

SEQ ID NO: 383 - DNA, 20 bp, synthetic construct
  source 1..20: mol_type = other DNA
catatgggtcattcagaaac

SEQ ID NO: 384 - DNA, 20 bp, synthetic construct
  source 1..20: mol_type = other DNA
cacacaggcctttcagcaac

SEQ ID NO: 385 - DNA, 20 bp, synthetic construct
  source 1..20: mol_type = other DNA
cacagatgccattcagatac

SEQ ID NO: 386 - DNA, 20 bp, synthetic construct
  source 1..20: mol_type = other DNA
cacataggtcattcacaaat

SEQ ID NO: 387 - DNA, 20 bp, synthetic construct
  source 1..20: mol_type = other DNA
cagatagtcctttcagaaac

SEQ ID NO: 388 - DNA, 20 bp, synthetic construct
  source 1..20: mol_type = other DNA
cacataggctattcaaaatc

SEQ ID NO: 389 - DNA, 20 bp, synthetic construct
  source 1..20: mol_type = other DNA
cacatggcccatacagaaac

SEQ ID NO: 390 - DNA, 20 bp, synthetic construct
  source 1..20: mol_type = other DNA
ctcaaaggccattcaggaac

SEQ ID NO: 391 - DNA, 20 bp, synthetic construct
  source 1..20: mol_type = other DNA
cacataggccattcagaaac

SEQ ID NO: 392 - DNA, 20 bp, synthetic construct
  source 1..20: mol_type = other DNA
ccctggctccctcacccacc

SEQ ID NO: 393 - DNA, 20 bp, synthetic construct
  source 1..20: mol_type = other DNA
catatgggtcattcagaaac

SEQ ID NO: 394 - DNA, 20 bp, synthetic construct
  source 1..20: mol_type = other DNA
cacacaggcctttcagcaac

SEQ ID NO: 395 - DNA, 20 bp, synthetic construct
  source 1..20: mol_type = other DNA
cacagatgccattcagatac

SEQ ID NO: 396 - DNA, 20 bp, synthetic construct
  source 1..20: mol_type = other DNA
cacataggtcattcacaaat

SEQ ID NO: 397 - DNA, 20 bp, synthetic construct
  source 1..20: mol_type = other DNA
cagatagtcctttcagaaac

SEQ ID NO: 398 - DNA, 20 bp, synthetic construct
  source 1..20: mol_type = other DNA
agcaccaccacgtaggtgcc

SEQ ID NO: 399 - DNA, 20 bp, synthetic construct
  source 1..20: mol_type = other DNA
ctggtgaagatgagtggcga

SEQ ID NO: 400 - DNA, 20 bp, synthetic construct
  source 1..20: mol_type = other DNA
aagttgccccatgtcgacta

SEQ ID NO: 401 - DNA, 20 bp, synthetic construct
  source 1..20: mol_type = other DNA
ccccatgtcgactacatcga

SEQ ID NO: 402 - DNA, 20 bp, synthetic construct
  source 1..20: mol_type = other DNA
cccagagcatcccgtggaac

SEQ ID NO: 403 - DNA, 20 bp, synthetic construct
  source 1..20: mol_type = other DNA
ccggtggtcactctgtatgc

SEQ ID NO: 404 - DNA, 20 bp, synthetic construct
  source 1..20: mol_type = other DNA
agcacgcgcaggctgcgcat

SEQ ID NO: 405 - DNA, 20 bp, synthetic construct
  source 1..20: mol_type = other DNA
gcggcaccctcataggtgag

SEQ ID NO: 406 - DNA, 20 bp, synthetic construct
  source 1..20: mol_type = other DNA
gggccaccaatgcccaggac

SEQ ID NO: 407 - DNA, 20 bp, synthetic construct
  source 1..20: mol_type = other DNA
gtggccccaactgtgatgac

SEQ ID NO: 408 - DNA, 20 bp, synthetic construct
  source 1..20: mol_type = other DNA
gtgcctccagcgactgcagc

SEQ ID NO: 409 - DNA, 20 bp, synthetic construct
  source 1..20: mol_type = other DNA
acccctgcaccaggcattgc

SEQ ID NO: 410 - DNA, 20 bp, synthetic construct
  source 1..20: mol_type = other DNA
cctgaggaccagcgggtact

SEQ ID NO: 411 - DNA, 20 bp, synthetic construct
  source 1..20: mol_type = other DNA
gtggcagtggacacgggtcc

SEQ ID NO: 412 - DNA, 20 bp, synthetic construct
  source 1..20: mol_type = other DNA
tctacggcgtaggcccccag

SEQ ID NO: 413 - DNA, 20 bp, synthetic construct
  source 1..20: mol_type = other DNA
caagtgtcctctgatggtca

SEQ ID NO: 414 - DNA, 20 bp, synthetic construct
  source 1..20: mol_type = other DNA
gtcaaagttctagatgctgt

SEQ ID NO: 415 - DNA, 20 bp, synthetic construct
  source 1..20: mol_type = other DNA
ctgtccgaggcagtcctgcc

SEQ ID NO: 416 - DNA, 20 bp, synthetic construct
  source 1..20: mol_type = other DNA
tggccgtgcatgtgttcaga

SEQ ID NO: 417 - DNA, 20 bp, synthetic construct
  source 1..20: mol_type = other DNA
tgttcagaaaggctgctgat

SEQ ID NO: 418 - DNA, 20 bp, synthetic construct
  source 1..20: mol_type = other DNA
acacctgggagccatttgcc

SEQ ID NO: 419 - DNA, 20 bp, synthetic construct
  source 1..20: mol_type = other DNA
caccggtgccctgggtgtag

SEQ ID NO: 420 - DNA, 20 bp, synthetic construct
  source 1..20: mol_type = other DNA
agaggacacttggattcacc

SEQ ID NO: 421 - DNA, 20 bp, synthetic construct
  source 1..20: mol_type = other DNA
tagaactttgaccatcagag

SEQ ID NO: 422 - DNA, 20 bp, synthetic construct
  source 1..20: mol_type = other DNA
gcaggactgcctcggacagc

SEQ ID NO: 423 - DNA, 20 bp, synthetic construct
  source 1..20: mol_type = other DNA
gatggcaggactgcctcgga

SEQ ID NO: 424 - DNA, 20 bp, synthetic construct
  source 1..20: mol_type = other DNA
cacggccacattgatggcag

SEQ ID NO: 425 - DNA, 20 bp, synthetic construct
  source 1..20: mol_type = other DNA
agcagcctttctgaacacat

SEQ ID NO: 426 - DNA, 20 bp, synthetic construct
  source 1..20: mol_type = other DNA
tgatggtcaaagttctagat

SEQ ID NO: 427 - DNA, 20 bp, synthetic construct
  source 1..20: mol_type = other DNA
atggtcaaagttctagatgc

SEQ ID NO: 428 - DNA, 20 bp, synthetic construct
  source 1..20: mol_type = other DNA
tccgaggcagtcctgccatc

SEQ ID NO: 429 - DNA, 20 bp, synthetic construct
  source 1..20: mol_type = other DNA
atgacacctgggagccattt

SEQ ID NO: 430 - DNA, 20 bp, synthetic construct
  source 1..20: mol_type = other DNA
ggagccatttgcctctgggt

SEQ ID NO: 431 - DNA, 20 bp, synthetic construct
  source 1..20: mol_type = other DNA
tgggtaagttgccaaagaac

SEQ ID NO: 432 - DNA, 20 bp, synthetic construct
  source 1..20: mol_type = other DNA
ggattcaccggtgccctggg

SEQ ID NO: 433 - DNA, 20 bp, synthetic construct
  source 1..20: mol_type = other DNA
tgaccatcagaggacacttg

SEQ ID NO: 434 - DNA, 20 bp, synthetic construct
  source 1..20: mol_type = other DNA
gaactttgaccatcagagga

SEQ ID NO: 435 - DNA, 20 bp, synthetic construct
  source 1..20: mol_type = other DNA
ggacagcatctagaactttg

SEQ ID NO: 436 - DNA, 20 bp, synthetic construct
  source 1..20: mol_type = other DNA
cctcggacagcatctagaac

SEQ ID NO: 437 - DNA, 20 bp, synthetic construct
  source 1..20: mol_type = other DNA
ccaggtgtcatcagcagcct

SEQ ID NO: 438 - DNA, 20 bp, synthetic construct
  source 1..20: mol_type = other DNA
acccagaggcaaatggctcc

SEQ ID NO: 439 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNSKRGAAGSLMNVLNYPGMNHRVEITEGILADECAALLCDFYRMPRQVFNAQKKAQSSIN

SEQ ID NO: 440 - protein, 199 aa, synthetic construct
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSAGCAGEVRAFLQENTHVRLRIFAARIFDYDPLYKEALQMLRDAGAQVSIMTYDEFKHCWDTFVDHQGCPFQPWDGLDEHSQALSGRLRAILQNQGN

SEQ ID NO: 441 - protein, 83 aa, synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 442 - protein, 16 aa, synthetic construct
SGSETPGTSESATPES

SEQ ID NO: 443 - protein, 18 aa, synthetic construct
KRTADGSEFESPKKKRKV

SEQ ID NO: 444 - protein, 7 aa, synthetic construct
PKKKRKV

SEQ ID NO: 445 - protein, 16 aa, synthetic construct
KRPAATKKAGQAKKKK

SEQ ID NO: 446 - DNA, 241 bp, synthetic construct
  source 1..241: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggac

SEQ ID NO: 447 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 448 - DNA, 208 bp, synthetic construct
  source 1..208: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctgggga

SEQ ID NO: 449 - protein, 291 aa, synthetic construct
MSKSWGKFIEEEEAEMASRRNLMIVDGTNLGFRFKHNNSKKPFASSYVSTIQSLAKSYSARTTIVLGDKGKSVFRLEHLPEYKGNRDEKYAQRTEEEKALDEQFFEYLKDAFELCKTTFPTFTIRGVEADDMAAYIVKLIGHLYDHVWLISTDGDWDTLLTDKVSRFSFTTRREYHLRDMYEHHNVDDVEQFISLKAIMGDLGDNIRGVEGIGAKRGYNIIREFGNVLDIIDQLPLPGKQKYIQNLNASEELLFRNLILVDLPTYCVDAIAAVGQDVLDKFTKDILEIAEQ

SEQ ID NO: 450 - DNA, 794 bp, synthetic construct
  source 1..794: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 451 - RNA, 30 nt, synthetic construct
  source 1..30: mol_type = other RNA
atgactcagaaatgtgtccccagttgacac

SEQ ID NO: 452 - RNA, 23 nt, synthetic construct
  source 1..23: mol_type = other RNA
agaaatgtgtccccagttgacac

SEQ ID NO: 453 - RNA, 23 nt, synthetic construct
  source 1..23: mol_type = other RNA
agaaatccgtccttagttgacgg

SEQ ID NO: 454 - RNA, 22 nt, synthetic construct
  source 1..22: mol_type = other RNA
agacatgtgtccccagtgacac

SEQ ID NO: 455 - RNA, 23 nt, synthetic construct
  source 1..23: mol_type = other RNA
agaaatgtttccccagttgaaac

SEQ ID NO: 456 - RNA, 23 nt, synthetic construct
  source 1..23: mol_type = other RNA
agaaatgtgttcccagttaacac

SEQ ID NO: 457 - RNA, 23 nt, synthetic construct
  source 1..23: mol_type = other RNA
agaaatttgtccccagttgacaa

SEQ ID NO: 458 - protein, 1080 aa, synthetic construct
MSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKELSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHADEIAAVNIAMYEVCCQTGAFGKKQKKSDELPG

SEQ ID NO: 459 - protein, 1080 aa, synthetic construct
MSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKRLSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNARRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHADEIAAVNIAMYEVCCQTGAFGKKQKKSDELPG

SEQ ID NO: 460 - protein, 1080 aa, synthetic construct
MSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKRLSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADRKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHADEIAAVNIAMYEVCCQTGAFGKKQKKSDELPG

SEQ ID NO: 461 - protein, 166 aa, synthetic construct
SEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSIN

SEQ ID NO: 462 - protein, 17 aa, synthetic construct
KRTADGSESEPKKKRKV

SEQ ID NO: 463 - protein, 1315 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSKRTADGSEFESPKKKRKVSGGSSGGSMSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKRLSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHAAEIAAVNIAMYEVCCQTGAFGKKQKKSDELPGKRPAATKKAGQAKKKK

SEQ ID NO: 464 - protein, 1453 aa, synthetic construct
MKRTADGSEFESPKKKRKVMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISYSPCFSAGCAGEVRAFLQENTHVRLRIFAARIFDYDPLYKEALQMLRDAGAQVSIMTYDEFKHCWDTFVDHQGCPFQPWDGLDEHSQALSGRLRAILQNQGNSGGSSGGSKRTADGSEFESPKKKRKVSGGSSGGSMSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKRLSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHAAEIAAVNIAMYEVCCQTGAFGKKQKKSDELPGGSPKKKRKVSGGSTNLSDIIEKETGKQLVIQESILMLPEEVEEVIGNKPESDILVHTAYDESTDENVMLLTSDAPEYKPWALVIQDSNGENKIKMLSGGSSGGSKRTADGSESEPKKKRKV

SEQ ID NO: 465 - protein, 34 aa, synthetic construct
SGGSSGGSKRTADGSEFESPKKKRKVSGGSSGGS

SEQ ID NO: 466 - protein, 1080 aa, synthetic construct
MSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKRLSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHAAEIAAVNIAMYEVCCQTGAFGKKQKKSDELPG

SEQ ID NO: 467 - protein, 1080 aa, synthetic construct
MSSDVVRPYNTKLLPDNRKHNMFLQTFKRLNSISLNHFDLLICLYAAITNKKAEEYKSEKEAHVTADSLCAINWFRPMSKRYSKYATTTFNMLELFKEYSGHEPDAYSKNYLMSNIDSDRFVWVDCRKFAKDFAYQMELGFHEFTVLAETLLANSILVLNESTKANWAWGTVSALYGGGDKEDSTLKSKILLAFVDALNNHELKTKREILNQVCESLKYQSYQDMYVDFRSVVDENGNKKSPRGSMPIVTKFETDDLISDNQRKAMISNFTKNAAAKAAKKPIPYLDRLKEHMVSLCDEYNVYAWAAAITNSNADVTARNTRNLTFIGEQNSRRKRLSVLQTTTNEKAKDILNKINDNLIQEVRYTPAPKHLGRDLANLFDTLKEKDINNIENEEEKQNVINDCIEQYVDDCRSLNRNPIAALLKHISRYYEDFSAKNFLDGAKLNVLTEVVNRQKAHPTIWSEKAYTWISKFDKNRRQANSSLVGWVVPPEEVHKEKIAGQQSMMWVTLTLLDDGKWVKHHIPFSDSRYYSEVYAYNPNLPYLDGGIPRQSKFGNKPTTNLTAESQALLANSKYKKANKSFLRAKENATHNVRVSPNTSLCIRLLKDSAGNQMFDKIGNVLFGMQINHKITVGKPNYKIEVGDRFLGFDQNQSENHTYAVLQRVSESSHDTHHFNGWDVKVLEKGKVTSDVIVRDEVYDQLSYEGVPYDSSKFAEWRDKRRRFVLENLSIQLEEGKTFLTEFDKLNKDSLYRWNMNYLKLLRKAIRAGGKEFAKIAKTEIFELAVERFGPINLGSLSQISLKMIASFKGVVQSYFSVSGCVDDASKKAHDSMLFTFMCAAEEKRTNKREEKTNRAASFILQKAYLHGCKMIVCEDDLPVADGKTGKAQNADRMDWCARALAKKVNDGCVAMSICYRAIPAYMSSHQDPFVHMQDKKTSVLRPRFMEVNKDSIRDYHVAGLRRMLNSKSDAGTSVYYRQAALHFCEALGVSPELVKNKKTHAAELGKHMGSAMLMPWRGGRVYIASKKLTSDAKSVKYCGEDMWQYHADEIAAVNIAMYEVCCQTGAFGKKQKKSDELPG

(19) United States
(12) Patent Application Publication
(10) Pub. No.: US 2025/0283063 A1
(43) Pub. Date: Sep. 11, 2025

(54) NOVEL CRISPR-CAS12I SYSTEMS AND USES THEREOF

(71) Applicant: HUIDAGENE THERAPEUTICS (SINGAPORE) PTE. LTD., Singapore (SG)

(72) Inventors: Hainan ZHANG, Shanghai (CN); Jingxing ZHOU, Shanghai (CN); Haoqiang WANG, Shanghai (CN); Weihong ZHANG, Shanghai (CN)

(21) Appl. No.: 18/859,842

(22) PCT Filed: May 25, 2023
(86) PCT No.: PCT/CN2023/090695

§ 371 Date: Oct. 24, 2024

(30) Foreign Application Priority Data
Apr. 25, 2022 (WO) PCT/CN2022/089074
Nov. 2, 2022 (WO) PCT/CN2022/129376
Jan. 20, 2023 (WO) PCT/CN2023/073420

(51) Int. Cl. C12N 9/22; A61K 48/00; C12N 15/11; C12N 15/82; C12N 15/88; C12N 15/90

(52) U.S. Cl. CPC C12N 9/226; C12N 15/11; C12N 15/8213; C12N 15/88; C12N 15/907; A61K 48/00; C07K 2319/09; C12N 2310/20; C12N 2800/80

(57) ABSTRACT

The disclosure provides Cas12i polypeptides, fusion proteins comprising such Cas12i polypeptides, CRISPR-Cas12i systems comprising such Cas12i polypeptides or fusion proteins, and methods of using the same.

### CROSS-REFERENCE TO RELATED APPLICATIONS

This application claims the benefits of and priorities to PCT Patent Application No. PCT/CN2022/089074, filed on Apr. 25, 2022, entitled “NOVEL CRISPR-CAS12I SYSTEMS”, PCT Patent Application No. PCT/CN2022/129376, filed on Nov. 2, 2022, entitled “NOVEL CRISPR-CAS12I SYSTEMS AND USES THEREOF”, and PCT Patent Application No. PCT/CN2023/073420, filed on Jan. 20, 2023, entitled “NOVEL CRISPR-CAS12I SYSTEMS AND USES THEREOF” the entire contents of which, including any sequence listing and drawings, are incorporated herein by reference in their entireties.

### REFERENCE TO AN ELECTRONIC SEQUENCE LISTING

The disclosure contains an electronic sequence listing (“HEP001PCT3. xml” created on Apr. 25, 2023, by software “WIPO Sequence” according to WIPO Standard ST. 26), which is incorporated herein by reference in its entirety. According to WIPO Standard ST. 26, symbol “t” is used to denote both T in DNA and U in RNA (See “Table 1: List of nucleotides symbols”, the definition of symbol “t” is “thymine in DNA/uracil in RNA (t/u)”). Thus, in a sequence listing prepared according to ST. 26, wherever a sequence is an RNA, the T in the sequence shall be deemed as U.

### BACKGROUND

The clustered regularly interspaced short palindromic repeats-Cas (CRISPR-Cas) systems, including type II Cas9 and type V Cas12 systems, which serve in the adaptive immunity of prokaryotes against viruses, have been developed into genome editing tools. Compared with type II systems, the type V systems including V-A to V-K showed more functional diversity. Amongst them, Cas12i has a relatively smaller size compared to SpCas9 and Cas12a. Cas12i is characterized by the capability of autonomously processing precursor crRNA (pre-crRNA) to form short mature crRNA.

Cas12i mediates cleavage of dsDNA with a single RuvC domain, by preferentially nicking the non-target strand and then cutting the target strand. These intrinsic features of Cas12i enable multiplex high-fidelity genome editing.

Citation or identification of any document in the disclosure is not an admission that such a document is available as prior art to the disclosure. Each of the references mentioned or cited in the disclosure is incorporated by reference in its entirety.

### SUMMARY

It is against the above background that the disclosure provides certain advantages and advancements over the prior art. Although the disclosure is not limited to specific advantages or functionalities, in one aspect, the disclosure provides a Cas12i polypeptide comprising an amino acid substitution at E336, V880, G883, D892, and/or M923 of SEQ ID NO: 458.

In another aspect, the disclosure provides a system comprising:
(1) the Cas12i polypeptide of the disclosure or a polynucleotide encoding the Cas12i polypeptide; and
(2) a guide nucleic acid or a polynucleotide encoding the guide nucleic acid, the guide nucleic acid comprising:
(i) a direct repeat (DR) sequence capable of forming a complex with the Cas12i polypeptide; and
(ii) a spacer sequence capable of hybridizing to a target sequence of a target DNA, thereby guiding the complex to the target DNA.

In yet another aspect, the disclosure provides a polynucleotide encoding the Cas12i polypeptide of the disclosure. In yet another aspect, the disclosure provides a vector comprising the polynucleotide the disclosure.

In yet another aspect, the disclosure provides a ribonucleoprotein (RNP) comprising the Cas12i polypeptide of the disclosure and a guide nucleic acid optionally as defined in the disclosure.

In yet another aspect, the disclosure provides a lipid nanoparticle (LNP) comprising the Cas12i polypeptide of the disclosure or the system of the disclosure.

In yet another aspect, the disclosure provides a method for modifying a target DNA, comprising contacting the target DNA with the system of the disclosure, the vector of the disclosure, the ribonucleoprotein of the disclosure, or the lipid nanoparticle of the disclosure, wherein the spacer sequence is capable of hybridizing to a target sequence of the target DNA, wherein the target DNA is modified by the complex.

In yet another aspect, the disclosure provides a cell modified by the method of the disclosure.

In yet another aspect, the disclosure provides a pharmaceutical composition comprising (1) the system of the disclosure, the vector of the disclosure, the ribonucleoprotein of the disclosure, the lipid nanoparticle of the disclosure, or the cell of the disclosure; and (2) a pharmaceutically acceptable excipient.

In yet another aspect, the disclosure provides a method for diagnosing, preventing, or treating a disease in a subject in need thereof, comprising administering to the subject the system of the disclosure, the vector of the disclosure, the ribonucleoprotein of the disclosure, the lipid nanoparticle of the disclosure, the cell of the disclosure, or the pharmaceutical composition of the disclosure, wherein the disease is associated with a target DNA, wherein the spacer sequence is capable of hybridizing to a target sequence of the target DNA, wherein the target DNA is modified by the complex, and wherein the modification of the target DNA diagnose, prevents, or treats the disease.

In yet another aspect, the disclosure provides a method of detecting a target DNA, comprising contacting the target DNA with the system of the disclosure, wherein the target DNA is modified by the complex, and wherein the modification detects the target DNA.

The details of one or more embodiments of the disclosure are set forth in the description below. Other features or advantages of the disclosure will be apparent from the following drawings and detailed description of several embodiments, and also from the appended claims. It is understood that any aspect or embodiment of the disclosure can be combined with any other aspect or embodiment of the disclosure to constitute another embodiment explicitly or implicitly disclosed herein unless otherwise indicated.

### OVERVIEW

Cas12i, as a subtype of Class 2, Type V CRISPR associated protein (Cas12), is capable of binding to or function on a target nucleic acid (e.g., a dsDNA) as guided by a guide nucleic acid (e.g., a guide RNA (gRNA, used interchangeably with single guide RNA or sgRNA in the disclosure)) comprising a guide sequence targeting the target nucleic acid. In some embodiments, the target nucleic acid is eukaryotic.

Without wishing to be bound by theory, in some embodiments, the guide nucleic acid comprises a scaffold sequence (used interchangeable with a direct repeat sequence in the disclosure) responsible for forming a complex with the Cas12i, and a guide sequence (used interchangeable with a spacer sequence in the disclosure) that is intentionally designed to be responsible for hybridizing to a target sequence of the target nucleic acid, thereby guiding the complex comprising the Cas12i and the guide nucleic acid to the target nucleic acid.

Referring to FIG. 20, an exemplary target dsDNA (e.g., a target gene) is depicted to comprise a 5′ to 3′ single DNA strand and a 3′ to 5′ single DNA strand.

An exemplary guide nucleic acid is depicted to comprise a guide sequence and a scaffold sequence. The guide sequence is designed to hybridize to a part of the 3′ to 5′ single DNA strand, and so the guide sequence “targets” that part. And thus, the 3′ to 5′ single DNA strand is referred to as a “target strand (TS)” of the target dsDNA, while the opposite 5′ to 3′ single DNA strand is referred to as a “nontarget strand (NTS)” of the target dsDNA. That part of the target strand based on which the guide sequence is designed and to which the guide sequence may hybridize is referred to as a “target sequence”, while the opposite part on the nontarget strand corresponding to that part is referred to as the “protospacer sequence”, which is 100% (fully) reversely complementary to the target sequence.

Generally, a nucleic acid sequence (e.g., a DNA sequence, an RNA sequence) is written in 5′ to 3′ direction/orientation.

For example, for a DNA sequence of ATGC, it is usually understood as 5′-ATGC-3′ unless otherwise indicated. Its reverse sequence is 5′-CGTA-3′, its fully complement sequence is 5′-TACG-3′, and its fully reverse complement sequence is 5′-GCAT-3′.

Generally, the double-strand sequence of a dsDNA may be represented with the sequence of its 5′ to 3′ single DNA strand conventionally written in 5′ to 3′ direction/orientation unless otherwise indicated.

For example, for a dsDNA having a 5′ to 3′single DNA strand of 5′-ATGC-3′a nd a 3′ to 5′ single DNA strand of 3′-TACG-5′, the dsDNA may be simply represented as 5′-ATGC-3′.

5′-----ATGC-----3′

3′-----TACG-----5′

It should be noted that either the 5′ to 3′ single DNA strand or the 3′ to 5′ single DNA strand of a dsDNA can be a nontarget strand from which a protospacer sequence is selected or a target strand to which the guide sequence is designed to hybridize.

Generally, for a gene as a dsDNA, the 5′ to 3′ single DNA strand is the sense strand of the gene, and the 3′ to 5′ single DNA strand is the antisense strand of the gene. But it should be noted that either the sense strand or the antisense strand of a gene can be a nontarget strand from which a protospacer sequence is selected or a target strand to which the guide sequence is designed to hybridize.

To hybridize to a target dsDNA, in one embodiment, the guide sequence of a guide nucleic acid (e.g., a guide RNA) is designed to have a RNA sequence of 5′-AUGC-3′ that is fully reversely complementary to the 3′ to 5′ strand of the target dsRNA, which would be set forth in ATGC in the electric sequence listing but annotated as RNA; and in another embodiment, the guide sequence of a guide nucleic acid (e.g., a guide RNA) is designed to have a RNA sequence of 5′-GCAU-3′ that is fully reversely complementary to the 5′ to 3′ strand of the target dsRNA, which would be set forth in GCAT in the electric sequence listing but annotated as RNA.

In the case that the guide sequence of a guide nucleic acid is fully reversely complementary to the target sequence and the target sequence is fully reversely complementary to the protospacer sequence, the guide sequence is identical to the protospacer sequence except for the U in the guide sequence if it is an RNA sequence and correspondingly the T in the protospacer sequence. According to WIPO standard ST. 26, symbol “t” is used to denote both T in DNA and U in RNA (See “Table 1: List of nucleotides symbols”, the definition of symbol “t” is “thymine in DNA/uracil in RNA (t/u)”). Thus, in the sequence listing of the disclosure prepared according to ST. 26, such a guide sequence could be set forth in the same sequence as a corresponding protospacer sequence. For convenience, a single SEQ ID NO in the sequence listing can be used to denote both such guide sequence and protospacer sequence, although such a single SEQ ID NO may be marked as either DNA or RNA in the sequence listing. When a reference is made to such a SEQ ID NO that sets forth a protospacer/guide sequence, it refers to either a protospacer sequence that is a DNA sequence or a guide sequence that may be an RNA sequence depending on the context, no matter whether it is marked as DNA or RNA in the sequence listing.

### Term

Unless otherwise specified, all technical and scientific terms used in the disclosure have the meaning commonly understood by one of ordinary skill in the art to which the disclosure belongs. Throughout the specification, several terms are employed that are defined in the following paragraphs. Other definitions are also found within the body of the specification.

As used herein, the terms “nucleic acid”, “nucleic acid molecule”, or “polynucleotide” are used interchangeably. They refer to a polymer of deoxyribonucleotides or ribonucleotides or their mixtures in either single- or double-stranded form, and unless otherwise stated, encompass known analogs of natural nucleotides that can function in a similar manner as naturally occurring nucleotides. The terms encompass nucleic acid-like structures with synthetic backbones, as well as amplification products. DNAs and RNAs are both polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O (6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

As used herein, the term “polypeptide” and “protein” are used interchangeably to refer to polymers of amino acids of any length. The polymer may be linear or branched, it may comprise modified amino acids, and it may be interrupted by non-amino acids. The terms also encompass an amino acid polymer that has been modified; for example, by disulfide bond formation, glycosylation, lipidation, acetylation, phosphorylation, or any other manipulation, such as conjugation with a labeling component.

As used herein, a “fusion protein” refers to a protein created through the joining of two or more originally separate proteins, or portions thereof. In some embodiments, a linker may be present between each protein.

As used herein, the term “heterologous,” in reference to polypeptide domains, refers to the fact that the polypeptide domains do not naturally occur together (e.g., in the same polypeptide). For example, in fusion proteins generated by the hand of man, a polypeptide domain from one polypeptide may be fused to a polypeptide domain from a different polypeptide. The two polypeptide domains would be considered “heterologous” with respect to each other, as they do not naturally occur together.

As used herein, the term “nuclease” refers to a polypeptide capable of cleaving the phosphodiester bonds between the nucleotide subunits of nucleic acids; the term “endonuclease” refers to a polypeptide capable of cleaving the phosphodiester bond within a polynucleotide chain.

As used herein, the term “Cas12i” is used interchangeably with Cas12i protein or Cas12i polypeptide in the disclosure and used in its broadest sense and includes parental or reference Cas12i proteins (e.g., Cas12i protein comprising any of SEQ ID NOs: 1-10), derivatives or variants thereof, and functional fragments such as nucleic acid-binding fragments thereof, including endonuclease deficient (dead) Cas12i polypeptides, and Cas12i nickases.

As used herein, the term “guide nucleic acid” refers to a nucleic acid-based molecule capable of forming a complex with a CRISPR-Cas protein (e.g., a Cas12i of the disclosure) (e.g., via a scaffold sequence of the guide nucleic acid), and comprises a sequence (e.g., guide sequences) that are sufficiently complementary to a target nucleic acid to hybridize to the target nucleic acid and guide the complex to the target nucleic acid, which include but are not limited to RNA-based molecules, e.g., guide RNA. As used herein, the term “crRNA” is used interchangeably with guide RNA (gRNA), single guide RNA (sgRNA), or RNA guide. As used in the disclosure, the term “guide sequence” is used interchangeably with the term “spacer sequence”, and the term “scaffold sequence” is used interchangeably with the term “direct repeat sequence”. The guide nucleic acid may be a DNA molecule, an RNA molecule, or a DNA/RNA mixture molecule. By “DNA/RNA mixture molecule” it refers to a nucleic acid comprising both one or more modified or unmodified ribonucleotides and one or more modified or unmodified deoxyribonucleotides, whether consecutive or not. However, by “DNA molecule” or “RNA molecule” it may also refer to a DNA molecule containing one or more modified or unmodified ribonucleotides, whether consecutive or not, or an RNA molecule containing one or more modified or unmodified deoxyribonucleotides, whether consecutive or not.

As used herein, the term “complex” refers to a grouping of two or more molecules. In some embodiments, the complex comprises a polypeptide and a nucleic acid interacting with (e.g., binding to, coming into contact with, adhering to) one another. As used herein, the term “complex” can refer to a grouping of a guide nucleic acid and a polypeptide (e.g., a Cas12i polypeptide). As used herein, the term “complex” can refer to a grouping of a guide nucleic acid, a polypeptide, and a target nucleic acid.

As used herein, the term “activity” refers to a biological activity. In some embodiments, the activity includes enzymatic activity, e.g., catalytic ability of an effector. For example, the activity can include nuclease activity, e.g., DNA nuclease activity, dsDNA endonuclease activity, guide sequence-specific (on-target) dsDNA endonuclease activity, guide sequence-independent (off-target) dsDNA endonuclease activity.

As used herein, the term “spacer sequence-specific (on-target) dsDNA cleavage” may be termed as “dsDNA cleavage” for short unless otherwise indicated.

As used herein, the term “cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or cohesive ends.

As used herein, the meanings of “cleaving a nucleic acid” or “modifying a nucleic acid” may overlap. Modifying a nucleic acid includes not only modification of a mononucleotide but also insertion or deletion of a nucleic acid fragment.

As used herein, the term “on-target” refers to binding, cleavage, and/or editing of an intended or expected region of DNA, for example, by Cas12i of the disclosure.

As used herein, the term “off-target” refers to binding, cleavage, and/or editing of an unintended or unexpected region of DNA, for example, by Cas12i of the disclosure. In some embodiments, a region of DNA is an off-target region when it differs from the region of DNA intended or expected to be bound, cleaved and/or edited by 1, 2, 3, 4, 5, 6, 7, 8, 9, 10 or more nucleotides.

As used herein, if a DNA sequence, for example, 5′-ATGC-3′ is transcribed to an RNA sequence, with each dT (deoxythymidine, or “T” for short) in the primary sequence of the DNA sequence replaced with a U (uridine) and each dA (deoxyadenosine, or “A” for short), dG (deoxyguanosine, or “G” for short), and dC (deoxycytidine, or “C” for short) replaced with A (adenosine), G (guanosine), and C (cytidine), respectively, for example, 5′-AUGC-3′, it is said in the disclosure that the DNA sequence “encodes” the RNA sequence.

As used herein, the term “protospacer adjacent motif” or “PAM” refers to a short sequence (or a motif) adjacent to a protospacer sequence on the nontarget strand of a dsDNA recognized by CRISPR complexes.

As used herein, the term “adjacent” includes instances wherein there is no nucleotide between the protospacer sequence and the PAM and also instances wherein there are a small number (e.g., 1, 2, 3, 4, or 5) of nucleotides between the protospacer sequence and the PAM. As used herein, A “immediately adjacent (to)” B, A “immediately 5′ to” B, and A “immediately 3′ to” B mean that there is no nucleotide between A and B.

As described herein, the guide sequence is so designed to be capable of hybridizing to a target sequence. As used herein, the term “hybridize”, “hybridizing”, or “hybridization” refers to a reaction in which one or more polynucleotide sequences react to form a complex that is stabilized via hydrogen bonding between the bases of the one or more polynucleotide sequences. The hydrogen bonding may occur by Watson Crick base pairing, Hoogstein binding, or in any other sequence specific manner. A polynucleotide sequence capable of hybridizing to a given polynucleotide sequence is referred to as the “complement” of the given polynucleotide sequence. As used herein, the hybridization of a guide sequence and a target sequence is so stabilized to permit a Cas12i polypeptide that is complexed with a guide nucleic acid comprising the guide sequence or a function domain (e.g., a deaminase domain) associated (e.g., fused) with the Cas12i polypeptide to act (e.g., cleave, deaminize) at or near the target sequence or its complement (e.g., a sequence of a target DNA or its complement).

For the purpose of hybridization, in some embodiments, the guide sequence is reversely complementary to a target sequence. As used herein, the term “complementary” refers to the ability of nucleobases of a first polynucleotide sequence, such as a guide sequence, to base pair with nucleobases of a second polynucleotide sequence, such as a target sequence, by traditional Watson-Crick base-pairing. Two complementary polynucleotide sequences are able to non-covalently bind under appropriate temperature and solution ionic strength conditions. In some embodiments, a first polynucleotide sequence (e.g., a guide sequence) comprises 100% (fully) complementarity to a second nucleic acid (e.g., a target sequence). In some embodiments, a first polynucleotide sequence (e.g., a guide sequence) is complementary to a second polynucleotide sequence (e.g., a target sequence) if the first polynucleotide sequence comprises at least about 80%, 81%, 82%, 83%, 84%, 85%, 86%, 87%, 88%, 89%, 90%, 91%, 92%, 93%, 94%, 95%, 96%, 97%, 98%, or 99% complementarity to the second nucleic acid. As used herein, the term “substantially complementary” refers to a polynucleotide sequence (e.g., a guide sequence) that has a certain level of complementarity to a second polynucleotide sequence (e.g., a target sequence) such that the first polynucleotide sequence (e.g., a guide sequence) can hybridize to the second polynucleotide sequence (e.g., a target sequence) with sufficient affinity to permit a Cas12i polypeptide that is complexed with the first polynucleotide sequence or a nucleic acid comprising the first polynucleotide sequence or a function domain associated (e.g., fused) with the Cas12i polypeptide to act (e.g., cleave, deaminize) on the target sequence or its complement (e.g., a sequence of a target DNA or its complement). In some embodiments, a guide sequence that is substantially complementary to a target sequence has 100% or less than 100% complementarity to the target sequence. In some embodiments, a guide sequence that is substantially complementary to a target sequence has at least about 80%, 81%, 82%, 83%, 84%, 85%, 86%, 87%, 88%, 89%, 90%, 91%, 92%, 93%, 94%, 95%, 96%, 97%, 98%, or 99% complementarity to the target sequence.

As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. The comparison of sequences and determination of percent identity between two sequences can be accomplished using a mathematical algorithm. As is well known in the art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. In some embodiments, the sequence identity is calculated by global alignment, for example, using the Needleman-Wunsch algorithm and an online tool at ebi. ac. uk/Tools/psa/emboss_needle/. In some embodiments, the sequence identity is calculated by local alignment, for example, using the Smith-Waterman algorithm and an online tool at ebi. ac. uk/Tools/psa/emboss_water/.

As used herein, the term “variant” refers to an entity that shows significant structural identity with a reference entity (e.g., a wild-type sequence) but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a polypeptide may have a characteristic sequence element comprising a plurality of amino acids having designated positions relative to one another in linear or three-dimensional space and/or contributing to a particular biological function; a nucleic acid may have a characteristic sequence element comprising a plurality of nucleotide residues having designated positions relative to one another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide (e.g., a nuclease described herein) that is at least 60%, 65%, 70%, 75%, 80%, 85%, 86%, 87%, 88%, 89%, 90%, 91%, 92%, 93%, 94%, 95%, 96%, 97%, 98% or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide, e.g., nuclease activity. In some embodiments, a variant polypeptide lacks one or more of the biological activities of the reference polypeptide. In some embodiments, a variant polypeptide shows a reduced level of one or more biological activities (e.g., nuclease activity, e.g., off-target nuclease activity) as compared with the reference polypeptide. In some embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2%, or 1% of the residues in the variant are substituted as compared with the parent or reference polypeptide. In some embodiments, a variant has 10, 9, 8, 7, 6, 5, 4, 3, 2, or 1 substituted residue as compared with a parent or reference polypeptide. Often, a variant has a very small number (e.g., fewer than 5, 4, 3, 2, or 1) of substituted functional residues (i.e., residues that participate in a particular biological activity). In some embodiments, a variant has not more than 5, 4, 3, 2, or 1 additions or deletions, and often has no additions or deletions, as compared with the parent or reference polypeptide. Moreover, any additions or deletions are typically fewer than about 25, about 20, about 19, about 18, about 17, about 16, about 15, about 14, about 13, about 12, about 11, about 10, about 9, about 8, about 7, about 6, and commonly are fewer than about 5, about 4, about 3, or about 2 residues. In some embodiments, the parent or reference polypeptide is a wild type. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to skilled artisans.

As used herein, the terms “non-naturally occurring” and “engineered” are used interchangeably and refer to artificial participation. When these terms are used to describe a nucleic acid or a polypeptide, it is meant that the nucleic acid or polypeptide is at least substantially freed from at least one other component of its association in nature or as found in nature.

Conservative substitutions of non-critical amino acids of a protein may be made without affecting the normal functions of the protein. Conservative substitutions refer to the substitution of amino acids with chemically or functionally similar amino acids. In some embodiments, a conservative amino acid substitution refers to an amino acid substitution that does not alter the relative charge or size characteristics of the protein in which the amino acid substitution was made. In some embodiments, a “conservative substitution” refers to a substitution of an amino acid made among amino acids within the following groups: i) methionine, isoleucine, leucine, valine, ii) phenylalanine, tyrosine, tryptophan, iii) lysine, arginine, histidine, iv) alanine, glycine, v) serine, threonine, vi) glutamine, asparagine and vii) glutamic acid, aspartic acid.

As used herein, the term “wild type” has the meaning commonly understood by those skilled in the art to mean a typical form of an organism, a strain, a gene, or a feature that distinguishes it from a mutant or variant when it exists in nature. It can be isolated from sources in nature and not intentionally modified.

As used herein, the description of “a variant (e.g., a Cas12i polypeptide) comprising an amino acid mutation (e.g., substitution) at a given position (e.g., E336) of a given polypeptide (e.g., SEQ ID NO: 458)” or similar description means that the polypeptide as set forth in the amino acid sequence of the given polypeptide serves as a parent or reference polypeptide, and the variant is a variant of the parent or reference polypeptide and comprises an amino acid mutation at a position of the amino acid sequence of the variant corresponding to the given position of the amino acid sequence of the given polypeptide. The position of the amino acid mutation in the amino acid sequence of the variant may be the same as the given position of the given polypeptide, for example, when the variant comprises just an amino acid substitution as compared with the given polypeptide and has the same length as the given polypeptide. The position of the amino acid mutation in the amino acid sequence of the variant may also be different from the given position of the given polypeptide, for example, when the variant comprises a N-terminal truncation as compared with the given polypeptide and the first N-terminal amino acid of the variant is not corresponding to the first N-terminal amino acid of the given polypeptide but to an amino acid within the given polypeptide, but the position of the amino acid mutation can be determined by alignment of the variant and the given polypeptide to identify the corresponding amino acids in their sequences as understood by a skilled in the art. For example, if the variant has a N-terminal truncation of 20 amino acids as compared with the given polypeptide, then the variant comprising an amino acid mutation at E336 of a given polypeptide means that the variant comprises an amino acid mutation at E316 of the variant since E316 in the variant is corresponding to E336 in the given polypeptide as determined by alignment of the variant and the given polypeptide.

As used herein, the description of “a variant (e.g., a Cas12i polypeptide) comprising a given amino acid substitution (e.g., E336R) relative to a given polypeptide (e.g., SEQ ID NO: 458)” means that the polypeptide as set forth in the amino acid sequence of the given polypeptide serves as a parent or reference polypeptide that does not comprise the given amino acid substitution, and the variant is a variant of the parent or reference polypeptide and comprises an amino acid substitution having the same type of substitution as the given amino acid substitution and at a position in the amino acid sequence of the variant corresponding to the position of the given amino acid substitution. For example, a Cas12i polypeptide comprising an amino acid substitution E336R relative to SEQ ID NO: 458 refers to the fact that the amino acid sequence of SEQ ID NO: 458 comprises amino acid E at position 336, and the Cas12i polypeptide comprises amino acid R at a position corresponding to position 336 of the amino acid sequence of SEQ ID NO: 458. The corresponding relationship of positions in two amino acid sequences as determined by alignment is explained in the previous paragraph.

As used herein, the terms “upstream” and “downstream” refer to relative positions within a single nucleic acid (e.g., DNA) sequence in a nucleic acid. “Upstream” and “downstream” relate to the 5′ to 3′ direction, respectively, in which transcription occurs. For a first sequence and a second sequence present on the same strand of a single nucleic acid written in 5′ to 3′ direction, the first sequence is upstream of the second sequence when the 3′ end of the first sequence is on the left side of the 5′ end of the second sequence, and the first sequence is downstream of the second sequence when the 5′ end of the first sequence is on the right side of the 3′ end of the second sequence. For example, a promoter is usually at the upstream of a sequence under the regulation of the promoter; and on the other hand, a sequence under the regulation of a promoter is usually at the downstream of the promoter.

As used herein, the term “regulatory element” refers to a DNA sequence that controls or impacts one or more aspects of transcription and/or expression is intended to include promoters, enhancers, silencers, termination signals, internal ribosome entry sites (IRES), and other expression control elements (e.g., transcription termination signals such as polyadenylation signals and poly-U sequences). Regulatory elements include those that direct constitutive expression of a nucleotide sequence in many types of host cells and those that direct expression of a nucleotide sequence only in certain host cells (e.g., tissue-specific regulatory sequences). Regulatory elements may also direct expression in a time-dependent manner, e.g., in a cell cycle-dependent or developmental stage-dependent manner, which may or may not be tissue or cell type specific.

As used herein, the term “operably linked” refers to a juxtaposition wherein the components described are in a relationship permitting them to function in their intended manner. A regulatory element “operably linked” to a functional element is associated in such a way that transcription, expression, and/or activity of the functional element is achieved under conditions compatible with the regulatory element. In some embodiments, “operably linked” regulatory elements are contiguous (e.g., covalently linked) with the functional elements of interest; in some embodiments, regulatory elements act in trans to or otherwise at a distance from the functional elements of interest.

As used herein, the term “cell” is understood to refer not only to a particular individual cell, but to the progeny or potential progeny of the cell. Because certain modifications may occur in succeeding generations due to either mutation or environmental influences, such progeny may not, in fact, be identical to the parent cell, but are still included within the scope of the term.

As used herein, the term “in vivo” means inside the body of an organism, and the terms “ex vivo” or “in vitro” means outside the body of an organism.

As used herein, the term “treat”, “treatment”, or “treating” is an approach for obtaining beneficial or desired results including clinical results. For purposes of the disclosure, the beneficial or desired clinical results include, but are not limited to, one or more of the following: alleviating one or more symptoms resulting from a disease, diminishing the extent of a disease, stabilizing a disease (e.g., preventing or delaying the worsening of a disease), preventing or delaying the spread (e.g., metastasis) of a disease, preventing or delaying the recurrence of a disease, reducing recurrence rate of a disease, delay or slowing the progression of a disease, ameliorating a disease state, providing a remission (partial or total) of a disease, decreasing the dose of one or more other medications required to treat a disease, delaying the progression of a disease, increasing the quality of life, and prolonging survival. Also encompassed by the term is a reduction of pathological consequence of a disease (such as cancer). The methods of the disclosure contemplate any one or more of these aspects of treatment.

As used herein, the term “disease” includes the terms “disorder” and “condition” and is not limited to those specific diseases that have been medically or clinically defined.

As used herein, reference to “not” a value or parameter generally means and describes “other than” a value or parameter. For example, the method is not used to treat cancer of type X means the method may be used to treat cancer of types other than X.

As used herein, the singular forms “a”, “an”, and “the” include plural referents unless the context clearly dictates otherwise. That is, articles “a/an” and “the” are used herein to refer to one or more than one (i.e., at least one) grammatical object of the article. For example, “an element” means one element or more than one element, e.g., two elements.

As used herein, the term “and/or” in a phrase such as “A and/or B” is intended to mean either or both of the alternatives, including both A and B, A or B, A (alone), and B (alone). Likewise, the term “and/or” in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, when the term “about” is ahead of a serious of numbers (for example, about 1, 2, 3), it is understood that each of the serious of numbers is modified by the term “about” (that is, about 1, about 2, about 3). The term “about X-Y” used herein has the same meaning as “about X to about Y.”

As used herein, the terms “about” and “approximately,” in reference to a number, is used herein to include numbers that fall within a range of 20%, 15%, 10%, 9%, 8%, 7%, 6%, 5%, 4%, 3%, 2%, or 1% in either direction (greater than or less than) of the number unless otherwise stated or otherwise evident from the context (except where such number would exceed 100% of a possible value).

As used herein, a numerical range includes the end values of the range, and each specific value within the range, for example, “16 to 100 nucleotides” includes 16 nucleotides and 100 nucleotides, and each specific value between 16 and 100, e.g., 17, 23, 34, 52, 78.

As used herein, the terms “comprise”, “include”, “contain”, and “have” are to be understood as implying that a stated element or a group of elements is included, but not excluding any other element or a group of elements, unless the context requires otherwise. In certain embodiments, the terms “comprise”, “include”, “contain”, and “have” are used synonymously.

As used herein, the phrase “consist essentially of” is intended to include any element listed after the phrase “consist essentially of” and is limited to other elements that do not interfere with or contribute to the activities or actions specified in the disclosure of the listed elements. Thus, the phrase “consist essentially of” is intended to indicate that the listed elements are required, but no other elements are optional, and may or may not be present depending on whether they affect the activities or actions of the listed elements.

As used herein, the phrase “consist of” means including but limited to any element after the phrase “consist of”. Thus, the phrase “consist of” indicates that the listed elements are required, and that no other elements can be present.

As used herein, the term “comprises” also encompasses the terms “consists essentially of” and “consists of”. It is understood that the “comprising” embodiments of the disclosure described herein also include “consisting essentially of” and “consisting” embodiments.

Throughout the specification, reference to “one embodiment”, “embodiment”, “a specific embodiment”, “a related embodiment”, “an embodiment”, “another embodiment”, or “a further embodiment” or a combination thereof means that specific elements, features, structures, or characteristics described in connection with the embodiment are included in at least one embodiment of the disclosure. Accordingly, the appearances of the foregoing phrases in various places throughout the specification are not necessarily all referring to the same embodiments. Furthermore, specific elements, features, structures, or characteristics may be combined in any suitable manner in one or more embodiments.

It is further noted that the claims may be drafted to exclude any optional element. As such, this statement is intended to serve as antecedent basis for use of such exclusive terminology as “solely”, “only”, and the like in connection with the recitation of claim elements, or use of a “negative” limitation.

### BRIEF DESCRIPTION OF THE DRAWINGS

An understanding of the features and advantages of the disclosure will be obtained by reference to the following detailed description that sets forth illustrative embodiments, in which the principles of the disclosure may be utilized, and the accompanying drawings of which:

FIG. 1 shows that hfCas12Max, an engineered variant of xCas12i, mediated high-efficient and high-specificity genome editing, and dCas12i base editor exhibited high base editing activity in mammalian cells. FIG. 1A, xCas12i mediated EGFP activation efficiency determined by flow cytometry. NC represents non-specific (non-targeting) control. FIG. 1B, Schematics of protein engineering strategy for mutants with high efficiency and high fidelity (specificity) using an activatable EGFP reporter screening system with on-targeted and off-targeted crRNA. FIG. 1C and FIG. 1D, Cas12Max exhibited significantly increased cleavage activity than xCas12i at reporter plasmids (FIG. 1C) or various genomic target sites (FIG. 1D). Each dot represents the mean indel frequency at one targeted site (n=3). FIG. 1E, NGS analysis showed that hfCas12Max retained comparable activity at TTR. 2-ON targets to Cas12Max and almost no activity at 6 OT sites. FIG. 1F, Both Cas12Max and hfCas12Max exhibited a broader PAM recognition profile including 5′-TN and 5′-TNN PAM than other Cas proteins. FIG. 1G, Comparison of indel activity of Cas12Max, hfCas12Max, LbCas12a, Ultra AsCas12a, SpCas9 and KKH-saCas9 at TTR locus. hfCas12Max retained comparable activity to Cas12Max, and higher gene-editing efficiency than other Cas proteins. Each dot represents one of three repeats of single target site. FIG. 1H, Schematics of different versions of dxCas12i adenine base editors. FIG. 1I, Comparison of A-to-G editing frequency and product purity at the KLF4 site of TadA8e. 1-dxCas12i-v1.2, v2.2 and v4.3, v4.3 showed a high editing activity of 80%. TadA8e-dxCas12i-v4.3, named as ABE-dCas12Max. TadA8e. 1 represents TadA8e-V106W. FIG. 1J, Schematics of different versions of dxCas12i cytosine base editors. FIG. 1K, Comparison of C-to-T editing frequency and product purity at the RUNX1 site of hA3A. 1-dxCas12i, -v1.2 v2.2 and v3.1, and also hA3A. 1-dLbCas12a, v3.1 showed a high editing activity of 50%. hA3A. 1-dxCas12i-v3.1, named as CBE-dCas12Max. hA3A. 1 represents human APOBEC3A-W104A.

FIG. 2 shows that hfCas12Max mediated high-efficiency gene editing ex vivo and in vivo. FIG. 2A, Schematics of hfCas12Max gene editing in primary human cells. FIG. 2B, Viability and indel activity of human CD3+T cells following delivery of hfCas12Max RNPs with three different TRAC targeting gRNAs at 1.6 μM and 3.2 μM respectively (n=2 or 3). NC represents blank control, untreated with RNP. FIG. 2C, Representative flow cytometric analysis of edited CD3+ T cell 5 days after RNP delivery. NC represents blank control, untreated with RNP. FIG. 2D, Schematics of in vivo non-liposome delivery containing IVT-mRNA, LNP packaging process. FIG. 2E, Editing efficiency of LNP packaging with hfCas12Max mRNA and Ttr targeting gRNA at increased concentrations in N2a cells (n=8). FIG. 2F, Schematics of Ttr locus. FIG. 2G, Indel rates of LNP packaging with hfCas12Max mRNA and Ttr targeting gRNA at three doses (0.1, 0.3 and 0.5 mpk) in C57 mouse (n=6). FIG. 2H, The A-to-G editing percentage of LNP packaging with dCas12i-ABE mRNA and Ttr targeting gRNA at 3 mpk in C57 mouse (n=2).

FIG. 3 shows screening for functional Cas12i in HEK293T cells. FIG. 3A, Transfection of plasmids coding Cas12i and gRNA mediate EGFP activation. FIG. 3B, Five of ten Cas12i nucleases mediated EGFP-activated efficiency in HEK293T cells.

FIG. 4 shows identification and characterization of type V-I systems. FIG. 4A, Nuclease domain organization of SpCas9, LbCas12a, and xCas12i. FIG. 4B, Effective spacer sequence length for xCas12i. FIG. 4C, PAM scope comparison of LbCas12a and xCas12i. xCas12i exhibited a higher dsDNA cleavage activity at 5′-TTN PAM than LbCas12a. FIG. 4D, Flow diagram for detection of genome cleavage activity by transfection of an all-in-one plasmid containing xCas12i and gRNA into HEK293T cells, followed by FACS and NGS analysis. FIG. 4E-FIG. 4F, xCas12i mediated robust genome cleavage (up to 90%) at Ttr locus in N2a cells and TTR and PCSK9 locus in HEK293T cells.

FIG. 5 shows screening for engineered xCas12i mutants with single point mutation and various dsDNA cleavage activity. FIG. 5A, The relative dsDNA cleavage activity of over 500 rationally engineered xCas12i mutants. v1.1 represents xCas12i with N243R, named as Cas12Max.

FIG. 6 shows additional xCas12i-N243 mutants mediated high-efficiency editing. FIG. 6A, Of all the saturated mutants of xCas12i-N243, xCas12i-N243R showed the mostly increased EGFP-activated fluorescence. FIG. 6B-FIG. 6C, xCas12i mutant with N243R increased 1.2, 5, and 20-fold activity at DMD. 1, DMD. 2 and DMD. 3 locus, respectively. FIG. 6D, Both Cas12Max (xCas12i-N243R) and Cas12Max-E336R (xCas12i-N243R+E336R) elevated EGFP-activated fluorescence at different PAM recognition sites.

FIG. 7 shows that Cas12Max induced off-target dsDNA cleavage activity at sites with mismatches using the reporter system (FIG. 7A) and targeted deep sequence (FIG. 7B).

FIG. 8 shows that hfCas12Max (xCas12i-N243R+E336R+D892R) mediates high-efficiency and high-specificity editing. FIG. 8A, Rational protein engineering screening of over 200 mutants for highly-fidelity (specificity) Cas12Max. Four mutants show significantly decreased cleavage activity at both OT (off-target) sites and retained cleavage activity at ON. 1 (on-target) site. FIG. 8B, Different versions of xCas12i mutants. FIG. 8C, v6.3 reduced off-target at OT. 1, OT. 2 and OT. 3 sites and retained indel activity at TTR-ON targets, compared to v1.1. FIG. 8D, v6.3 exhibited comparable indel activity at DMD. 1, DMD. 2, and higher at DMD. 3 locus, than v1.1. v1.1, i.e., Cas12Max. v6.3, named as hfCas12Max.

FIG. 9 shows comparison of the gene-editing efficiency of hfCas12Max with LbCas12a, Ultra AsCas12a, ABR001, and Cas12i^{HiFi} at TTR locus.

FIG. 10 shows that hfCas12Max mediated high-efficient and high-specific editing. FIG. 10A-FIG. 10B, Off-target efficiency of hfCas12Max, LbCas12a, and UltraAsCas12a at in-silico predicted off-target sites, determined by targeted deep sequencing. Sequences of on-target and predicted off-target sites are shown, PAM sequences are in blue and mismatched bases are in red.

FIG. 11 shows conserved cleavage sites of Cas12i. FIG. 11A, Sequence alignment of xCas12i, Cas12i1 and Cas12i2 shows that D650, D700, E875 and D1049 are conserved cleavage sites at RuvC domain. FIG. 11B, Introducing point mutations of D700A, D650A, E875A, or D1049A result in abolished activity of xCas12i.

FIG. 12 and FIG. 13 shows engineering for highly efficient dxCas12i-ABE. FIG. 12 and FIG. 13A, Engineering schematic of TadA8e. 1-dxCas12i. Four parts for engineering are indicated. FIG. 13B, TadA8e. 1-dxCas12i-v1.2 and v1.3 exhibit significantly increased A-to-G editing activity among various variants at KLKF4 site of genome. FIG. 13C, Increased A-to-G editing activity of TadA8e-dxCas12i-v2.2 by combining v1.2 and v1.3. FIG. 13D, Unchanged or even decreased editing activity from various dCas12-ABEs carrying different NLS at N-terminal. FIG. 13E, Increased A-to-G editing activity of TadA8e-dxCas12i-v4.3 by combining v2.2, changed-NLS linker and high-activity Tade8e.

FIG. 14 shows additional strategies for highly efficient dxCas12i-ABE. FIG. 14A, Schematics of different versions of dxCas12i ABEs. FIG. 14B, dxCas12i-ABE-N by TadA at the C-terminus of the dxCas12i slightly increased editing activity.

FIG. 15 shows comparison of editing frequencies induced by various dCas12-ABEs at different genomic target sites. FIG. 15A-FIG. 15B, Comparison of A-to-G editing frequencies induced by indicated TadA8e. 1-dxCas12i-v1.2, v2.2, and TadA8e. 1-dLbCas12a at PCSK9 and TTR genomic locus.

FIG. 16 shows characterization of dxCas12i-ABE in HEK293T cells. A-C, dCas12Max-ABE base editing of the target sites with TTN (FIG. 16A), ATN (FIG. 16B), and CTN (FIG. 16C) PAMs. FIG. 16D, dCas12Max-ABE base editing product purity at each target site with TTN PAM in FIG. 16A.

FIG. 17 shows comparison of editing frequencies induced by various dCas12-CBEs at different genomic target sites. FIG. 17A-FIG. 17B, Comparison of C-to-T editing frequencies and product purity induced by indicated hA3A. 1-dxCas12i, v1.2, v2.2, and hA3A. 1-dLbCas12a at DYRK1A and SITE4 genomic locus. hA3A. 1 represents human APOBEC3A-W104A.

FIG. 18 shows that hfCas12Max mediated high editing efficiency in HEK293 cells. FIG. 18A-FIG. 18C, Unchanged viability and proliferation and increased indel activity of HEK293 cells following delivery of hfCas12Max RNPs with TTR or TRAC targeting gRNA at increasing concentration (n=1).

FIG. 19 shows that hfCas12Max mediated high editing efficiency in mouse blastocyst. FIG. 19A, Schematics of hfCas12Max gene editing in mouse blastocyst. hfCas12Max mRNA and Ttr targeting gRNA were injected into mouse zygotes, and the injected zygotes were cultured into blastocyst stage for genotyping analysis by targeted deep sequencing. FIG. 19B, Indel rates of hfCas12Max targeting Ttr. 3 and Ttr. 12 in mouse blastocyst (n=12).

FIG. 20 is a schematic illustrating an exemplary target dsDNA, an exemplary guide nucleic acid having one DR sequence 5′ to one spacer sequence, and an exemplary Cas12i.

FIG. 21 shows the dsDNA cleavage activity of xCas12i when using various DR sequence variants.

FIG. 22 is a schematic illustrating the secondary structures of direct repeat sequences of the guide RNAs of the disclosure.

FIG. 23 shows another exemplary guide nucleic acid having three DR sequences and two spacer sequences, and each of the two spacer sequences is flanked by two DR sequences.

The figures herein are for illustrative purposes only and are not necessarily drawn to scale.

### DETAILED DESCRIPTION OF THE EXAMPLE EMBODIMENTS

### Overview

The disclosure provides Cas12i polypeptides with high spacer sequence-specific (on-target) dsDNA cleavage activity and/or low spacer sequence-independent (off-target) dsDNA cleavage activity based on parent or reference Cas12i polypeptides, and fusions and uses thereof.

In some embodiments, the parent or reference Cas12i polypeptide may be: (i) any one of SEQ ID NOs: 1-10 (Cas12i3 to Cas12i12) of the disclosure and Cas12i polypeptides (such as, Cas12i1 and Cas12i2) in PCT/CN2022/089074, PCT/CN2022/129376, PCT/CN2023/073420, WO2019090173A1, WO2019178033A1, WO2019222555A1, WO2020018142A1, WO2020180699A1, WO2020252378A1, WO2021007563A1, WO2021041569A1, WO2021046442A1, WO2021050534A1, WO2021113522A1, WO2021202800A1, WO2021243267A3, WO2021257730A3, WO2022040224A1, WO2022094313A1, WO2022094309A1, WO2022094329A1, WO2022094323A8, WO2022150608A1, WO2022159585A1, WO2022159741A1, WO2022162623A1, WO2022162622A1, WO2022174099A3, WO2022192391A1, WO2022192381A1, WO2022256440A3, WO2022256619A3, WO2022256655A3, WO2022256642A3, WO2023004422A3, WO2023010084A3, WO2023018856A1, WO2023018858A1, WO2023019243A1, WO2023034475A1, WO2023039472A2, and WO2023039534A2, (ii) a naturally-occurring ortholog, paralog, or homolog of any one of (i); (iii) a Cas12i polypeptide having a sequence identity of at least about 40%, 50%, 60%, 70%, 80%, 85%, 90%, 91%, 92%, 93%, 94%, 95%, 96%, 97%, 98%, or 99% to any one of (i) and (ii); or (iv) any mutant or variant of (i) to (iii). The parent or reference Cas12i polypeptide may be a wild type or not.

### Representative Cas12i Polypeptides and Characterization of Cas12i Polypeptide

In some aspects of the disclosure, the Cas12i polypeptide of the disclosure has or retains or has improved endonuclease activity against a target DNA for on-target DNA cleavage. Still for the purpose of on-target DNA cleavage, the Cas12i polypeptide of the disclosure may not only have on-target endonuclease activity but also substantially lack off-target endonuclease activity such that it can have specificity for a target DNA. On the other hand, the Cas12i polypeptide of the disclosure can be engineered to substantially lack endonuclease activity (either on-target or off-target) but retain its ability of complexing with a guide nucleic acid and thus being guided to a target DNA, so as to indirectly guide a functional domain associated with the Cas12i polypeptide to the target DNA. Therefore, the characterization of the Cas12i polypeptide of the disclosure is not limited to its ability of on-target DNA cleavage.

In some embodiments, the Cas12i polypeptide has spacer sequence-specific (on-target) dsDNA cleavage activity.

In some embodiments, the Cas12i polypeptide substantially retains the spacer sequence-specific (on-target) dsDNA cleavage activity of SEQ ID NO: 458 or SEQ ID NO: 1.

### Increased On-Target Cleavage

As representatives of the disclosure, in an aspect, the disclosure provides an Cas12i polypeptide comprising an amino acid substitution at E336, V880, G883, D892, and/or M923 of SEQ ID NO: 458. The polypeptide as set forth in the amino acid sequence of SEQ ID NO: 458 (Cas12Max; xCas12i-N243R) serves as a parent or reference polypeptide, based on which the Cas12i polypeptide of the disclosure is engineered.

In some embodiments, the Cas12i polypeptide has an increased spacer sequence-specific (on-target) dsDNA cleavage activity compared to that of SEQ ID NO: 458 or SEQ ID NO: 1 when both are used in combination with a same guide nucleic acid, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more.

In some embodiments, the Cas12i polypeptide has a sequence identity of at least about 80%, at least about 85%, at least about 90%, at least about 95%, or at least about 99% to SEQ ID NO: 458. In some embodiments, the Cas12i polypeptide has a sequence identity of at least about 80%, at least about 85%, at least about 90%, at least about 95%, or at least about 99% to any one of SEQ ID NOs: 1-10.

Typically, amino acid substitution is a substitution with a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F), a polar amino acid residue (such as, Serine (Ser/S), Threonine (Thr/T), Tyrosine (Tyr/Y), Asparagine (Asn/N), Glutamine (Gln/Q)), a positively charged amino acid residue (such as, Lysine (Lys/K), Arginine (Arg/R), Histidine (His/H)), or a negatively charged amino acid residue (such as, Aspartic Acid (Asp/D), Glutamic Acid (Glue/E)).

In some embodiments, the amino acid substitution is a substitution with a positively charged amino acid residue (such as, Lysine (Lys/K), Arginine (Arg/R), Histidine (His/H)). In some embodiments, the amino acid substitution is a substitution with Arginine (Arg/R).

In some embodiments, the amino acid substitution is a substitution with a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F)). In some embodiments, the amino acid substitution is a substitution with Alanine (Ala/A).

In some aspects, the disclosure provides Cas12i polypeptide comprises one indicated amino acid substitution based on the parent or reference Cas12i polypeptide.

In some embodiments, the Cas12i polypeptide comprises an amino acid substitution at one position selected from the group consisting of E336, V880, G883, D892, and M923 of SEQ ID NO: 458. In some embodiments, the amino acid substitution is a substitution with a positively charged amino acid residue (such as, Lysine (Lys/K), Arginine (Arg/R), Histidine (His/H)), and optionally a substitution with Arginine (Arg/R).

In some embodiments, the Cas12i polypeptide comprises an amino acid substitution E336R relative to SEQ ID NO:458. In some embodiments, the Cas12i polypeptide comprises the amino acid sequence of SEQ ID NO:467 (xCas12i-N243R+E336R), or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 467.

In some other aspects, the disclosure provides Cas12i polypeptide comprises one indicated amino acid substitution based on the parent or reference Cas12i polypeptide.

In some embodiments, the Cas12i polypeptide comprises one amino acid substitution selected from the group consisting of V880R, G883R, D892R, and M923R relative to SEQ ID NO: 458.

In some aspects, the disclosure provides Cas12i polypeptide comprises two indicated amino acid substitutions based on the parent or reference Cas12i polypeptide.

In some embodiments, the Cas12i polypeptide comprises two amino acid substitutions at any two positions of E336, V880, G883, D892, and M923 of SEQ ID NO: 458. In some embodiments, each of the two amino acid substitutions is independently a substitution with a positively charged amino acid residue (such as, Lysine (Lys/K), Arginine (Arg/R), Histidine (His/H)). In some embodiments, each of the two amino acid substitutions is independently a substitution with Arginine (Arg/R).

In some embodiments, the Cas12i polypeptide comprises amino acid substitutions E336R and one amino acid substitution selected from the group consisting of V880R, G883R, D892R, and M923R relative to SEQ ID NO: 458.

In some embodiments, the Cas12i polypeptide comprises amino acid substitutions E336R and D892R relative to SEQ ID NO: 458. In some embodiments, the Cas12i polypeptide comprises the amino acid sequence of SEQ ID NO: 459 (hfCas12Max; xCas12i-N243R+E336R+D892R), or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 459.

In some aspects, the disclosure provides Cas12i polypeptide further comprise an indicated amino acid substitutions based on the parent or reference Cas12i polypeptide or the Cas12i polypeptide, e.g., for increased spacer-sequence specific dsDNA cleavage activity.

In some embodiments, the Cas12i polypeptide further comprises an additional amino acid substitution at a position selected from the group consisting of K109, L112, D125, 127, F144, L147, A148, L151, L157, V195, Y226, F252, I258, M293, W305, A308, I309, S312, A314, D315, V316, A318, L324, 1327, A348, L352, Y365, L372, L376, L379, L383, I405, L424, I427, A436, F439, A443, V447, A457, H458, P459, T460, S463, S814, F859, A864, H867, Y977, S1031, A1053, and F1068 of SEQ ID NO: 458. In some embodiments, the additional amino acid substitution is a substitution with a positively charged amino acid residue (such as, Lysine (Lys/K), Arginine (Arg/R), Histidine (His/H)), and optionally a substitution with Arginine (Arg/R).

### Decreased Off-Target Cleavage

In some embodiments, the Cas12i polypeptide substantially lacks spacer sequence-independent (off-target) dsDNA cleavage activity.

In some embodiments, the Cas12i polypeptide substantially lacks the spacer sequence-independent (off-target) dsDNA cleavage activity of SEQ ID NO: 458 or SEQ ID NO: 1.

In some embodiments, the Cas12i polypeptide has a decreased spacer sequence-independent (off-target) dsDNA cleavage activity compared to that of SEQ ID NO: 458 or SEQ ID NO: 1 when both are used in combination with a same guide nucleic acid, e.g., a decrease by at least about 5%, 10%, 15%, 20%, 25%, 30%, 35%, 40%, 45%, 50%, 55%, 60%, 65%, 70%, 75%, 80%, 85%, 90%, 95%, or 100%.

### Endonuclease Deficient (Dead) Cas12i Polypeptide

In some aspects, the disclosure provides a Cas12i polypeptide that is endonuclease deficient, which means the Cas12i polypeptide is substantially incapable of functioning as an endonuclease to cleave (either double strands or a single strand of) a dsDNA or a ssDNA, either against a target DNA or against a non-target DNA (For convenience of experiment design, performance, and evaluation, the defect of endonuclease activity is usually indicated by substantial loss of spacer sequence-specific dsDNA cleavage activity against a target DNA). Such a Cas12i polypeptide is named as “dead Cas12i (dCas12i)” and may be generated based on the parent or reference Cas12i polypeptide, for example, by mutating one or more functional domains of the parent or reference Cas12i polypeptide that is/are responsible for endonuclease activity.

In some embodiments, the Cas12i polypeptide is further engineered to substantially lack spacer sequence-specific (on-target) dsDNA cleavage activity.

In some embodiments, the Cas12i polypeptide substantially lacks the spacer sequence-specific (on-target) dsDNA cleavage activity of SEQ ID NO: 458 or SEQ ID NO: 1.

In some embodiments, the Cas12i polypeptide has a decreased spacer sequence-specific (on-target) dsDNA cleavage activity compared to that of SEQ ID NO: 458 or SEQ ID NO: 1 when both used in combination with a same guide nucleic acid, e.g., a decrease by at least about 5%, 10%, 15%, 20%, 25%, 30%, 35%, 40%, 45%, 50%, 55%, 60%, 65%, 70%, 75%, 80%, 85%, 90%, 95%, or 100%.

In some embodiments, the Cas12i polypeptide comprise a further amino acid substitution at a position selected from the group consisting of D650, D700, E875, and D1049 of SEQ ID NO: 458. In some embodiments, the amino acid substitution is a substitution with a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F)) In some embodiments, the amino acid substitution is a substitution with Alanine (Ala/A).

In some embodiments, the Cas12i polypeptide comprises amino acid substitutions E336R and D1049A relative to SEQ ID NO: 458. In some embodiments, the Cas12i polypeptide comprises the amino acid sequence of SEQ ID NO: 466 (xCas12i-N243R+E336R+D1049A), or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 466.

### Cas12i Nickase

In some aspects, the disclosure provides a Cas12i polypeptide that is not completely endonuclease deficient but the endonuclease activity is not against the double strand of a dsDNA but against one strand (the sense or nonsense strand; or the target or nontarget strand) of a dsDNA or a ssDNA, which means the Cas12i polypeptide is substantially incapable of functioning as a dsDNA endonuclease to cleave double strands of a dsDNA, either against a target DNA or against a non-target DNA, but is substantially capable of functioning as a ssDNA endonuclease to cleave a ssDNA or “nick” one strand of a dsDNA. Such a Cas12i polypeptide is named as “nickase” and may be generated based on the parent or reference Cas12i polypeptide, for example, by mutating one or more functional domains of the parent or reference Cas12i polypeptide that is/are responsible for endonuclease activity.

In some embodiments, the Cas12i polypeptide is further engineered to be a nickase.

In some embodiments, the Cas12i polypeptide comprise an amino acid substitution at a position selected from the group consisting of W896, S924, and S925 of SEQ ID NO: 458.

In some embodiments, the Cas12i polypeptide comprise an amino acid substitution selected from the group consisting of W896R, W896P, W896K, S924R, S924F, S924D, S924E, S924H, S925R, and S925T relative to SEQ ID NO: 458.

### Fusion Protein

In some aspects, the disclosure provides a fusion protein comprising the Cas12i polypeptide and a functional domain. In some embodiments, the functional domain is a heterologous functional domain. Such a function protein may also be regarded as a Cas12i polypeptide further comprising a functional domain fused to the Cas12i polypeptide.

In some embodiments, the Cas12i polypeptide further comprises a functional domain fused to the Cas12i polypeptide.

In some embodiments, the functional domain is selected from the group consisting of a nuclear localization signal (NLS), a nuclear export signal (NES), a base editing domain, for example, a deaminase or a catalytic domain thereof, a base excising domain, an uracil glycosylase inhibitor (UGI) or a catalytic domain thereof, an uracil glycosylase (UNG) or a catalytic domain thereof, a methylpurine glycosylase (MPG) or a catalytic domain thereof, a methylase or a catalytic domain thereof, a demethylase or a catalytic domain thereof, an transcription activating domain (e.g., VP64 or VPR), an transcription inhibiting domain (e.g., KRAB moiety or SID moiety), a reverse transcriptase or a catalytic domain thereof, an exonuclease (e.g., T5E (SEQ ID NO: 449)) or a catalytic domain thereof, a destabilized domain (e.g., destabilized domains (DD) of E. coli dihydrofolate reductase (ecDHFR)), a histone residue modification domain, a nuclease catalytic domain (e.g., FokI), a transcription modification factor, a light gating factor, a chemical inducible factor, a chromatin visualization factor, a targeting polypeptide for providing binding to a cell surface portion on a target cell or a target cell type, a reporter (e.g., fluorescent) polypeptide or a detection label (e.g., GST, HRP, CAT, GFP, HcRed, DsRed, CFP, YFP, BFP), a localization signal, a polypeptide targeting moiety, a DNA binding domain (e.g., MBP, Lex A DBD, Gal4 DBD), an epitope tag (e.g., His, myc, V5, FLAG, HA, VSV-G, Trx, etc), a transcription release factor, an HDAC, a moiety having ssRNA cleavage activity, a moiety having dsRNA cleavage activity, a moiety having ssDNA cleavage activity, a moiety having dsDNA cleavage activity, a DNA or RNA ligase, a functional domain exhibiting activity to modify a target DNA, selected from the group consisting of: methyltransferase activity, DNA repair activity, DNA damage activity, dismutase activity, alkylation activity, dealkylation activity, depurination activity, oxidation activity, deoxidation activity, pyrimidine dimer forming activity, integrase activity, transposase activity, recombinase activity, polymerase activity, ligase activity, helicase activity, photolyase activity, glycosylase activity, acetyl transferase activity, deacetylase activity, kinase activity, phosphatase activity, ubiquitin ligase activity, deubiquitination activity, adenylation activity, deadenylation activity, SUMOylation activity, deSUMOylation activity, ribosylation activity, deribosylation activity, myristoylation activity, demyristoylation activity, glycosylation activity (e.g., from O-GlcNAc transferase), deglycosylation activity, and a catalytic domain thereof, and a functional fragment (e.g., a functional truncation) thereof, and any combination thereof.

In some embodiments, the NLS comprises or is SV40 NLS (SEQ ID NO: 444), bpSV40 NLS (BP NLS, bpNLS, SEQ ID NO: 443 or 462), or NP NLS (Xenopus laevis Nucleoplasmin NLS, nucleoplasmin NLS, SEQ ID NO: 445).

### Base Editing

In some embodiments, the base editing domain is capable of substituting a base of a nucleotide with a different base.

In some embodiments, the base editing domain is capable of deaminating a base of a nucleotide.

In some embodiments, the base editing domain comprises a deaminase domain capable of deaminating a base (e.g., an adenine, a guanine, a cytosine, a thymine, an uracil) of a nucleotide. In some embodiments, the deaminase domain is capable of deaminating an adenine (A) to a hypoxanthine (I). In some embodiments, the deamination of the adenine to the hypoxanthine converts the adenosine (A) or deoxyadenosine (dA) containing the adenine to a guanosine (G) or deoxyguanosine (dG). In some embodiments, the deaminase domain is capable of deaminating a cytosine (C) to an uracil (U). In some embodiments, the deamination of the cytosine to the uracil converts the cytidine (C) or deoxycytidine (dC) containing the cytosine to a uridine (U) or a deoxythymidine (dT).

In some embodiments, the base editing domain is capable of excising a base (e.g., an adenine, a guanine, a cytosine, a thymine, an uracil) of a nucleotide.

In some embodiments, the base editing domain comprises a base excising domain capable of excising a base of a nucleotide.

In some embodiments, the base editing domain comprises a deaminase domain and a base excising domain.

In some embodiments, the deaminase domain is tRNA adenosine deaminase (TadA), or the deaminase domain thereof, or a functional variant or fragment thereof, e.g., TadA8e (SEQ ID NO: 3), TadA8.17, TadA8.20, TadA9, TadA8E^{V106W}, TadA8E^{V106W+D108Q} TadA-CDa, TadA-CDb, TadA-CDc, TadA-CDd, TadA-CDe, TadA-dual, T_{AD} AC-1.2, T_{AD}AC-1.14, T_{AD}AC-1.17, T_{AD}AC-1.19, T_{AD}AC-2.5, T_{AD}AC-2.6, T_{AD}AC-2.9, T_{AD}AC-2.19, T_{AD}AC-2.23, TadA8e-N46L, TadA8e-N46P.

In some embodiments, the deaminase domain is an apolipoprotein B mRNA-editing complex (APOBEC) family deaminase, an activation induced deaminase (AID), a cytidine deaminase 1 from Petromyzon marinus (pmCDA1), or the deaminase domain thereof, or a functional variant or fragment thereof, e.g., APOBEC1, APOBEC2, APOBEC3A, APOBEC3B, APOBEC3C, APOBEC3D, APOBEC3F, APOBEC3G, APOBEC3H.

In some embodiments, the deaminase or catalytic domain thereof is an adenine deaminase (e.g., TadA, such as, TadA8e, TadA8.17, TadA8.20, TadA9) or a catalytic domain thereof, for example, TadA8e-V106W (SEQ ID NO: 439), TadA8e-W106V (SEQ ID NO: 461).

In some embodiments, the deaminase or catalytic domain thereof is a cytidine deaminase (e.g., APOBEC, such as, APOBEC3, for example, APOBEC3A, APOBEC3B, APOBEC3C; DddA) or a catalytic domain thereof, for example, hAPOBEC3-W104A (SEQ ID NO: 440).

In some embodiments, the UGI is human UGI domain (such as, SEQ ID NO: 441).

In some embodiments, the Cas12i polypeptide comprises amino acid substitutions E336R and D1049A relative to SEQ ID NO: 458, and a base editing domain, for example, a deaminase or a catalytic domain thereof.

In some embodiments, the Cas12i polypeptide comprises the amino acid sequence of SEQ ID NO: 463 (dCas12Max-ABE), or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 463.

In some embodiments, the Cas12i polypeptide comprises the amino acid sequence of SEQ ID NO: 464 (dCas12Max-CBE), or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 464.

In some embodiments, the functional domain comprises a reverse transcriptase (RT) or a catalytic domain thereof. In some embodiments, the guide nucleic acid further comprises or is used in combination with a reverse transcription donor RNA (RT donor RNA) comprising a primer binding site (PBS) and a template sequence. For details of prime editing with Class 2, Type V Cas proteins, references is made to WO2022256440A3, which is incorporated herein by reference in its entirety.

### System

The Cas12i polypeptide of the disclosure may be used in combination with and guided by a guide nucleic acid to a target DNA to function on the target DNA. In another aspect, the disclosure provides a system comprising:
(1) the Cas12i polypeptide of the disclosure or a polynucleotide (e.g., a DNA, an RNA) encoding the Cas12i polypeptide; and
(2) a guide nucleic acid or a polynucleotide (e.g., a DNA or an RNA) encoding the guide nucleic acid, the guide nucleic acid comprising:
(i) a direct repeat (DR) sequence capable of forming a complex with the Cas12i polypeptide; and
(ii) a spacer sequence capable of hybridizing to a target sequence of a target DNA, thereby guiding the complex to the target DNA.

In some embodiments, the system is a non-naturally occurring or engineered system.

In some embodiments, the system is a complex comprising the Cas12i polypeptide complexed with the guide nucleic acid. In some embodiments, the complex further comprises the target DNA hybridized with the target sequence.

In another aspect, the disclosure provides a guide nucleic acid comprising:
(1) a direct repeat (DR) sequence capable of forming a complex with the Cas12i polypeptide of the disclosure, and
(2) a spacer sequence capable of hybridizing to a target sequence of a target DNA, thereby guiding the complex to the target DNA.

In some embodiments, the guide nucleic acid is a guide RNA (gRNA). In some embodiments, the guide nucleic acid comprises a crRNA. In some embodiments, the guide nucleic acid does not comprise a tracrRNA.

In some embodiments, the direct repeat sequence is 5′ to the spacer sequence.

### Design of Protospacer Sequence/Target Sequence; Target Site

For the purpose of the disclosure, in some embodiments, the protospacer sequence or target sequence is located such that the target DNA is specifically modified by the Cas12i polypeptide.

To facilitate the evaluation of selected protospacer sequences or target sequence and designed guide sequences in mouse models, in some embodiments, the protospacer sequence or target sequence is located such that a mouse target DNA is specifically modified by the Cas12i polypeptide. In some embodiments, the protospacer sequence or target sequence is located such that both a human target DNA and a mouse target DNA are specifically modified by the Cas12i polypeptide. That is, the protospacer sequence or target sequence is selected to be cross-reactive to both human and mouse species.

In some embodiments, the protospacer sequence is a stretch of contiguous nucleotides identified from the nontarget strand of the target DNA by identifying the stretch of contiguous nucleotides immediately 3′ to the PAM on the nontarget strand. In some embodiments, the PAM is 5′-TN, 5′-TTN, or 5′-GCC, wherein N is A, T, G, or C. In some embodiments, the PAM is 5′-TTN, wherein N is A, T, G, or C. The protospacer sequence is the reversely complementary sequence of the target sequence.

In some embodiments, the protospacer sequence is a stretch of about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of the target DNA, or a stretch of contiguous nucleotides of the target DNA in a numerical range between any two of the preceding values, e.g., a stretch of from about 16 to about 50, or from about 17 to about 22 contiguous nucleotides. In some embodiments, the protospacer sequence is a stretch of about 20 contiguous nucleotides of the target DNA.

In some embodiments, the protospacer sequence comprises about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of the target DNA, or contiguous nucleotides in a numerical range between any two of the preceding values, e.g., from about 16 to about 50, or from about 17 to about 22 contiguous nucleotides of the target DNA. In some embodiments, the protospacer sequence comprises about 20 contiguous nucleotides of the target DNA.

In some embodiments, the target sequence is a stretch of contiguous nucleotides identified from the target strand of the target DNA. The target sequence is the reversely complementary sequence of the protospacer sequence.

In some embodiments, the target sequence is a stretch of about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides on the target strand of the target DNA, or a stretch of contiguous nucleotides on the target strand of the target DNA in a numerical range between any two of the preceding values, e.g., a stretch of from about 16 to about 50, or from about 17 to about 22 contiguous nucleotides. In some embodiments, the target sequence is a stretch of about 20 contiguous nucleotides on the target strand of the target DNA.

In some embodiments, the target sequence comprises about or at least about 16 contiguous nucleotides of the target DNA, e.g., about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of the target DNA, or in a numerical range between any two of the preceding values, e.g., from about 16 to about 50, or from about 17 to about 22 contiguous nucleotides of the target DNA. In some embodiments, the target sequence comprises about 20 contiguous nucleotides of the target DNA.

In some embodiments, the target sequence comprises about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides on the target strand of the target DNA, or contiguous nucleotides in a numerical range between any two of the preceding values, e.g., from about 16 to about 50, or from about 17 to about 22 contiguous nucleotides on the target strand of the target DNA. In some embodiments, the target sequence comprises about 20 contiguous nucleotides on the target strand of the target DNA.

In some embodiments, the reversely complementary sequence of the target sequence is immediately 3′ to a protospacer adjacent motif (PAM); optionally, wherein the PAM is 5′-TN, 5′-TTN, or 5′-GCC, wherein N is A, T, G, or C, wherein N is A, T, G, or C.

In some embodiments, the nontarget strand is the sense strand of the target DNA.

In some embodiments, the nontarget strand is the antisense strand of the target DNA.

In some embodiments, the target strand is the sense strand of the target DNA.

In some embodiments, the target strand is the antisense strand of the target DNA.

In some embodiments, the protospacer sequence or target sequence is located within Exon 1 of the target DNA.

In some embodiments, the protospacer sequence or target sequence is located within about 50, 100, 150, 200, 250, 300, or more 5′ end nucleotides of Exon 1 of the target DNA.

In some embodiments, the target DNA comprises a pathogenic mutation.

In some embodiments, the target DNA comprises a premature stop codon (e.g., TAG).

In some embodiments, the target DNA is a dsDNA, such as, a eukaryotic dsDNA, e.g., a gene in a eukaryotic cell.

In some embodiments, the target DNA is human target DNA, non-human primate target DNA, or mouse target DNA.

In some embodiments, the target DNA is in a eukaryotic cell, for example, a human cell, a non-human primate cell, or a mouse cell.

### Design of Guide Sequence According to Protospacer/Target Sequence

In some embodiments, the spacer sequence is about or at least about 16 nucleotides in length, e.g., about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides in length, or in a length of a numerical range between any two of the preceding values, e.g., in a length of from about 16 to about 50 nucleotides, or from about 17 to about 22 nucleotides. In some embodiments, the spacer sequence is about 20 nucleotides in length.

In some embodiments, (1) the guide sequence is at least about 50%, 55%, 60%, 65%, 70%, 75%, 80%, 85%, 90%, 95%, or 100% (fully), optionally about 100% (fully), reversely complementary to the target sequence; (2) the guide sequence contains no more than 5, 4, 3, 2, or 1 mismatch or contains no mismatch with the target sequence; or (3) the guide sequence comprises no mismatch with the target sequence in the first 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 nucleotides at the 5′ end of the guide sequence. In some embodiments, the guide sequence is about 100% (fully), reversely complementary to the target sequence.

### Selection of Protospacer/Target/Guide Sequence; Effect of System

In some embodiments, the protospacer sequence, the target sequence, or the guide sequence is selected such that the target DNA is modified by the system of the disclosure. In some embodiments, the modification decreases or eliminates the transcription of the target DNA and/or translation of a transcript (e.g., mRNA) of the target DNA.

In some embodiments, the level of the transcript (e.g., mRNA) of the target DNA is decreased in a cell model (e.g., HEK293T cell model) or an animal model (e.g., a mouse model, a non-human primate model) by at least about 10%, about 15%, about 20%, about 25%, about 30%, about 35%, about 40%, about 45%, about 50%, about 55%, about 60%, about 65%, about 70%, about 75%, about 80%, about 85%, or more, upon administration of the system of the disclosure to the cell model or the animal model, compared to the level of the transcript (e.g., mRNA) of the target DNA in the same cell model or animal model that does not receive the administration.

In some embodiments, the level of the transcript (e.g., mRNA) of the target DNA is increased in a cell model (e.g., HEK293T cell model) or an animal model (e.g., a mouse model, a non-human primate model) by at least about 10%, about 15%, about 20%, about 25%, about 30%, about 35%, about 40%, about 45%, about 50%, about 55%, about 60%, about 65%, about 70%, about 75%, about 80%, about 85%, or more, upon administration of the system of the disclosure to the cell model or the animal model, compared to the level of the transcript (e.g., mRNA) of the target DNA in the same cell model or animal model that does not receive the administration.

In some embodiments, the level of the expression product (e.g., protein) of the target DNA is decreased in a cell model (e.g., HEK293T cell model) or an animal model (e.g., a mouse model, a non-human primate model) by at least about 10%, about 15%, about 20%, about 25%, about 30%, about 35%, about 40%, about 45%, about 50%, about 55%, about 60%, about 65%, about 70%, about 75%, about 80%, about 85%, or more, upon administration of the system of the disclosure to the cell or the animal model, compared to the level of the expression product (e.g., protein) of the target DNA in the same cell model or animal model that does not receive the administration.

In some embodiments, the level of the expression product (e.g., protein) of the target DNA is increased in a cell model (e.g., HEK293T cell model) or an animal model (e.g., a mouse model, a non-human primate model) by at least about 10%, about 15%, about 20%, about 25%, about 30%, about 35%, about 40%, about 45%, about 50%, about 55%, about 60%, about 65%, about 70%, about 75%, about 80%, about 85%, or more, upon administration of the system of the disclosure to the cell or the animal model, compared to the level of the expression product (e.g., protein) of the target DNA in the same cell model or animal model that does not receive the administration. In some embodiments, the expression product is a functional mutant of the expression product of the target DNA.

### Overall Structure of Guide Nucleic Acid

In some embodiments, the guide nucleic acid is a single molecule.

In some embodiments, the guide nucleic acid comprises one spacer sequence capable of hybridizing to one target sequence.

In some embodiments, the guide nucleic acid comprises a plurality (e.g., 2, 3, 4, 5 or more) of the spacer sequences capable of hybridizing to a plurality of the target sequences, respectively.

In some embodiments, the guide nucleic acid comprises, from 5′ to 3′, the direct repeat sequence, the spacer sequence, the direct repeat sequence, the spacer sequence, and the direct repeat sequence.

In some embodiments, the guide nucleic acid comprises one scaffold sequence and one guide sequence.

In some embodiments, the guide nucleic acid comprises one scaffold sequence 5′ to one guide sequence. In some embodiments, the guide nucleic acid comprises one scaffold sequence 3′ to one guide sequence.

In some embodiments, the guide nucleic acid comprises one or more scaffold sequence and/or one or more guide sequence, provided that the guide nucleic acid does not comprise one scaffold sequence and one guide sequence.

In some embodiments, the guide nucleic acid comprises, from 5′ to 3′, one scaffold sequence, one guide sequence, and one scaffold sequence, wherein scaffold sequences are the same or different.

In some embodiments, the guide nucleic acid comprises, from 5′ to 3′, one guide sequence, one scaffold sequence, and one guide sequence, wherein guide sequences are the same or different.

In some embodiments, the guide nucleic acid comprises, from 5′ to 3′, one scaffold sequence, one guide sequence, one scaffold sequence, and one guide sequence, wherein scaffold sequences are the same or different, and wherein guide sequences are the same or different.

In some embodiments, the guide nucleic acid comprises, from 5′ to 3′, one guide sequence, one scaffold sequence, one guide sequence, and one scaffold sequence, wherein scaffold sequences are the same or different, and wherein guide sequences are the same or different.

In some embodiments, the guide nucleic acid comprises, from 5′ to 3′, one scaffold sequence, one guide sequence, one scaffold sequence, one guide sequence, and one scaffold sequence, wherein scaffold sequences are the same or different, and wherein guide sequences are the same or different.

In some embodiments, the guide nucleic acid comprises, from 5′ to 3′, one guide sequence, one scaffold sequence, one guide sequence, one scaffold sequence, and one guide sequence, wherein scaffold sequences are the same or different, and wherein guide sequences are the same or different.

In some embodiments, the guide nucleic acid comprises, from 5′ to 3′, one scaffold sequence, one guide sequence, one scaffold sequence, one guide sequence, one scaffold sequence, and one guide sequence, wherein scaffold sequences are the same or different, and wherein guide sequences are the same or different.

In some embodiments, the guide nucleic acid comprises, from 5′ to 3′, one guide sequence, one scaffold sequence, one guide sequence, one scaffold sequence, one guide sequence, and one scaffold sequence, wherein scaffold sequences are the same or different, and wherein guide sequences are the same or different.

In some embodiments, the guide nucleic acid comprises a linker or no linker between any adjacent scaffold sequence and guide sequence. In some embodiments, the guide nucleic acid comprises no linker between any adjacent scaffold sequence and guide sequence.

### Multiple Guide Nucleic Acid

The system of the disclosure may comprise or encode one guide nucleic acid or comprise or encode multiple (e.g., 2, 3, 4, or more) guide nucleic acids, e.g., for the purpose of improving the editing efficiency of the system on target DNA.

In some embodiments, the system further comprises one or more additional guide nucleic acids, or the first polynucleotide sequence further comprises one or more additional sequences encoding one or more additional guide nucleic acids, each of the additional guide nucleic acids comprising:
(1) an additional scaffold sequence capable of forming a complex with the Cas12i polypeptide, and
(2) an additional guide sequence capable of hybridizing to an additional target sequence on a target strand of the target DNA or an additional target sequence on the transcript thereof, thereby guiding the complex to the target DNA or the transcript.

In some embodiments, the additional protospacer sequence is on the same strand as the protospacer sequence.

In some embodiments, the additional protospacer sequence is on the different strand from the protospacer sequence.

In some embodiments, the additional protospacer sequence is the same or different from the protospacer sequence.

In some embodiments, the additional target sequence is the same or different from the target sequence.

In some embodiments, the additional guide sequence is the same or different from the guide sequence.

In some embodiments, the additional scaffold sequence is the same or different from the scaffold sequence. In some embodiments wherein the system comprises the same Cas12i polypeptide and multiple guide nucleic acids, the scaffold sequences of the multiple guide nucleic acids may be the same or different (e.g., different by no more than 5, 4, 3, 2, or 1 nucleotide) to be compatible to the same Cas12i polypeptide. In some embodiments wherein that the system comprises different Cas12i polypeptides and multiple guide nucleic acids, the scaffold sequences of the multiple guide nucleic acids may be different to be compatible to the different Cas12i polypeptides.

In some embodiments, the additional guide nucleic acid and the guide nucleic acid are operably linked to or under the regulation of the same regulatory element (e.g., promoter) or separate regulatory elements (e.g., promoters).

### Nature and Modification of Guide Nucleic Acid

In some embodiments, the guide nucleic acid (e.g., the guide nucleic acid, the additional guide nucleic acid) is an RNA. In some embodiments, the guide nucleic acid is an unmodified guide RNA. In some embodiments, the guide nucleic acid is a modified guide RNA. In some embodiments, the guide nucleic acid comprises a modification. In some embodiments, the guide nucleic acid is a modified RNA containing a modified ribonucleotide. In some embodiments, the guide nucleic acid is a modified RNA containing a deoxyribonucleotide. In some embodiments, the guide nucleic acid is a modified RNA containing a modified deoxyribonucleotide. In some embodiments, the guide nucleic acid comprises a modified or unmodified deoxyribonucleotide and a modified or unmodified ribonucleotide.

### Scaffold Sequence

For the purpose of the disclosure, the scaffold sequence is compatible with the Cas12i polypeptide of the disclosure and is capable of complexing with the Cas12i polypeptide. The scaffold sequence may be a naturally occurring scaffold sequence identified along with the Cas12i polypeptide, or a variant thereof maintaining the ability to complex with the Cas12i polypeptide. Generally, the ability to complex with the Cas12i polypeptide is maintained as long as the secondary structure of the variant is substantially identical to the secondary structure of the naturally occurring scaffold sequence. A nucleotide deletion, insertion, or substitution in the primary sequence of the scaffold sequence may not necessarily change the secondary structure of the scaffold sequence (e.g., the relative locations and/or sizes of the stems, bulges, and loops of the scaffold sequence do not significantly deviate from that of the original stems, bulges, and loops). For example, the nucleotide deletion, insertion, or substitution may be in a bulge or loop region of the scaffold sequence so that the overall symmetry of the bulge and hence the secondary structure remains largely the same. The nucleotide deletion, insertion, or substitution may also be in the stems of the scaffold sequence so that the lengths of the stems do not significantly deviate from that of the original stems (e.g., adding or deleting one base pair in each of two stems correspond to 4 total base changes).

In some embodiments, the direct repeat sequence or the additional scaffold sequence has substantially the same secondary structure as the secondary structure of any one of SEQ ID NOs: 11 and 451-457.

In some embodiments, the direct repeat sequence or the additional scaffold sequence:
(i) comprises the polynucleotide sequence of any one of SEQ ID NOs: 11 and 451-457; or
(ii) comprises a polynucleotide sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 11 and 451-457.

In some embodiments, the scaffold sequence or the additional scaffold sequence comprises a sequence having a sequence identity of at least about 80% (e.g., at least about 80%, 85%, 90%, 91%, 92%, 93%, 94%, 95%, 96%, 97%, 98%, 99%, or 100%) to the sequence of any one of SEQ ID NOs: 11 and 451-457; or a sequence having at most 1, 2, 3, 4, 5, 6, 7, 8, 9, or 10 nucleotide differences, whether consecutive or not, compared to the sequence of any one of SEQ ID NOs: 11 and 451-457.

In some embodiments, the scaffold sequence or the additional scaffold sequence comprises the sequence of SEQ ID NO: 452.

### Regulation of Guide Nucleic Acid

In some embodiments, the polynucleotide encoding the guide nucleic acid is a DNA, a RNA, or a DNA/RNA mixture. By “DNA/RNA mixture” it refers to a nucleic acid comprising both one or more modified or unmodified ribonucleotides and one or more modified or unmodified deoxyribonucleotides, whether consecutive or not. However, by “DNA” or “RNA” it may also refer to a DNA containing one or more modified or unmodified ribonucleotides, whether consecutive or not, or an RNA containing one or more modified or unmodified deoxyribonucleotides, whether consecutive or not.

In some embodiments, the guide nucleic acid is operably linked to or under the regulation of a promoter.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1α short (EFS) promoter, a Bglucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, and a myelin basic protein (MBP) promoter.

### Regulation of Cas12i Polypeptide

In some embodiments, the polynucleotide encoding the Cas12i polypeptide is a DNA, a RNA, or a DNA/RNA mixture. By “DNA/RNA mixture” it refers to a nucleic acid comprising both one or more modified or unmodified ribonucleotides and one or more modified or unmodified deoxyribonucleotides, whether consecutive or not. However, by “DNA” or “RNA” it may also refer to a DNA containing one or more modified or unmodified ribonucleotides, whether consecutive or not, or an RNA containing one or more modified or unmodified deoxyribonucleotides, whether consecutive or not.

In some embodiments, the polynucleotide encoding the Cas12i polypeptide is operably linked to or under the regulation of a promoter.

In some embodiments, the promoter is a ubiquitous, tissue-specific, cell-type specific, constitutive, or inducible promoter.

Suitable promoters are known in the art and include, for example, a Cbh promoter, a Cba promoter, a pol I promoter, a pol II promoter, a pol III promoter, a T7 promoter, a U6 promoter, a H1 promoter, a retroviral Rous sarcoma virus LTR promoter, a cytomegalovirus (CMV) promoter, a SV40 promoter, a dihydrofolate reductase promoter, a β-actin promoter, an elongation factor 1a short (EFS) promoter, a βglucuronidase (GUSB) promoter, a cytomegalovirus (CMV) immediate-early (Ie) enhancer and/or promoter, a chicken β-actin (CBA) promoter or derivative thereof such as a CAG promoter, CB promoter, a (human) elongation factor 1α-subunit (EF1α) promoter, a ubiquitin C (UBC) promoter, a prion promoter, a neuron-specific enolase (NSE), a neurofilament light (NFL) promoter, a neurofilament heavy (NFH) promoter, a platelet-derived growth factor (PDGF) promoter, a platelet-derived growth factor B-chain (PDGF-β) promoter, a synapsin (Syn) promoter, a human synapsin (hSyn) promoter, a synapsin 1 (Syn1) promoter, a methyl-CpG binding protein 2 (MeCP2) promoter, a Ca2+/calmodulin-dependent protein kinase II (CaMKII) promoter, a metabotropic glutamate receptor 2 (mGluR2) promoter, a neurofilament light (NFL) promoter, a neurofilament heavy (NFH) promoter, a β-globin minigene nβ2 promoter, a preproenkephalin (PPE) promoter, an enkephalin (Enk) promoter, an excitatory amino acid transporter 2 (EAAT2) promoter, a glial fibrillary acidic protein (GFAP) promoter, a myelin basic protein (MBP) promoter, a OTOF promoter, a GRK1 promoter, a CRX promoter, a NRL promoter, a MECP2 promoter, a mMECP2 promoter, a hMECP2 promoter, an APP promoter, and a RCVRN promoter.

### Delivery

Various ways of delivery can be applied to the Cas12i polypeptide of the disclosure or the system of the disclosure as needed in practices.

In yet another aspect, the disclosure provides a polynucleotide encoding the Cas12i polypeptide of the disclosure.

In yet another aspect, the disclosure provides a delivery system comprising (1) the Cas12i polypeptide of the disclosure, the polynucleotide of the disclosure, or the system of the disclosure; and (2) a delivery vehicle.

In yet another aspect, the disclosure provides a vector comprising the polynucleotide of the disclosure. In some embodiments, the vector encodes a guide nucleic acid as defined in the disclosure. In some embodiments, the vector is a plasmid vector, a recombinant AAV (rAAV) vector (vector genome), or a recombinant lentivirus vector.

In yet another aspect, the disclosure provides a recombinant AAV (rAAV) particle comprising the rAAV vector genome of the disclosure. A simple introduction of AAV for delivery may refer to “Adeno-associated Virus (AAV) Guide” (addgene. org/guides/aav/).

Adeno-associated virus (AAV), when engineered to delivery, e.g., a protein-encoding sequence of interest, may be termed as a (r) AAV vector, a (r) AAV vector particle, or a (r) AAV particle, where “r” stands for “recombinant”. And the genome packaged in AAV vectors for delivery may be termed as a (r) AAV vector genome, vector genome, or vg for short, while viral genome may refer to the original viral genome of natural AAVs.

The serotypes of the capsids of rAAV particles can be matched to the types of target cells. For example, Table 2 of WO2018002719A1 lists exemplary cell types that can be transduced by the indicated AAV serotypes (incorporated herein by reference).

In some embodiments, the rAAV particle comprising a capsid with a serotype suitable for delivery into ear cells (e.g., inner hair cells). In some embodiments, the rAAV particle comprising a capsid with a serotype of AAV1, AAV2, AAV3A, AAV3B, AAV4, AAV5, AAV6, AAV7, AAVrh74, AAV8, AAV9, AAV10, AAV11, AAV12, AAV13, AAV-DJ, or AAV. PHP. eB, a member of the Clade to which any of the AAV1-AAV13 belong, or a functional variant (e.g., a functional truncation) thereof, encapsidating the rAAV vector genome. In some embodiments, the serotype of the capsid is AAV9 or a functional variant thereof.

General principles of rAAV particle production are known in the art. In some embodiments, rAAV particles may be produced using the triple transfection method (described in detail in U.S. Pat. No. 6,001,650).

The vector titers are usually expressed as vector genomes per ml (vg/ml). In some embodiments, the vector titer is above 1×10⁹, above 5×10¹⁰, above 1×10¹¹, above 5×10¹¹, above 1×10¹², above 5×10¹², or above 1×10¹³ vg/ml.

Instead of packaging a single strand (ss) DNA sequence as a vector genome of a rAAV particle, systems and methods of packaging an RNA sequence as a vector genome into a rAAV particle is recently developed and applicable herein. See PCT/CN2022/075366, which is incorporated herein by reference in its entirety.

When the vector genome is RNA as in, for example, PCT/CN2022/075366, for simplicity of description and claiming, sequence elements described herein for DNA vector genomes, when present in RNA vector genomes, should generally be considered to be applicable for the RNA vector genomes except that the deoxyribonucleotides in the DNA sequence are the corresponding ribonucleotides in the RNA sequence (e.g., dT is equivalent to U, and dA is equivalent to A) and/or the element in the DNA sequence is replaced with the corresponding element with a corresponding function in the RNA sequence or omitted because its function is unnecessary in the RNA sequence and/or an additional element necessary for the RNA vector genome is introduced.

As used herein, a coding sequence, e.g., as a sequence element of rAAV vector genomes herein, is construed, understood, and considered as covering and covers both a DNA coding sequence and an RNA coding sequence. When it is a DNA coding sequence, an RNA sequence can be transcribed from the DNA coding sequence, and optionally further a protein can be translated from the transcribed RNA sequence as necessary. When it is an RNA coding sequence, the RNA coding sequence per se can be a functional RNA sequence for use, or an RNA sequence can be produced from the RNA coding sequence, e.g., by RNA processing, or a protein can be translated from the RNA coding sequence.

For example, a Cas13 coding sequence encoding a Cas13 polypeptide covers either a Cas13 DNA coding sequence from which a Cas13 polypeptide is expressed (indirectly via transcription and translation) or a Cas13 RNA coding sequence from which a Cas13 polypeptide is translated (directly).

For example, a gRNA coding sequence encoding a gRNA covers either a gRNA DNA coding sequence from which a gRNA is transcribed or a gRNA RNA coding sequence (1) which per se is the functional gRNA for use, or (2) from which a gRNA is produced, e.g., by RNA processing.

In some embodiments for rAAV RNA vector genomes, 5′-ITR and/or 3′-ITR as DNA packaging signals may be unnecessary and can be omitted at least partly, while RNA packaging signals can be introduced.

In some embodiments for rAAV RNA vector genomes, a promoter to drive transcription of DNA sequences may be unnecessary and can be omitted at least partly.

In some embodiments for rAAV RNA vector genomes, a sequence encoding a polyA signal may be unnecessary and can be omitted at least partly, while a polyA tail can be introduced.

Similarly, other DNA elements of rAAV DNA vector genomes can be either omitted or replaced with corresponding RNA elements and/or additional RNA elements can be introduced, in order to adapt to the strategy of delivering an RNA vector genome by rAAV particles.

In yet another aspect, the disclosure provides a ribonucleoprotein (RNP) comprising the Cas12i polypeptide of the disclosure and a guide nucleic acid optionally as defined in the disclosure.

In yet another aspect, the disclosure provides a lipid nanoparticle (LNP) comprising an RNA (e.g., mRNA) encoding the Cas12i polypeptide of the disclosure and a guide nucleic acid of the disclosure.

### Method of Modification

The CRISPR-Cas12i system of the disclosure comprising the Cas12i polypeptide of the disclosure has a wide variety of utilities, including modifying (e.g., cleaving, deleting, inserting, translocating, inactivating, or activating) a target DNA in a multiplicity of cell types. The CRISPR-Cas12i systems have a broad spectrum of applications requiring high cleavage activity and low collateral activity, e.g., drug screening, disease diagnosis and prognosis, and treating various genetic disorders.

The methods and/or the systems of the disclosure can be used to modify a target DNA, for example, to modify the translation and/or transcription of one or more genes of the cells. For example, the modification may lead to increased transcription/translation/expression of a gene. In other embodiments, the modification may lead to decreased transcription/translation/expression of a gene.

In yet another aspect, the disclosure provides a method for modifying a target DNA, comprising contacting the target DNA with the system of the disclosure, the vector of the disclosure, the ribonucleoprotein of the disclosure, or the lipid nanoparticle of the disclosure, wherein the spacer sequence is capable of hybridizing to a target sequence of the target DNA, wherein the target DNA is modified by the complex.

In some embodiments, the target DNA is in a cell.

In some embodiments, the modification comprises one or more of cleavage, base editing, repairing, and exogenous sequence insertion or integration of the target DNA.

### Cells

The methods of the disclosure can be used to introduce the systems of the disclosure into a cell and cause the cell to alter the production of one or more cellular produces, such as antibody, starch, ethanol, or any other desired products. Such cells and progenies thereof are within the scope of the disclosure.

In yet another aspect, the disclosure provides a cell comprising the system of the disclosure. In some embodiments, the cell is a eukaryote. In some embodiments, the cell is a human cell.

In yet another aspect, the disclosure provides a cell modified by the system of the disclosure or the method of the disclosure. In some embodiments, the cell is a eukaryote. In some embodiments, the cell is a human cell. In some embodiments, the cell is modified in vitro, in vivo, or ex vivo.

In some embodiments, the cell is a stem cell. In some embodiments, the cell is not a human embryonic stem cell. In some embodiments, the cell is not a human germ cell.

In some embodiments, the cell is a prokaryotic cell.

In some embodiments, the cell is a eukaryotic cell (e.g., an animal cell, a vertebrate cell, a mammalian cell, a non-human mammalian cell, a non-human primate cell, a rodent (e.g., mouse or rat) cell, a human cell, a plant cell, or a yeast cell) or a prokaryotic cell (e.g., a bacteria cell).

In some embodiments, the cell is from a plant or an animal.

In some embodiments, the plant is a dicotyledon. In some embodiments, the dicotyledon is selected from the group consisting of soybean, cabbage (e.g., Chinese cabbage), rapeseed, Brassica, watermelon, melon, potato, tomato, tobacco, eggplant, pepper, cucumber, cotton, alfalfa, eggplant, grape.

In some embodiments, the plant is a monocotyledon. In some embodiments, the monocotyledon is selected from the group consisting of rice, corn, wheat, barley, oat, sorghum, millet, grasses, Poaceae, Zizania, Avena, Coix, Hordeum, Oryza, Panicum (e.g., Panicum miliaceum), Secale, Setaria (e.g., Setaria italica), Sorghum, Triticum, Zea, Cymbopogon, Saccharum (e.g., Saccharum officinarum), Phyllostachys, Dendrocalamus, Bambusa, Yushania.

In some embodiments, the animal is selected from the group consisting of pig, ox, sheep, goat, mouse, rat, alpaca, monkey, rabbit, chicken, duck, goose, fish (e.g., zebra fish).

In some embodiments, the cell is a eukaryotic cell, such as a mammalian cell, including a human cell (a primary human cell or an established human cell line). In some embodiments, the cell is a non-human mammalian cell, such as a cell from a non-human primate (e.g., monkey), a cow/bull/cattle, sheep, goat, pig, horse, dog, cat, rodent (such as rabbit, mouse, rat, hamster, etc.). In some embodiments, the cell is from fish (such as salmon), bird (such as poultry bird, including chick, duck, goose), reptile, shellfish (e.g., oyster, claim, lobster, shrimp), insect, worm, yeast, etc. In some embodiments, the cell is from a plant, such as monocot or dicot. In certain embodiment, the plant is a food crop such as barley, cassava, cotton, groundnuts or peanuts, maize, millet, oil palm fruit, potatoes, pulses, rapeseed or canola, rice, rye, sorghum, soybeans, sugar cane, sugar beets, sunflower, and wheat. In certain embodiment, the plant is a cereal (barley, maize, millet, rice, rye, sorghum, and wheat). In certain embodiment, the plant is a tuber (cassava and potatoes). In certain embodiment, the plant is a sugar crop (sugar beets and sugar cane). In certain embodiment, the plant is an oil-bearing crop (soybeans, groundnuts or peanuts, rapeseed or canola, sunflower, and oil palm fruit). In certain embodiment, the plant is a fiber crop (cotton). In certain embodiment, the plant is a tree (such as a peach or a nectarine tree, an apple or pear tree, a nut tree such as almond or walnut or pistachio tree, or a citrus tree, e.g., orange, grapefruit or lemon tree), a grass, a vegetable, a fruit, or an algae. In certain embodiment, the plant is a nightshade plant; a plant of the genus Brassica; a plant of the genus Lactuca; a plant of the genus Spinacia; a plant of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa, etc.

### Pharmaceutical Composition

In yet another aspect, the disclosure provides a pharmaceutical composition comprising (1) the system of the disclosure, the vector of the disclosure, the ribonucleoprotein of the disclosure, the lipid nanoparticle of the disclosure, or the cell of the disclosure; and (2) a pharmaceutically acceptable excipient.

In some embodiments, the pharmaceutical composition comprises the rAAV particle in a concentration selected from the group consisting of about 1×10¹⁰ vg/mL, 2×10¹⁰ vg/mL, 3×10¹⁰ vg/mL, 4×10¹⁰ vg/mL, 5×10¹⁰ vg/mL, 6×10¹⁰ vg/mL, 7×10¹⁰ vg/mL, 8×10¹⁰ vg/mL, 9×10¹⁰ vg/mL, 1×10¹¹ vg/mL, 2×10¹¹ vg/mL, 3×10¹¹ vg/mL, 4×10¹¹ vg/mL, 5×10¹¹ vg/mL, 6×10¹¹ vg/mL, 7×10¹¹ vg/mL, 8×10¹¹ vg/mL, 9×10¹¹ vg/mL, 1×10¹² vg/mL, 2×10¹² vg/mL, 3×10¹² vg/mL, 4×10¹² vg/mL, 5×10¹² vg/mL, 6×10¹² vg/mL, 7×10¹² vg/mL, 8×10¹² vg/mL, 9×10¹² vg/mL, 1×10¹³ vg/mL, or in a concentration of a numerical range between any of two preceding values, e.g., in a concentration of from about 9×10¹⁰ vg/mL to about 8×10¹¹ vg/mL.

In some embodiments, the pharmaceutical composition is an injection.

In some embodiments, the volume of the injection is selected from the group consisting of about 1 microliter, 10 microliters, 50 microliters, 100 microliters, 150 microliters, 200 microliters, 250 microliters, 300 microliters, 350 microliters, 400 microliters, 450 microliters, 500 microliters, 550 microliters, 600 microliters, 650 microliters, 700 microliters, 750 microliters, 800 microliters, 850 microliters, 900 microliters, 950 microliters, 1000 microliters, and a volume of a numerical range between any of two preceding values, e.g., in a concentration of from about 10 microliters to about 750 microliters.

### Method of Treatment

In yet another aspect, the disclosure provides a method for diagnosing, preventing, or treating a disease in a subject in need thereof, comprising administering to the subject (e.g., a therapeutically effective dose) the system of the disclosure, the vector of the disclosure, the ribonucleoprotein of the disclosure, the lipid nanoparticle of the disclosure, the cell of the disclosure, or the pharmaceutical composition of the disclosure, wherein the disease is associated with a target DNA, wherein the spacer sequence is capable of hybridizing to a target sequence of the target DNA, wherein the target DNA is modified by the complex, and wherein the modification of the target DNA diagnose, prevents, or treats the disease.

In some embodiments, the disease is selected from the group consisting of Angelman syndrome (AS), Alzheimer's disease (AD), transthyretin amyloidosis (ATTR), transthyretin amyloid cardiomyopathy (ATTR-CM), cystic fibrosis (CF), hereditary angioedema, diabetes, progressive pseudohypertrophic muscular dystrophy, Duchenne muscular dystrophy (DMD), Becker muscular dystrophy (BMD), spinal muscular atrophy (SMA), alpha-1-antitrypsin deficiency, Pompe disease, myotonic dystrophy, Huntington's disease (HTT), fragile X syndrome, Friedreich ataxia, amyotrophic lateral sclerosis (ALS), frontotemporal dementia, hereditary chronic kidney disease, hyperlipidemia, Leber congenital amaurosis (LCA), sickle cell disease, thalassemia (e.g., β-thalassemia), Parkinson's disease (PD), myelodysplastic syndrome (MDS), retinitis pigmentosa (RP), age-related macular degeneration (AMD), Hepatitis B, nonalcoholic fatty liver disease (NAFLD), Acquired Immune Deficiency Syndrome, corneal dystrophy (CD), hypercholesterolemia, familial hypercholesterolemia (FH), heart disease (e.g., hypertrophic cardiomyopathy (HCM)), and cancer.

In some embodiments, the target DNA encodes a mRNA, a tRNA, a ribosomal RNA (rRNA), a microRNA (miRNA), a non-coding RNA, a long non-coding (Inc) RNA, a nuclear RNA, an interfering RNA (iRNA), a small interfering RNA (siRNA), a ribozyme, a riboswitch, a satellite RNA, a microswitch, a microzyme, or a viral RNA.

In some embodiments, the target DNA is a eukaryotic DNA.

In some embodiments, the eukaryotic DNA is a mammal DNA, such as a non-human mammalian DNA, a non-human primate DNA, a human DNA, a plant DNA, an insect DNA, a bird DNA, a reptile DNA, a rodent (e.g., mouse, rat) DNA, a fish DNA, a nematode DNA, or a yeast DNA.

In some embodiments, the target DNA is in a eukaryotic cell, for example, a human cell, a non-human primate cell, or a mouse cell.

In some embodiments, the administrating comprises local administration or systemic administration.

In some embodiments, the administrating comprises intrathecal administration, intramuscular administration, intravenous administration, transdermal administration, intranasal administration, oral administration, mucosal administration, intraperitoneal administration, intracranial administration, intracerebroventricular administration, or stereotaxic administration.

In some embodiments, the administration is injection or infusion.

In some embodiments, the subject is a human, a non-human primate, or a mouse.

In some embodiments, the level of the transcript (e.g., mRNA) of the target DNA is decreased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the transcript (e.g., mRNA) of the target DNA in the subject prior to the administration.

In some embodiments, the level of the transcript (e.g., mRNA) of the target DNA is increased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the transcript (e.g., mRNA) of the target DNA in the subject prior to the administration.

In some embodiments, the level of the expression product (e.g., protein) of the target DNA is decreased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the expression product (e.g., protein) of the target DNA in the subject prior to the administration.

In some embodiments, the level of the expression product (e.g., protein) of the target DNA is increased in the subject by at least about 10%, about 15%, about 20%, about 25%, about 30%, about 35%, about 40%, about 45%, about 50%, about 55%, about 60%, about 65%, about 70%, about 75%, about 80%, about 85%, or more compared to the level of the expression product (e.g., protein) of the target DNA in the subject prior to the administration. In some embodiments, the expression product is a functional mutant of the expression product of the target DNA.

In some embodiments, the median survival of the subject suffering from the disease but receiving the administration is 5 days, 10 days, 20 days, 30 days, 2 months, 3 months, 4 months, 5 months, 6 months, 7 months, 8 months, 9 months, 10 months, 11 months, 12 months, 1.5 year, 2 years, 2.5 years, 3 years, 4 years, 5 years, 6 years, 7 years, 8 years, 9 years, 10 years or more longer than that of a subject or a population of subjects suffering from the disease and not receiving the administration.

The therapeutically effective dose may be either via a single dose, or multiple doses. One skilled in the art understands that the actual dose may vary greatly depending upon a variety of factors, such as the vector choices, the target cells, organisms, tissues, the general conditions of the subject to be treated, the degrees of transformation/modification sought, the administration routes, the administration modes, the types of transformation/modification sought, etc.

For example, the therapeutically effective dose of the rAAV particle may be about 1.0E+8, 2.0E+8, 3.0E+8, 4.0E+8, 6.0E+8, 8.0E+8, 1.0E+9, 2.0E+9, 3.0E+9, 4.0E+9, 6.0E+9, 8.0E+9, 1.0E+10, 2.0E+10, 3.0E+10, 4.0E+10, 6.0E+10, 8.0E+10, 1.0E+11, 2.0E+11, 3.0E+11, 4.0E+11, 6.0E+11, 8.0E+11, 1.0E+12, 2.0E+12, 3.0E+12, 4.0E+12, 6.0E+12, 8.0E+12, 1.0E+13, 2.0E+13, 3.0E+13, 4.0E+13, 6.0E+13, 8.0E+13, 1.0E+14, 2.0E+14, 3.0E+14, 4.0E+14, 6.0E+14, 8.0E+14, 1.0E+15, 2.0E+15, 3.0E+15, 4.0E+15, 6.0E+15, 8.0E+15, 1.0E+16, 2.0E+16, 3.0E+16, 4.0E+16, 6.0E+16, 8.0E+16, or 1.0E+17 vg, or within a range of any two of the those point values. vg stands for vector genomes of rAAV particles for administration.

### Method of Detection

In yet another aspect, the disclosure provides a method of detecting a target DNA, comprising contacting the target DNA with the system of the disclosure, wherein the target DNA is modified by the complex, and wherein the modification detects the target DNA. In some embodiments, the modification generates a detectable signal, e.g., a fluorescent signal.

### Kits

In yet another aspect, the disclosure provides a kit comprising the Cas12i polypeptide of the disclosure, the system of the disclosure, the polynucleotide of the disclosure, the vector of the disclosure, the RNP of the disclosure, the LNP of the disclosure, the delivery system of the disclosure, the cell of the disclosure, or the pharmaceutical composition of the disclosure, or any one, two, or all components of the same.

In some embodiments, the kit further comprises an instruction to use the component(s) contained therein, and/or instructions for combining with additional component(s) that may be available or necessary elsewhere.

In some embodiments, the kit further comprises one or more buffers that may be used to dissolve any of the component(s) contained therein, and/or to provide suitable reaction conditions for one or more of the component(s). Such buffers may include one or more of PBS, HEPES, Tris, MOPS, Na₂CO₃, NaHCO₃, NaB, or combinations thereof. In some embodiments, the reaction condition includes a proper pH, such as a basic pH. In some embodiments, the pH is between 7-10.

In some embodiments, any one or more of the kit components may be stored in a suitable container or at a suitable temperature, e.g., 4 Celsius degree.

Further embodiments are illustrated in the following Examples which are given for illustrative purposes only and are not intended to limit the scope of the disclosure.

### EXAMPLES

### Material and Methods

Unless otherwise specified, the experimental methods used in the Examples are conventional.

Unless otherwise specified, the materials, reagents, etc., used in the Examples are commercially available.

Unless otherwise specified, the following materials and experimental methods were used in the Examples.

### Plasmid Vector Construction.

Human codon-optimized Cas12i, TadA8e and human APOBEC3A genes were synthesized by the GenScript Co., Ltd., and cloned to generate pCAG_NLS-Cas12i-NLS_pA_pU6_BpiI_pCMV_mCherry_pA by Gibson Assembly. crRNA oligos were synthesized by HuaGene Co., Ltd., annealed and ligated into BpiI site to produce the pCAG_NLS-Cas12i-NLS_pA_pU6_crRNA_pCMV_mCherry_pA.

### Cell Culture, Transfection, and Flow Cytometry Analysis.

The mammalian cell lines used in this study were HEK293T and N2A. Cells were cultured in Dulbecco's modified Eagle's medium (DMEM) supplemented with 10% FBS, penicillin/streptomycin and GlutMAX. Transfections were performed using Polyetherimide (PEI). For variant/mutant screening, HEK293T cells were cultured in 24-well plates, and after 12 hours 2 μg of the plasmids (1 μg of an expression plasmid and 1 μg of a reporter plasmid) were transfected into these cells with 4 μL PEI. 48 hours after transfection, BFP, mCherry, and EGFP fluorescence were analyzed using a Beckman CytoFlex flow-cytometer. For assay of mutations in target sites of endogenous genes, 1 μg of expression plasmid was transfected into HEK293T or N2A cells, which were then sorted using a BD FACS Aria III, BD LSRFortessa X-20 flow cytometer, 48 hours after transfection.

### Detection of Gene Editing Frequency.

Six thousand sorted cells were lysed in 20 μl of lysis buffer (Vazyme). Targeted sequence primers were synthesized and used in nested PCR amplification by Phanta Max Super-Fidelity DNA Polymerase (Vazyme). Targeted deep sequence analysis was used to determine indel frequencies. A-to-G or C-to-T editing frequencies were calculated by targeted deep sequence analysis or Sanger sequencing and EditR. A-to-G editing purity were calculated as A-to-G editing efficiency/(A-to-T editing efficiency+A-to-C editing efficiency+A-to-G editing efficiency). C-to-T editing purity were calculated as C-to-T editing efficiency/(C-to-A editing efficiency+C-to-G editing efficiency+C-to-T editing efficiency).

### Pem-Seq.

PEM-seq in HEK293 cells was performed as previously described. Briefly, all-in-one plasmids containing LbCas12a, Ultra-AsCas12a, hfCas12Max, ABR001 or Cas12i2HiFi with targeting TTR. 2 crRNA were transfected into HEK293 cells by PEI respectively, and after 48 hrs, positive cells were harvested for DNA extraction. The 20 μg genomic DNA was fragmented with a peak length of 300-700 bp by Covaris sonication. DNA fragments was tagged with biotin by a one-round biotinylated primer extension at 5′-end, and then primer removal by AMPure XP beads and purified by streptavidin beads. The single-stranded DNA on streptavidin beads is ligased with a bridge adapter containing 14-bp RMB, and PCR product was performed nested PCR for enriching DNA fragment containing the bait DSB and tagged with illumine adapter sequences. The prepared sequencing library was sequencing on an Hi-seq 2500, with a 2×150 bp.

### RNP Delivery and Ex Vivo Editing.

RNP was complexed by mixing purified hfCas12Max proteins with chemically synthesized RNA oligonucleotides (Genscript) at a 1:2 molar ratio in 1×PBS. RNP was incubated at room temperature for >15 min prior to electroporation with Lonza® 4D-Nucleofector™. 0.2×10⁶ cells were resuspended in 20 μL of Lonza buffer and mixed with 5 μL RNP with different concentrations electroporated according to Lonza specifications. HEK293 or CD3+ T cells were harvested 72 hrs post-electroporation for targeted deep sequence analysis.

### LNP Delivery and In Vivo Editing.

LNPs were formulated with ALC0315, cholesterol, DMG-PEG2k, DSPC in 100% ethanol, carrying in vitro transcription (IVT) mRNA and chemically synthesized RNA oligonucleotides (Genscript) with a 1:1 weight ratio. LNPs were formed according to the manufacturer's protocol, by microfluidic mixing the lipid with RNA solutions using a Precision Nano-systems NanoAssemblr Benchtop Instrument. LNPs diluted in PBS were transfected into N2a cells at 0.1, 0.3, 0.5, 1 μg RNA, or delivered into C57 mouse with different dose by through tail intravenous injection. Cells were harvested 48 hrs post-transfection for lysis and targeted deep sequence analysis. For in vivo editing, liver tissue was collected from the left or median lateral lobe of each mouse 7 days post-injection for DNA extraction and targeted deep sequence analysis.

### Zygote Injection and Embryo Culturing.

Super ovulated female C57 mice (7-8 weeks old) by injecting 5 IU of pregnant mare serum gonadotropin (PMSG), followed by 5 IU of human chorionic gonadotropin (hCG) 48 hrs later were mated to B6D2F1 males, and fertilized embryos were collected from oviducts 20 hrs post hCG injection. For zygote injection, hfCas12Max mRNA (100 ng/μL) and gRNA (100 ng/μL) were mixed and injected into the cytoplasm of fertilized eggs in a droplet of HEPES-CZB medium containing 5 mg/ml cytochalasin B (CB) using a FemtoJet microinjector (Eppendorf) with constant flow settings. The injected zygotes were cultured in KSOM medium with amino acids at 37° C. under 5% CO₂ in air to blastocysts and harvested for targeted deep sequence analysis.

### Example 1 Identification of Cas12i Proteins and Evaluation of their dsDNA Cleavage Activity

In order to identify more Cas12i proteins, the applicant developed and employed a bioinformatics pipeline to annotate Cas12i proteins, CRISPR arrays, DR sequences, and predicted PAM preferences, and identified 10 Cas12i proteins and associated sequences in Table 1 below.

TABLE 1

| | | SEQ ID NO: | | | |
|---|---|---|---|---|---|
| Name | Name | | | | Codon-optimized |
| Cas12i | Cas12i | Cas12i amino acid | Corresponding | Cas12i coding | Cas12i coding |
| protein | protein | sequence | DR sequence | sequences | sequence |
| SiCas12i | Cas12i12 | 1 | 11 | 21 | 31 |
| | (xCas12i) | | | | |
| Si2Cas12i | Cas12i3 | 2 | 12 | 22 | 32 |
| WiCas12i | Cas12i7 | 3 | 13 | 23 | 33 |
| Wi2Cas12i | Cas12i8 | 4 | 14 | 24 | 34 |
| Wi3Cas12i | Cas12i9 | 5 | 15 | 25 | 35 |
| SaCas12i | Cas12i11 | 6 | 16 | 26 | 36 |
| Sa2Cas12i | Cas12i4 | 7 | 17 | 27 | 37 |
| Sa3Cas12i | Cas12i5 | 8 | 18 | 28 | 38 |
| WaCas12i | Cas12i6 | 9 | 19 | 29 | 39 |
| Wa2Cas12i | Cas12i10 | 10 | 20 | 30 | 40 |

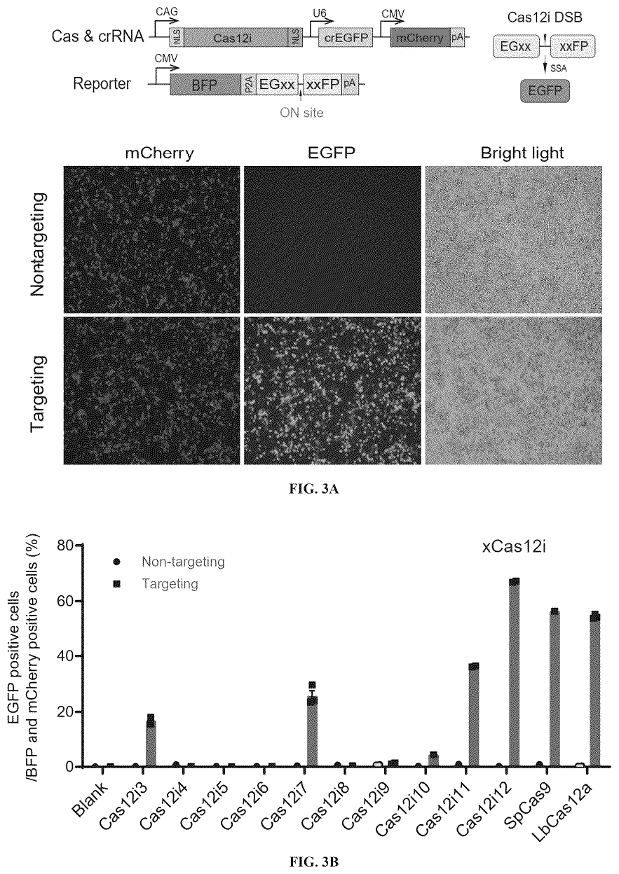

To evaluate the guide sequence-specific dsDNA cleavage activity (“dsDNA cleavage activity” for short as used in the disclosure) of these Cas12i proteins in mammalian cells, the applicant designed a dual plasmid fluorescent reporter system, which detected the increased enhanced green fluorescent protein (EGFP) signal intensity activated by Cas-mediated dsDNA cleavage or double strand breaks (FIG. 3A). This system relied on the co-transfection of an expression plasmid encoding mCherry, a nuclear localization signal (NLS)-tagged Cas protein, and a guide RNA (gRNA, or crRNA), and a reporter plasmid encoding BFP and activatable EGxxFP cassette, which is EGxx-target site-xxFP. EGFP activation was carried out by Cas mediated DSB and single-strand annealing (SSA)-mediated repair.

Specifically, referring to FIG. 3A, the reporter plasmid comprised a polynucleotide encoding, from 5′ to 3′, BFP-P2A-activatable EGxxxxFP (SEQ ID NO: 41) (EGxx-insertion sequence (SEQ ID NO: 42) (containing, from 5′ to 3′, a protospacer adjacent motif (PAM)) of [symbol] for Cas12i protein, a protospacer sequence (SEQ ID NO: 43) (which is the reverse complementary sequence of a target sequence (SEQ ID NO: 44)), and a protospacer adjacent motif (PAM)) of [symbol] for Cas9 protein-xxFP), followed by a bGH polyA (SEQ ID NO: 448) coding sequence, operably linked to human CMV promoter (SEQ ID NO: 447). The protospacer sequence (SEQ ID NO: 43) contained a premature stop codon [symbol] that prevented the expression of EGFP and hence emission of green fluorescent signals. The BFP coding sequence expresses BFP to indicate the successful transfection and expression of the reporter plasmid into host cells through blue fluorescence.

Most of the known Cas12i proteins recognize a 5′-T-rich PAM 5′ to protospacer sequence in dsDNA, while Cas9 recognizes a 3′-G-rich PAM 3′ to protospacer sequence in dsDNA. The co-existence of the 5′ PAM of [symbol] for Cas12i protein and the 3′ PAM of [symbol] for Cas9 protein flanking the protospacer sequence (SEQ ID NO: 43) allows the simultaneous evaluation and comparison of dsDNA cleavage activity of Cas12i protein and Cas9 protein.

Activatable EGxxxxFP coding sequence, SEQ ID NO: 41
atgagcgagctgattaaggagaacatgcacatgaagctgtatatggagggcaccgtggacaaccatcacttcaagtgcacatccgagggcgaaggcaag
ccctacgagggcacccagaccatgagaatcaaggtggtcgagggcggccctctccccttcgccttcgacatcctggctactagcttcctctacggcagc
aagaccttcatcaaccacacccagggcatccccgacttcttcaagcagtccttccctgagggcttcacatgggagagagtcaccacatacgaggacgggg
gcgtgctgaccgctacccaggacaccagcctccaggacggctgcctcatctacaacgtcaagatcagaggggtgaacttcacatccaacggccctg
tgatgcagaagaaaacactcggctgggaggccttcaccgagacactgtaccccgctgacggcggcctggaaggcagaaacgacatggccctgaagctcgt
gggcgggagccatctgatcgcaaacatcaagaccacatatagatccaagaaacccgctaagaacctcaagatgcctggcgtctacatgtggactacagac
tggaaagaatcaaggaggccaacaacgagacatacgtcgagcagcacgaggtggcagtggccagatactgcgacctccctagcaaactggggcacaagc
tgaatgaattcgagggcaggggcagcctgctgacctgcggcgacgtggaggagaaccccggccccatggtgagcaagggcgaggagctgttcaccgggg
tggtgcccatcctggtcgagctggacggcgacgtaaacggccacaagttcagcgtgtccggcgagggcgagggcgatgccacctacggcaagctgaccct
gaagttcatctgcaccaccggcaagctgcccgtgccctggcccaccctcgtgaccaccctgacctacggcgtgcagtgcttcagccgctaccccgacca
catgaagcagcacgacttcttcaagtccgccatgcccgaaggctacgtccaggagcgcaccatcttcttcaaggacgacggcaactacaagacccgcgcc
gaggtgaagttcgagggcgacaccctggtgaaccgcatcgagctgaagggcatcgacttcaaggaggacggcaacatcctggggcacaagctggagtac
aactacaacagccacaacgtctatatcatggccgacaagcagaagaacggcatcaaggtgaacttcaag

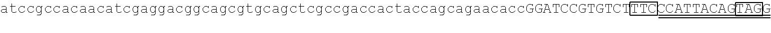

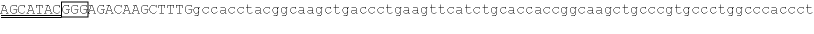

cgtgaccaccctgacctacggcgtgcagtgcttcagccgctaccccgaccacatgaagcagcacgacttcttcaagtccgccatgcccgaaggctacgtc
caggagcgcaccatcttcttcaaggacgacggcaactacaagacccgcgccgaggtgaagttcgagggcgacaccctggtgaa
ccgcatcgagctgaagggcatcgacttcaaggaggacggcaacatcctggggcacaagctggagtacaactacaacagccacaacg
tctatatcatggccgacaagcagaagaacggcatcaaggtgaacttcaagatccgccacaacatcgaggacggcagcgtgcagctcgc
cgaccactaccagcagaacacccccatcggcgacggccccgtgctgctgcccgacaaccactacctgagcacccagtccgccctgagcaaa
gaccccaacgagaagcgcgatcacatggtcctgctggagttcgtgaccgccgccgggatcactctcggcatggacg
agctgtacaagtaa
Insertion sequence, SEQ ID NO: 42

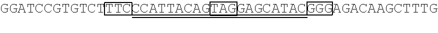

Protospacer sequence (Reverse complementary sequence of the target sequence),
20bp, SEQ ID NO: 43

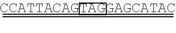

Target sequence, 20 nt, SEQ ID NO: 44

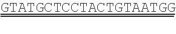

EGxxxxFP-targeting spacer sequence, 20 nt, SEQ ID NO: 45

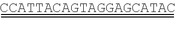

Non-targeting (“NT”) spacer sequence, 20 nt SEQ ID NO: 46
GGTCTTCGATAAGAAGACCT

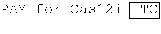

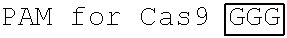

Also referring to FIG. 3A, the expression plasmid comprised from 5′ to 3′ i) a Cas12i coding sequence codon optimized for expression in mammalian cells (one of SEQ ID NOs: 31-40) encoding a Cas12i protein (one of SEQ ID NOs: 1-10) flanked by a SV40 NLS (SEQ ID NO: 444) coding sequence on its 5′ end and a NP NLS (SEQ ID NO: 445) coding sequence on its 3′ end, followed by a bGH polyA (SEQ ID NO: 448) coding sequence, operably linked to CAG promoter (SEQ ID NO: 500), ii) a sequence encoding a guide RNA (gRNA) composed of 5′-DR sequence-spacer sequence-3′ operably linked to human U6 promoter (SEQ ID NO: 446); and iii) a coding sequence for mCherry followed by a bGH polyA (SEQ ID NO: 448) coding sequence operably linked to human CMV promoter (SEQ ID NO: 447). The mCherry coding sequence expresses mCherry to indicate the successful transfection and expression of the expression plasmid into host cells through red fluorescence.

In the event that both the target sequence on the target strand and the protospacer sequence on the nontarget strand of the target dsDNA are successfully cleaved by a Cas12i protein guided by a gRNA to generate a double-strand break (DSB), the subsequent DNA repairing such as single-strand annealing (SSA)-mediated repair trigged by the DSB would restore the EGFP coding sequence to express EGFP with green fluorescence emission indicative of dsDNA cleavage activity.

For test groups, the spacer sequence comprised in the gRNA (“crEGFP”, one of SEQ ID NOs: 51-60) for use with each corresponding tested Cas12i protein (one of SEQ ID NOs: 1-10) is a EGxxxxFP-targeting spacer sequence (SEQ ID NO: 45) designed to target and hybridize to the target sequence (SEQ ID NO: 44), and the DR sequence in the gRNA (one of SEQ ID NOs: 51-60) is a DR sequence (one of SEQ ID NOs: 11-20) corresponding to each tested Cas12i protein (one of SEQ ID NOs: 1-10), as shown in Table 2.

TABLE 2

| | SEQ ID NO: | | |
|---|---|---|---|
| Cas12i protein | DR sequence | Spacer sequence | Guide RNA |
| SiCas12i (xCas12i) | 11 | 45 | 51 |
| Si2Cas12i | 12 | 45 | 52 |
| WiCas12i | 13 | 45 | 53 |
| Wi2Cas12i | 14 | 45 | 54 |
| Wi3Cas12i | 15 | 45 | 55 |
| SaCas12i | 16 | 45 | 56 |
| Sa2Cas12i | 17 | 45 | 57 |
| Sa3Cas12i | 18 | 45 | 58 |
| WaCas12i | 19 | 45 | 59 |
| Wa2Cas12i | 20 | 45 | 60 |

For negative control (“NT”) for each tested Cas12/9 protein (Cas12i, SpCas9, LbCas12a), a non-targeting spacer sequence (“NT”, SEQ ID NO: 46) incapable of hybridizing to the target sequence (SEQ ID NO: 44) was used in place of the EGxxxxFP-targeting spacer sequence (SEQ ID NO: 45), while the other elements of each tested CRISPR-Cas12/9 system remained.

For positive control, CRISPR-SpCas9 and CRISPR-LbCas12a systems each comprising a Cas protein and a guide RNA as shown in Table 3 below were used in place of the tested CRISPR-Cas12i systems in Tables 1 and 2 above, using the same EGxxxxFP-targeting spacer sequence (SEQ ID NO: 45). Note that the gRNA for the CRISPR-SpCas9 system was composed of 5′-spacer sequence-scaffold sequence-3′, and the gRNA for the CRISPR-LbCas12a system was composed of 5′-DR sequence-spacer sequence-3′.

TABLE 3

| | Control Cas amino | |
|---|---|---|
| Control Cas protein | acid sequence | Guide RNA |
| SpCas9 | SEQ ID NO: 47 | SEQ ID NO: 48 |
| LbCas12a | SEQ ID NO: 49 | SEQ ID NO: 50 |

HEK293T cells were cultured in 24-well tissue culture plates according to standard methods for 12 hours, before the reporter and expression plasmids were co-transfected into the cells using standard polyethyleneimine (PEI) transfection. The transfected cells were then cultured at 37° C. under 5% CO₂ for 48 hours. Then the cultured cells were analyzed by flow cytometry for BFP, EGFP, and mCherry fluorescent signals. A “blank” control group was also set up, where only the reporter plasmid was transfected, and no expression plasmid was introduced.

The dsDNA cleavage activities of the tested Cas proteins were calculated as the percentage of EGFP positive cells in BFP &mCherry dual-positive cells (“EGFP⁺”, indicating dsDNA cleavage at the indicated target site on the reporter plasmid; “mCherry⁺ BFP⁺”, indicating successful co-transfection and co-expression of the expression and reporter plasmids). The higher the % EGFP⁺/mCherry⁺ BFP⁺ is, the higher the dsDNA cleavage activity would be.

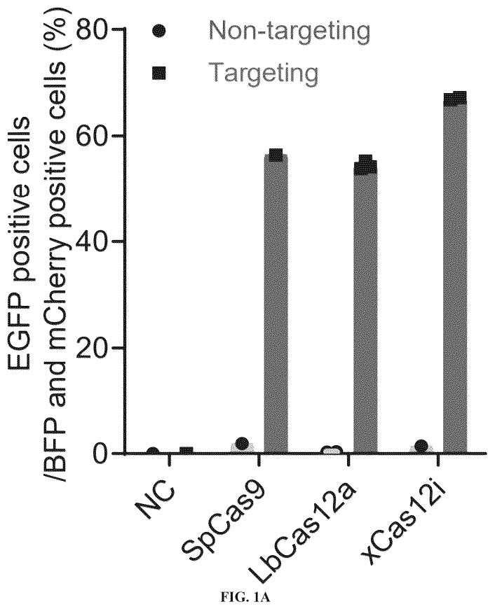

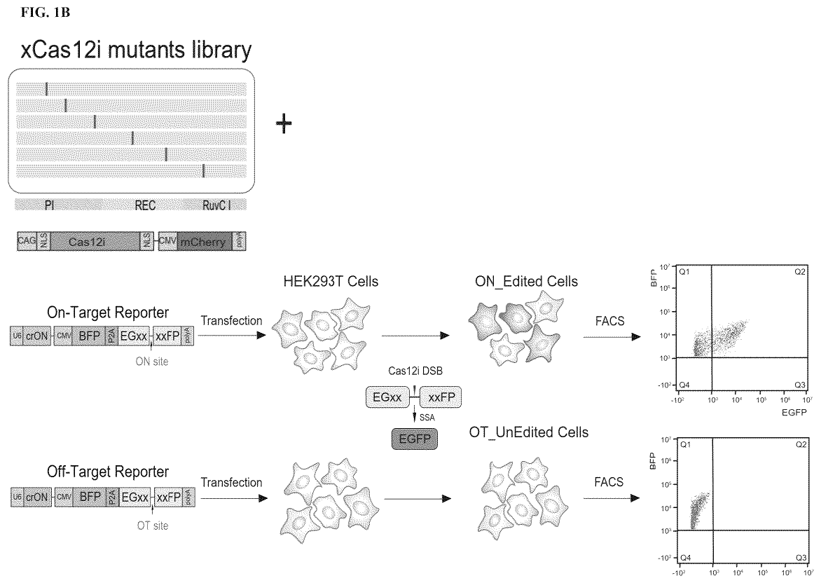

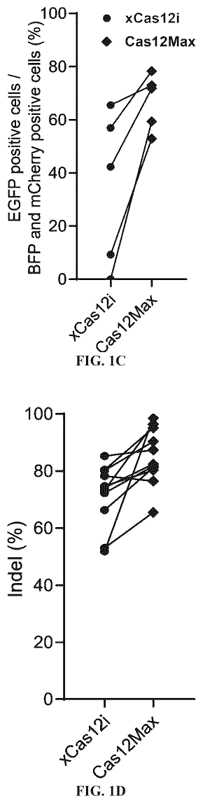

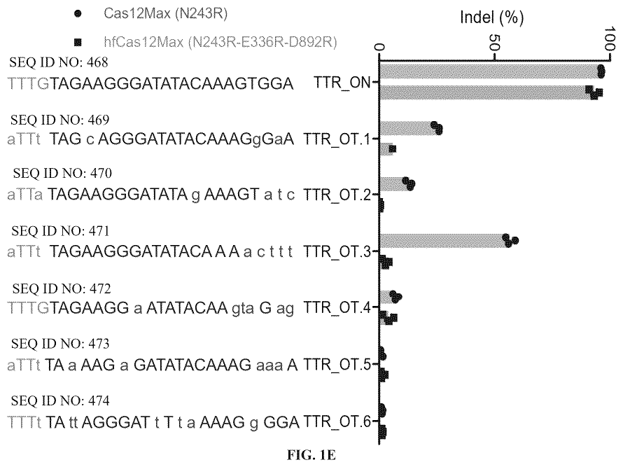

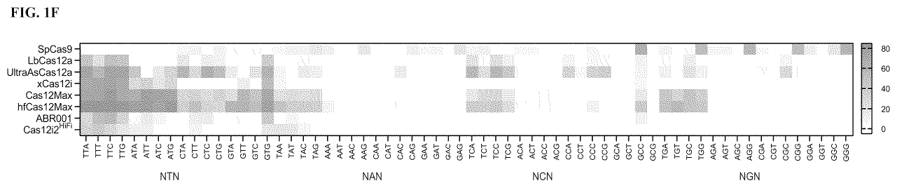

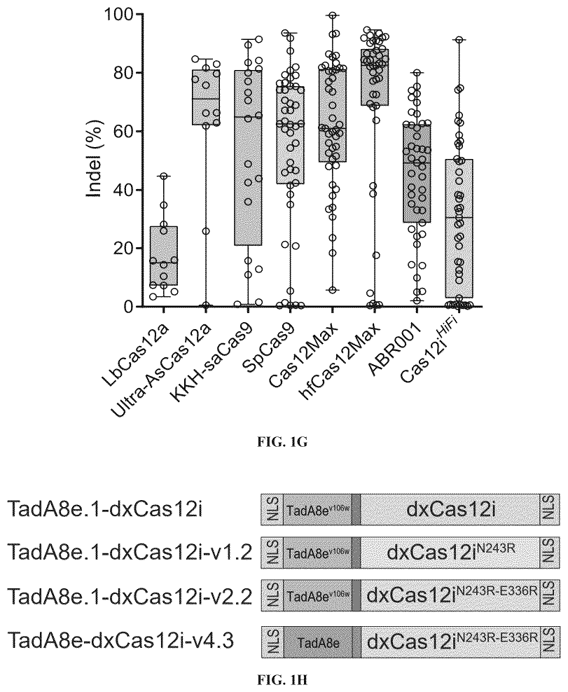

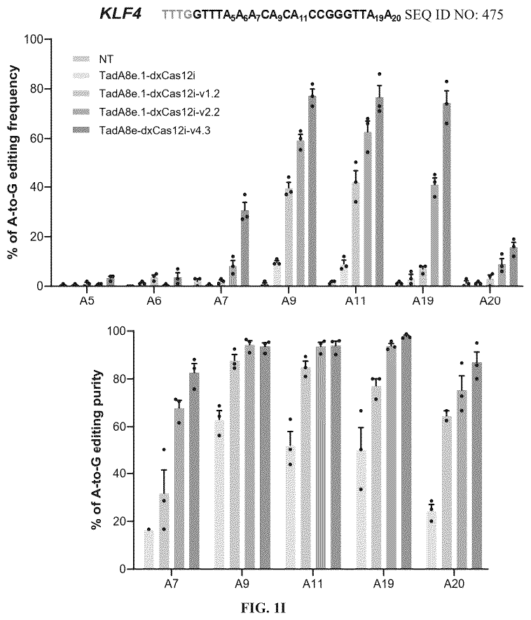

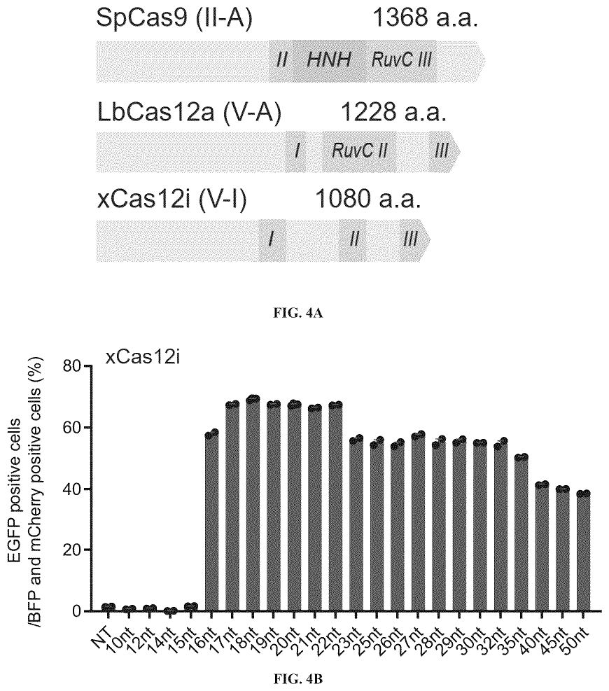

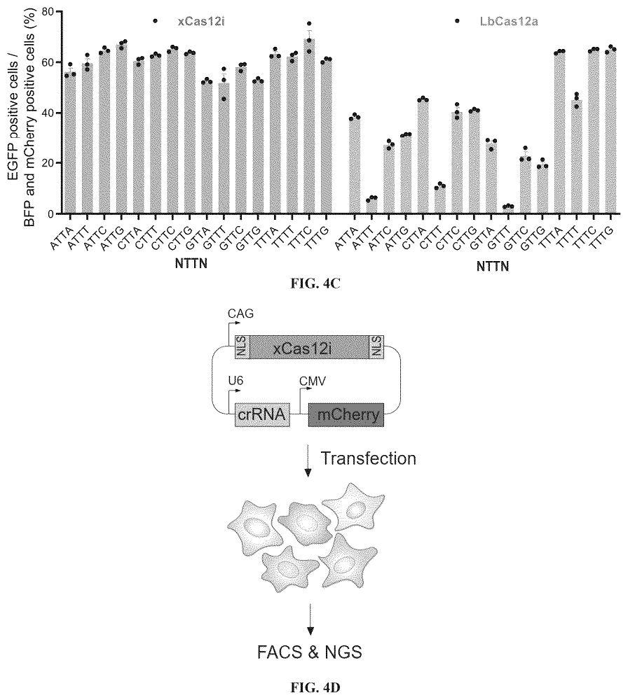

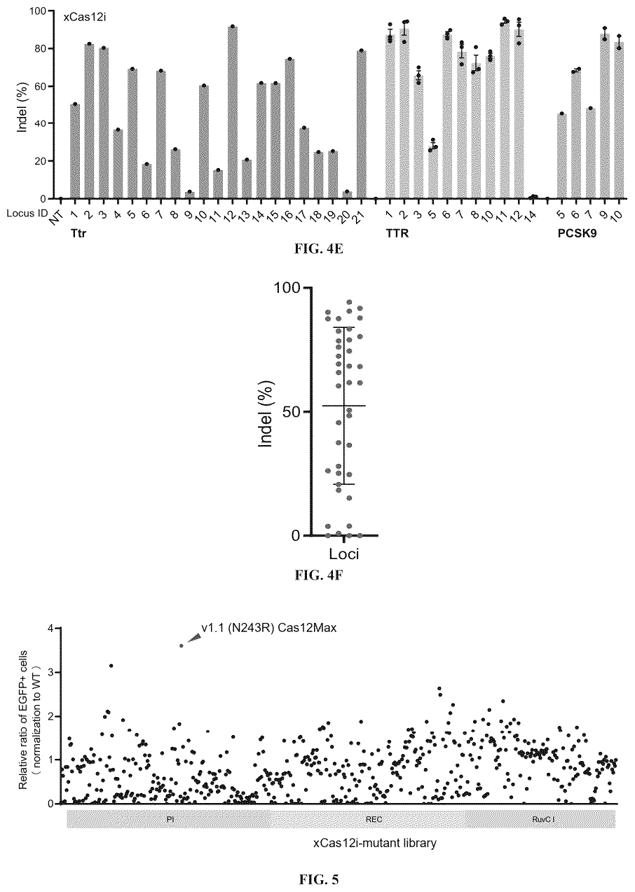

Using this dual plasmid fluorescent reporter system, it was observed that five Cas12i proteins (Cas12i3 (SEQ ID NO: 2), Cas12i7 (SEQ ID NO: 3), Cas12i10 (SEQ ID NO: 10), Cas12i11 (SEQ ID NO: 6), and Cas12i12 (SEQ ID NO: 1, also referred to as SiCas12i or xCas12i in the disclosure)) exhibited targeted gRNA induced significant activation of EGFP expression indicative of significant dsDNA cleavage (FIG. 1A, FIG. 3B), and among them, Cas12i12 even exhibited a higher dsDNA cleavage activity than both LbCas12a and SpCas9 as determined by Fluorescence Activated Cell Sorter (FACS) analysis (FIG. 1A, FIG. 3B). The xCas12i (1080 aa) was smaller in size compared to SpCas9 (1368 aa) and LbCas12a (1228 aa) (FIG. 4A).

### Example 2 Evaluation of Effective Spacer Sequence Length for xCas12i

Using the dual plasmid fluorescent reporter system in Example 1, to test the effective spacer sequence length for xCas12i, 22 spacer sequences of different lengths ranging from 10 to 50 nt (SEQ ID NOs: 45 and 61-81 as shown in Table 4 below) were designed to target and hybridize to the reverse complementary sequence of a protospacer sequence (SEQ ID NO: 43, or one of SEQ ID NOs: 61-81) comprised in the insertion sequence (SEQ ID NO: 42) of the GFxxxxFP reporter plasmid in Example 1, wherein the 20 nt spacer sequence in Table 4 is exactly the EGxxxxFP-targeting spacer sequence (SEQ ID NO: 45) in Example 1. To evaluate the additional spacer sequence lengths, the EGxxxxFP targeting spacer sequence (SEQ ID NO: 45) in the guide RNA encoded in the expression plasmid was replaced with the spacer sequence in respective length (one of SEQ ID NOs: 61-81) in Table 4, while the other elements of the dual plasmid fluorescent reporter system remained. To save drafting, the sequences in Table 4 refer to both the protospacer sequence (a DNA sequence) and the corresponding spacer sequence (an RNA sequence) with any “T” in the sequence when referring to a protospacer sequences standing for “T” and when referring to such a spacer sequence standing for “U”, although the assigned SEQ ID NOs: 61-81 in the sequence listing are annotated as RNA.

TABLE 4

| | Protospacer sequence/Spacer sequence | SEQ ID NO: |
|---|---|---|
| 10-nt | CCATTACAGT | 61 |
| 12-nt | CCATTACAGTAG | 62 |
| 14-nt | CCATTACAGTAGGA | 63 |
| 15-nt | CCATTACAGTAGGAG | 64 |
| 16-nt | CCATTACAGTAGGAGC | 65 |
| 17-nt | CCATTACAGTAGGAGCA | 66 |
| 18-nt | CCATTACAGTAGGAGCAT | 67 |
| 19-nt | CCATTACAGTAGGAGCATA | 68 |
| 20-nt | CCATTACAGTAGGAGCATAC | 45 |
| 21-nt | CCATTACAGTAGGAGCATACG | 69 |
| 22-nt | CCATTACAGTAGGAGCATACGG | 70 |
| 23-nt | CCATTACAGTAGGAGCATACGGG | 71 |
| 24-nt | CCATTACAGTAGGAGCATACGGGA | 72 |
| 26-nt | CCATTACAGTAGGAGCATACGGGAGA | 73 |
| 27-nt | CCATTACAGTAGGAGCATACGGGAGAC | 74 |
| 28-nt | CCATTACAGTAGGAGCATACGGGAGACA | 75 |
| 30-nt | CCATTACAGTAGGAGCATACGGGAGACAAG | 76 |
| 32-nt | CCATTACAGTAGGAGCATACGGGAGACAAGCT | 77 |
| 35-nt | CCATTACAGTAGGAGCATACGGGAGACAAGCTTTG | 78 |
| 40-nt | CCATTACAGTAGGAGCATACGGGAGACAAGCTTTGGCCAC | 79 |
| 45-nt | CCATTACAGTAGGAGCATACGGGAGACAAGCTTTGGCCACCTACG | 80 |
| 50-nt | CCATTACAGTAGGAGCATACGGGAGACAAGCTTTGGCCACCTACGGCAAG | 81 |

By using the experimental procedure in Example 1, it was observed that a spacer sequence length range of at least 16 nucleotides is effective for xCas12i's spacer sequence-specific cleavage activity, and among that range, 17-22 nt is optimal (FIG. 4B).

### Example 3 Evaluation of PAM Recognition for xCas12i

Considering the 5′-TTN PAM preference of Cas12i, the applicant performed a NTTN PAM identification assay (wherein N is A, T, C, or G) using the dual plasmid fluorescent reporter system in Example 1, in which various 5′ PAM was used in place of the original 5′ PAM of [symbol] , while the other elements of the dual plasmid fluorescent reporter system remained.

By using the experimental procedure in Example 1, it was observed that xCas12i showed a consistently high frequency of EGFP activation at target sites with 5′-NTTN

PAM sequences, wherein N is A, T, C, or G, while LbCas12a had comparable activity at just 5′-TTTN PAM, respectively (FIG. 4C), showing the much more broad PAM site recognition of xCas12i.

### Example 4 Tolerance of Variation in DR Sequence of xCas12i System

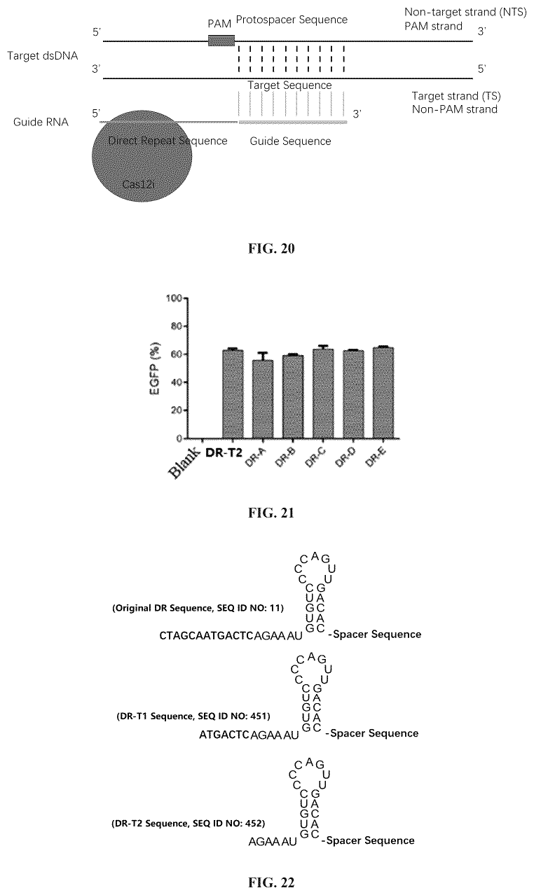

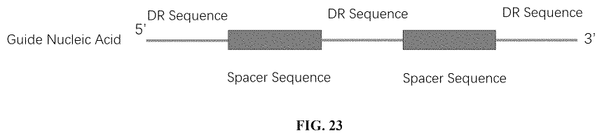

To test whether the original direct repeat (DR) sequence (SEQ ID NO: 11, 36 nt) identified together with xCas12i could tolerate variation, the applicant truncated the original DR sequence to generate two functional fragments DR-T1 (30 nt) and DR-T2 (23 nt) of SEQ ID NOs: 451 and 452, respectively, without destroying the secondary structure of the original DR sequence (FIG. 22), and then designed five DR variants of DR-T2 to generate DR-A, DR-B, DR-C, DR-D, and DR-E sequences of SEQ ID NOs: 453-457, respectively, each containing 5% to 30% mutations in the stem-loop regions without destroying the secondary structure of the original DR sequence. That is, the secondary structures of the 7 DR variants were substantially the same as that of the original DR sequence.

DR-T1, 30 nt
SEQ ID NO: 451
ATGACTCAGAAATGTGTCCCCAGTTGACAC
DR-T2 sequence, 23 nt
SEQ ID NO: 452
AGAAATGTGTCCCCAGTTGACAC
DR-A sequence, 23 nt
SEQ ID NO: 453
AGAAATCCGTCCTTAGTTGACGG
DR-B sequence, 22 nt
SEQ ID NO: 454
AGACATGTGTCCCCAGTGACAC
DR-C sequence, 23 nt
SEQ ID NO: 455
AGAAATGTTTCCCCAGTTGAAAC
DR-D sequence, 23 nt
SEQ ID NO: 456
AGAAATGTGTTCCCAGTTAACAC
DR-E sequence, 23 nt
SEQ ID NO: 457
AGAAATTTGTCCCCAGTTGACAA

By using the dual plasmid fluorescent reporter system for xCas12i in Example 1 with the original DR sequence (SEQ ID NO: 11) replaced with each of the DR variants (DR-T1, DR-T2, DR-A, DR-B, DR-C, DR-D, and DR-E), while the other element of the reporter system remained, the results (FIG. 21) show that xCas12i still exhibited high dsDNA cleavage activity mediated by gRNAs with various DR sequence variants. It can be seen that under the condition that the secondary structure of the DR sequence is maintained (i.e., the secondary structures of the DR variants are substantially the same as that of the original DR sequence), the CRISPR-SiCas12i system tolerated mismatching or deletion on DR sequence without substantial loss of dsDNA cleavage activity, indicating wide adaptability to variations in the DR sequence. These data also demonstrated that the two truncations of the original xCas12i DR sequence of SEQ ID NO: 11 (36 nt), i.e., DR-T1 (SEQ ID NO: 451, 30 nt) and DR-T2 (SEQ ID NO: 452, 23 nt), could still mediate high dsDNA cleavage activity of xCas12i.

### Example 5 Evaluation of dsDNA Cleavage Activity of xCas12i at Endogenous Gene

To further verify the dsDNA cleavage activity of xCas12i at an endogenous gene (genome cleavage) in mammalian cells, the applicant transfected the expression plasmid (FIG. 3A, FIG. 4D) in Example 1 encoding NLS tagged xCas12i with gRNAs targeting 37 sites from human TTR gene and human PCSK9 gene in HEK293T (human embryonic kidney 293 cells) or mouse Ttr gene in N2a cells (Neuro2a cells, a fast-growing mouse neuroblastoma cell line). The EGxxxxFP targeting spacer sequence (SEQ ID NO: 45) in Example 1 was replaced with respective gene-targeting spacer sequence (SEQ ID NOs: 82-119 and 121-125 in Table 5), the DR-T1 sequence (SEQ ID NO: 451) was used in place of the original DR sequence (SEQ ID NO: 11) (and also in the Examples below unless otherwise specified), while the other elements of the CRISPR-xCas12i system in Example 1 remained. The dsDNA cleavage activity, i.e., indel (insertion and/or deletion) formation, at these loci was measured 48 hours after transfection using FACS and targeted deep sequencing. To save drafting, the sequences in Table 5 refer to both the protospacer sequence (a DNA sequence) and the corresponding spacer sequence (an RNA sequence) with any “T” in the sequence when referring to a protospacer sequences standing for “T” and when referring to a such spacer sequence standing for “U”, although the assigned SEQ ID NOs: 82-119 and 121-125 in the sequence listing are annotated as DNA.

It was observed that xCas12i mediated a high frequency, up to 90%, of indel formation at most sites from Ttr, TTR and PCSK9, with a mean indel formation rate of over 50% (FIG. 4E-F). These data indicate that xCas12i exhibits a robust genome editing efficiency in mammalian cells, suggesting that it has excellent potential for therapeutic genome editing applications.

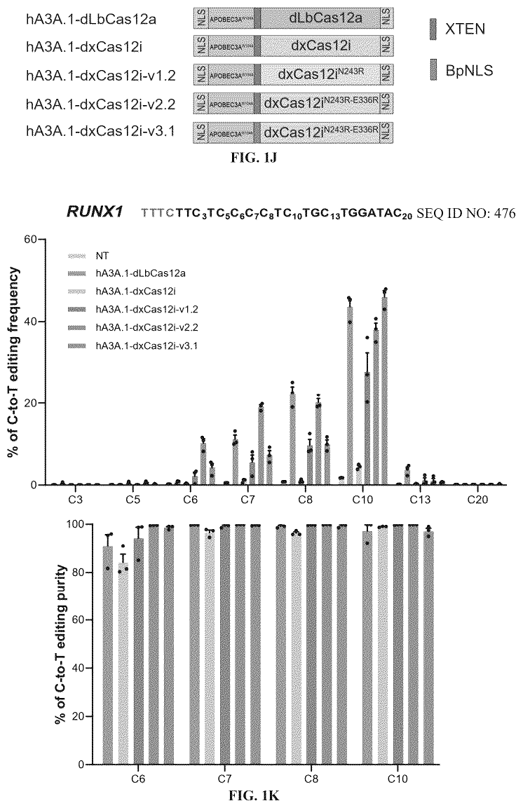

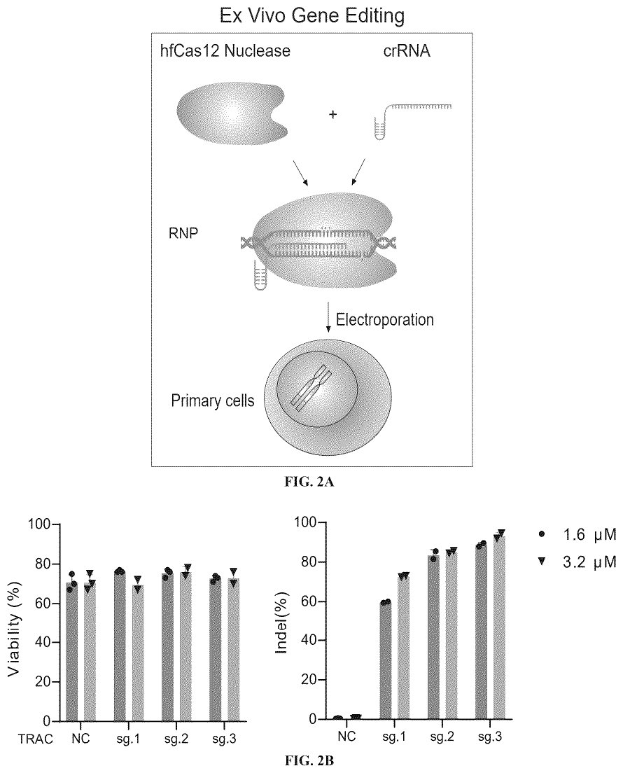

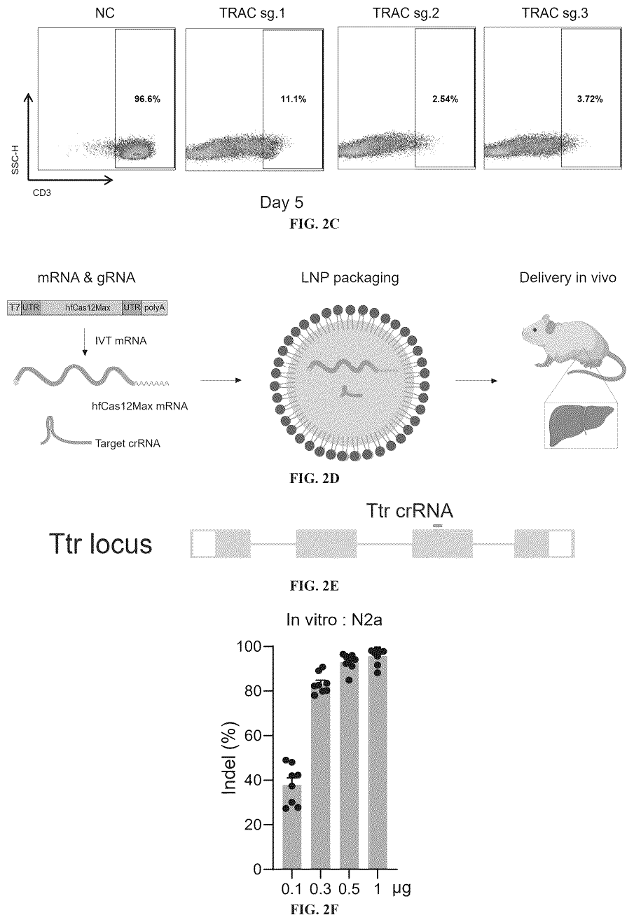

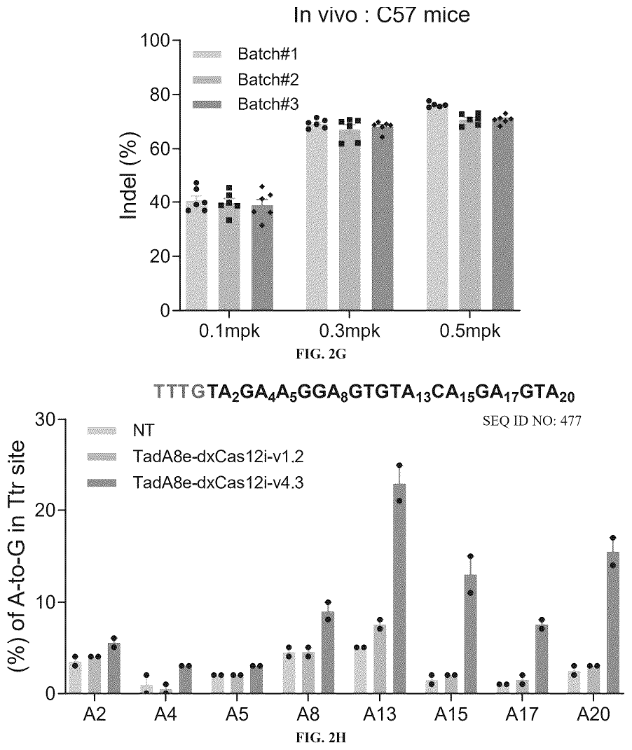

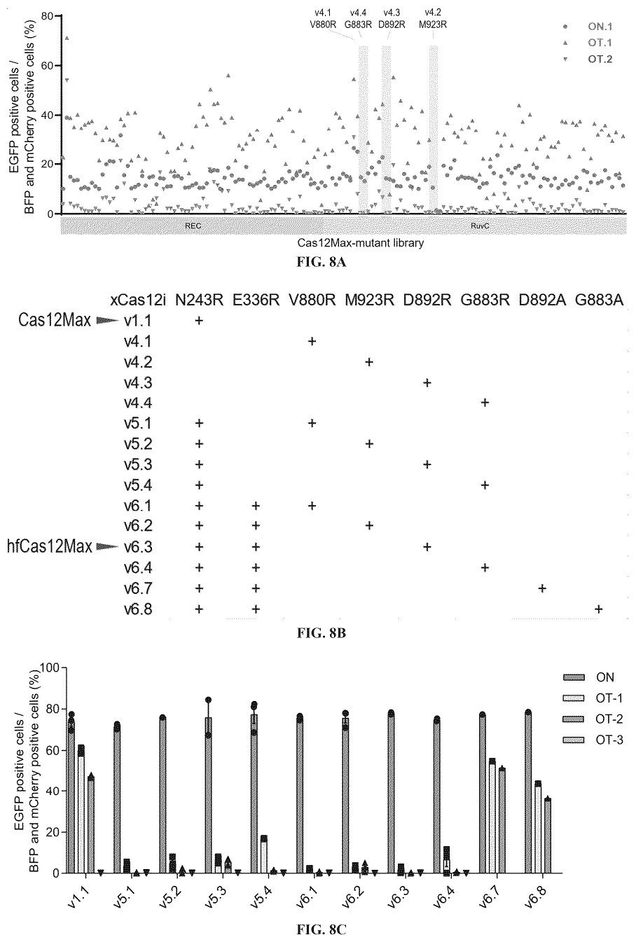

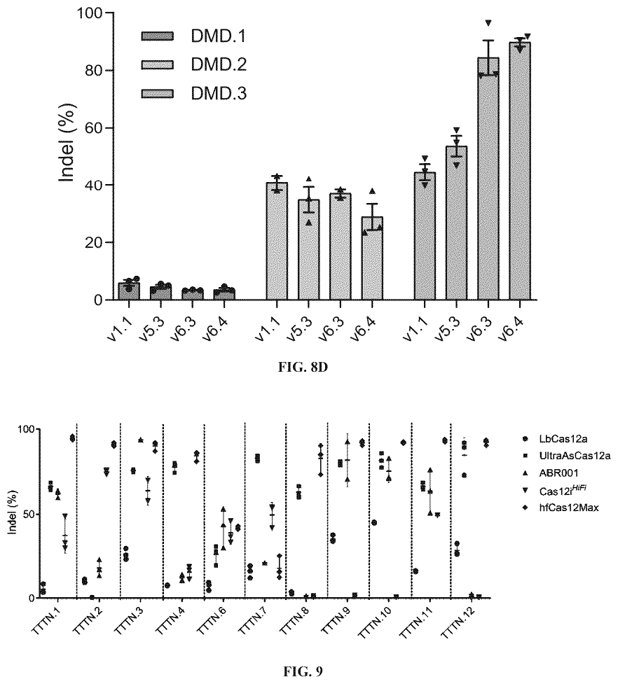

TABLE 5
Sequences for testing genome cleavage at target loci

| | | | | SEQ ID | |
|---|---|---|---|---|---|
| | | | | NO of | |
| Genomic | | | Protospacer sequence/ | protospacer/ | |
| loci | Guide RNA | PAM | Spacer sequences | sequence | Figure |
| DMD | DMD_sg1 | TTTG | CAAAAACCCAAAATATTTTA | 82 | FIG. 1D, DIG. 6B-C |
| | | | | | and FIG. 8D |
| | DMD_sg2 | TTTA | GCTCCTACTCAGACTGTTAC | 83 | FIG. 1D, DIG. 6B-C |
| | | | | | and FIG. 8D |
| | DMD_sg3 | GTTG | TGTCACCAGAGTAACAGTC | 84 | FIG. 1D, DIG. 6B-C |
| | | | | | and FIG. 8D |
| Ttr | Ttr_sg1 | TTTG | CCTCGCTGGACTGGTATTTG | 85 | FIG. 4E-F |
| | Ttr_sg2 | TTTG | TGTCTGAAGCTGGCCCCGCG | 86 | FIG. 1D and. FIG. |
| | | | | | 4E-F |
| | Ttr_sg3 | CTTC | CCTTCGACTCTTCCTCCTTTG | 87 | FIG. 1D and FIG. |
| | | | | | 4E-F, 19B |
| | Ttr_sg4 | CTTC | CTCCTTTGCCTCGCTGGACTG | 88 | FIG. 4E-F |
| | Ttr_sg5 | TTTG | ACCATCAGAGGACATTTGGA | 89 | FIG. 4E-F |
| | Ttr_sg6 | TTTG | GATTCTCCAGCACCCTGGGC | 90 | FIG. 4E-F |
| | Ttr_sg7 | TTTA | CAGCCACGTCTACAGCAGGG | 91 | FIG. 4E-F |
| | Ttr_sg8 | TTTT | ACAGCCACGTCTACAGCAGG | 92 | FIG. 4E-F |
| | Ttr_sg9 | TTTT | GAACACTTTTACAGCCACGT | 93 | FIG. 4E-F |
| | Ttr_sg10 | GTTC | AAAAAGACCTCTGAGGGATC | 94 | FIG. 4E-F |
| | Ttr_sg11 | TTTG | AACACTTTTACAGCCACGTC | 95 | FIG. 4E-F |
| | Ttr_sg12 | TTTG | TAGAAGGAGTGTACAGAGTA | 96 | FIG. 1D, FIG. 2F-H |
| | | | | | and FIG. 4E-F, 19B |
| | Ttr_sg13 | CTTG | GCATTTCCCCGTTCCATGAA | 97 | FIG. 4E-F |
| | Ttr_sg14 | CTTC | TCATCTGTGGTGAGCCCGTG | 98 | FIG. 4E-F |
| | Ttr_sg15 | TTTG | GTGTCCAGTTCTACTCTGTA | 99 | FIG. 4E-F |
| | Ttr_sg16 | CTTC | CAGTACGATTTGGTGTCCAG | 100 | FIG. 4E-F |
| | Ttr_sg17 | CTTC | TACAAACTTCTCATCTGTGG | 101 | FIG. 4E-F |
| | Ttr_sg18 | TTTT | CACAGCCAACGACTCTGGCC | 102 | FIG. 4E-F |
| | Ttr_sg19 | TTTC | ACAGCCAACGACTCTGGCCA | 103 | FIG. 4E-F |
| | Ttr_sg20 | GTTG | CTGACGACAGCCGTGGTGCTG | 104 | FIG. 4E-F |
| | | | T | | |
| | Ttr_sg21 | GTTC | AAAAAGACCTCTGAGGGATCC | 105 | FIG. 4E-F |
| | | | T | | |
| TTR | TTR_sg1 | GTTC | AGAAAGGCTGCTGATGACACC | 106 | FIG. 1D and FIG. |
| | | | T | | 4E-F |
| | TTR_sg2 | TTTG | TAGAAGGGATATACAAAGTGG | 107 | FIG. 1D and. FIG. |
| | | | A | | 4E-F, 16 |
| | TTR_sg3 | ATTC | CACCACGGCTGTCGTCACCAA | 108 | FIG. 4E-F |
| | | | T | | |
| | TTR_sg5 | TTTG | AATCCAAGTGTCCTCTGATGGT | 109 | FIG. 4E-F |
| | TTR_sg6 | TTTC | AATGTGGCCGTGCATGTGTTCA | 110 | FIG. 4E-F |
| | TTR_sg7 | GTTC | TAGATGCTGTCCGAGGCAGTC | 111 | FIG. 4E-F |
| | | | C | | |
| | TTR_sg8 | ATTC | GCATGGGCTCACAACTGAGGA | 112 | FIG. 4E-F |
| | | | G | | |
| | TTR_sg10 | TTTG | TATACAAAGTGGAAATAGACA | 113 | FIG. 4E-F |
| | | | C | | |
| | TTR_sg11 | CTTA | CTGGAAGGCACTTGGCATCTC | 114 | FIG. 1D and FIG. |
| | | | C | | 4E-F |
| | TTR_sg12 | CTTG | GCATCTCCCCATTCCATGAGCA | 115 | FIG. 1D and FIG. |
| | | | | | 4E-F |
| | TTR_sg14 | ATTC | ACAGCCAACGACTCCGGCCCC | 116 | FIG. 4E-F |
| | | | C | | |
| PCSK9 | PCSK9_sg5 | GTTG | CCTGGCACCTACGTGGTGG | 117 | FIG. 4E-F |
| | PCSK9_sg6 | CTTC | CATGGCCTTCTTCCTGGC | 118 | FIG. ID and FIG. |
| | | | | | 4E-F |
| | PCSK9_sg7 | CTTC | TTCCTGGCTTCCTGGTGAAG | 119 | FIG. 4E-F |
| | PCSK9_sg9 | CTTG | AAGTTGCCCCATGTCGACTA | 121 | FIG. 1D and FIG. |
| | | | | | 4E-F |
| | PCSK9_sg10 | TTTG | CCCAGAGCATCCCGTGGAAC | 122 | FIG. 1D and FIG. |
| | | | | | 4E-F |
| TRAC | TRAC_sg1 | TTTA | CAGATACGAACCTAAACTTT | 123 | FIG. 2B-C |
| | TRAC_sg2 | TTTA | GAGTCTCTCAGCTGGTACAC | 124 | FIG. 2B-C |
| | TRAC_sg3 | TTTG | TCTGTGATATACACATCAGA | 125 | FIG. 2B-C |

### Example 6 Development of xCas12i Mutants and Evaluation of their dsDNA Cleavage Activity

To vary xCas12i's dsDNA cleavage activity and/or expand its scope of PAM site recognition, the applicant engineered xCas12i protein via mutagenesis and screened for mutants with various dsDNA cleavage activity and broader PAM using a dual plasmid fluorescent reporter system similar to the dual plasmid fluorescent reporter system in Example 1, except that the EGxxxxFP-targeting guide RNA (SEQ ID NO: 51; “crON/crRNA On-target”) coding sequence operably linked to U6 promoter was not located on the expression plasmid together with the xCas12i (or its mutant) coding sequence (SEQ ID NO: 31) but located on the reporter plasmid together with the BFP-P2A-EGxxxxFP coding sequence (SEQ ID NO: 41) (referring to “On-Target Reporter” in FIG. 1B). Combined with predictive structural analysis of xCas12i, the applicant performed an arginine (R) scanning mutagenesis approach in the domains including PI domain (amino acid residue position 173-291), REC-I domain (amino acid residue position 427-473), and RuvC-II domain (amino acid residue position 800-1082) of xCas12i, generating a library of 599 xCas12i mutants with a single non-R amino acid substitution with R. The xCas12i (SEQ ID NO: 1) coding sequence on the expression plasmid was replaced with a sequence encoding each of the xCas12i mutants in Table 6, the DR-T1 sequence (SEQ ID NO: 451) was used in place of the original DR sequence (SEQ ID NO: 11), while the other elements of the reporter system remained. The applicant then individually transfected the expression plasmid and the reporter plasmid into HEK293T cells and analyzed them by FACS (FIG. 1B).

For negative control (“NT”), a non-targeting spacer sequence (“NT”, SEQ ID NO: 46) incapable of hybridizing to the target sequence (SEQ ID NO: 44) was used in place of the EGxxxxFP-targeting spacer sequence (SEQ ID NO: 45) and used in combination with xCas12i (SEQ ID NO: 1), while the other elements of the reporter system remained. For positive control (“WT”), the original xCas12i (SEQ ID NO: 1) was used.

TABLE 6
Mutants of xCas12i and dsDNA cleavage activity thereof

| | dsDNA |
|---|---|
| | cleavage |
| Mutant | activity |
| K109R | 0.034 |
| N110R | 0.778 |
| Y111R | 0.634 |
| L112R | 0.041 |
| M113R | 0.062 |
| S114R | 0.837 |
| N115R | 0.312 |
| I116R | 0.836 |
| D117R | 0.499 |
| S118R | 1.481 |
| D119R | 1.337 |
| F121R | 1.356 |
| V122R | 0.737 |
| W123R | 1.010 |
| V124R | 0.119 |
| D125R | 0.040 |
| C126R | 0.051 |
| K128R | 0.844 |
| F129R | 0.802 |
| A130R | 0.064 |
| K131R | 0.728 |
| D132R | 0.839 |
| F133R | 0.990 |
| A134R | 0.076 |
| Y135R | 0.863 |
| Q136R | 1.067 |
| M137R | 0.128 |
| E138R | 1.010 |
| L139R | 0.194 |
| G140R | 0.957 |
| F141R | 0.429 |
| H142R | 0.941 |
| E143R | 1.240 |
| F144R | 0.007 |
| T145R | 0.951 |
| V146R | 1.106 |
| L147R | 0.038 |
| A148R | 0.013 |
| E149R | 0.319 |
| T150R | 0.686 |
| L151R | 0.038 |
| L152R | 0.097 |
| A153R | 1.000 |
| N154R | 0.307 |
| S155R | 1.577 |
| I156R | 0.531 |
| L157R | 0.041 |
| V158R | 1.990 |
| L159R | 0.085 |
| N160R | 0.860 |
| E161R | 2.115 |
| S162R | 2.096 |
| T163R | 1.054 |
| K164R | 0.760 |
| A165R | 3.151 |
| N166R | 1.548 |
| W167R | 0.775 |
| A168R | 0.058 |
| W169R | 0.161 |
| G170R | 0.572 |
| T171R | 0.211 |
| V172R | 0.564 |
| S173R | 0.202 |
| A174R | 0.398 |
| L175R | 0.170 |
| Y176R | 0.215 |
| G177R | 0.135 |
| G178R | 1.920 |
| G179R | 0.737 |
| D180R | 1.025 |
| K181R | 0.172 |
| E182R | 0.235 |
| D183R | 0.279 |
| S184R | 0.987 |
| T185R | 1.685 |
| L186R | 0.641 |
| K187R | 0.193 |
| S188R | 0.234 |
| K189R | 1.010 |
| I190R | 0.070 |
| L191R | 0.118 |
| L192R | 0.910 |
| A193R | 1.566 |
| F194R | 0.194 |
| V195R | 0.019 |
| D196R | 1.317 |
| A197R | 0.791 |
| L198R | 0.204 |
| N199R | 1.354 |
| N200R | 1.417 |
| H201R | 0.183 |
| E202R | 1.102 |
| L203R | 1.344 |
| K204R | 0.817 |
| T205R | 0.973 |
| K206R | 0.871 |
| E208R | 0.279 |
| I209R | 0.108 |
| L210R | 0.346 |
| N211R | 0.499 |
| Q212R | 0.650 |
| V213R | 0.114 |
| C214R | 0.166 |
| E215R | 0.329 |
| S216R | 0.591 |
| L217R | 0.465 |
| K218R | 0.294 |
| Y219R | 0.375 |
| Q220R | 0.371 |
| S221R | 1.150 |
| Y222R | 0.417 |
| Q223R | 0.574 |
| D224R | 0.301 |
| M225R | 0.099 |
| Y226R | 0.000 |
| V227R | 0.177 |
| D228R | 0.168 |
| F229R | 0.190 |
| S231R | 0.284 |
| V232R | 0.559 |
| V233R | 1.253 |
| D234R | 0.217 |
| E235R | 1.727 |
| N236R | 1.242 |
| G237R | 0.470 |
| N238R | 0.069 |
| K239R | 0.988 |
| K240R | 0.908 |
| S241R | 1.828 |
| P242R | 0.167 |
| N243R | 3.606 |
| G244R | 0.060 |
| S245R | 1.293 |
| M246R | 0.124 |
| P247R | 0.240 |
| I248R | 0.962 |
| V249R | 0.114 |
| T250R | 0.140 |
| K251R | 1.434 |
| F252R | 0.009 |
| E253R | 0.321 |
| T254R | 0.927 |
| D255R | 1.182 |
| D256R | 0.595 |
| L257R | 1.162 |
| I258R | 0.044 |
| S259R | 0.531 |
| D260R | 0.293 |
| N261R | 0.484 |
| Q262R | 0.498 |
| K264R | 0.671 |
| A265R | 0.725 |
| M266R | 0.250 |
| I267R | 0.933 |
| S268R | 0.959 |
| N269R | 0.401 |
| F270R | 0.131 |
| T271R | 0.450 |
| K272R | 0.383 |
| N273R | 1.652 |
| A274R | 0.207 |
| A275R | 0.713 |
| A276R | 0.309 |
| K277R | 0.282 |
| A278R | 0.471 |
| A279R | 0.683 |
| K280R | 0.556 |
| K281R | 0.671 |
| P282R | 0.575 |
| I283R | 0.390 |
| P284R | 0.274 |
| Y285R | 0.287 |
| L286R | 0.745 |
| D287R | 1.084 |
| L289R | 0.400 |
| K290R | 0.403 |
| E291R | 0.363 |
| M293R | 0.019 |
| V294R | 0.665 |
| S295R | 1.172 |
| L296R | 0.752 |
| C297R | 0.061 |
| D298R | 0.719 |
| Y300R | 0.168 |
| N301R | 0.359 |
| V302R | 1.517 |
| Y303R | 0.324 |
| A304R | 0.067 |
| W305R | 0.026 |
| A306R | 0.187 |
| A307R | 0.265 |
| A308R | 0.030 |
| I309R | 0.009 |
| T310R | 0.163 |
| N311R | 0.120 |
| S312R | 0.037 |
| N313R | 0.246 |
| A314R | 0.030 |
| D315R | 0.046 |
| V316R | 0.007 |
| T317R | 0.143 |
| A318R | 0.037 |
| N320R | 0.098 |
| T321R | 0.156 |
| L324R | 0.035 |
| T325R | 0.209 |
| F326R | 0.183 |
| I327R | 0.031 |
| G328R | 0.879 |
| E329R | 0.249 |
| Q330R | 0.159 |
| N331R | 0.538 |
| S332R | 1.136 |
| K335R | 0.577 |
| E336R | 1.463 |
| L337R | 0.613 |
| S338R | 1.505 |
| V339R | 1.183 |
| L340R | 0.419 |
| Q341R | 0.766 |
| T342R | 0.322 |
| T343R | 0.710 |
| T344R | 0.646 |
| N345R | 0.218 |
| E346R | 0.554 |
| K347R | 0.684 |
| A348R | 0.048 |
| K349R | 0.461 |
| D350R | 0.474 |
| I351R | 0.146 |
| L352R | 0.023 |
| N353R | 0.553 |
| K354R | 0.681 |
| N356R | 0.542 |
| D357R | 0.472 |
| N358R | 0.554 |
| L359R | 0.398 |
| I360R | 0.580 |
| Q361R | 0.676 |
| E362R | 1.430 |
| V363R | 0.696 |
| Y365R | 0.016 |
| T366R | 0.973 |
| P367R | 0.195 |
| A368R | 0.709 |
| K370R | 0.648 |
| H371R | 0.068 |
| L372R | 0.006 |
| G373R | 0.430 |
| D375R | 1.408 |
| L376R | 0.006 |
| A377R | 1.097 |
| N378R | 1.113 |
| L379R | 0.008 |
| F380R | 0.087 |
| D381R | 1.502 |
| T382R | 1.517 |
| L383R | 0.006 |
| K384R | 0.941 |
| E385R | 1.424 |
| K386R | 0.980 |
| D387R | 1.050 |
| I388R | 0.317 |
| N389R | 0.895 |
| N390R | 1.066 |
| I391R | 0.685 |
| E392R | 0.996 |
| N393R | 0.662 |
| E394R | 0.871 |
| E395R | 1.144 |
| E396R | 1.214 |
| K397R | 0.918 |
| Q398R | 1.043 |
| N399R | 1.050 |
| V400R | 1.222 |
| I401R | 0.754 |
| N402R | 0.934 |
| D403R | 1.712 |
| C404R | 0.689 |
| I405R | 0.048 |
| E406R | 1.758 |
| Q407R | 1.735 |
| Y408R | 0.064 |
| V409R | 1.004 |
| D410R | 0.771 |
| D411R | 1.447 |
| C412R | 1.852 |
| L415R | 0.650 |
| N416R | 1.541 |
| N418R | 1.292 |
| P419R | 0.171 |
| I420R | 0.058 |
| A421R | 0.910 |
| A422R | 0.674 |
| L423R | 0.092 |
| L424R | 0.013 |
| K425R | 0.745 |
| H426R | 0.742 |
| I427R | 0.005 |
| S428R | 0.075 |
| Y430R | 0.359 |
| Y431R | 0.856 |
| E432R | 0.670 |
| D433R | 0.605 |
| F434R | 0.161 |
| S435R | 0.981 |
| A436R | 0.033 |
| K437R | 0.880 |
| N438R | 0.309 |
| F439R | 0.010 |
| L440R | 1.379 |
| D441R | 0.671 |
| G442R | 0.051 |
| A443R | 0.033 |
| K444R | 0.547 |
| L445R | 0.107 |
| N446R | 0.410 |
| V447R | 0.004 |
| L448R | 1.369 |
| T449R | 0.514 |
| E450R | 0.887 |
| V451R | 1.883 |
| V452R | 0.735 |
| N453R | 0.895 |
| Q455R | 1.190 |
| K456R | 0.887 |
| A457R | 0.004 |
| H458R | 0.008 |
| P459R | 0.008 |
| T460R | 0.009 |
| I461R | 0.801 |
| W462R | 0.358 |
| S463R | 0.020 |
| E464R | 1.127 |
| I800R | 0.596 |
| S801R | 0.204 |
| L802R | 0.398 |
| K803R | 0.436 |
| M804R | 0.130 |
| I805R | 0.325 |
| S806R | 1.214 |
| D807R | 0.899 |
| F808R | 0.261 |
| K809R | 0.905 |
| G810R | 0.954 |
| V811R | 0.178 |
| V812R | 0.187 |
| Q813R | 0.161 |
| S814R | 0.023 |
| Y815R | 0.284 |
| F816R | 0.299 |
| S817R | 1.290 |
| V818R | 1.410 |
| S819R | 1.130 |
| G820R | 0.407 |
| C821R | 0.801 |
| V822R | 0.699 |
| D823R | 0.911 |
| D824R | 0.939 |
| A825R | 0.884 |
| S826R | 0.707 |
| K827R | 0.654 |
| K828R | 0.917 |
| A829R | 0.954 |
| H830R | 0.593 |
| D831R | 0.318 |
| S832R | 1.010 |
| M833R | 1.088 |
| L834R | 0.835 |
| F835R | 1.280 |
| T836R | 1.402 |
| F837R | 1.270 |
| M838R | 0.961 |
| C839R | 1.700 |
| A840R | 1.412 |
| A841R | 0.245 |
| E842R | 1.540 |
| E843R | 1.710 |
| K844R | 1.520 |
| T846R | 1.620 |
| N847R | 1.180 |
| K848R | 1.230 |
| E850R | 0.867 |
| E851R | 0.977 |
| K852R | 0.337 |
| T853R | 0.928 |
| N854R | 1.031 |
| A856R | 1.262 |
| A857R | 0.384 |
| S858R | 1.117 |
| F859R | 0.000 |
| I860R | 0.146 |
| L861R | 0.770 |
| Q862R | 1.882 |
| K863R | 1.427 |
| A864R | 0.000 |
| Y865R | 1.179 |
| L866R | 1.417 |
| H867R | 0.000 |
| G868R | 1.613 |
| C869R | 0.131 |
| K870R | 1.510 |
| M871R | 1.334 |
| I872R | 0.163 |
| V873R | 0.306 |
| C874R | 0.519 |
| E875R | 0.100 |
| D876R | 2.637 |
| D877R | 2.492 |
| L878R | 0.132 |
| P879R | 0.132 |
| V880R | 1.458 |
| A881R | 0.236 |
| D882R | 0.356 |
| G883R | 1.303 |
| K884R | 1.624 |
| T885R | 0.464 |
| G886R | 1.856 |
| K887R | 1.606 |
| A888R | 2.077 |
| Q889R | 0.720 |
| N890R | 0.151 |
| A891R | 2.265 |
| D892R | 1.417 |
| M894R | 1.386 |
| D895R | 0.539 |
| W896R | 0.265 |
| C897R | 0.873 |
| A898R | 0.192 |
| A900R | 1.324 |
| L901R | 0.376 |
| A902R | 0.621 |
| K903R | 1.115 |
| K904R | 1.106 |
| V905R | 0.203 |
| N906R | 1.606 |
| D907R | 0.238 |
| G908R | 0.244 |
| C909R | 0.499 |
| V910R | 1.406 |
| A911R | 0.222 |
| M912R | 1.106 |
| S913R | 1.471 |
| I914R | 1.000 |
| C915R | 1.663 |
| Y916R | 1.356 |
| A918R | 1.882 |
| P920R | 0.831 |
| A921R | 0.338 |
| Y922R | 0.446 |
| M923R | 1.044 |
| S924R | 0.351 |
| S925R | 1.276 |
| H926R | 1.440 |
| Q927R | 1.933 |
| D928R | 0.164 |
| P929R | 0.179 |
| F930R | 0.203 |
| V931R | 1.547 |
| H932R | 0.229 |
| M933R | 1.827 |
| Q934R | 2.147 |
| D935R | 1.413 |
| K936R | 1.489 |
| K937R | 1.442 |
| T938R | 1.452 |
| S939R | 1.413 |
| V940R | 1.333 |
| L941R | 0.988 |
| P943R | 0.812 |
| F945R | 1.055 |
| M946R | 1.207 |
| E947R | 0.885 |
| V948R | 1.231 |
| N949R | 1.893 |
| K950R | 1.640 |
| D951R | 2.347 |
| S952R | 1.500 |
| I953R | 0.382 |
| D955R | 1.221 |
| Y956R | 1.768 |
| H957R | 0.681 |
| V958R | 0.541 |
| A959R | 1.635 |
| G960R | 1.840 |
| L961R | 0.152 |
| L965R | 0.443 |
| N966R | 1.933 |
| S967R | 1.529 |
| K968R | 1.241 |
| S969R | 1.548 |
| D970R | 1.451 |
| A971R | 1.848 |
| G972R | 1.152 |
| T973R | 0.641 |
| S974R | 1.180 |
| V975R | 1.097 |
| Y976R | 1.148 |
| Y977R | 0.007 |
| Q979R | 1.421 |
| A980R | 1.057 |
| A981R | 0.341 |
| L982R | 1.146 |
| H983R | 1.372 |
| F984R | 0.580 |
| C985R | 1.076 |
| E986R | 1.137 |
| A987R | 1.220 |
| L988R | 0.954 |
| G989R | 1.420 |
| V990R | 1.094 |
| S991R | 1.211 |
| P992R | 1.128 |
| E993R | 1.154 |
| L994R | 1.148 |
| V995R | 1.109 |
| K996R | 1.038 |
| N997R | 1.211 |
| K998R | 1.171 |
| K999R | 1.348 |
| T1000R | 1.128 |
| H1001R | 1.209 |
| A1002R | 1.171 |
| A1003R | 1.241 |
| E1004R | 1.460 |
| L1005R | 0.665 |
| G1006R | 1.031 |
| M1009R | 0.980 |
| G1010R | 1.172 |
| S1011R | 0.558 |
| A1012R | 1.098 |
| M1013R | 1.207 |
| L1014R | 1.044 |
| M1015R | 0.535 |
| P1016R | 0.088 |
| W1017R | 1.744 |
| G1019R | 0.387 |
| G1020R | 0.396 |
| V1022R | 1.260 |
| Y1023R | 0.814 |
| I1024R | 0.296 |
| A1025R | 0.062 |
| S1026R | 0.971 |
| K1027R | 0.978 |
| K1028R | 1.550 |
| L1029R | 0.444 |
| T1030R | 0.824 |
| S1031R | 0.000 |
| D1032R | 1.230 |
| A1033R | 0.563 |
| K1034R | 1.301 |
| S1035R | 0.790 |
| V1036R | 0.627 |
| K1037R | 1.750 |
| Y1038R | 0.666 |
| C1039R | 1.430 |
| G1040R | 1.077 |
| E1041R | 0.920 |
| D1042R | 0.928 |
| M1043R | 0.930 |
| W1044R | 0.870 |
| Q1045R | 1.560 |
| Y1046R | 0.708 |
| H1047R | 1.430 |
| A1048R | 0.739 |
| D1049R | 0.699 |
| E1050R | 0.788 |
| I1051R | 0.678 |
| A1052R | 0.114 |
| A1053R | 0.035 |
| V1054R | 0.122 |
| N1055R | 0.108 |
| I1056R | 0.078 |
| A1057R | 0.285 |
| M1058R | 0.354 |
| Y1059R | 0.762 |
| E1060R | 0.623 |
| V1061R | 0.947 |
| C1062R | 0.699 |
| C1063R | 1.137 |
| Q1064R | 0.948 |
| T1065R | 0.781 |
| G1066R | 0.906 |
| A1067R | 0.994 |
| F1068R | 0.010 |
| G1069R | 1.067 |
| K1070R | 0.969 |
| K1071R | 0.833 |
| Q1072R | 0.879 |
| K1073R | 0.464 |
| K1074R | 0.286 |
| S1075R | 0.971 |
| D1076R | 0.777 |
| E1077R | 0.709 |
| L1078R | 0.915 |
| P1079R | 0.860 |
| G1080R | 0.996 |
| WT | 1.000 |
| NT | 0.0084 |

Based on the fluorescence intensity of cells with activated EGFP, it was observed that almost 200 xCas12i mutants showed an increased dsDNA cleavage activity relative to xCas12i (WT; SEQ ID NO: 1) (FIG. 5A, Table 6), and among them, one mutant, xCas12i-N243R, referred to as Cas12Max, showed about 3.6-fold improvement (FIG. 5A). In addition, about 50 xCas12i mutants has no more than 5% dsDNA cleavage activity relative to WT xCas12i (SEQ ID NO: 1) (FIG. 5A, Table 6).

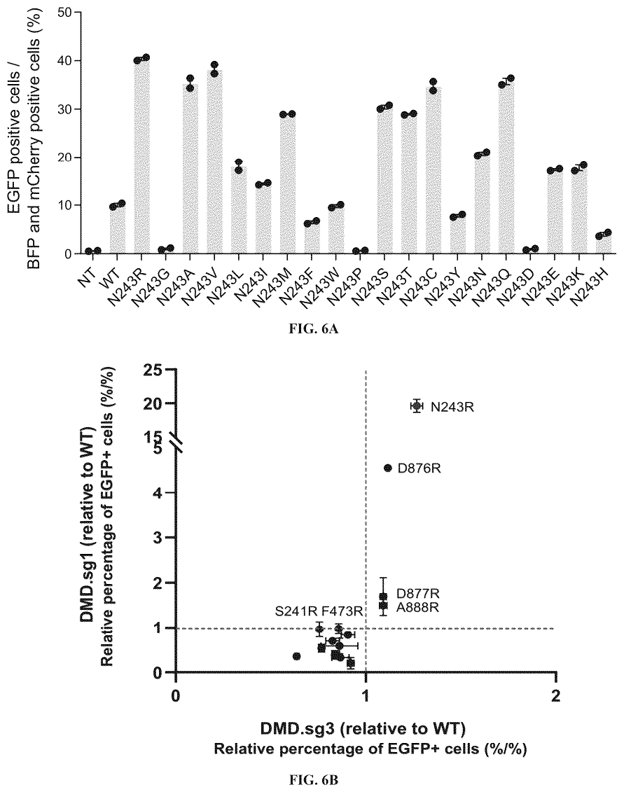

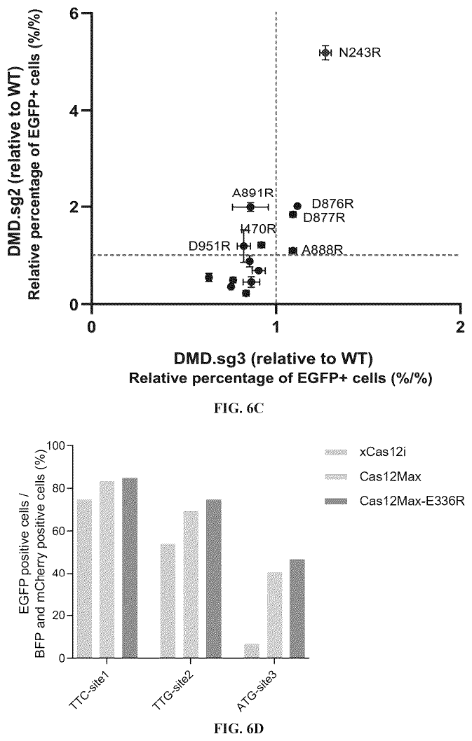

The applicant then performed saturation mutagenesis of N243 and observed that the mutation to R indeed showed the highest dsDNA cleavage activity (FIG. 6A).

The applicant next targeted DMD or Ttr sites using the fluorescent reporter system (replacing the insertion sequence (SEQ ID NO: 42) with an insertion sequence containing DMD or Ttr protospacer and corresponding 5′ PAM as listed in Table 5) and observed that Cas12Max displayed a markedly increased frequency of EGFP activation, relative to xCas12i (WT) (FIG. 1C, FIG. 6B-C). In addition, it was observed that the incorporation of E336R into Cas12Max (resulting in mutant xCas12i-N243R+E336R; SEQ ID NO: 467) further increased the dsDNA cleavage activity of Cas12Max at all three sites with different PAMs (TTC-site 1, TTG-site 2, ATG-site 3) (FIG. 6D).

To further test the efficacy of Cas12Max in targeting genomic loci, the applicant designed a total of eight gRNAs to target sites TTR and PCSK9 in HEK293T cells and three more to target Ttr in N2a cells (Table 5), in which DR-T2 (SEQ ID NO: 452) was used. Consistent with the previous results, Cas12Max exhibited a significantly increased frequency of indels compared to WT xCas12i (FIG. 1D).

### Example 7 Further Development of Mutants Based on Cas12Max and Evaluation of their Off-Target dsDNA Cleavage Activity

To examine the specificity of Cas12Max, the applicant transfected a construct designed to express it with a gRNA targeting TTR (with TTR-targeting (on-target) spacer sequence of SEQ ID NO: 130), and performed indel frequency analysis of on- and off-target (OT) sites predicted by Cas-OFFinder.

TABLE 7

| | Off-target protospacer sequence | |
|---|---|---|
| | (with 5′ PAM of TTTG) | SEQ ID NO: |
| TTR off-target.3 (OT.3) | CAGCAGGCTTCTACAAAGTGGA | 127 |
| TTR off-target.2 (OT.2) | TAAAAGGGATATACAATATGTA | 128 |
| TTR off-target.1 (OT.1) | TAGAAGGGATATAGAAAGTATC | 129 |
| | On-target protospacer sequence/ | |
| | spacer sequence (with 5′ PAM of TTTG) | |
| TTR on-target.1 (ON.1) | TAGAAGGGATATACAAAGTGGA | 130 |

A dual plasmid fluorescent reporter system for evaluation of off-target dsDNA cleavage activity (off-target reporter system; referring to “Off-Target Reporter” in FIG. 1B) was established, which was similar to the dual plasmid fluorescent reporter system in Example 6 for evaluation of (on-target) dsDNA cleavage activity, except that the insertion sequence of the EGxxxxFP coding sequence contains an TTR off-target protospacer sequence (one of SEQ ID NOs: 127-129) containing one or more mismatches (bold, underlined) with a TTR-targeting spacer sequence (SEQ ID NO: 130) in the gRNA, rather than containing a TTR on-target protospacer sequence (SEQ ID NO: 130; which is the same as SEQ ID NO: 107 in Example 5); DR-T1 sequence (SEQ ID NO: 451) was used. To save drafting, the on-target protospacer sequence/spacer sequence in Table 7 refer to both the protospacer sequence (a DNA sequence) and the corresponding spacer sequence (an RNA sequence) with any “T” in the sequence when referring to a protospacer sequences standing for “T” and when referring to a such spacer sequence standing for “U”, although the assigned SEQ ID NO: 130 in the sequence listing is annotated as DNA.

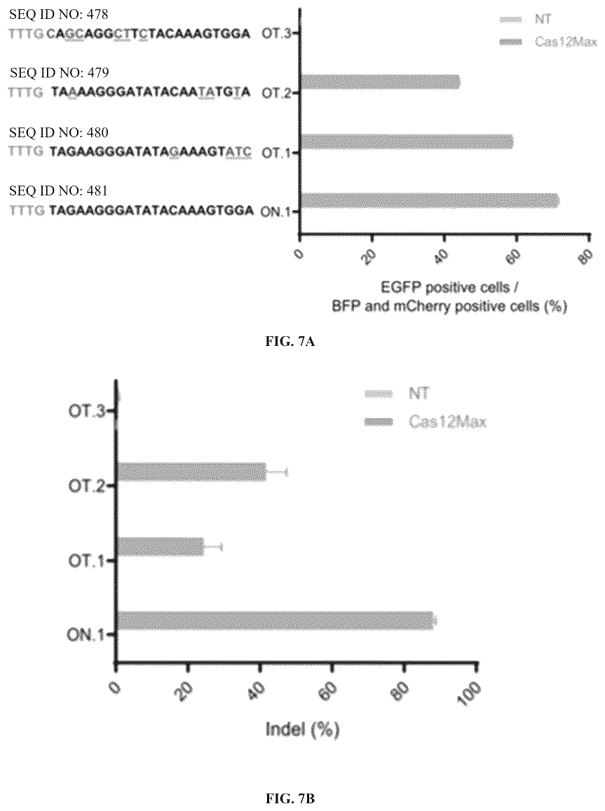

Using the on-target and off-target reporter systems (FIG. 7A) or targeted deep sequence analysis on endogenous gene (FIG. 7B), the applicant observed that Cas12Max efficiently edited the target site (“ON. 1”), while resulting in indel formation at 2 (“OT. 1” and “OT. 2”) of the 3 predicted off-target sites (“OT. 1”, “OT. 2”, and “OT. 3”), indicating off-target dsDNA cleavage activity.

To eliminate the off-target activity of Cas12Max, the applicant selected those mutants in Example 5 with a single mutation in the REC and RuvC domains and undiminished on-target cleavage activity (comparable to xCas12i (WT)), and then tested their off-target dsDNA cleavage activity by using two off-target reporter systems above with TTR OT1 and OT2, respectively (FIG. 1B). It was observed that four xCas12i mutants (xCas12i-V880R (v4.1), xCas12i-M923R (v4.2), xCas12i-D892R (v4.3), and xCas12i-G883R (v4.4); FIG. 8B) maintained a high level of on-target dsDNA cleavage activity and showed substantially no off-target dsDNA cleavage activity at both TTR OT1 and OT2 (FIG. 8A).

The applicant further combined one or more of these four amino acid substitutions with N243R or N243R+E336R (FIG. 8B). As shown in FIG. 8C, all the four mutants v5.1, v5.2, v5.3, and v5.4 with two amino acid substitutions of N243R and one of V880R, G883R, D892R, and M923R, respectively, had comparable or higher on-target cleavage activity and greatly reduced off-target cleavage activity compared with Cas12Max; and all the four mutants v6.1, v6.2, v6.3, and v6.4 with three amino acid substitutions of N243R and E336R and one of V880R, G883R, D892R, and M923R, respectively, had comparable or higher on-target cleavage activity and greatly reduced off-target cleavage activity compared with Cas12Max.

In particular, it was observed that the mutant v6.3 (N243R+E336R+D892R) showed the best overall performance of both on-target and off-target cleavage activities (FIG. 8B-C). Targeted deep sequencing analysis of endogenous TTR. 2 site and its off-target sites in HEK293T showed that v6.3 (N243R+E336R+D892R) significantly reduced off-target indel frequencies at all the six OT sites and retained on-target indel frequency at ON site, compared to Cas12Max (FIG. 1E). In addition, relative to Cas12Max (v1.1), v6.3 (N243R+E336R+D892R) retained comparable or even higher on-target activity at DMD. 1, DMD. 2 and DMD. 3 sites (FIG. 8D). Therefore, the applicant named v6.3 as high-fidelity Cas12Max (hfCas12Max).

TABLE 8

| Mutant | Version | ON | OT-1 | OT-2 | OT-3 |
|---|---|---|---|---|---|
| N243R | v1.1 | 73.80 | 60.17 | 47.50 | 0.11 |
| N243R + V880R | v5.1 | 71.60 | 3.82 | 0.24 | 0.15 |
| N243R + M923R | v5.2 | 76.10 | 4.90 | 0.92 | 0.15 |
| N243R + D892R | v5.3 | 75.85 | 6.66 | 5.46 | 0.21 |
| N243R + G883R | v5.4 | 77.30 | 16.80 | 1.36 | 0.15 |
| N243R + E336R + V880R | v6.1 | 75.70 | 2.04 | 0.44 | 0.15 |
| N243R + E336R + M923R | v6.2 | 75.57 | 2.41 | 2.90 | 0.05 |
| N243R + E336R + D892R | v6.3 | 77.73 | 1.55 | 0.25 | 0.13 |
| (hfCas12Max) | | | | | |
| N243R + E336R + G883R | v6.4 | 74.75 | 6.65 | 0.64 | 0.03 |
| N243R + E336R + D892A | v6.7 | 77.30 | 54.80 | 51.50 | |
| N243R + E336R + G883A | v6.8 | 78.50 | 44.00 | 36.40 | |
| NT | | 0.028 | 0.048 | 0.067 | 0.014 |

Additionally, to investigate hfCas12Max's PAM preference, the applicant performed a 5′-NNN PAM recognition assay by designing reporter plasmids with the same target sequence but different PAM, similar to Example 3. Besides showing a consistent or higher cleavage activity at sites with a 5′-TTN PAM, hfCas12Max and Cas12Max showed a similarly high cleavage activity for targets with 5′-TNN, 5′-ATN, 5′-GTN, and 5′-CTN PAM sites, compared with the commonly used Cas12 (LbCas12a, Ultra-AsCas12a) and recently reported improved Cas12i2 (ABR001, Cas12i2^{HiFi}) (FIG. 1F). Taken together, these results demonstrate that hfCas12Max exhibits high-efficiency editing activity with highly flexible 5′-TN (TTN/ATN/GTN/CTN) or 5′-TNN (TAN/TCN/TGN/TTN) PAM recognition, along with some discrete 5′ PAM, such as, 5′-GCC, which advantageously expands the application scope of this tool.

### Example 8 Verification and Comparison of hfCas12Max's On- and Off-Target dsDNA Cleavage Activity at TTR Gene

To comprehensively evaluate the performance of hfCas12Max in human cells, the applicant designed a large number of target sites in the exons of TTR for various Cas nucleases. DR-T2 (SEQ ID NO: 452) was used in this and subsequent Example unless otherwise specified.

In total, cleavage activity was monitored at 43 sites for hfCas12Max with 5′-TTN PAMs, 43 sites for ABR001 (engineered Cas12i2 from Arbor Biotechnologies) with TTN PAMs, 43 sites for Cas12i2-1 with TTN PAMs, 45 sites for SpCas9 with NGG PAMs, 12 sites for LbCas12a with TTTN PAMs, 12 sites for Ultra AsCas12a with TTTN PAMs, and 20 sites for KKH-saCas9 with NNNRRT PAMs (Table 9). Indel analysis showed that hfCas12Max exhibited a higher average on-target dsDNA cleavage activity than all the other Cas nucleases and Cas12Max (FIG. 1G, FIG. 9). To save drafting, the sequences in Table 9 refer to both the protospacer sequence (aDNA sequence) and the corresponding spacer sequence (an RNA sequence) with any “T” in the sequence when referring to a protospacer sequences standing for “T” and when referring to a such spacer sequence standing for “U”, although the assigned SEQ ID NOs: 131-381 in the sequence listing are annotated as DNA.

TABLE 9
Sequences for testing genome cleavage at target loci (FIG. 1G, FIG. 9)

| | | | | | SEQ ID NO of |
|---|---|---|---|---|---|
| | | | | | protospacer |
| Genomic | | | | Protospacer sequence/ | sequence/ |
| loci | Cas | SITE | 5′/3′PAM | Spacer Sequence | spacer sequence |
| TTR | LbCas12a | TTTN.1 | TTTG | TGTCTGAGGCTGGCCCTACGGTG | 131 |
| | | TTTN.2 | TTTG | ACCATCAGAGGACACTTGGATTC | 132 |
| | | TTTN.3 | TTTC | TGAACACATGCACGGCCACATTG | 133 |
| | | TTTN.4 | TTTG | CCTCTGGGTAAGTTGCCAAAGAA | 134 |
| | | TTTN.5 | TTTG | GCAACTTACCCAGAGGCAAATGG | 135 |
| | | TTTN.6 | TTTC | ACACCTTATAGGAAAACCAGTGA | 136 |
| | | TTTN.7 | TTTC | CTATAAGGTGTGAAAGTCTGGAT | 137 |
| | | TTTN.8 | TTTT | CCTATAAGGTGTGAAAGTCTGGA | 138 |
| | | TTTN.9 | TTTG | TAGAAGGGATATACAAAGTGGAA | 139 |
| | | TTTN.10 | TTTG | TATATCCCTTCTACAAATTCCTC | 140 |
| | | TTTN.11 | TTTC | CACTTTGTATATCCCTTCTACAA | 141 |
| | | TTTN.12 | TTTG | GTGTCTATTTCCACTTTGTATAT | 142 |
| | UltraCas12a | TTTN.1 | TTTG | TGTCTGAGGCTGGCCCTACGGTG | 143 |
| | | TTTN.2 | TTTG | ACCATCAGAGGACACTTGGATTC | 144 |
| | | TTTN.3 | TTTC | TGAACACATGCACGGCCACATTG | 145 |
| | | TTTN.4 | TTTG | CCTCTGGGTAAGTTGCCAAAGAA | 146 |
| | | TTTN.5 | TTTG | GCAACTTACCCAGAGGCAAATGG | 147 |
| | | TTTN.6 | TTTC | ACACCTTATAGGAAAACCAGTGA | 148 |
| | | TTTN.7 | TTTC | CTATAAGGTGTGAAAGTCTGGAT | 149 |
| | | TTTN.8 | TTTT | CCTATAAGGTGTGAAAGTCTGGA | 150 |
| | | TTTN.9 | TTTG | TAGAAGGGATATACAAAGTGGAA | 151 |
| | | TTTN.10 | TTTG | TATATCCCTTCTACAAATTCCTC | 152 |
| | | TTTN.11 | TTTC | CACTTTGTATATCCCTTCTACAA | 153 |
| | | TTTN.12 | TTTG | GTGTCTATTTCCACTTTGTATAT | 154 |
| | KKH-SaCas9 | NNGRRT.1 | ACAGAT | CCACCTATGAGAGAAGACAG | 155 |
| | | NNGRRT.2 | AGGAAT | GGCTGTCGTCACCAATCCCA | 156 |
| | | NNGRRT.3 | AGGAGT | GACGACAGCCGTGGTGGAAT | 157 |
| | | NNGRRT.4 | ATTGAT | CTGAACACATGCACGGCCAC | 158 |
| | | NNGRRT.5 | CCAAGT | CACCCAGGGCACCGGTGAAT | 159 |
| | | NNGRRT.6 | CGGAGT | AATGGTGTAGCGGCGGGGGC | 160 |
| | | NNGRRT.7 | GCAAAT | CTTTGGCAACTTACCCAGAG | 161 |
| | | NNGRRT.8 | GTGAGT | TGTCTGAGGCTGGCCCTACG | 162 |
| | | NNGRRT.9 | TACGGT | TTTGTGTCTGAGGCTGGCCC | 163 |
| | | NNGRRT.10 | TGGAAT | ATTGGTGACGACAGCCGTGG | 164 |
| | | NNGRRT.11 | TGGGAT | AGGAGAAGTCCCTCATTCCT | 165 |
| | | NNGRRT.12 | TTTGGT | CCAAGTGCCTTCCAGTAAGA | 166 |
| | SpCas9 | NGG.1 | AGG | ACACAAATACCAGTCCAGCA | 167 |
| | | NGG.2 | AGG | CCAGTCCAGCAAGGCAGAGG | 168 |
| | | NGG.3 | AGG | GAAGTCCACTCATTCTTGGC | 169 |
| | | NGG.4 | AGG | AAAGTTCTAGATGCTGTCCG | 170 |
| | | NGG.5 | AGG | CCCAGAGGCAAATGGCTCCC | 171 |
| | | NGG.6 | AGG | TTCTTTGGCAACTTACCCAG | 172 |
| | | NGG.7 | AGG | ACTGAGGAGGAATTTGTAGA | 173 |
| | | NGG.8 | AGG | CCCATTCCATGAGCATGCAG | 174 |
| | | NGG.9 | AGG | GCATGGGCTCACAACTGAGG | 175 |
| | | NGG.10 | AGG | AATAGGAGTAGGGGCTCAGC | 176 |
| | | NGG.11 | AGG | GACGACAGCCGTGGTGGAAT | 177 |
| | | NGG.12 | AGG | GGCTGTCGTCACCAATCCCA | 178 |
| | | NGG.13 | AGG | GTCACCAATCCCAAGGAATG | 179 |
| | | NGG.14 | CGG | TGTGTCTGAGGCTGGCCCTA | 180 |
| | | NGG.15 | CGG | AGCCTTTCTGAACACATGCA | 181 |
| | | NGG.16 | CGG | CAGAGGACACTTGGATTCAC | 182 |
| | | NGG.17 | CGG | CATTGATGGCAGGACTGCCT | 183 |
| | | NGG.18 | CGG | CTTCTCTACACCCAGGGCAC | 184 |
| | | NGG.19 | CGG | AATGGTGTAGCGGCGGGGGC | 185 |
| | | NGG.20 | CGG | CCCCTACTCCTATTCCACCA | 186 |
| | | NGG.21 | CGG | GCAGGGCGGCAATGGTGTAG | 187 |
| | | NGG.22 | CGG | GGAGTAGGGGCTCAGCAGGG | 188 |
| | | NGG.23 | CGG | GTATTCACAGCCAACGACTC | 189 |
| | | NGG.24 | GGG | TCACAGAAACACTCACCGTA | 190 |
| | | NGG.25 | GGG | AAAGGCTGCTGATGACACCT | 191 |
| | | NGG.26 | GGG | CTTGGATTCACCGGTGCCCT | 192 |
| | | NGG.27 | GGG | GCCGTGGTGGAATAGGAGTA | 193 |
| | | NGG.28 | GGG | GCGGCAATGGTGTAGCGGCG | 194 |
| | | NGG.29 | GGG | GGAGAAGTCCCTCATTCCTT | 195 |
| | | NGG.30 | GGG | GGCGGCAATGGTGTAGCGGC | 196 |
| | | NGG.31 | GGG | TCACCAATCCCAAGGAATGA | 197 |
| | | NGG.32 | TGG | GCAACTTACCCAGAGGCAAA | 198 |
| | | NGG.33 | TGG | AAGTGCCTTCCAGTAAGATT | 199 |
| | | NGG.34 | TGG | ACCTCTGCATGCTCATGGAA | 200 |
| | | NGG.35 | TGG | TACTCACCTCTGCATGCTCA | 201 |
| | | NGG.36 | TGG | TGTAGAAGGGATATACAAAG | 202 |
| | | NGG.37 | TGG | AGGAGAAGTCCCTCATTCCT | 203 |
| | | NGG.38 | TGG | ATTGGTGACGACAGCCGTGG | 204 |
| | | NGG.39 | TGG | GCGGCGGGGGCCGGAGTCGT | 205 |
| | | NGG.40 | TGG | GGGATTGGTGACGACAGCCG | 206 |
| | | NGG.41 | TGG | GGGGCTCAGCAGGGCGGCAA | 207 |
| | Cas12Max | TTTN.1 | TTTG | TGTCTGAGGCTGGCCCTACGGTG | 208 |
| | | TTTN.2 | TTTG | ACCATCAGAGGACACTTGGATTC | 209 |
| | | TTTN.3 | TTTC | TGAACACATGCACGGCCACATTG | 210 |
| | | TTTN.4 | TTTG | CCTCTGGGTAAGTTGCCAAAGAA | 211 |
| | | TTTN.5 | TTTG | GCAACTTACCCAGAGGCAAATGG | 212 |
| | | TTTN.6 | TTTC | ACACCTTATAGGAAAACCAGTGA | 213 |
| | | TTTN.7 | TTTC | CTATAAGGTGTGAAAGTCTGGAT | 214 |
| | | TTTN.8 | TTTT | CCTATAAGGTGTGAAAGTCTGGA | 215 |
| | | TTTN.9 | TTTG | TAGAAGGGATATACAAAGTGGAA | 216 |
| | | TTTN.10 | TTTG | TATATCCCTTCTACAAATTCCTC | 217 |
| | | TTTN.11 | TTTC | CACTTTGTATATCCCTTCTACAA | 218 |
| | | TTTN.12 | TTTG | GTGTCTATTTCCACTTTGTATAT | 219 |
| | | VTTN.1 | CTTA | CTGGAAGGCACTTGGCATCT | 220 |
| | | VTTN.2 | CTTA | TAGGAAAACCAGTGAGTCTG | 221 |
| | | VTTN.3 | CTTC | TCATCGTCTGCTCCTCCTCT | 222 |
| | | VTTN.4 | ATTC | TTGGCAGGATGGCTTCTCAT | 223 |
| | | VTTN.5 | ATTC | ACCGGTGCCCTGGGTGTAGA | 224 |
| | | VTTN.6 | GTTç | AGAAAGGCTGCTGATGACAC | 225 |
| | | VTTN.7 | GTTC | TAGATGCTGTCCGAGGCAGT | 226 |
| | | VTTN.8 | CTTC | TCTACACCCAGGGCACCGGT | 227 |
| | | VTTN.9 | GTTC | TTTGGCAACTTACCCAGAGG | 228 |
| | | VTTN.10 | CTTC | CAGTAAGATTTGGTGTCTAT | 229 |
| | | VTTN.11 | ATTC | CATGAGCATGCAGAGGTGAG | 230 |
| | | VTTN.12 | ATTC | CTCCTCAGTTGTGAGCCCAT | 231 |
| | | VTTN.13 | CTTC | TACAAATTCCTCCTCAGTTG | 232 |
| | | VTTN.14 | ATTC | ACAGCCAACGACTCCGGCCC | 233 |
| | | VTTN.15 | ATTC | CACCACGGCTGTCGTCACCA | 234 |
| | | VTTN.16 | ATTC | CTTGGGATTGGTGACGACAG | 235 |
| | | VTTN.17 | CTTC | TCTCATAGGTGGTATTCAçA | 236 |
| | | VTTN.18 | CTTG | CTGGACTGGTATTTGTGTCT | 237 |
| | | VTTN.19 | CTTG | GCAGGATGGCTTCTCATCGT | 238 |
| | | VTTN.20 | ATTG | ATGGCAGGACTGCCTCGGAC | 239 |
| | | VTTN,21 | CTTG | GATTCACCGGTGCCCTGGGT | 240 |
| | | VTTN.22 | CTTG | GCATCTCCCCATTCCATGAG | 241 |
| | | VTTN.23 | GTTG | TGAGCCCATGCAGCTCTCCA | 242 |
| | | VTTN.24 | ATTG | CCGCCCTGCTGAGCCCCTAC | 243 |
| | | VTTN.25 | GTTG | GCTGTGAATACCACCTATGA | 244 |
| | | VTTN.26 | CTTG | GGATTGGTGACGACAGCCGT | 245 |
| | | VTTN.27 | ATTG | GTGACGACAGCCGTGGTGGA | 246 |
| | | VTTN.28 | ATTT | GTGTCTGAGGCTGGCCCTAC | 247 |
| | | VTTN.29 | CTTT | GACCATCAGAGGACACTTGG | 248 |
| | | VTTN.30 | ATTT | GCCTCTGGGTAAGTTGCCAA | 249 |
| | | VTTN.31 | CTTT | GGCAACTTACCCAGAGGCAA | 250 |
| | | VTTN.32 | ATTT | GGTGTCTATTTCCACTTTGT | 251 |
| | | VTTN.33 | CTTT | GTATATCCCTTCTACAAATT | 252 |
| | hfCas12Max | TTTN.1 | TTTG | TGTCTGAGGCTGGCCCTACGGTG | 253 |
| | | TTTN.2 | TTTG | ACCATCAGAGGACACTTGGATTC | 254 |
| | | TTTN.3 | TTTC | TGAACACATGCACGGCCACATTG | 255 |
| | | TTTN.4 | TTTG | CCTCTGGGTAAGTTGCCAAAGAA | 256 |
| | | TTTN.6 | TTTC | ACACCTTATAGGAAAACCAGTGA | 257 |
| | | TTTN.7 | TTTC | CTATAAGGTGTGAAAGTCTGGAT | 258 |
| | | TTTN.8 | TTTT | CCTATAAGGTGTGAAAGTCTGGA | 259 |
| | | TTTN.9 | TTTG | TAGAAGGGATATACAAAGTGGAA | 260 |
| | | TTTN.10 | TTTG | TATATCCCTTCTACAAATTCCTC | 261 |
| | | TTTN.11 | TTTC | CACTTTGTATATCCCTTCTACAA | 262 |
| | | TTTN.12 | TTTG | GTGTCTATTTCCACTTTGTATAT | 263 |
| | | VTTN.1 | CTTA | CTGGAAGGCACTTGGCATCT | 264 |
| | | VTTN.2 | CTTA | TAGGAAAACCAGTGAGTCTG | 265 |
| | | VTTN.3 | CTTC | TCATCGTCTGCTCCTCCTCT | 266 |
| | | VTTN.4 | ATTC | TTGGCAGGATGGCTTCTCAT | 267 |
| | | VTTN.5 | ATTC | ACCGGTGCCCTGGGTGTAGA | 268 |
| | | VTTN.6 | GTTC | AGAAAGGCTGCTGATGACAC | 269 |
| | | VTTN.7 | GTTC | TAGATGCTGTCCGAGGCAGT | 270 |
| | | VTTN.9 | GTTC | TTTGGCAACTTACCCAGAGG | 271 |
| | | VTTN.10 | CTTC | CAGTAAGATTTGGTGTCTAT | 272 |
| | | VTTN.11 | ATTC | CATGAGCATGCAGAGGTGAG | 273 |
| | | VTTN.12 | ATTC | CTCCTCAGTTGTGAGCCCAT | 274 |
| | | VTTN.13 | CTTC | TACAAATTCCTCCTCAGTTG | 275 |
| | | VTTN.14 | ATTC | ACAGCCAACGACTCCGGCCC | 276 |
| | | VTTN.15 | ATTC | CACCACGGCTGTCGTCACCA | 277 |
| | | VTTN.16 | ATTC | CTTGGGATTGGTGACGACAG | 278 |
| | | VTTN.17 | CTTC | TCTCATAGGTGGTATTCACA | 279 |
| | | VTTN.18 | CTTG | CTGGACTGGTATTTGTGTCT | 280 |
| | | VTTN.19 | CTTG | GCAGGATGGCTTCTCATCGT | 281 |
| | | VTTN.20 | ATTG | ATGGCAGGACTGCCTCGGAC | 282 |
| | | VTTN.21 | CTTG | GATTCACCGGTGCCCTGGGT | 283 |
| | | VITN.22 | CTTG | GCATCTCCCCATTCCATGAG | 284 |
| | | VTTN.23 | GTTG | TGAGCCCATGCAGCTCTCCA | 285 |
| | | VTTN.24 | ATTG | CCGCCCTGCTGAGCCCCTAC | 286 |
| | | VTTN.25 | GTTG | GCTGTGAATACCACCTATGA | 287 |
| | | VTTN,26 | CTTG | GGATTGGTGACGACAGCCGT | 288 |
| | | VTTN.27 | ATTG | GTGACGACAGCCGTGGTGGA | 289 |
| | | VTTN.28 | ATTT | GTGTCTGAGGCTGGCCCTAC | 290 |
| | | VTTN.29 | CTTT | GACCATCAGAGGACACTTGG | 291 |
| | | VTTN.30 | ATTT | GCCTCTGGGTAAGTTGCCAA | 292 |
| | | VTTN.31 | CTTT | GGCAACTTACCCAGAGGCAA | 293 |
| | | VTTN.32 | ATTT | GGTGTCTATTTCCACTTTGT | 294 |
| | | VTTN.33 | CTTT | GTATATCCCTTCTACAAATT | 295 |
| | ABR001 | TTTN.1 | TTTG | TGTCTGAGGCTGGCCCTACGGTG | 296 |
| | | TTTN.2 | TTTG | ACCATCAGAGGACACTTGGATTC | 297 |
| | | TTTN.3 | TTTC | TGAACACATGCACGGCCACATTG | 298 |
| | | TTTN.4 | TTTG | CCTCTGGGTAAGTTGCCAAAGAA | 299 |
| | | TTTN.6 | TTTC | ACACCTTATAGGAAAACCAGTGA | 300 |
| | | TTTN.7 | TTTC | CTATAAGGTGTGAAAGTCTGGAT | 301 |
| | | TTTN.8 | TTTT | CCTATAAGGTGTGAAAGTCTGGA | 302 |
| | | TTTN.9 | TTTG | TAGAAGGGATATACAAAGTGGAA | 303 |
| | | TTTN.10 | TTTG | TATATCCCTTCTACAAATTCCTC | 304 |
| | | TTTN.11 | TTTC | CACTTTGTATATCCCTTCTACAA | 305 |
| | | TTTN.12 | TTTG | GTGTCTATTTCCACTTTGTATAT | 306 |
| | | VTTN.1 | CTTA | CTGGAAGGCACTTGGCATCT | 307 |
| | | VTTN.2 | CTTA | TAGGAAAACCAGTGAGTCTG | 308 |
| | | VTTN.3 | CTTC | TCATCGTCTGCTCCTCCTCT | 309 |
| | | VTTN.4 | ATTC | TTGGCAGGATGGCTTCTCAT | 310 |
| | | VTTN.5 | ATTC | ACCGGTGCCCTGOGTGTAGA | 311 |
| | | VTTN.6 | GTTC | AGAAAGGCTGCTGATGACAC | 312 |
| | | VTTN.7 | GTTC | TAGATGCTGTCCGAGGCAGT | 313 |
| | | VTTN.9 | GTTC | TTTGGCAACTTACCCAGAGG | 314 |
| | | VTTN.10 | CTTC | CAGTAAGATTTGGTGTCTAT | 315 |
| | | VTTN.11 | ATTC | CATGAGCATGCAGAGGTGAG | 316 |
| | | VTTN.12 | ATTC | CTCCTCAGTTGTGAGCCCAT | 317 |
| | | VTTN.13 | CTTC | TACAAATTCCTCCTCAGTTG | 318 |
| | | VTTN.14 | ATTC | ACAGCCAACCACTCCGGCCC | 319 |
| | | VTTN.15 | ATTC | CACCACGGCTGTCGTCACCA | 320 |
| | | VTTN.16 | ATTC | CTTGGGATTGGTGACGACAG | 321 |
| | | VTTN.17 | CTTC | TCTCATAGGTGGTATTCACA | 322 |
| | | VTTN.18 | CTTG | CTGGACTCGTATTTGTGTCT | 323 |
| | | VTTN.19 | CTTG | GCAGGATGGCTTCTCATCGT | 324 |
| | | VTTN.20 | ATTG | ATGGCAGGACTGCCTCGGAC | 325 |
| | | VTTN.21 | CTTG | GATTCACCGGTGCCCTGGGT | 326 |
| | | VTTN.22 | CTTG | GCATCTCCCCATTCCATGAG | 327 |
| | | VTTN.23 | GTTG | TGAGCCCATGCAGCTCTCCA | 328 |
| | | VTTN.24 | ATTG | CCGCCCTGCTGAGCCCCTAC | 329 |
| | | VTTN.25 | GTTG | GCTGTGAATACCACCTATGA | 330 |
| | | VTTN.26 | CTTG | GGATTGGTGACGACAGCCGT | 331 |
| | | VTTN.27 | ATTG | GTGACGACAGCCGTGGTGGA | 332 |
| | | VTTN.28 | ATTT | GTGTCTGAGGCTGGCCCTAC | 333 |
| | | VTTN.29 | CTTT | GACCATCAGAGGACACTTGG | 334 |
| | | VTTN.30 | ATTT | GCCTCTGGGTAAGTTGCCAA | 335 |
| | | VTTN.31 | CTTT | GGCAACTTACCCAGAGGCAA | 336 |
| | | VTTN.32 | ATTT | GGTGTCTATTTCCACTTTGT | 337 |
| | | VTTN.33 | CTTT | GTATATCCCTTCTACAAATT | 338 |
| | Cas12i2_{H1F1} | TTTN.1 | TTTG | TGTCTGAGGCTGGCCCTACGGTG | 339 |
| | | TTTN.2 | TTTG | ACCATCAGAGGACACTTGGATTC | 340 |
| | | TTTN.3 | TTTC | TGAACACATGCACGGCCACATTG | 341 |
| | | TTTN.4 | TTTG | CCTCTGGGTAAGTTGCCAAAGAA | 342 |
| | | TTTN.6 | TTTC | ACACCTTATAGGAAAACCAGTGA | 343 |
| | | TTTN.7 | TTTC | CTATAAGGTGTGAAAGTCTGGAT | 344 |
| | | TTTN.8 | TTTT | CCTATAAGGTGTGAAAGTCTGGA | 345 |
| | | TTTN.9 | TTTG | TAGAAGGGATATACAAAGTGGAA | 346 |
| | | TTTN.10 | TTTG | TATATCCCTTCTACAAATTCCTC | 347 |
| | | TTTN.11 | TTTC | CACTTTGTATATCCCTTCTACAA | 348 |
| | | TTTN.12 | TTTG | GTGTCTATTTCCACTTTGTATAT | 349 |
| | | VTTN.1 | CTTA | CTGGAAGGCACTTGGCATCT | 350 |
| | | VTTN.2 | CTTA | TAGGAAAACCAGTGAGTCTG | 351 |
| | | VTTN.3 | CTTC | TCATCGTGTGCTCCTCCTCT | 352 |
| | | VTTN.4 | ATTC | TTGGCAGGATGGCTTCTCAT | 353 |
| | | VTTN.5 | ATTC | ACCGGTGCCCTGGGTGTAGA | 354 |
| | | VTTN.6 | GTTC | AGAAAGGCTGCTGATGACAC | 355 |
| | | VTTN.7 | GTTC | TAGATGCTGTCCGAGGCAGT | 356 |
| | | VTTN.9 | GTTC | TTTGGCAACTTACCCAGAGG | 357 |
| | | VTTN.10 | CTTC | CAGTAAGATTTGGTGTCTAT | 358 |
| | | VTTN.11 | ATTO | CATGAGCATGCAGAGGTGAG | 359 |
| | | VTTN.12 | ATTC | CTCCTCAGTTGTGAGCCCAT | 360 |
| | | VTTN.13 | CTTC | TACAAATTCCTCCTCAGTTG | 361 |
| | | VTTN.14 | ATTC | ACAGCCAACGACTCCGGCCC | 362 |
| | | VTTN.15 | ATTC | CACCACGGCTGTCGTCACCA | 363 |
| | | VTTN.16 | ATTC | CTTGGGATTGGTGACGACAG | 364 |
| | | VTTN.17 | CTTC | TCTCATAGGTGGTATTCACA | 365 |
| | | VTTN.18 | CTTG | CTGGACTGGTATTTGTGTCT | 366 |
| | | VTTN.19 | CTTG | GCAGGATGGCTTCTCATCGT | 367 |
| | | VTTN.20 | ATTG | ATGGCAGGACTGCCTCGGAC | 368 |
| | | VTTN.21 | CTTG | GATTCACCGGTGCCCTGGGT | 369 |
| | | VTTN.22 | CTTG | GCATCTCCCCATTCCATGAG | 370 |
| | | VTTN.23 | GTTG | TGAGCCCATGCAGCTCTCCA | 371 |
| | | VTTN.24 | ATTG | CCGCCCTGCTGAGCCCCTAC | 372 |
| | | VTTN.25 | GTTG | GCTGTGAATACCACCTATGA | 373 |
| | | VTTN.26 | CTTG | GGATTGGTGACGACAGCCGT | 374 |
| | | VTTN.27 | ATTG | GTGACGACAGCCGTGGTGGA | 375 |
| | | VTTN.28 | ATTT | GTGTCTGAGGCTGGCCCTAC | 376 |
| | | VTTN.29 | CTTT | GACCATCAGAGGACACTTGG | 377 |
| | | VTTN.30 | ATTT | GCCTCTGGGTAAGTTGCCAA | 378 |
| | | VTTN.31 | CTTT | GGCAACTTACCCAGAGGCAA | 379 |
| | | VTTN.32 | ATTT | GGTGTCTATTTCCACTTTGT | 380 |
| | | VTTN.33 | CTTT | GTATATCCCTTCTACAAATT | 381 |

To further evaluate the specificity of hfCas12Max on endogenous genes in human cells, the applicant determined indel frequencies of P2RX5 and NLRC4 on-target and their corresponding in silico predicted off-target sites. Targeted deep sequence analysis showed that hfCas12Max had a higher on-target editing efficiency and similarly almost no indel activity at potential off target sites, compared to Ultra AsCas12a and LbCas12a (FIG. 10A-B; protospacer sequences/spacer sequences of SEQ ID NOs: 382-390 (not including the 5′ PAM TTTN in blue) from upside to downside in FIG. 10A; protospacer sequences/spacer sequences of SEQ ID NOs: 391-397 (not including the 5′ PAM TTTN in blue) from upside to downside in FIG. 10B. To save drafting, the sequences in black in FIGS. 10A and 10B refer to both the protospacer sequence (a DNA sequence) and the corresponding spacer sequence (an RNA sequence) with any “T” in the sequence when referring to a protospacer sequences standing for “T” and when referring to a such spacer sequence standing for “U”, although the assigned SEQ ID NOs: 382-397 in the sequence listing are annotated as DNA. To sufficiently detect off-target of hfCas12Max and to compare to other Cas proteins, the applicant used PEM-seq to quantify germline events (uncut or perfect rejoining) and editing events including indels and translocations events of TTR. 2 libraries.

Overall, these results demonstrate that hfCas12Max has high efficiency and specificity and is superior to SpCas9 and other Cas12 nucleases.

### Example 9 Development and Evaluation of Base Editor Based on Dead xCas12i

The applicant further explored the base editing of xCas12i by generating a nuclease-deactivated xCas12i mutant (dead xCas12i, dxCas12i). This was done by introducing single mutations (D650A, D700A, E875A, or D1049A) in the conserved active site of xCas12i based on alignment to Cas12i1 and Cas12i2 (FIG. 11A). The dsDNA cleavage activity (Indel %) of each of the four dxCas12i mutants (xCas12i-D650A, xCas12i-D700A, xCas12i-E875A, and xCas12i-D1049A) was measured in comparison to dead LbCpf1 (dLbCpf1-D832A) and xCas12i (WT), with N-terminally fusion of TadA8e^{V106W} (SEQ ID NO: 439, TadA8e. 1), and the results confirmed that all the four dxCas12i mutants had none or little dsDNA cleavage activity (FIG. 11B). xCas12i-D1049A had the lowest overall dsDNA cleavage activity and thus used in further base editor designs.

Then, initial versions of adenine base editor (ABE) and cytidine base editor (CBE) were constructed based on dxCas12i-D1049A (FIGS. 1H and 1J). dxCas12i-D1049A was C-terminally fused to TadA8e^{V106W} (SEQ ID NO: 439, TadA8e. 1) via a GS linker containing a XTEN linker (SEQ ID NO: 442) to form an initial version of ABE named TadA8e. 1-dxCas12i. dxCas12i-D1049A was C-terminally fused to human APOBEC3A^{W104A} (SEQ ID NO: 440, hA3A. 1) via a GS linker containing a XTEN linker (SEQ ID NO: 442), and fused to one UGI (SEQ ID NO: 441), to form an initial version of CBE named hA3A. 1-dxCas12i (FIGS. 1H and 1J). For the ABE, it further contained a N-terminal SV40 NLS (SEQ ID NO: 444) and a C-terminal BP NLS (SEQ ID NO: 443) flanking the fusion of the TadA8e^{V106W} and the dxCas12i-D1049A. For the CBE, it further contained a N-terminal BP NLS (SEQ ID NO: 443) and a C-terminal BP NLS (SEQ ID NO: 443) flanking the fusion of the hA3A. 1, the dxCas12i-D1049A, and the UGI.

TadA8e^{V106W},
SEQ ID NO: 439
SEVEFSHEYWMRHALTLAKRARDEREVPVGAVLVLNNRVIGEGWNRAIGLHDPTAHAEIMALRQGGLV
MQNYRLIDATLYVTFEPCVMCAGAMIHSRIGRVVFGWRNSKRGAAGSLMNVLNYPGMNHRVEITEGILA
DECAALLCDFYRMPRQVFNAQKKAQSSIN
TadA8e^{W106V},
SEQ ID NO: 461
SEVEFSHEYWMRHALTLAKRARDEREVPVGAVLVLNNRVIGEGWNRAIGLHDPTAHAEIMALRQGGLV
MQNYRLIDATLYVTFEPCVMCAGAMIHSRIGRVVFGVRNSKRGAAGSLMNVLNYPGMNHRVEITEGILA
DECAALLCDFYRMPRQVFNAQKKAQSSIN
hAPOBEC3^{W104A},
SEQ ID NO: 440
MEASPASGPRHLMDPHIFTSNFNNGIGRHKTYLCYEVERLDNGTSVKMDQHRGFLHNQAKNLLCGFYG
RHAELRFLDLVPSLQLDPAQIYRVTWFISYSPCFSAGCAGEVRAFLQENTHVRLRIFAARIFDYDPLYKEAL
QMLRDAGAQVSIMTYDEFKHCWDTFVDHQGCPFQPWDGLDEHSQALSGRLRAILQNQGN
UGI,
SEQ ID NO: 441
TNLSDIEKETGKQLVIQESILMLPEEVEEVIGNKPESDILVHTAYDESTDENVMLLTSDAPEYKPWALVIQD
SNGENKIKML
XTEN linker,
SEQ ID NO: 442
SGSETPGTSESATPES
bpNLS (also known as BP NLS or bpSV40 NLS) 1, (doi: 10.1038/nature20565.),
SEQ ID NO: 443
KRTADGSEFESPKKKRKV
bpNLS 2,
SEQ ID NO: 462
KRTADGSESEPKKKRKV
SV40 NLS, from Betapolyomavirus macacae,
SEQ ID NO: 444
PKKKRKV
NP NLS (also known as Xenopus laevis Nucleoplasmin NLS or nucleoplasmin
NLS), (doi: 10.1126/science.abj6856.), also a bipartite NLS,
SEQ ID NO: 445
KRPAATKKAGQAKKKK
human U6 promoter, 241 bp,
SEQ ID NO: 446
gagggcctatttcccatgattccttcatatttgcatatacgatacaaggctgttagagagataattggaattaatttgactgtaaacacaaagat
attagtacaaaatacgtgacgtagaaagtaataatttcttgggtagtttgcagttttaaaattatgttttaaaatggactatcatatgcttaccc
gtaacttgaaagtatttcgatttcttggctttatatatcttgtggaaaggac
human CMV promoter, 204 bp,
SEQ ID NO: 447
gtgatgcggttttggcagtacatcaatgggcgtggatagcggtttgactcacggggatttccaagtctccaccccattgacgtcaatgggagttt
gttttggcaccaaaatcaacgggactttccaaaatgtcgtaacaactccgccccattgacgcaaatgggcggtaggcgtgtacggtgggaggtct
atataagcagagct
bGH polyA signal, 208 bp,
SEQ ID NO: 448
ctgtgccttctagttgccagccatctgttgtttgcccctcccccgtgccttccttgaccctggaaggtgccactcccactgtcctttcctaataa
aatgaggaaattgcatcgcattgtctgagtaggtgtcattctattctggggggtggggtggggcaggacagcaagggggaggattgggaagagaa
tagcaggcatgctgggga
T5 EXO,
SEQ ID NO: 449
MSKSWGKFIEEEEAEMASRRNLMIVDGTNLGFRFKHNNSKKPFASSYVSTIQSLAKSYSARTTIVLGDKG
KSVFRLEHLPEYKGNRDEKYAQRTEEEKALDEQFFEYLKDAFELCKTTFPTFTIRGVEADDMAAYIVKLI
GHLYDHVWLISTDGDWDTLLTDKVSRFSFTTRREYHLRDMYEHHNVDDVEQFISLKAIMGDLGDNIRGV
EGIGAKRGYNIIREFGNVLDIIDQLPLPGKQKYIQNLNASEELLERNLILVDLPTYCVDALAAVGQDVLDKF
TKDILEIAEQ
CAG promoter (human CMV enhancer+ chicken β-actin promoter) (containing a hybrid intron),
SEQ ID NO: 450
cgttacataacttacggtaaatggcccgcctggctgaccgcccaacgacccccgcccattgacgtcaatagtaacgccaatagggactttccatt
gacgtcaatgggtggagtatttacggtaaactgcccacttggcagtacatcaagtgtatcatatgccaagtacgccccctattgacgtcaatgac
ggtaaatggcccgcctggcattGtgcccagtacatgaccttatgggactttcctacttggcagtacatctacgtattagtcatcgctattaccat
ggtcgaggtgagccccacgttctgcttcactctccccatctcccccccctccccacccccaattttgtatttatttattttttaattattttgtg
cagcgatgggggcggggggggggggggggcgcgcgccaggcggggcggggcggggcgaggggcggggcggggcgaggcggagaggtgcggcggca
gccaatcagagcggcgcgctccgaaagtttccttttatggcgaggcggcggcggcggcggccctataaaaagcgaagcgcgcggggggggagtcg
ctgcgacgctgccttcgccccgtgccccgctccgccgccgcctcgcgccgcccgccccggctctgactgaccgcgttactcccacaggtgagcgg
gcgggacggcccttctcctccgggctgtaattagctgagcaagaggtaagggtttaagggatggttggttggtggggtattaatgtttaattacc
tggagcacctgcctgaaatcactttttttcag

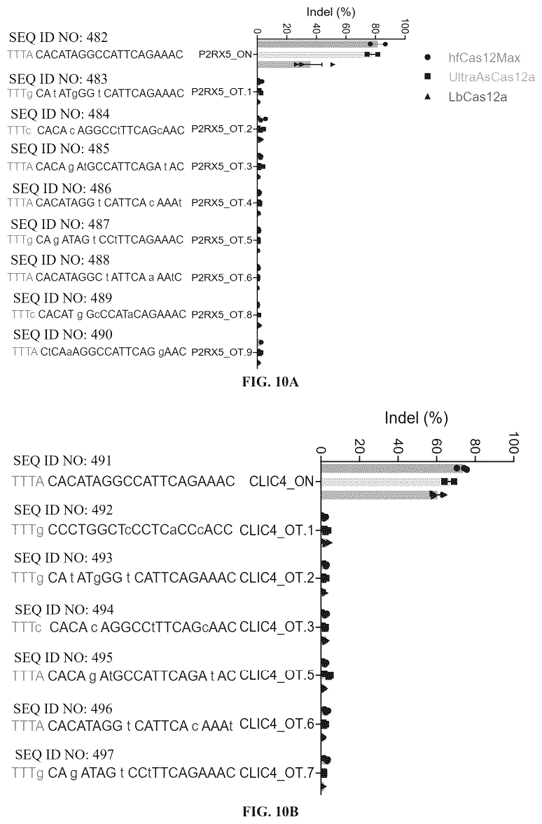

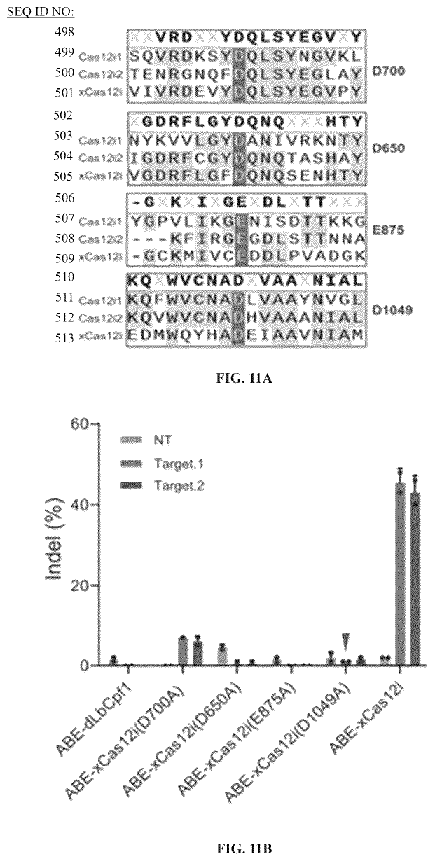

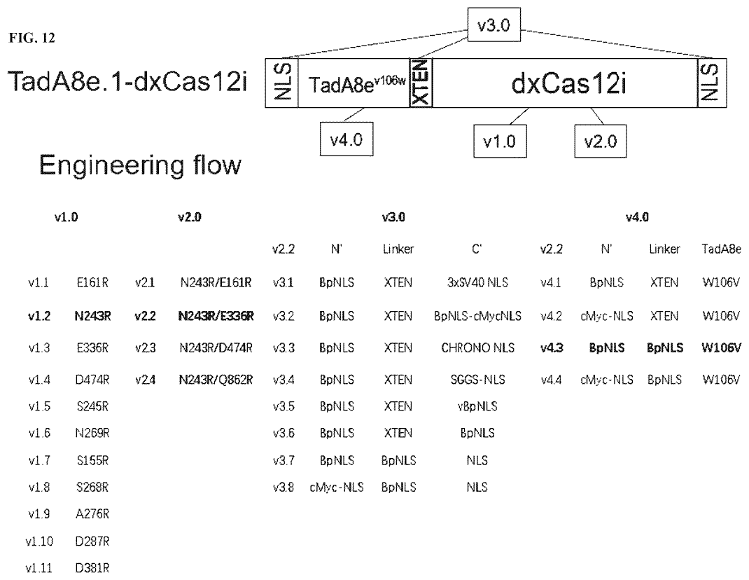

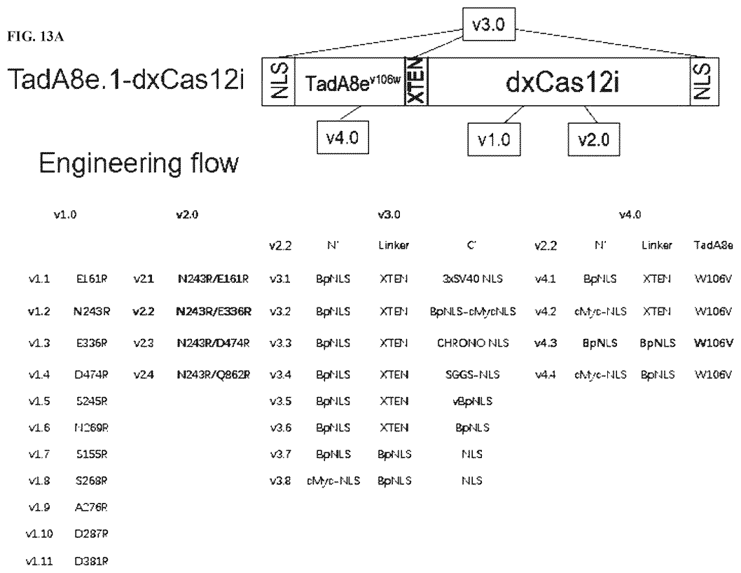

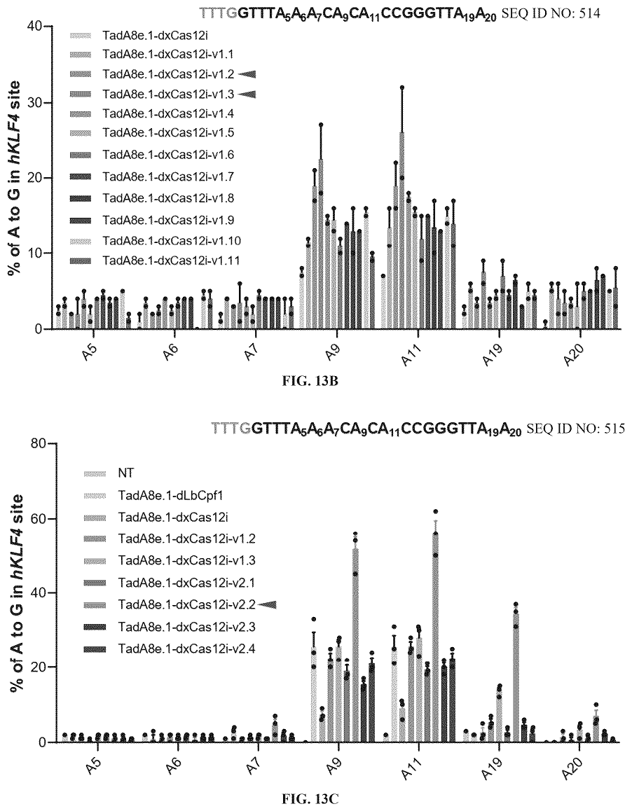

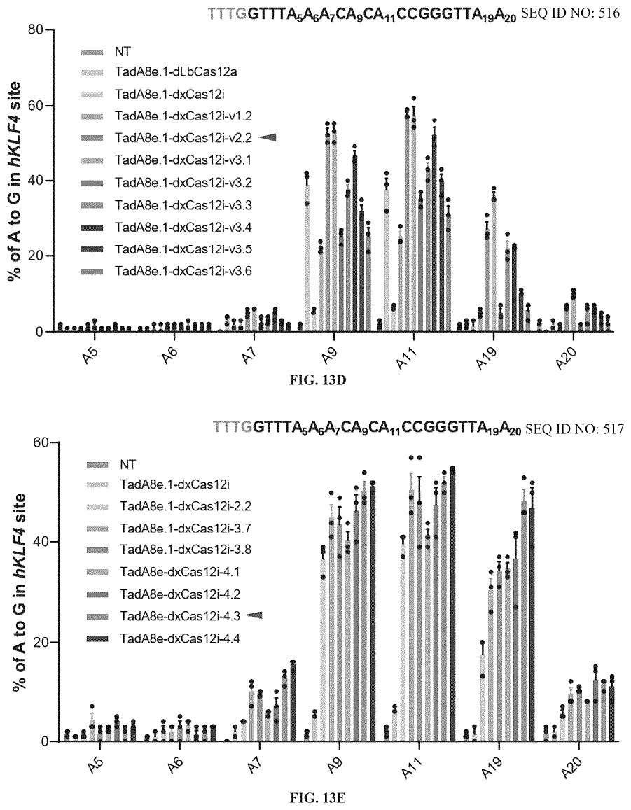

The initial versions of ABE and CBE showed low base editing activity with frequencies of about 8% A-to-G and about 2% C-to-T, respectively (FIG. 1I, 1K). To address this, the applicant conducted a series of designs, including introduction of single and combined mutations for high cleavage activity into the PI and Rec domains of dxCas12i (FIG. 12 and FIG. 13A), which resulted in significantly increased A-to-G editing activity.

As shown in FIG. 1I, TadA8e. 1-dxCas12i-v1.2 (N243R) achieved significantly higher A-to-G base editing efficiency than TadA8e. 1-dxCas12i (initial version) at sites A9, A11, A19, and A20 of the KLF4 locus, indicating that the introduction of a mutation (e.g., N243R) that has been demonstrated to improve on-target dsDNA cleavage activity can also improve the A-to-G base editing of the base editor comprising the dCas12i and a deaminase domain. Further, TadA8e. 1-dxCas12i-v2.2 (N243R+E336R) achieved significantly higher A-to-G base editing efficiency than TadA8e. 1-dxCas12i-v1.2 (N243R) at sites A7, A9, A11, A19, and A20 of KLF4, further confirming that the introduction of a mutation (e.g., E336R) that has been demonstrated to improve on-target dsDNA cleavage activity can also improve the A-to-G base editing of the base editor comprising the dCas12i and a deaminase domain.

TadA8e. 1-dxCas12i-v2.2 (D1049A+N243R+E336R) achieved 50% activity at A9 and A11 sites of the KLF4 locus, markedly higher than the 30% activity of TadA8e. 1-dLbCas12a (FIG. 11, FIG. 13B-C). At target sites within PCSK9 and TTR, TadA8e. 1-dxCas12i-v2.2 showed a similarly increased efficiency to mediate A-to-G transitions, and higher than TadA8e. 1-dLbCas12a at PCSK9 site (FIG. 15).

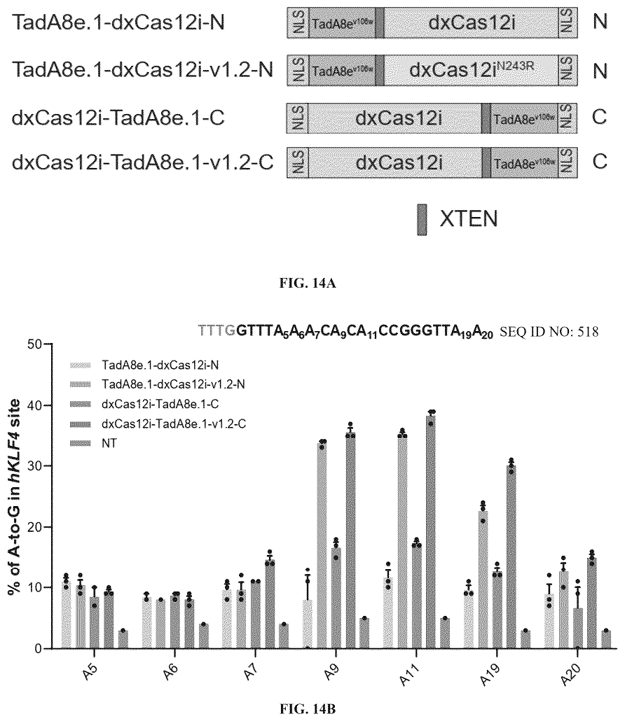

To test whether the orientation of deaminase fusion affects the base editing efficiency, the applicant constructed dxCas12i-ABE by fusing the TadA8e. 1 to N or C terminus of dxCas12i and found that TadA8e. 1 at C terminus of dxCas12i showed slightly higher activity than N terminus (FIG. 14).

The applicant then further engineered the NLS, linker, and TadA8e. 1 protein (return back to TadA8e (SEQ ID NO: 461; TadA8e^{W106V})) (FIG. 12; FIG. 13A) to produce v3.1-v3.8 and v4.1-v4.4, where TadA8e-dxCas12i-v4.3 exhibited a nearly 80% A-to-G editing efficiency and >95% editing purity, which is significantly higher than TadA8e. 1-dxCas12i-v2.2, indicating that the base editing efficiency can also be improved by specific selections of the NLS, linker, and deaminase domain (FIG. 1H-1I, FIG. 13D-13E). The applicant named TadA8e-dxCas12i-v4.3 as dCas12Max-ABE (SEQ ID NO: 463), which contains, from N-terminal to C-terminal, Methionine (M), bpNLS 1 (SEQ ID NO: 443), TadA8e-W106V (SEQ ID NO: 461), bpNLS 1-containing GS linker (SEQ ID NO: 465), xCas12i-N243R+E336R+D1049A (SEQ ID NO: 466), and npNLS (SEQ ID NO: 445).

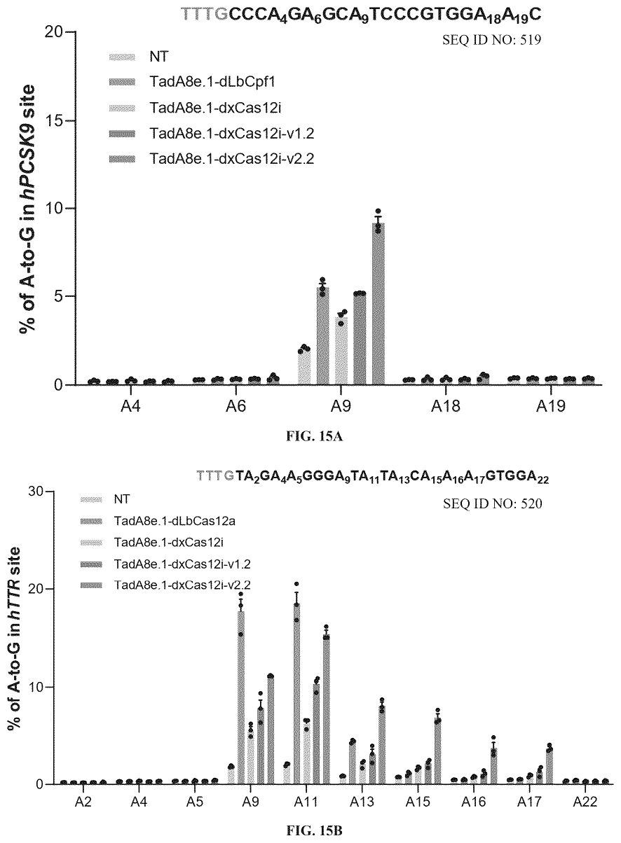

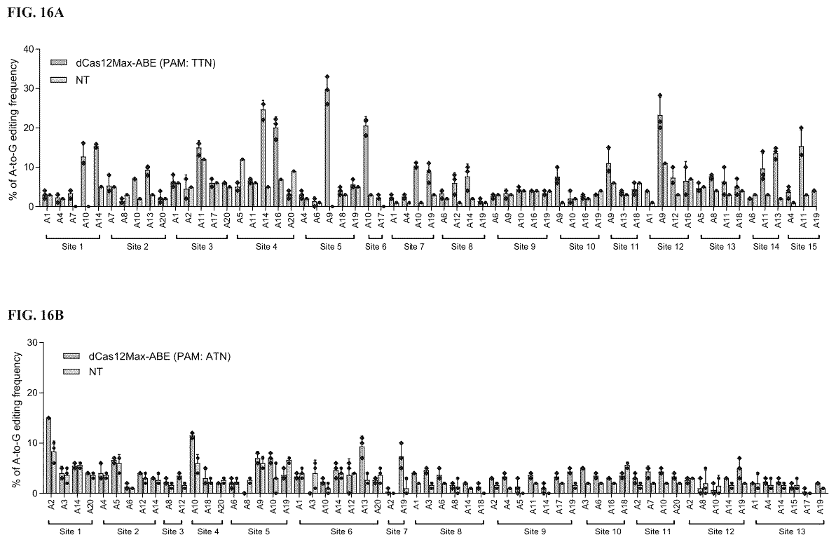

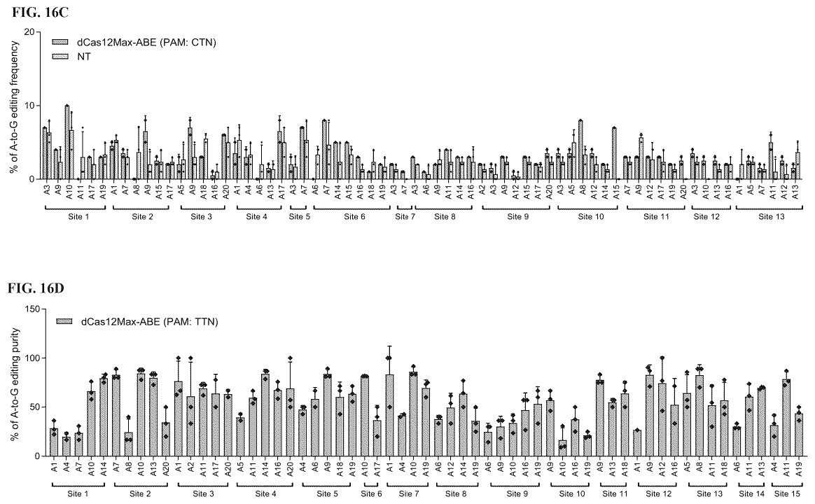

To further characterize the base editing activity of dCas12Max-ABE, the applicant performed 21 sites with TTN PAM, 13 sites with ATN PAMs and 13 sites with CTN PAMs (Table 10). It was observed that dCas12Max-ABE exhibited significant A-to-G activity at sites with TTN PAM (FIG. 16).

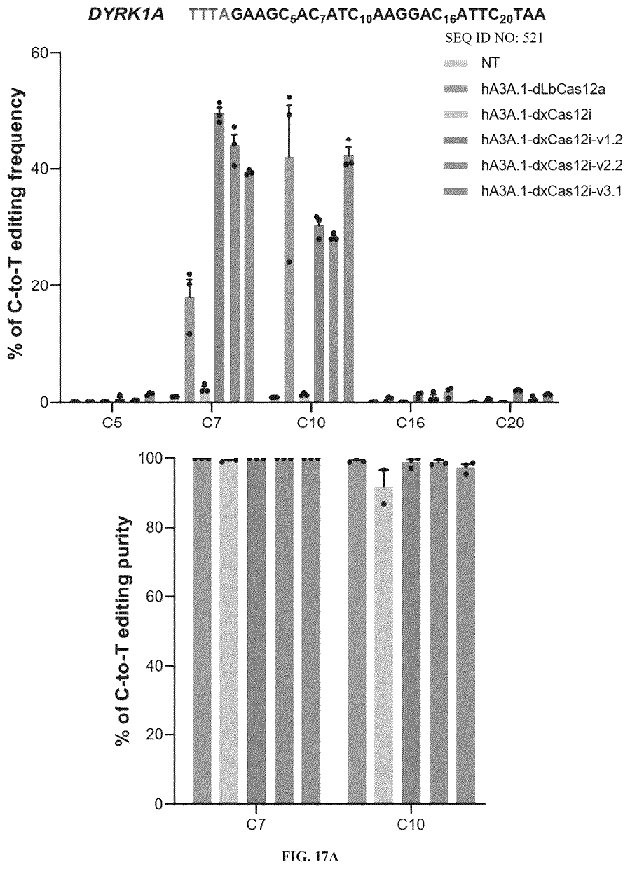

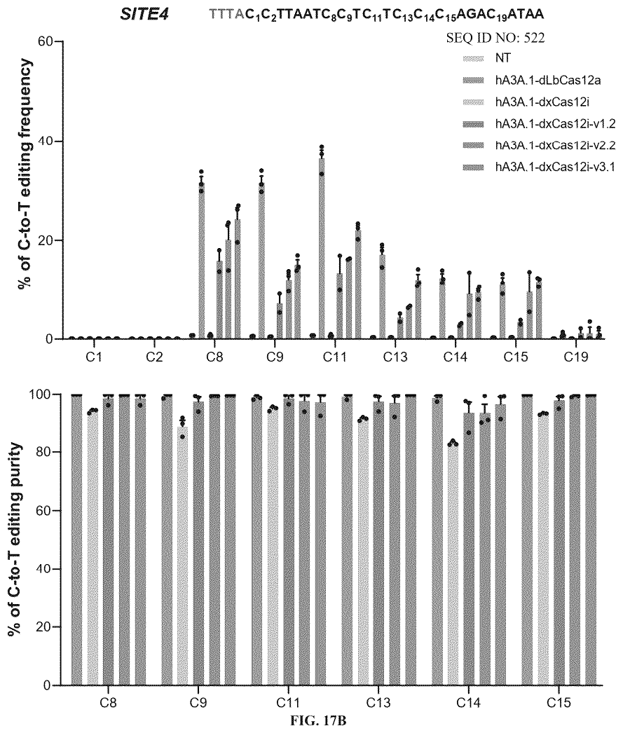

Similarly for CBE, hA3A. 1-dxCas12i-v1.2 (N243R), hA3A. 1-dxCas12i-v2.2 (N243R+E336R), and hA3A. 1-dxCas12i-v3.1 (N243R+E336R-bpNLS) showed consistently elevated C-to-T editing efficiency along with >95% editing purity at RUNX1, DYRKIA, and SITE4 locus, even higher than hA3A. 1-dLbCas12a at RUNX1 and DYRK1A (FIG. 1J-K and FIG. 17). The applicant named hA3A. 1-dxCas12i-v3.1 (N243R+E336R-bpNLS) as dCas12Max-CBE (SEQ ID NO: 464), which contains, from N-terminal to C-terminal, Methionine (M), bpNLS 1 (SEQ ID NO: 443), hAPOBEC3_{W104A} (SEQ ID NO: 440), bpNLS 1-containing GS linker (SEQ ID NO: 465), xCas12i-N243R+E336R+D1049A (SEQ ID NO: 466), a short GS linker, SV40 NLS (SEQ ID NO: 444), a short GS linker, UGI (SEQ ID NO: 441), a short GS linker, and bpNLS 2 (SEQ ID NO: 462).

These results together demonstrate that both the engineered dxCas12i-based ABE and CBE exhibited high base editing activity in mammalian cells.

To save drafting, the sequences in Table 10 refer to both the protospacer sequence (a DNA sequence) and the corresponding spacer sequence (an RNA sequence) with any “T” in the sequence when referring to a protospacer sequences standing for “T” and when referring to a such spacer sequence standing for “U”, although the assigned SEQ ID NOs: 398-438 in the sequence listing are annotated as DNA.

TABLE 10
Sequence of target loci for A to G frequency at different sites (FIG. 16)

| | | | | | SEQ ID NO of |
|---|---|---|---|---|---|
| Genomic | | | | Protospacer/ | Protospacer/ |
| loci | ABE | SITE | 5′/3′PAM | Spacer Sequence | Spacer Sequence |
| TTR | TTN | site1 | CTTC | AGCACCACCACGTAGGTGCC | 398 |
| | | site2 | CTTC | CTGGTGAAGATGAGTGGCGA | 399 |
| | | site3 | CTTG | AAGTTGCCCCATGTCGACTA | 400 |
| | | site4 | GTTG | CCCCATGTCGACTACATCGA | 401 |
| | | site5 | TTTG | CCCAGAGCATCCCGTGGAAC | 402 |
| | | site6 | TTTC | CCGGTGGTCACTCTGTATGC | 403 |
| | | site7 | GTTG | AGCACGCGCAGGCTGCGCAT | 404 |
| | | site8 | GTTA | GCGGCACCCTCATAGGTGAG | 405 |
| | | site9 | GTTG | GGGCCACCAATGCCCAGGAC | 406 |
| | | site10 | ATTG | GTGGCCCCAACTGTGATGAC | 407 |
| | | site11 | ATTG | GTGCCTCCAGCGACTGCAGC | 408 |
| | | site12 | ATTC | ACCCCTGCACCAGGCATTGC | 409 |
| | | site13 | GTTC | CCTGAGGACCAGCGGGTACT | 410 |
| | | site14 | GTTG | GTGGCAGTGGACACGGGTCC | 411 |
| | | site15 | GTTG | TCTACGGCGTAGGCCCCCAG | 412 |
| | ATN | site1 | AATC | CAAGTGTCCTCTGATGGTCA | 413 |
| | | site2 | GATG | GTCAAAGTTCTAGATGCTGT | 414 |
| | | site3 | GATG | CTGTCCGAGGCAGTCCTGCC | 415 |
| | | site4 | AATG | TGGCCGTGCATGTGTTCAGA | 416 |
| | | site5 | CATG | TGTTCAGAAAGGCTGCTGAT | 417 |
| | | site6 | GATG | ACACCTGGGAGCCATTTGCC | 418 |
| | | site7 | GATT | CACCGGTGCCCTGGGTGTAG | 419 |
| | | site8 | CATC | AGAGGACACTTGGATTCACC | 420 |
| | | site9 | CATC | TAGAACTTTGACCATCAGAG | 421 |
| | | site10 | GATG | GCAGGACTGCCTCGGACAGC | 422 |
| | | site11 | CATT | GATGGCAGGACTGCCTCGGA | 423 |
| | | site12 | CATG | CACGGCCACATTGATGGCAG | 424 |
| | | site13 | CATC | AGCAGCCTTTCTGAACACAT | 425 |
| | CTN | site1 | CCTC | TGATGGTCAAAGTTCTAGAT | 426 |
| | | site2 | TCTG | ATGGTCAAAGTTCTAGATGC | 427 |
| | | site3 | GCTG | TCCGAGGCAGTCCTGCCATC | 428 |
| | | site4 | GCTG | ATGACACCTGGGAGCCATTT | 429 |
| | | site5 | CCTG | GGAGCCATTTGCCTCTGGGT | 430 |
| | | site6 | CCTC | TGGGTAAGTTGCCAAAGAAC | 431 |
| | | site7 | ACTT | GGATTCACCGGTGCCCTGGG | 432 |
| | | site8 | ACTT | TGACCATCAGAGGACACTTG | 433 |
| | | site9 | TCTA | GAACTTTGACCATCAGAGGA | 434 |
| | | site10 | CCTC | GGACAGCATCTAGAACTTTG | 435 |
| | | site11 | ACTG | CCTCGGACAGCATCTAGAAC | 436 |
| | | site12 | GCTC | CCAGGTGTCATCAGCAGCCT | 437 |
| | | site13 | ACTT | ACCCAGAGGCAAATGGCTCC | 438 |

### Example 10 Evaluation of RNP Delivery of hfCas12Max in T Cells

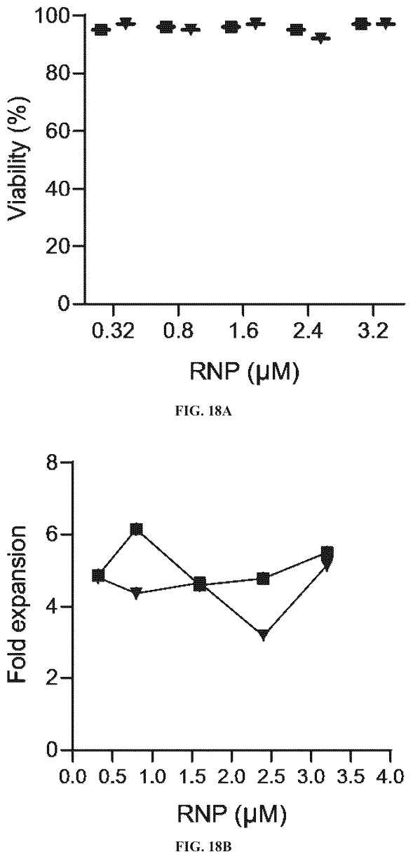

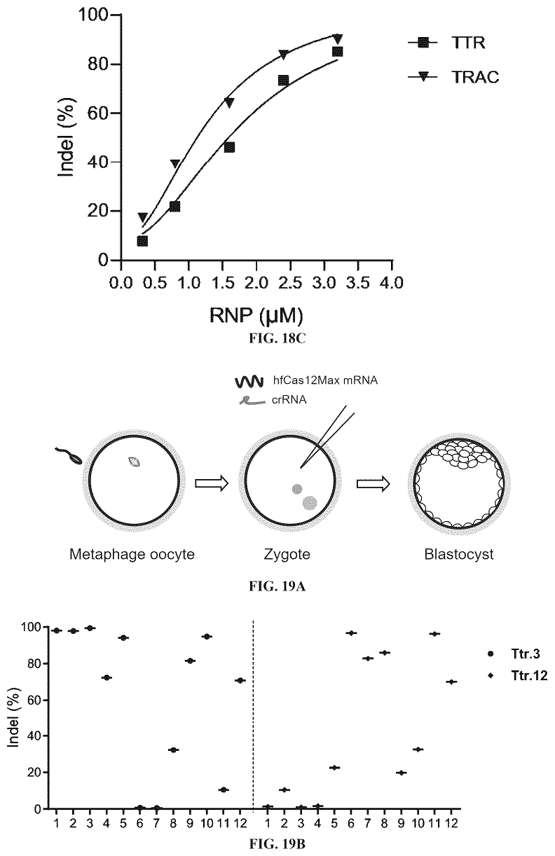

To explore the therapeutic potential application of hfCas12Max, the applicant delivered hfCas12Max RNP targeting TRAC in CD3+ T cells (FIG. 2A). Beforehand, the applicant tested hfCas12Max RNP targeting TTR and TRAC in HEK293 cells, and it was found that the gene editing efficiency was increased following increasing dose of RNPs, with unaffected cellular viability and proliferation (FIG. 18A-C). The applicant achieved about 90% dsDNA cleavage activity and >95% viability at 3.2 μM dose for TRAC (FIG. 18A-C) in HEK293 cells. Three spacer sequences (TRAC_sg. 1, TRAC_sg. 2, and TRAC_sg. 3) were designed to target TRAC (Table 5), and both TRAC_sg. 2 and TRAC_ssg. 3 generated ˜90% editing at both 1.6 and 3.2 μM doses along with ˜80% viability (FIG. 2B) in CD3+ T cells. Flow cytometric analysis showed that TRAC expression was detected to be reduced to a level of 2.54% and 3.72% in CD3+ T cells post 5 days post electroporation treated with RNPs comprising TRAC_sg. 2 or TRAC_sg. 3, respectively, compared to 96.6% in untreated cells (FIG. 2C). The guide RNA used in this Example was composed of 5′ DR-T1-spacer sequence-DR-T2-spacer sequence-3′.

### Example 11 Evaluation of LNP Delivery of hfCas12Max In Vivo

To assess the feasibility of the hfCas12Max or the base editor of in vivo gene editing, the applicant delivered a guide RNA and a mRNA encoding hfCas12Max or the base editor by LNP packaging to the liver of C57 mouse via tail intravenous injection (FIG. 2D). The applicant targeted the exon 3 in the murine transthyretin (Ttr) gene (Ttr_sg12 in Table 5) by gene editing (dsDNA cleavage) and base editing (FIG. 2E). Robust editing efficiencies were detected at four concentration and nearly 100% at 1 μg dose in N2a cells (FIG. 2F). Similarly, targeted deep sequence analysis indicated that the editing efficiencies of murine liver were approximately 70% at the dose of 0.3 and 0.5 milligrams per kilogram (mpk), equivalent to saturation (FIG. 2G). Further, through the LNP packaging delivery, TadA8e-dxCas12i-v4.3 (dCas12Max-ABE) achieved approximately 25% A-to-G efficiency at A13 in Ttr locus in murine liver at 3 mpk dose (FIG. 2H). The guide RNA used in this Example was composed of 5′ DR-T1-spacer sequence-DR-T2-spacer sequence-3′.

In addition, the applicant injected hfCas12Max mRNA with two gRNAs (with spacer sequences of Ttr_sg3 and Ttr_sg12 in Table 5) targeting Ttr gene into murine zygotes, which were cultured to blastocyst stage for genotyping analysis (FIG. 19A). Targeted deep sequence analysis showed that most zygotes were edited and some up to 100% (FIG. 19B). These results indicate that hfCas12Max mediated robust ex vivo and in vivo gene editing, showing significant potential for disease modeling and therapies.

Mis-folding and aggregation of transthyretin (TTR) is associated with amyloid diseases, including transthyretin-related wild-type amyloidosis (ATTRwt), transthyretin-related hereditary amyloidosis (ATTRm), familial amyloid polyneuropathy (FAP), and familial amyloid cardiomyopathy (FAC). Gene silencing of TTR to reduce TTR protein production may have therapeutic effects in TTR-associated amyloid diseases. The high-efficiency cleavage of TTR target sites in mice in this Example demonstrates that the CRISPR-Cas12i system of the disclosure has very promising prospects for the treatment of TTR-related amyloid diseases, such as ATTR (e.g., ATTRwt or ATTRm).

### Example 12: Screening of xCas12i Mutant with Nickase Activity

To screen xCas12i mutant with nickase activity (i.e., having ssDNA cleavage activity and substantially lacking dsDNA cleavage activity), xCas12i mutant in Tables 11-14 were designed and tested for their nickase activity and dsDNA cleavage activity, by using the reporter system for dsDNA cleavage activity in Example 1 and a reporter system for nickase activity established based on the reporter system for dsDNA cleavage activity in Example 1 wherein the insertion sequence was replaced with an insertion sequence containing, from 5′ to 3′, a 5′ PAM, a protospacer sequence (SEQ ID NO: 43), a linker, a target sequence (SEQ ID NO: 44), and a reverse complementary sequence of the 5′ PAM.

When the xCas12i mutant has only nickase activity, it does not generate green fluorescence with the reporter system for dsDNA cleavage activity but can generate green fluorescence with the reporter system for nickase activity. When the xCas12i mutant has dsDNA cleavage activity, it can generate green fluorescence with both the reporter systems for nickase activity and dsDNA cleavage activity. So the reporter system for nickase activity indicates the sum of the dsDNA cleavage activity and nickase activity. The nickase activity is calculated as green fluorescence from the reporter system for nickase activity minus green fluorescence from the reporter system for dsDNA cleavage activity. Nickase preference was calculated as nickase activity/dsDNA cleavage activity.

It was observed that xCas12i-W896R, xCas12i-S924R, and xCas12i-S925R exhibited significant nickase activity relative to xCas12i (WT) and substantially lacked dsDNA cleavage activity compared with xCas12i (WT).

TABLE 11

| | Nickase (ssDNA | dsDNA | Nickase activity/ |
|---|---|---|---|
| | cleavage) | cleavage | dsDNA cleavage |
| Mutant | activity (%) | activity (%) | activity |
| NT | 0.000 | 0.020 | 0.000 |
| Blank | 0.000 | 0.020 | 0.000 |
| xCas12i | −0.300 | 76.100 | −0.004 |
| xCas12i-W896R | 30.130 | 4.970 | 6.062 |
| xCas12i-S924R | 22.300 | 26.800 | 0.832 |
| xCas12i-S925R | 6.650 | 5.350 | 1.243 |

Further mutagenesis was conducted at W896, S924, or S925 of xCas12i to generate the mutants in Tables 12-14. It was observed that eight xCas12i mutants, W896R, W896P, W896K, S924F, S924D, S924E, S924H, and S925T, achieved both significant nickase preference (Nickase activity/dsDNA cleavage activity >1.0) and high nickase activity (higher than 20%).

TABLE 12
xCas12i-W896 mutants

| | Nickase | | |
|---|---|---|---|
| | (ssDNA | | Nickase activity/ |
| | cleavage) | dsDNA cleavage | dsDNA cleavage |
| Mutant | activity (%) | activity (%) | activity |
| W896G | −3.100 | 72.900 | −0.043 |
| W896A | 6.500 | 75.700 | 0.086 |
| W896V | −0.300 | 64.300 | −0.005 |
| W896L | 13.900 | 61.300 | 0.227 |
| W896I | −0.600 | 74.700 | −0.008 |
| W896M | 0.500 | 76.800 | 0.007 |
| W896F | 5.800 | 74.100 | 0.078 |
| W896W | −0.400 | 80.300 | −0.005 |
| W896P | 32.170 | 8.030 | 4.006 |
| W896S | 0.000 | 72.000 | 0.000 |
| W896T | 0.600 | 67.200 | 0.009 |
| W896C | 2.200 | 72.800 | 0.030 |
| W896Y | 2.300 | 67.700 | 0.034 |
| W896N | 0.700 | 63.700 | 0.011 |
| W896Q | 1.500 | 69.800 | 0.021 |
| W896D | −1.900 | 49.200 | −0.039 |
| W896E | 11.900 | 58.400 | 0.204 |
| W896K | 37.500 | 14.700 | 2.551 |
| W896H | 3.100 | 68.000 | 0.046 |

TABLE 13
xCas12i-S924 mutants

| | Nickase | | |
|---|---|---|---|
| | (ssDNA | | Nickase activity/ |
| | cleavage) | dsDNA cleavage | dsDNA cleavage |
| Mutant | activity (%) | activity (%) | activity |
| S924G | 0.100 | 70.900 | 0.001 |
| S924A | 18.000 | 53.400 | 0.337 |
| S924V | 11.100 | 53.500 | 0.207 |
| S924L | 2.800 | 54.500 | 0.051 |
| S924I | 14.900 | 41.800 | 0.356 |
| S924M | 8.100 | 49.600 | 0.163 |
| S924F | 26.600 | 15.500 | 1.716 |
| S924W | 3.530 | 8.670 | 0.407 |
| S924P | 15.500 | 10.100 | 1.535 |
| S924S | −5.000 | 82.200 | −0.061 |
| S924T | 2.800 | 78.200 | 0.036 |
| S924C | 2.700 | 70.700 | 0.038 |
| S924Y | 11.000 | 11.000 | 1.000 |
| S924N | 8.400 | 71.800 | 0.117 |
| S924Q | 23.400 | 29.200 | 0.801 |
| S924D | 29.000 | 12.700 | 2.283 |
| S924E | 22.800 | 15.400 | 1.481 |
| S924K | 14.600 | 41.600 | 0.351 |
| S924H | 36.000 | 25.300 | 1.423 |

TABLE 14
xCas12i-S925 mutants

| | Nickase | | |
|---|---|---|---|
| | (ssDNA | | Nickase activity/ |
| | cleavage) | dsDNA cleavage | dsDNA cleavage |
| Mutant | activity (%) | activity (%) | activity |
| S925G | 28.700 | 40.900 | 0.702 |
| S925A | −0.600 | 12.700 | −0.047 |
| S925V | 3.000 | 3.560 | 0.843 |
| S925L | 6.650 | 5.750 | 1.157 |
| S9251 | 9.000 | 5.800 | 1.552 |
| S925M | 5.350 | 5.150 | 1.039 |
| S925F | 7.530 | 6.870 | 1.096 |
| S925W | 3.330 | 9.770 | 0.341 |
| S925P | 4.700 | 9.700 | 0.485 |
| S925S | −0.300 | 76.300 | −0.004 |
| S925T | 32.000 | 21.200 | 1.509 |
| S925C | 7.600 | 8.000 | 0.950 |
| S925Y | 7.780 | 5.820 | 1.337 |
| S925N | 1.300 | 12.300 | 0.106 |
| S925Q | 6.230 | 5.970 | 1.044 |
| S925D | 9.320 | 6.180 | 1.508 |
| S925E | 11.690 | 6.610 | 1.769 |
| S925K | 6.700 | 10.800 | 0.620 |
| S925H | 6.100 | 10.600 | 0.575 |

In the Examples, it was demonstrated that the Type V-I Cas12i system enables versatile and efficient genome editing in mammalian cells. Among others, xCas12i that shows high editing efficiency at TTN-PAM sites was identified. By semi-rational design and protein engineering of its PI, REC, RuvC domains, a high-efficiency, high-fidelity variant, hfCas12Max, was obtained which contains N243R, E336R, and D892R substitutions. It was demonstrated that the introduction of N243R in the PI domain and E336R at REC domain significantly increased editing activity and expanded PAM recognition. Interestingly, D892R or G883R substitutions in the RuvC domain reduced off-target and retained on-target cleavage activity. The D892R substituted hfCas12Max was obviously more sensitive to mismatch, which suggests that D892R or G883R improved gRNA binding specificity. According to sequence alignment and predicted structure of xCas12i to Cas12i2, asparagine 892 is located on NUC domain, together with RuvC domain to form a cleft, in which crRNA: DNA heteroduplex was located. The variant with D892R did not alter the on-target but eliminated off-target activity, probably due to arginine substitution of asparagine affecting the binding of non-target gRNA. The data of the disclosure suggests that a semi-rational engineering strategy with arginine substitutions based on the EGFP-activated reporter system could be used as a general approach to improve the activity of CRISPR editing tools.

Through engineering, the Cas12i system of the disclosure has achieved high editing activity, high specificity, and a broad PAM range, comparable to SpCas9, and better than other Cas12 systems. Given its smaller size, short crRNA guide, and self-processing features, the type V-I Cas12i system is suitable for in vivo multiplexed gene-editing applications, including AAV or LNP. Indeed, the data of the disclosure indicates type V-I Cas12i system mediates the robust ex vivo or in vivo genome-editing efficiencies via ribonucleoprotein (RNP) delivery and lipid nanoliposomes (LNP) delivery, respectively, demonstrating the great potential for therapeutic genome-editing applications.

In addition, it was confirmed that the type V-I Cas12i system can be used in base editing applications. For base editor, the dCas12i system shows high A-to-G editing at A9-A11 sites and even A19 site of KLF locus, and C-to-T editing at C7-C10 sites, which is similar to the dCas12a system but is distinct from the dCas9/nCas9 system. Comparable to dCas12a, dCas12i-BE exhibited higher base editing activity at KLF4, PCSK9, and DYRK1A loci, suggesting it may have more potential as a base editor. This suggests that the dCas12i system of the disclosure is useful for broad genome engineering applications, including epigenome editing, genome activation, and chromatin imaging.

In summary, the Cas12i system of the disclosure, which has robust editing activity and high specificity, is a versatile platform for genome editing or base editing in mammalian cells and could be useful in the future for in vivo or ex vivo therapeutic applications.

Various modifications and variations of the described products, methods, and uses of the disclosure will be apparent to those skilled in the art without departing from the scope and spirit of the disclosure. Although the disclosure has been described in connection with specific embodiments, it will be understood that it is capable of further modifications and that the disclosure as claimed should not be unduly limited to such specific embodiments. Indeed, various modifications of the described modes for carrying out the disclosure that are obvious to those skilled in the art are intended to be within the scope of the disclosure. This application is intended to cover any variations, uses, or adaptations of the disclosure following, in general, the principles of the disclosure and including such departures from the present disclosure come within known customary practice within the art to which the disclosure pertains and may be applied to the essential features herein before set forth.

1. A Cas12i polypeptide comprising an amino acid substitution at E336, V880, G883, D892, and/or M923 of SEQ ID NO: 458; optionally, wherein the Cas12i polypeptide has a sequence identity of at least about 80%, at least about 85%, at least about 90%, at least about 95%, or at least about 99% to SEQ ID NO: 458.

2. The Cas12i polypeptide of claim 1, wherein the Cas12i polypeptide comprises an amino acid substitution at one position selected from the group consisting of E336, V880, G883, D892, and M923 of SEQ ID NO: 458; optionally, wherein the amino acid substitution is a substitution with a positively charged amino acid residue (such as, Lysine (Lys/K), Arginine (Arg/R), Histidine (His/H)), and optionally a substitution with Arginine (Arg/R).

3. The Cas12i polypeptide of claim 2, wherein the Cas12i polypeptide comprises an amino acid substitution E336R relative to SEQ ID NO: 458; optionally, wherein the Cas12i polypeptide comprises the amino acid sequence of SEQ ID NO: 467, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 467.

4. The Cas12i polypeptide of claim 2, wherein the Cas12i polypeptide comprises one amino acid substitution selected from the group consisting of V880R, G883R, D892R, and M923R relative to SEQ ID NO: 458.

5. The Cas12i polypeptide of claim 1, wherein the Cas12i polypeptide comprises two amino acid substitutions at any two positions of E336, V880, G883, D892, and M923 of SEQ ID NO: 458; optionally, wherein each of the two amino acid substitutions is independently a substitution with a positively charged amino acid residue (such as, Lysine (Lys/K), Arginine (Arg/R), Histidine (His/H)), and optionally each a substitution with Arginine (Arg/R).

6. The Cas12i polypeptide of claim 5, wherein the Cas12i polypeptide comprises amino acid substitutions E336R and one amino acid substitution selected from the group consisting of V880R, G883R, D892R, and M923R relative to SEQ ID NO: 458.

7. The Cas12i polypeptide of claim 6, wherein the Cas12i polypeptide comprises amino acid substitutions E336R and D892R relative to SEQ ID NO: 458; optionally, wherein the Cas12i polypeptide comprises the amino acid sequence of SEQ ID NO: 459, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 459.

8. The Cas12i polypeptide of claim 1, wherein the Cas12i polypeptide further comprises an additional amino acid substitution at a position selected from the group consisting of K109, L112, D125, 127, F144, L147, A148, L151, L157, V195, Y226, F252, I258, M293, W305, A308, I309, S312, A314, D315, V316, A318, L324, I327, A348, L352, Y365, L372, L376, L379, L383, I405, L424, I427, A436, F439, A443, V447, A457, H458, P459, T460, S463, S814, F859, A864, H867, Y977, S1031, A1053, and F1068 of SEQ ID NO: 458; optionally, wherein the additional amino acid substitution is a substitution with a positively charged amino acid residue (such as, Lysine (Lys/K), Arginine (Arg/R), Histidine (His/H)), and optionally a substitution with Arginine (Arg/R).

9. The Cas12i polypeptide of claim 1, wherein the Cas12i polypeptide has spacer sequence-specific (on-target) dsDNA cleavage activity; optionally, wherein the Cas12i polypeptide substantially retains the spacer sequence-specific (on-target) dsDNA cleavage activity of SEQ ID NO: 458 or SEQ ID NO: 1; and/or optionally, wherein the Cas12i polypeptide has an increased spacer sequence-specific (on-target) dsDNA cleavage activity compared to that of SEQ ID NO: 458 or SEQ ID NO: 1 when both are used in combination with a same guide nucleic acid, e.g., an increase by at least about 5%, 10%, 15%, 20%, 25%, 30%, 35%, 40%, 45%, 50%, 55%, 60%, 65%, 70%, 75%, 80%, 85%, 90%, 95%, 100%, 110%, 120%, 130%, 140%, 150%, 160%, 170%, 180%, 190%, 200%, 210%, 220%, 230%, 240%, 250%, 260%, 270%, 280%, 290%, 300%, or more.

10. The Cas12i polypeptide of claim 1, wherein the Cas12i polypeptide substantially lacks spacer sequence-independent (off-target) dsDNA cleavage activity; optionally, wherein the Cas12i polypeptide substantially lacks the spacer sequence-independent (off-target) dsDNA cleavage activity of SEQ ID NO: 458 or SEQ ID NO: 1; and/or optionally, wherein the Cas12i polypeptide has a decreased spacer sequence-independent (off-target) dsDNA cleavage activity compared to that of SEQ ID NO: 458 or SEQ ID NO: 1 when both are used in combination with a same guide nucleic acid, e.g., a decrease by at least about 5%, 10%, 15%, 20%, 25%, 30%, 35%, 40%, 45%, 50%, 55%, 60%, 65%, 70%, 75%, 80%, 85%, 90%, 95%, or 100%.

11. The Cas12i polypeptide of claim 1, wherein the Cas12i polypeptide is further engineered to substantially lack spacer sequence-specific (on-target) dsDNA cleavage activity; optionally, wherein the Cas12i polypeptide substantially lacks the spacer sequence-specific (on-target) dsDNA cleavage activity of SEQ ID NO: 458 or SEQ ID NO: 1; and/or optionally, wherein the Cas12i polypeptide has a decreased spacer sequence-specific (on-target) dsDNA cleavage activity compared to that of SEQ ID NO: 458 or SEQ ID NO: 1 when both used in combination with a same guide nucleic acid, e.g., a decrease by at least about 5%, 10%, 15%, 20%, 25%, 30%, 35%, 40%, 45%, 50%, 55%, 60%, 65%, 70%, 75%, 80%, 85%, 90%, 95%, or 100%.

12. The Cas12i polypeptide of claim 11, wherein the Cas12i polypeptide comprise a further amino acid substitution at a position selected from the group consisting of D650, D700, E875, and D1049 of SEQ ID NO: 458; optionally, wherein the further amino acid substitution is a substitution with a non-polar amino acid residue (such as, Glycine (Gly/G), Alanine (Ala/A), Valine (Val/V), Cysteine (Cys/C), Proline (Pro/P), Leucine (Leu/L), Isoleucine (Ile/I), Methionine (Met/M), Tryptophan (Trp/W), Phenylalanine (Phe/F)), and optionally a substitution with Alanine (Ala/A).

13. The Cas12i polypeptide of claim 12, wherein the Cas12i polypeptide comprises amino acid substitutions E336R and D1049A relative to SEQ ID NO: 458; optionally, wherein the Cas12i polypeptide comprises the amino acid sequence of SEQ ID NO: 466, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 466.

14. The Cas12i polypeptide of claim 1, wherein the Cas12i polypeptide is further engineered to be a nickase.

15. The Cas12i polypeptide of claim 14, wherein the Cas12i polypeptide comprise a further amino acid substitution at a position selected from the group consisting of W896, S924, and S925 to of SEQ ID NO: 458; optionally, wherein the Cas12i polypeptide comprise a further amino acid substitution selected from the group consisting of W896R, W896P, W896K, S924R, S924F, S924D, S924E, S924H, S925R, and S925T relative to SEQ ID NO: 458.

16. The Cas12i polypeptide of claim 1, wherein the Cas12i polypeptide further comprises a functional domain fused to the Cas12i polypeptide; optionally, wherein the functional domain is selected from the group consisting of a nuclear localization signal (NLS), a nuclear export signal (NES), a base editing domain, for example, a deaminase or a catalytic domain thereof, a base excising domain, an uracil glycosylase inhibitor (UGI) or a catalytic domain thereof, an uracil glycosylase (UNG) or a catalytic domain thereof, a methylpurine glycosylase (MPG) or a catalytic domain thereof, a methylase or a catalytic domain thereof, a demethylase or a catalytic domain thereof, an transcription activating domain (e.g., VP64 or VPR), an transcription inhibiting domain (e.g., KRAB moiety or SID moiety), a reverse transcriptase or a catalytic domain thereof, an exonuclease (e.g., T5E (SEQ ID NO: 449)) or a catalytic domain thereof, a destabilized domain (e.g., destabilized domains (DD) of E. coli dihydrofolate reductase (ecDHFR)), a histone residue modification domain, a nuclease catalytic domain (e.g., FokI), a transcription modification factor, a light gating factor, a chemical inducible factor, a chromatin visualization factor, a targeting polypeptide for providing binding to a cell surface portion on a target cell or a target cell type, a reporter (e.g., fluorescent) polypeptide or a detection label (e.g., GST, HRP, CAT, GFP, HcRed, DsRed, CFP, YFP, BFP), a localization signal, a polypeptide targeting moiety, a DNA binding domain (e.g., MBP, Lex A DBD, Gal4 DBD), an epitope tag (e.g., His, myc, V5, FLAG, HA, VSV-G, Trx, etc), a transcription release factor, an HDAC, a moiety having ssRNA cleavage activity, a moiety having dsRNA cleavage activity, a moiety having ssDNA cleavage activity, a moiety having dsDNA cleavage activity, a DNA or RNA ligase, a functional domain exhibiting activity to modify a target DNA, selected from the group consisting of: methyltransferase activity, DNA repair activity, DNA damage activity, dismutase activity, alkylation activity, dealkylation activity, depurination activity, oxidation activity, deoxidation activity, pyrimidine dimer forming activity, integrase activity, transposase activity, recombinase activity, polymerase activity, ligase activity, helicase activity, photolyase activity, glycosylase activity, acetyl transferase activity, deacetylase activity, kinase activity, phosphatase activity, ubiquitin ligase activity, deubiquitination activity, adenylation activity, deadenylation activity, SUMOylation activity, deSUMOylation activity, ribosylation activity, deribosylation activity, myristoylation activity, demyristoylation activity, glycosylation activity (e.g., from O-GlcNAc transferase), deglycosylation activity, and a catalytic domain thereof, and a functional fragment (e.g., a functional truncation) thereof, and any combination thereof;
optionally, wherein the NLS comprises or is SV40 NLS (SEQ ID NO: 444), bpSV40 NLS (BP NLS, bpNLS, SEQ ID NO: 443 or 462), or NP NLS (Xenopus laevis Nucleoplasmin NLS, nucleoplasmin NLS, SEQ ID NO: 445);
optionally, wherein the deaminase or catalytic domain thereof is an adenine deaminase (e.g., TadA, such as, TadA8e, TadA8.17, TadA8.20, TadA9) or a catalytic domain thereof, for example, TadA8e-V106W (SEQ ID NO: 439), TadA8e-W106V (SEQ ID NO: 461);
optionally, wherein the deaminase or catalytic domain thereof is a cytidine deaminase (e.g., APOBEC, such as, APOBEC3, for example, APOBEC3A, APOBEC3B, APOBEC3C; DddA) or a catalytic domain thereof, for example, hAPOBEC3-W104A (SEQ ID NO: 440); and/or
optionally, wherein the UGI is human UGI domain (such as, SEQ ID NO: 441).

17. The Cas12i polypeptide of claim 16, wherein the Cas12i polypeptide comprises amino acid substitutions E336R and D1049A relative to SEQ ID NO: 458, and a base editing domain, for example, a deaminase or a catalytic domain thereof.

18. The Cas12i polypeptide of claim 17, wherein the Cas12i polypeptide comprises the amino acid sequence of SEQ ID NO: 463, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 463.

19. The Cas12i polypeptide of claim 17, wherein the Cas12i polypeptide comprises the amino acid sequence of SEQ ID NO: 464, or an amino acid sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to the amino acid sequence of SEQ ID NO: 464.

20. A system comprising:
(1) the Cas12i polypeptide of claim 1 or a polynucleotide encoding the Cas12i polypeptide; and
(2) a guide nucleic acid or a polynucleotide encoding the guide nucleic acid, the guide nucleic acid comprising:
(i) a direct repeat (DR) sequence capable of forming a complex with the Cas12i polypeptide; and
(ii) a spacer sequence capable of hybridizing to a target sequence of a target DNA, thereby guiding the complex to the target DNA;
optionally, wherein the direct repeat sequence is 5′ to the spacer sequence; and/or
optionally, wherein the guide nucleic acid is a guide RNA (gRNA).

21. The system of claim 20, wherein the direct repeat sequence has substantially the same secondary structure as the secondary structure of any one of SEQ ID NOs: 11 and 451-457;
optionally, wherein the direct repeat sequence:
(1) comprises the polynucleotide sequence of any one of SEQ ID NOs: 11 and 451-457; or
(2) comprises a polynucleotide sequence having a sequence identity of at least about 80% (e.g., at least about 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to any one of SEQ ID NOs: 11 and 451-457;
optionally, wherein the direct repeat sequence comprises the polynucleotide sequence of SEQ ID NO: 452.

22. The system of claim 20, wherein the target sequence comprises about or at least about 16 contiguous nucleotides of the target DNA, e.g., about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more contiguous nucleotides of the target DNA, or in a numerical range between any two of the preceding values, e.g., from about 16 to about 50, or from about 17 to about 22 contiguous nucleotides of the target DNA; optionally, wherein the target sequence comprises about 20 contiguous nucleotides of the target DNA.

23. The system of claim 20, wherein the reversely complementary sequence of the target sequence is immediately 3′ to a protospacer adjacent motif (PAM); optionally the PAM is 5′-TN, 5′-TTN, or 5′-GCC, wherein N is A, T, G, or C.

24. The system of claim 20, wherein the spacer sequence is about or at least about 16 nucleotides in length, e.g., about or at least about 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, or more nucleotides in length, or in a length of a numerical range between any two of the preceding values, e.g., in a length of from about 16 to about 50 nucleotides, or from about 17 to about 22 nucleotides; optionally, wherein the spacer sequence is about 20 nucleotides in length.

25. The system of claim 20, wherein the guide nucleic acid comprises a plurality (e.g., 2, 3, 4, 5 or more) of the spacer sequences capable of hybridizing to a plurality of the target sequences, respectively.

26. The system of claim 25, wherein the guide nucleic acid comprises, from 5′ to 3′, the direct repeat sequence, the spacer sequence, the direct repeat sequence, the spacer sequence, and the direct repeat sequence.

27. The system of claim 20, wherein the target DNA is a dsDNA, such as, a eukaryotic dsDNA, e.g., a gene in a eukaryotic cell.

28. A polynucleotide encoding the Cas12i polypeptide of claim 1.

29. A vector comprising the polynucleotide of claim 28; optionally wherein the vector is a plasmid vector, a recombinant AAV (rAAV) vector, or a recombinant lentivirus vector.

30. A ribonucleoprotein (RNP) comprising the Cas12i polypeptide of claim 1 and a guide nucleic acid.

31. A lipid nanoparticle (LNP) comprising the Cas12i polypeptide of claim 1.

32. A method for modifying a target DNA, comprising contacting the target DNA with the system of claim 20, wherein the spacer sequence is capable of hybridizing to a target sequence of the target DNA, wherein the target DNA is modified by the complex.

33. The method of claim 32, wherein the target DNA is in a cell;
optionally, wherein the cell is a eukaryotic cell (e.g., an animal cell, a vertebrate cell, a mammalian cell, a non-human mammalian cell, a non-human primate cell, a rodent (e.g., mouse or rat) cell, a human cell, a plant cell, or a yeast cell) or a prokaryotic cell (e.g., a bacteria cell);
optionally, wherein the cell is from a plant or an animal;
optionally, wherein the plant is a dicotyledon; optionally selected from the group consisting of soybean, cabbage (e.g., Chinese cabbage), rapeseed, brassica, watermelon, melon, potato, tomato, tobacco, eggplant, pepper, cucumber, cotton, alfalfa, eggplant, grape;
optionally, wherein the plant is a monocotyledon; optionally selected from the group consisting of rice, corn, wheat, barley, oat, sorghum, millet, grasses, Poaceae, Zizania, Avena, Coix, Hordeum, Oryza, Panicum (e.g., Panicum miliaceum), Secale, Setaria (e.g., Setaria italica), Sorghum, Triticum, Zea, Cymbopogon, Saccharum (e.g., Saccharum officinarum), Phyllostachys, Dendrocalamus, Bambusa, Yushania; and/or
optionally, wherein the animal is selected from the group consisting of pig, ox, sheep, goat, mouse, rat, alpaca, monkey, rabbit, chicken, duck, goose, fish (e.g., zebra fish).

34. The method of claim 32, wherein the modification comprises one or more of cleavage, base editing, repairing, and exogenous sequence insertion or integration of the target DNA.

35. A cell modified by the method of claim 32.

36. A pharmaceutical composition comprising (1) the system of claim 20; and (2) a pharmaceutically acceptable excipient.

37. A method for diagnosing, preventing, or treating a disease in a subject in need thereof, comprising administering to the subject the pharmaceutical composition of claim 36, wherein the disease is associated with a target DNA, wherein the spacer sequence is capable of hybridizing to a target sequence of the target DNA, wherein the target DNA is modified by the complex, and wherein the modification of the target DNA diagnose, prevents, or treats the disease.

38. The method of claim 37, wherein the disease is selected from the group consisting of Angelman syndrome (AS), Alzheimer's disease (AD), transthyretin amyloidosis (ATTR), transthyretin amyloid cardiomyopathy (ATTR-CM), cystic fibrosis (CF), hereditary angioedema, diabetes, progressive pseudohypertrophic muscular dystrophy, Duchenne muscular dystrophy (DMD), Becker muscular dystrophy (BMD), spinal muscular atrophy (SMA), alpha-1-antitrypsin deficiency, Pompe disease, myotonic dystrophy, Huntington's disease (HTT), fragile X syndrome, Friedreich ataxia, amyotrophic lateral sclerosis (ALS), frontotemporal dementia, hereditary chronic kidney disease, hyperlipidemia, Leber congenital amaurosis (LCA), sickle cell disease, thalassemia (e.g., β-thalassemia), Parkinson's disease (PD), myelodysplastic syndrome (MDS), retinitis pigmentosa (RP), age-related macular degeneration (AMD), Hepatitis B, nonalcoholic fatty liver disease (NAFLD), Acquired Immune Deficiency Syndrome, corneal dystrophy (CD), hypercholesterolemia, familial hypercholesterolemia (FH), heart disease (e.g., hypertrophic cardiomyopathy (HCM)), and cancer.

39. A method of detecting a target DNA, comprising contacting the target DNA with the system of claim 20, wherein the target DNA is modified by the complex, and wherein the modification detects the target DNA; optionally wherein the modification generates a detectable signal, e.g., a fluorescent signal.
